SEQUENCE LISTING (1026 sequences)

SEQ ID NO: 1 - protein, 932 aa, Severe acute respiratory syndrome coronavirus 2
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 2 - DNA, 2795 bp, Severe acute respiratory syndrome coronavirus 2
tcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacag

SEQ ID NO: 3 - DNA, 2777 bp, Severe acute respiratory syndrome coronavirus 2
tttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacag

SEQ ID NO: 4 - protein, 866 aa, Severe acute respiratory syndrome coronavirus 2
FLNGFAVVQPVLHRAAQALVLMSYTGLLTSTMIKLVLLNSKLIVVASKKRTKMTILILTLLRDTLSLTTNMKKQFIIYLRIVQLLLNMTSLSLETVTWYHIYHVNVLLNTQWQTSSMLGILMKVIVTHKKYLSHTIVVMMIISIKRTGMILKTQIYYAYTPTVNVYAKLCKQYNSVMPCEMLVLLVYHIIKISMVTGMISVISYKPRQVVEFLLILIIHCCLYPPGLLQSHMLTLTQSLTLSGICNMTSRKRGNSLTVILNIGIRHTTQIVLTVWMTDAFCIVQTLMFYSLQCSHLQVLDHEKYLLMVFHLFQLDTTSESVLYIIRMTYIALDLVLRNYLCMLLTLLCTLLLVIYYINALRAFQLHLLTMLLFKLSNPVILTKTSMTLLCLRVSLRKEVLLNNTSSLLRMVMLLSAIMTTIVIIYQQCVISDNYYLLKLLISTLIVTMVAVLMLTKSSSTTTNQLVFHLINGVRLDFIMIQVMRIKMHFSHIQNVMSSLLLKILSMPLVQRIELAPLVSLSVVLPIDSFIKNYNQPPLEELLLEQANSMVVGTTCKLFIVMKTLTLWVGIILNVIEPCLTCLELWPHLFLLANIQRVVACHTVSIDLMSVLKYVKWSCVAVHYMLNQVEPHQEMPQLLMLIVFLTFVKLSRPMLMHFYLLMVTKLPISMSAIYNTDFMSVSIEIEMLTQTLMSFTHICVNISQYSLTMLLCVSIALMHLKVWLARTLSQFFIIKTMFLCLKQNVGLRLTLLKDLMNFALNIQCLNRVMIMCTFLTQIHQESGPAVLMISKQMVHLLNGSCLLMLTHLLNILIRSMLMSFICTYNTESYMMSQDTCTCILLCLLMITLQGIGNLSFMRLCTHRIQSY

SEQ ID NO: 5 - protein, 926 aa, Severe acute respiratory syndrome coronavirus 2
FLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 6 - DNA, 48 bp, Severe acute respiratory syndrome coronavirus 2
ggcgattttgttaaagccacttgcgaattttgtggcactgagaatttg

SEQ ID NO: 7 - DNA, 57 bp, Severe acute respiratory syndrome coronavirus 2
tttaaacttaatgaagagatcgccattattttggcatctttttctgcttccacaagt

SEQ ID NO: 8 - DNA, 51 bp, Severe acute respiratory syndrome coronavirus 2
ttcaaacaaattgttgaatcctgtggtaattttaaagttacaaaaggaaaa

SEQ ID NO: 9 - DNA, 60 bp, Severe acute respiratory syndrome coronavirus 2
gcctggaatattggtgaacagaaatcaatactgagtcctctttatgcatttgcatcagag

SEQ ID NO: 10 - DNA, 45 bp, Severe acute respiratory syndrome coronavirus 2
actaacaatctagttgtaatggcctacattacaggtggtgttgtt

SEQ ID NO: 11 - DNA, 45 bp, Severe acute respiratory syndrome coronavirus 2
aaacttaaagccttgaatttaggtgaaacatttgtcacgcactca

SEQ ID NO: 12 - DNA, 60 bp, Severe acute respiratory syndrome coronavirus 2
ttggttggtacaccagtttgtattaacgggcttatgttgctcgaaatcaaagacacagaa

SEQ ID NO: 13 - DNA, 54 bp, Severe acute respiratory syndrome coronavirus 2
ccaacaaaggttacttttggtgatgacactgtgatagaagtgcaaggttacaag

SEQ ID NO: 14 - DNA, 48 bp, Severe acute respiratory syndrome coronavirus 2
tatacagttgaactcggtacagaagtaaatgagttcgcctgtgttgtg

SEQ ID NO: 15 - DNA, 75 bp, Severe acute respiratory syndrome coronavirus 2
gctacatactacttatttgatgagtctggtgagtttaaattggcttcacatatgtattgttctttctaccctcca

SEQ ID NO: 16 - DNA, 45 bp, Severe acute respiratory syndrome coronavirus 2
gaggatgaagaagaaggtgattgtgaagaagaagagtttgagcca

SEQ ID NO: 17 - DNA, 51 bp, Severe acute respiratory syndrome coronavirus 2
gaagaagagcaagaagaagattggttagatgatgatagtcaacaaactgtt

SEQ ID NO: 18 - DNA, 48 bp, Severe acute respiratory syndrome coronavirus 2
aaaaaggtaaaaccaacagtggttgttaatgcagccaatgtttacctt

SEQ ID NO: 19 - DNA, 54 bp, Severe acute respiratory syndrome coronavirus 2
catggaggaggtgttgcaggagccttaaataaggctactaacaatgccatgcaa

SEQ ID NO: 20 - DNA, 57 bp, Severe acute respiratory syndrome coronavirus 2
gttcgcacaaatgtctacttagctgtctttgataaaaatctctatgacaaacttgtt

SEQ ID NO: 21 - DNA, 45 bp, Severe acute respiratory syndrome coronavirus 2
aaaatcaaagcttgtgttgaagaagttacaacaactctggaagaa

SEQ ID NO: 22 - DNA, 60 bp, Severe acute respiratory syndrome coronavirus 2
cttcatccagattctgccactcttgttagtgacattgacatcactttcttaaagaaagat

SEQ ID NO: 23 - DNA, 51 bp, Severe acute respiratory syndrome coronavirus 2
gacaattatataaccacttacccgggtcagggtttaaatggttacactgta

SEQ ID NO: 24 - DNA, 129 bp, Severe acute respiratory syndrome coronavirus 2
tacattctaccatctattatctctaatgagaagcaagaaattcttggaactgtttcttggaatttgcgagaaatgcttgcacatgcagaagaaacacgcaaattaatgcctgtctgtgtggaaactaaa

SEQ ID NO: 25 - DNA, 93 bp, Severe acute respiratory syndrome coronavirus 2
atagtttcaactatacagcgtaaatataagggtattaaaatacaagagggtgtggttgattatggtgctagattttacttttacaccagtaaa

SEQ ID NO: 26 - DNA, 93 bp, Severe acute respiratory syndrome coronavirus 2
gctacagtttctgtttcttcacctgatgctgttacagcgtataatggttatcttacttcttcttctaaaacacctgaagaacattttattgaa

SEQ ID NO: 27 - DNA, 45 bp, Severe acute respiratory syndrome coronavirus 2
atctcacttgctggttcctataaagattggtcctattctggacaa

SEQ ID NO: 28 - DNA, 72 bp, Severe acute respiratory syndrome coronavirus 2
tcaatgacatatggacaacagtttggtccaacttatttggatggagctgatgttactaaaataaaacctcat

SEQ ID NO: 29 - DNA, 60 bp, Severe acute respiratory syndrome coronavirus 2
catgaaggtaaaacattttatgttttacctaatgatgacactctacgtgttgaggctttt

SEQ ID NO: 30 - DNA, 60 bp, Severe acute respiratory syndrome coronavirus 2
ccacaagttaatggtttaacttctattaaatgggcagataacaactgttatcttgccact

SEQ ID NO: 31 - DNA, 60 bp, Severe acute respiratory syndrome coronavirus 2
ctccaacaaatagagttgaagtttaatccacctgctctacaagatgcttattacagagca

SEQ ID NO: 32 - DNA, 66 bp, Severe acute respiratory syndrome coronavirus 2
gtcttgaacgtggtgtgtaaaacttgtggacaacagcagacaacccttaagggtgtagaagctgtt

SEQ ID NO: 33 - DNA, 69 bp, Severe acute respiratory syndrome coronavirus 2
tataaattggatggtgttgtttgtacagaaattgaccctaagttggacaattattataagaaagacaat

SEQ ID NO: 34 - DNA, 69 bp, Severe acute respiratory syndrome coronavirus 2
ggttataagaaacctgcttcaagagagcttaaagttacatttttccctgacttaaatggtgatgtggtg

SEQ ID NO: 35 - DNA, 120 bp, Severe acute respiratory syndrome coronavirus 2
ttagaaactatacaaattaccatttcatcttttaaatgggatttaactgcttttggcttagttgcagagtggtttttggcatatattcttttcactaggtttttctatgtacttggattg

SEQ ID NO: 36 - DNA, 48 bp, Severe acute respiratory syndrome coronavirus 2
tcattttattatgtatggaaaagttatgtgcatgttgtagacggttgt

SEQ ID NO: 37 - DNA, 78 bp, Severe acute respiratory syndrome coronavirus 2
tcatcaacttgtatgatgtgttacaaacgtaatagagcaacaagagtcgaatgtacaactattgttaatggtgttaga

SEQ ID NO: 38 - DNA, 48 bp, Severe acute respiratory syndrome coronavirus 2
gacttgtcactacagtttaaaagaccaataaatcctactgaccagtct

SEQ ID NO: 39 - DNA, 60 bp, Severe acute respiratory syndrome coronavirus 2
aataacactaaaggttcattgcctattaatgttatagtttttgatggtaaatcaaaatgt

SEQ ID NO: 40 - DNA, 54 bp, Severe acute respiratory syndrome coronavirus 2
tgtcaacctatactgttactagatcaggcattagtgtctgatgttggtgatagt

SEQ ID NO: 41 - DNA, 51 bp, Severe acute respiratory syndrome coronavirus 2
gttaaaatgtttgatgcttacgttaatacgttttcatcaacttttaacgta

SEQ ID NO: 42 - DNA, 66 bp, Severe acute respiratory syndrome coronavirus 2
ttagacaatgtcttatctacttttatttcagcagctcggcaagggtttgttgattcagatgtagaa

SEQ ID NO: 43 - DNA, 69 bp, Severe acute respiratory syndrome coronavirus 2
aaagatttcatgtcattgtctgaacaactacgaaaacaaatacgtagtgctgctaaaaagaataactta

SEQ ID NO: 44 - DNA, 72 bp, Severe acute respiratory syndrome coronavirus 2
tttaagttgacatgtgcaactactagacaagttgttaatgttgtaacaacaaagatagcacttaagggtggt

SEQ ID NO: 45 - DNA, 51 bp, Severe acute respiratory syndrome coronavirus 2
gtgttcctttttgttgctgctattttctatttaataacacctgttcatgtc

SEQ ID NO: 46 - DNA, 87 bp, Severe acute respiratory syndrome coronavirus 2
tatactaatgacaaagcttgcccattgattgctgcagtcataacaagagaagtgggttttgtcgtgcctggtttgcctggcacgata

SEQ ID NO: 47 - DNA, 84 bp, Severe acute respiratory syndrome coronavirus 2
acaactaatggtgactttttgcatttcttacctagagtttttagtgcagttggtaacatctgttacacaccatcaaaacttata

SEQ ID NO: 48 - DNA, 54 bp, Severe acute respiratory syndrome coronavirus 2
tttgcaacatcagcttgtgttttggctgctgaatgtacaatttttaaagatgct

SEQ ID NO: 49 - DNA, 63 bp, Severe acute respiratory syndrome coronavirus 2
ggtaagccagtaccatattgttatgataccaatgtactagaaggttctgttgcttatgaaagt

SEQ ID NO: 50 - DNA, 69 bp, Severe acute respiratory syndrome coronavirus 2
ccactaattcaacctattggtgctttggacatatcagcatctatagtagctggtggtattgtagctatc

SEQ ID NO: 51 - DNA, 57 bp, Severe acute respiratory syndrome coronavirus 2
gcacatattcagtggatggttatgttcacacctttagtacctttctggataacaatt

SEQ ID NO: 52 - DNA, 123 bp, Severe acute respiratory syndrome coronavirus 2
agacgtgtagtctttaatggtgtttcctttagtacttttgaagaagctgcgctgtgcacctttttgttaaataaagaaatgtatctaaagttgcgtagtgatgtgctattacctcttacgcaa

SEQ ID NO: 53 - DNA, 45 bp, Severe acute respiratory syndrome coronavirus 2
aatagatacttagctctttataataagtacaagtattttagtgga

SEQ ID NO: 54 - DNA, 69 bp, Severe acute respiratory syndrome coronavirus 2
ggacagactttttcagtgttagcttgttacaatggttcaccatctggtgtttaccaatgtgctatgagg

SEQ ID NO: 55 - DNA, 63 bp, Severe acute respiratory syndrome coronavirus 2
attaagggttcattccttaatggttcatgtggtagtgttggttttaacatagattatgactgt

SEQ ID NO: 56 - DNA, 66 bp, Severe acute respiratory syndrome coronavirus 2
ttagcttggttgtacgctgctgttataaatggagacaggtggtttctcaatcgatttaccacaact

SEQ ID NO: 57 - DNA, 54 bp, Severe acute respiratory syndrome coronavirus 2
aagtacaattatgaacctctaacacaagaccatgttgacatactaggacctctt

SEQ ID NO: 58 - DNA, 51 bp, Severe acute respiratory syndrome coronavirus 2
acttcacttttagttttagtccagagtactcaatggtctttgttctttttt

SEQ ID NO: 59 - DNA, 48 bp, Severe acute respiratory syndrome coronavirus 2
gccactgtagcttattttaatatggtctatatgcctgctagttgggtg

SEQ ID NO: 60 - DNA, 111 bp, Severe acute respiratory syndrome coronavirus 2
tgttactttggcctcttttgtttactcaaccgctactttagactgactcttggtgtttatgattacttagtttctacacaggagtttagatatatgaattcacagggacta

SEQ ID NO: 61 - DNA, 57 bp, Severe acute respiratory syndrome coronavirus 2
tctgaggacaagagggcaaaagttactagtgctatgcagacaatgcttttcactatg

SEQ ID NO: 62 - DNA, 57 bp, Severe acute respiratory syndrome coronavirus 2
tgggaaatccaacaggttgtagatgcagatagtaaaattgttcaacttagtgaaatt

SEQ ID NO: 63 - DNA, 75 bp, Severe acute respiratory syndrome coronavirus 2
atggacaattcacctaatttagcatggcctcttattgtaacagctttaagggccaattctgctgtcaaattacag

SEQ ID NO: 64 - DNA, 54 bp, Severe acute respiratory syndrome coronavirus 2
cagatgtcttgtgctgccggtactacacaaactgcttgcactgatgacaatgcg

SEQ ID NO: 65 - DNA, 51 bp, Severe acute respiratory syndrome coronavirus 2
aaagtgaagtatttatactttattaaaggattaaacaacctaaatagaggt

SEQ ID NO: 66 - DNA, 135 bp, Severe acute respiratory syndrome coronavirus 2
agtgggggacaaccaatcactaattgtgttaagatgttgtgtacacacactggtactggtcaggcaataacagttacaccggaagccaatatggatcaagaatcctttggtggtgcatcgtgttgtctgtactgc

SEQ ID NO: 67 - DNA, 66 bp, Severe acute respiratory syndrome coronavirus 2
tgccacatagatcatccaaatcctaaaggattttgtgacttaaaaggtaagtatgtacaaatacct

SEQ ID NO: 68 - DNA, 69 bp, Severe acute respiratory syndrome coronavirus 2
cccattagttttccattgtgtgctaatggacaagtttttggtttatataaaaatacatgtgttggtagc

SEQ ID NO: 69 - DNA, 63 bp, Severe acute respiratory syndrome coronavirus 2
aaccgaaattatgtctttactggttatcgtgtaactaaaaacagtaaagtacaaataggagag

SEQ ID NO: 70 - DNA, 57 bp, Severe acute respiratory syndrome coronavirus 2
ctagtgccacaagagcactatgttagaattactggcttatacccaacactcaatatc

SEQ ID NO: 71 - DNA, 45 bp, Severe acute respiratory syndrome coronavirus 2
ttttctagcaatgttgcaaattatcaaaaggttggtatgcaaaag

SEQ ID NO: 72 - DNA, 102 bp, Severe acute respiratory syndrome coronavirus 2
acagcttgctctcatgccgctgttgatgcactatgtgagaaggcattaaaatatttgcctatagataaatgtagtagaattatacctgcacgtgctcgtgta

SEQ ID NO: 73 - DNA, 66 bp, Severe acute respiratory syndrome coronavirus 2
cctgcaccacgcacattgctaactaagggcacactagaaccagaatatttcaattcagtgtgtaga

SEQ ID NO: 74 - DNA, 66 bp, Severe acute respiratory syndrome coronavirus 2
catgatgtttcatctgcaattaacaggccacaaataggcgtggtaagagaattccttacacgtaac

SEQ ID NO: 75 - DNA, 54 bp, Severe acute respiratory syndrome coronavirus 2
gactatgtcatattcactcaaaccactgaaacagctcactcttgtaatgtaaac

SEQ ID NO: 76 - DNA, 48 bp, Severe acute respiratory syndrome coronavirus 2
ccgcctggagatcaatttaaacacctcataccacttatgtacaaagga

SEQ ID NO: 77 - DNA, 51 bp, Severe acute respiratory syndrome coronavirus 2
tgggcacatggctttgagttgacatctatgaagtattttgtgaaaatagga

SEQ ID NO: 78 - DNA, 48 bp, Severe acute respiratory syndrome coronavirus 2
gattacgtctataatccgtttatgattgatgttcaacaatggggtttt

SEQ ID NO: 79 - DNA, 51 bp, Severe acute respiratory syndrome coronavirus 2
gttcttcacgacattggtaaccctaaagctattaagtgtgtacctcaagct

SEQ ID NO: 80 - DNA, 45 bp, Severe acute respiratory syndrome coronavirus 2
gctaattccattgtttgtagatttgacactagagtgctatctaac

SEQ ID NO: 81 - DNA, 45 bp, Severe acute respiratory syndrome coronavirus 2
aataaacatgcattccacacaccagcttttgataaaagtgctttt

SEQ ID NO: 82 - DNA, 75 bp, Severe acute respiratory syndrome coronavirus 2
ggaaaacaagtagtgtcagatatagattatgtaccactaaagtctgctacgtgtataacacgttgcaatttaggt

SEQ ID NO: 83 - DNA, 54 bp, Severe acute respiratory syndrome coronavirus 2
caacaattacctgaaacttactttactcagagtagaaatttacaagaatttaaa

SEQ ID NO: 84 - DNA, 69 bp, Severe acute respiratory syndrome coronavirus 2
gaattcattgaacggtataaattagaaggctatgccttcgaacatatcgtttatggagattttagtcat

SEQ ID NO: 85 - DNA, 60 bp, Severe acute respiratory syndrome coronavirus 2
cctaaaggcataatgatgaatgtcgcaaaatatactcaactgtgtcaatatttaaacaca

SEQ ID NO: 86 - DNA, 57 bp, Severe acute respiratory syndrome coronavirus 2
ggtacgctgcttgtcgattcagatcttaatgactttgtctctgatgcagattcaact

SEQ ID NO: 87 - DNA, 60 bp, Severe acute respiratory syndrome coronavirus 2
tggtggacagcctttgttactaatgtgaatgcgtcatcatctgaagcatttttaattgga

SEQ ID NO: 88 - DNA, 51 bp, Severe acute respiratory syndrome coronavirus 2
tttgacatgagtaaatttccccttaaattaaggggtactgctgttatgtct

SEQ ID NO: 89 - DNA, 78 bp, Severe acute respiratory syndrome coronavirus 2
tcgaagacccagtccctacttattgttaataacgctactaatgttgttattaaagtctgtgaatttcaattttgtaat

SEQ ID NO: 90 - DNA, 66 bp, Severe acute respiratory syndrome coronavirus 2
agggaatttgtgtttaagaatattgatggttattttaaaatatattctaagcacacgcctattaat

SEQ ID NO: 91 - DNA, 66 bp, Severe acute respiratory syndrome coronavirus 2
ttagaaccattggtagatttgccaataggtattaacatcactaggtttcaaactttacttgcttta

SEQ ID NO: 92 - DNA, 132 bp, Severe acute respiratory syndrome coronavirus 2
gcagcttattatgtgggttatcttcaacctaggacttttctattaaaatataatgaaaatggaaccattacagatgctgtagactgtgcacttgaccctctctcagaaacaaagtgtacgttgaaatccttc

SEQ ID NO: 93 - DNA, 135 bp, Severe acute respiratory syndrome coronavirus 2
ccaacagaatctattgttagatttcctaatattacaaacttgtgcccttttggtgaagtttttaacgccaccagatttgcatctgtttatgcttggaacaggaagagaatcagcaactgtgttgctgattattct

SEQ ID NO: 94 - DNA, 57 bp, Severe acute respiratory syndrome coronavirus 2
tctcctactaaattaaatgatctctgctttactaatgtctatgcagattcatttgta

SEQ ID NO: 95 - DNA, 63 bp, Severe acute respiratory syndrome coronavirus 2
attgctgattataattataaattaccagatgattttacaggctgcgttatagcttggaattct

SEQ ID NO: 96 - DNA, 105 bp, Severe acute respiratory syndrome coronavirus 2
actgtttgtggacctaaaaagtctactaatttggttaaaaacaaatgtgtcaatttcaacttcaatggtttaacaggcacaggtgttcttactgagtctaacaaa

SEQ ID NO: 97 - DNA, 72 bp, Severe acute respiratory syndrome coronavirus 2
tttctgcctttccaacaatttggcagagacattgctgacactactgatgctgtccgtgatccacagacactt

SEQ ID NO: 98 - DNA, 87 bp, Severe acute respiratory syndrome coronavirus 2
attcttgacattacaccatgttcttttggtggtgtcagtgttataacaccaggaacaaatacttctaaccaggttgctgttctttat

SEQ ID NO: 99 - DNA, 57 bp, Severe acute respiratory syndrome coronavirus 2
gtcaacaactcatatgagtgtgacatacccattggtgcaggtatatgcgctagttat

SEQ ID NO: 100 - DNA, 108 bp, Severe acute respiratory syndrome coronavirus 2
aagacatcagtagattgtacaatgtacatttgtggtgattcaactgaatgcagcaatcttttgttgcaatatggcagtttttgtacacaattaaaccgtgctttaact

SEQ ID NO: 101 - DNA, 66 bp, Severe acute respiratory syndrome coronavirus 2
atgcaaatggcttataggtttaatggtattggagttacacagaatgttctctatgagaaccaaaaa

SEQ ID NO: 102 - DNA, 174 bp, Severe acute respiratory syndrome coronavirus 2
ggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagt

SEQ ID NO: 103 - DNA, 66 bp, Severe acute respiratory syndrome coronavirus 2
cagacatatgtgactcaacaattaattagagctgcagaaatcagagcttctgctaatcttgctgct

SEQ ID NO: 104 - DNA, 57 bp, Severe acute respiratory syndrome coronavirus 2
aaaatgtcagagtgtgtacttggacaatcaaaaagagttgatttttgtggaaagggc

SEQ ID NO: 105 - DNA, 60 bp, Severe acute respiratory syndrome coronavirus 2
ggtacgttaatagttaatagcgtacttctttttcttgctttcgtggtattcttgctagtt

SEQ ID NO: 106 - DNA, 81 bp, Severe acute respiratory syndrome coronavirus 2
gcttctttcagactgtttgcgcgtacgcgttccatgtggtcattcaatccagaaactaacattcttctcaacgtgccactc

SEQ ID NO: 107 - DNA, 51 bp, Severe acute respiratory syndrome coronavirus 2
ctaactgagaataaatattctcaattagatgaagagcaaccaatggagatt

SEQ ID NO: 108 - DNA, 45 bp, Severe acute respiratory syndrome coronavirus 2
ctcagtccaagatggtatttctactacctaggaactgggccagaa

SEQ ID NO: 109 - DNA, 51 bp, Severe acute respiratory syndrome coronavirus 2
caagctttcggcagacgtggtccagaacaaacccaaggaaattttggggac

SEQ ID NO: 110 - DNA, 48 bp, Severe acute respiratory syndrome coronavirus 2
cccagcgcttcagcgttcttcggaatgtcgcgcattggcatggaagtc

SEQ ID NO: 111 - protein, 16 aa, Severe acute respiratory syndrome coronavirus 2
GDFVKATCEFCGTENL

SEQ ID NO: 112 - protein, 19 aa, Severe acute respiratory syndrome coronavirus 2
FKLNEEIAIILASFSASTS

SEQ ID NO: 113 - protein, 17 aa, Severe acute respiratory syndrome coronavirus 2
FKQIVESCGNFKVTKGK

SEQ ID NO: 114 - protein, 20 aa, Severe acute respiratory syndrome coronavirus 2
AWNIGEQKSILSPLYAFASE

SEQ ID NO: 115 - protein, 15 aa, Severe acute respiratory syndrome coronavirus 2
TNNLVVMAYITGGVV

SEQ ID NO: 116 - protein, 15 aa, Severe acute respiratory syndrome coronavirus 2
KLKALNLGETFVTHS

SEQ ID NO: 117 - protein, 20 aa, Severe acute respiratory syndrome coronavirus 2
LVGTPVCINGLMLLEIKDTE

SEQ ID NO: 118 - protein, 18 aa, Severe acute respiratory syndrome coronavirus 2
PTKVTFGDDTVIEVQGYK

SEQ ID NO: 119 - protein, 16 aa, Severe acute respiratory syndrome coronavirus 2
YTVELGTEVNEFACVV

SEQ ID NO: 120 - protein, 25 aa, Severe acute respiratory syndrome coronavirus 2
ATYYLFDESGEFKLASHMYCSFYPP

SEQ ID NO: 121 - protein, 15 aa, Severe acute respiratory syndrome coronavirus 2
EDEEEGDCEEEEFEP

SEQ ID NO: 122 - protein, 17 aa, Severe acute respiratory syndrome coronavirus 2
EEEQEEDWLDDDSQQTV

SEQ ID NO: 123 - protein, 16 aa, Severe acute respiratory syndrome coronavirus 2
KKVKPTVVVNAANVYL

SEQ ID NO: 124 - protein, 18 aa, Severe acute respiratory syndrome coronavirus 2
HGGGVAGALNKATNNAMQ

SEQ ID NO: 125 - protein, 19 aa, Severe acute respiratory syndrome coronavirus 2
VRTNVYLAVFDKNLYDKLV

SEQ ID NO: 126 - protein, 15 aa, Severe acute respiratory syndrome coronavirus 2
KIKACVEEVTTTLEE

SEQ ID NO: 127 - protein, 20 aa, Severe acute respiratory syndrome coronavirus 2
LHPDSATLVSDIDITFLKKD

SEQ ID NO: 128 - protein, 17 aa, Severe acute respiratory syndrome coronavirus 2
DNYITTYPGQGLNGYTV

SEQ ID NO: 129 - protein, 43 aa, Severe acute respiratory syndrome coronavirus 2
YILPSIISNEKQEILGTVSWNLREMLAHAEETRKLMPVCVETK

SEQ ID NO: 130 - protein, 31 aa, Severe acute respiratory syndrome coronavirus 2
IVSTIQRKYKGIKIQEGVVDYGARFYFYTSK

SEQ ID NO: 131 - protein, 31 aa, Severe acute respiratory syndrome coronavirus 2
ATVSVSSPDAVTAYNGYLTSSSKTPEEHFIE

SEQ ID NO: 132 - protein, 15 aa, Severe acute respiratory syndrome coronavirus 2
ISLAGSYKDWSYSGQ

SEQ ID NO: 133 - protein, 24 aa, Severe acute respiratory syndrome coronavirus 2
SMTYGQQFGPTYLDGADVTKIKPH

SEQ ID NO: 134 - protein, 20 aa, Severe acute respiratory syndrome coronavirus 2
HEGKTFYVLPNDDTLRVEAF

SEQ ID NO: 135 - protein, 20 aa, Severe acute respiratory syndrome coronavirus 2
PQVNGLTSIKWADNNCYLAT

SEQ ID NO: 136 - protein, 20 aa, Severe acute respiratory syndrome coronavirus 2
LQQIELKFNPPALQDAYYRA

SEQ ID NO: 137 - protein, 22 aa, Severe acute respiratory syndrome coronavirus 2
VLNVVCKTCGQQQTTLKGVEAV

SEQ ID NO: 138 - protein, 23 aa, Severe acute respiratory syndrome coronavirus 2
YKLDGVVCTEIDPKLDNYYKKDN

SEQ ID NO: 139 - protein, 23 aa, Severe acute respiratory syndrome coronavirus 2
GYKKPASRELKVTFFPDLNGDVV

SEQ ID NO: 140 - protein, 40 aa, Severe acute respiratory syndrome coronavirus 2
LETIQITISSFKWDLTAFGLVAEWFLAYILFTRFFYVLGL

SEQ ID NO: 141 - protein, 16 aa, Severe acute respiratory syndrome coronavirus 2
SFYYVWKSYVHVVDGC

SEQ ID NO: 142 - protein, 26 aa, Severe acute respiratory syndrome coronavirus 2
SSTCMMCYKRNRATRVECTTIVNGVR

SEQ ID NO: 143 - protein, 16 aa, Severe acute respiratory syndrome coronavirus 2
DLSLQFKRPINPTDQS

SEQ ID NO: 144 - protein, 20 aa, Severe acute respiratory syndrome coronavirus 2
NNTKGSLPINVIVFDGKSKC

SEQ ID NO: 145 - protein, 18 aa, Severe acute respiratory syndrome coronavirus 2
CQPILLLDQALVSDVGDS

SEQ ID NO: 146 - protein, 17 aa, Severe acute respiratory syndrome coronavirus 2
VKMFDAYVNTFSSTFNV

SEQ ID NO: 147 - protein, 22 aa, Severe acute respiratory syndrome coronavirus 2
LDNVLSTFISAARQGFVDSDVE

SEQ ID NO: 148 - protein, 23 aa, Severe acute respiratory syndrome coronavirus 2
KDFMSLSEQLRKQIRSAAKKNNL

SEQ ID NO: 149 - protein, 24 aa, Severe acute respiratory syndrome coronavirus 2
FKLTCATTRQVVNVVTTKIALKGG

SEQ ID NO: 150 - protein, 17 aa, Severe acute respiratory syndrome coronavirus 2
VFLFVAAIFYLITPVHV

SEQ ID NO: 151 - protein, 29 aa, Severe acute respiratory syndrome coronavirus 2
YTNDKACPLIAAVITREVGFVVPGLPGTI

SEQ ID NO: 152 - protein, 28 aa, Severe acute respiratory syndrome coronavirus 2
TTNGDFLHFLPRVFSAVGNICYTPSKLI

SEQ ID NO: 153 - protein, 18 aa, Severe acute respiratory syndrome coronavirus 2
FATSACVLAAECTIFKDA

SEQ ID NO: 154 - protein, 21 aa, Severe acute respiratory syndrome coronavirus 2
GKPVPYCYDTNVLEGSVAYES

SEQ ID NO: 155 - protein, 23 aa, Severe acute respiratory syndrome coronavirus 2
PLIQPIGALDISASIVAGGIVAI

SEQ ID NO: 156 - protein, 19 aa, Severe acute respiratory syndrome coronavirus 2
AHIQWMVMFTPLVPFWITI

SEQ ID NO: 157 - protein, 41 aa, Severe acute respiratory syndrome coronavirus 2
RRVVFNGVSFSTFEEAALCTFLLNKEMYLKLRSDVLLPLTQ

SEQ ID NO: 158 - protein, 15 aa, Severe acute respiratory syndrome coronavirus 2
NRYLALYNKYKYFSG

SEQ ID NO: 159 - protein, 23 aa, Severe acute respiratory syndrome coronavirus 2
GQTFSVLACYNGSPSGVYQCAMR

SEQ ID NO: 160 - protein, 21 aa, Severe acute respiratory syndrome coronavirus 2
IKGSFLNGSCGSVGFNIDYDC

SEQ ID NO: 161 - protein, 22 aa, Severe acute respiratory syndrome coronavirus 2
LAWLYAAVINGDRWFLNRFTTT

SEQ ID NO: 162 - protein, 18 aa, Severe acute respiratory syndrome coronavirus 2
KYNYEPLTQDHVDILGPL

SEQ ID NO: 163 - protein, 17 aa, Severe acute respiratory syndrome coronavirus 2
TSLLVLVQSTQWSLFFF

SEQ ID NO: 164 - protein, 16 aa, Severe acute respiratory syndrome coronavirus 2
ATVAYFNMVYMPASWV

SEQ ID NO: 165 - protein, 37 aa, Severe acute respiratory syndrome coronavirus 2
CYFGLFCLLNRYFRLTLGVYDYLVSTQEFRYMNSQGL

SEQ ID NO: 166 - protein, 19 aa, Severe acute respiratory syndrome coronavirus 2
SEDKRAKVTSAMQTMLFTM

SEQ ID NO: 167 - protein, 19 aa, Severe acute respiratory syndrome coronavirus 2
WEIQQVVDADSKIVQLSEI

SEQ ID NO: 168 - protein, 25 aa, Severe acute respiratory syndrome coronavirus 2
MDNSPNLAWPLIVTALRANSAVKLQ

SEQ ID NO: 169 - protein, 18 aa, Severe acute respiratory syndrome coronavirus 2
QMSCAAGTTQTACTDDNA

SEQ ID NO: 170 - protein, 17 aa, Severe acute respiratory syndrome coronavirus 2
KVKYLYFIKGLNNLNRG

SEQ ID NO: 171 - protein, 45 aa, Severe acute respiratory syndrome coronavirus 2
SGGQPITNCVKMLCTHTGTGQAITVTPEANMDQESFGGASCCLYC

SEQ ID NO: 172 - protein, 22 aa, Severe acute respiratory syndrome coronavirus 2
CHIDHPNPKGFCDLKGKYVQIP

SEQ ID NO: 173 - protein, 23 aa, Severe acute respiratory syndrome coronavirus 2
PISFPLCANGQVFGLYKNTCVGS

SEQ ID NO: 174 - protein, 21 aa, Severe acute respiratory syndrome coronavirus 2
NRNYVFTGYRVTKNSKVQIGE

SEQ ID NO: 175 - protein, 19 aa, Severe acute respiratory syndrome coronavirus 2
LVPQEHYVRITGLYPTLNI

SEQ ID NO: 176 - protein, 15 aa, Severe acute respiratory syndrome coronavirus 2
FSSNVANYQKVGMQK

SEQ ID NO: 177 - protein, 34 aa, Severe acute respiratory syndrome coronavirus 2
TACSHAAVDALCEKALKYLPIDKCSRIIPARARV

SEQ ID NO: 178 - protein, 22 aa, Severe acute respiratory syndrome coronavirus 2
PAPRTLLTKGTLEPEYFNSVCR

SEQ ID NO: 179 - protein, 22 aa, Severe acute respiratory syndrome coronavirus 2
HDVSSAINRPQIGVVREFLTRN

SEQ ID NO: 180 - protein, 18 aa, Severe acute respiratory syndrome coronavirus 2
DYVIFTQTTETAHSCNVN

SEQ ID NO: 181 - protein, 16 aa, Severe acute respiratory syndrome coronavirus 2
PPGDQFKHLIPLMYKG

SEQ ID NO: 182 - protein, 17 aa, Severe acute respiratory syndrome coronavirus 2
WAHGFELTSMKYFVKIG

SEQ ID NO: 183 - protein, 16 aa, Severe acute respiratory syndrome coronavirus 2
DYVYNPFMIDVQQWGF

SEQ ID NO: 184 - protein, 17 aa, Severe acute respiratory syndrome coronavirus 2
VLHDIGNPKAIKCVPQA

SEQ ID NO: 185 - protein, 15 aa, Severe acute respiratory syndrome coronavirus 2
ANSIVCRFDTRVLSN

SEQ ID NO: 186 - protein, 15 aa, Severe acute respiratory syndrome coronavirus 2
NKHAFHTPAFDKSAF

SEQ ID NO: 187 - protein, 25 aa, Severe acute respiratory syndrome coronavirus 2
GKQVVSDIDYVPLKSATCITRCNLG

SEQ ID NO: 188 - protein, 18 aa, Severe acute respiratory syndrome coronavirus 2
QQLPETYFTQSRNLQEFK

SEQ ID NO: 189 - protein, 23 aa, Severe acute respiratory syndrome coronavirus 2
EFIERYKLEGYAFEHIVYGDFSH

SEQ ID NO: 190 - protein, 20 aa, Severe acute respiratory syndrome coronavirus 2
PKGIMMNVAKYTQLCQYLNT

SEQ ID NO: 191 - protein, 19 aa, Severe acute respiratory syndrome coronavirus 2
GTLLVDSDLNDFVSDADST

SEQ ID NO: 192 - protein, 20 aa, Severe acute respiratory syndrome coronavirus 2
WWTAFVTNVNASSSEAFLIG

SEQ ID NO: 193 - protein, 17 aa, Severe acute respiratory syndrome coronavirus 2
FDMSKFPLKLRGTAVMS

SEQ ID NO: 194 - protein, 26 aa, Severe acute respiratory syndrome coronavirus 2
SKTQSLLIVNNATNVVIKVCEFQFCN

SEQ ID NO: 195 - protein, 22 aa, Severe acute respiratory syndrome coronavirus 2
REFVFKNIDGYFKIYSKHTPIN

SEQ ID NO: 196 - protein, 22 aa, Severe acute respiratory syndrome coronavirus 2
LEPLVDLPIGINITRFQTLLAL

SEQ ID NO: 197 - protein, 44 aa, Severe acute respiratory syndrome coronavirus 2
AAYYVGYLQPRTFLLKYNENGTITDAVDCALDPLSETKCTLKSF

SEQ ID NO: 198 - protein, 45 aa, Severe acute respiratory syndrome coronavirus 2
PTESIVRFPNITNLCPFGEVFNATRFASVYAWNRKRISNCVADYS

SEQ ID NO: 199 - protein, 19 aa, Severe acute respiratory syndrome coronavirus 2
SPTKLNDLCFTNVYADSFV

SEQ ID NO: 200 - protein, 21 aa, Severe acute respiratory syndrome coronavirus 2
IADYNYKLPDDFTGCVIAWNS

SEQ ID NO: 201 - protein, 35 aa, Severe acute respiratory syndrome coronavirus 2
TVCGPKKSTNLVKNKCVNFNFNGLTGTGVLTESNK

SEQ ID NO: 202 - protein, 24 aa, Severe acute respiratory syndrome coronavirus 2
FLPFQQFGRDIADTTDAVRDPQTL

SEQ ID NO: 203 - protein, 29 aa, Severe acute respiratory syndrome coronavirus 2
ILDITPCSFGGVSVITPGTNTSNQVAVLY

SEQ ID NO: 204 - protein, 19 aa, Severe acute respiratory syndrome coronavirus 2
VNNSYECDIPIGAGICASY

SEQ ID NO: 205 - protein, 36 aa, Severe acute respiratory syndrome coronavirus 2
KTSVDCTMYICGDSTECSNLLLQYGSFCTQLNRALT

SEQ ID NO: 206 - protein, 22 aa, Severe acute respiratory syndrome coronavirus 2
MQMAYRFNGIGVTQNVLYENQK

SEQ ID NO: 207 - protein, 58 aa, Severe acute respiratory syndrome coronavirus 2
GKLQDVVNQNAQALNTLVKQLSSNFGAISSVLNDILSRLDKVEAEVQIDRLITGRLQS

SEQ ID NO: 208 - protein, 22 aa, Severe acute respiratory syndrome coronavirus 2
QTYVTQQLIRAAEIRASANLAA

SEQ ID NO: 209 - protein, 19 aa, Severe acute respiratory syndrome coronavirus 2
KMSECVLGQSKRVDFCGKG

SEQ ID NO: 210 - protein, 20 aa, Severe acute respiratory syndrome coronavirus 2
GTLIVNSVLLFLAFVVFLLV

SEQ ID NO: 211 - protein, 27 aa, Severe acute respiratory syndrome coronavirus 2
ASFRLFARTRSMWSFNPETNILLNVPL

SEQ ID NO: 212 - protein, 17 aa, Severe acute respiratory syndrome coronavirus 2
LTENKYSQLDEEQPMEI

SEQ ID NO: 213 - protein, 15 aa, Severe acute respiratory syndrome coronavirus 2
LSPRWYFYYLGTGPE

SEQ ID NO: 214 - protein, 17 aa, Severe acute respiratory syndrome coronavirus 2
QAFGRRGPEQTQGNFGD

SEQ ID NO: 215 - protein, 16 aa, Severe acute respiratory syndrome coronavirus 2
PSASAFFGMSRIGMEV

SEQ ID NO: 216 - protein, 16 aa, Severe acute respiratory syndrome coronavirus 2
GDFVKATCEFCGTENL

SEQ ID NO: 217 - protein, 19 aa, Severe acute respiratory syndrome coronavirus 2
FKLNEEIAIILASFSASTS

SEQ ID NO: 218 - protein, 17 aa, Severe acute respiratory syndrome coronavirus 2
FKQIVESCGNFKVTKGK

SEQ ID NO: 219 - protein, 20 aa, Severe acute respiratory syndrome coronavirus 2
AWNIGEQKSILSPLYAFASE

SEQ ID NO: 220 - protein, 15 aa, Severe acute respiratory syndrome coronavirus 2
TNNLVVMAYITGGVV

SEQ ID NO: 221 - protein, 15 aa, Severe acute respiratory syndrome coronavirus 2
KLKALNLGETFVTHS

SEQ ID NO: 222 - protein, 20 aa, Severe acute respiratory syndrome coronavirus 2
LVGTPVCINGLMLLEIKDTE

SEQ ID NO: 223 - protein, 18 aa, Severe acute respiratory syndrome coronavirus 2
PTKVTFGDDTVIEVQGYK

SEQ ID NO: 224 - protein, 16 aa, Severe acute respiratory syndrome coronavirus 2
YTVELGTEVNEFACVV

SEQ ID NO: 225 - protein, 25 aa, Severe acute respiratory syndrome coronavirus 2
ATYYLFDESGEFKLASHMYCSFYPP

SEQ ID NO: 226 - protein, 15 aa, Severe acute respiratory syndrome coronavirus 2
EDEEEGDCEEEEFEP

SEQ ID NO: 227 - protein, 17 aa, Severe acute respiratory syndrome coronavirus 2
EEEQEEDWLDDDSQQTV

SEQ ID NO: 228 - protein, 16 aa, Severe acute respiratory syndrome coronavirus 2
KKVKPTVVVNAANVYL

SEQ ID NO: 229 - protein, 18 aa, Severe acute respiratory syndrome coronavirus 2
HGGGVAGALNKATNNAMQ

SEQ ID NO: 230 - protein, 19 aa, Severe acute respiratory syndrome coronavirus 2
VRTNVYLAVFDKNLYDKLV

SEQ ID NO: 231 - protein, 15 aa, Severe acute respiratory syndrome coronavirus 2
KIKACVEEVTTTLEE

SEQ ID NO: 232 - protein, 20 aa, Severe acute respiratory syndrome coronavirus 2
LHPDSATLVSDIDITFLKKD

SEQ ID NO: 233 - protein, 17 aa, Severe acute respiratory syndrome coronavirus 2
DNYITTYPGQGLNGYTV

SEQ ID NO: 234 - protein, 43 aa, Severe acute respiratory syndrome coronavirus 2; identical to SEQ ID NO: 129

SEQ ID NO: 235 - protein, 31 aa, Severe acute respiratory syndrome coronavirus 2; identical to SEQ ID NO: 130

SEQ ID NO: 236 - protein, 31 aa, Severe acute respiratory syndrome coronavirus 2; identical to SEQ ID NO: 131

SEQ ID NO: 237 - protein, 15 aa, Severe acute respiratory syndrome coronavirus 2
ISLAGSYKDWSYSGQ

SEQ ID NO: 238 - protein, 24 aa, Severe acute respiratory syndrome coronavirus 2
SMTYGQQFGPTYLDGADVTKIKPH

SEQ ID NO: 239 - protein, 20 aa, Severe acute respiratory syndrome coronavirus 2
HEGKTFYVLPNDDTLRVEAF

SEQ ID NO: 240 - protein, 20 aa, Severe acute respiratory syndrome coronavirus 2
PQVNGLTSIKWADNNCYLAT

SEQ ID NO: 241 - protein, 20 aa, Severe acute respiratory syndrome coronavirus 2
LQQIELKFNPPALQDAYYRA

SEQ ID NO: 242 - protein, 22 aa, Severe acute respiratory syndrome coronavirus 2
VLNVVCKTCGQQQTTLKGVEAV

SEQ ID NO: 243 - protein, 23 aa, Severe acute respiratory syndrome coronavirus 2
YKLDGVVCTEIDPKLDNYYKKDN

SEQ ID NO: 244 - protein, 23 aa, Severe acute respiratory syndrome coronavirus 2
GYKKPASRELKVTFFPDLNGDVV

SEQ ID NO: 245 - protein, 40 aa, Severe acute respiratory syndrome coronavirus 2; identical to SEQ ID NO: 140

SEQ ID NO: 246 - protein, 16 aa, Severe acute respiratory syndrome coronavirus 2
SFYYVWKSYVHVVDGC

SEQ ID NO: 247 - protein, 26 aa, Severe acute respiratory syndrome coronavirus 2
SSTCMMCYKRNRATRVECTTIVNGVR

SEQ ID NO: 248 - protein, 16 aa, Severe acute respiratory syndrome coronavirus 2
DLSLQFKRPINPTDQS

SEQ ID NO: 249 - protein, 20 aa, Severe acute respiratory syndrome coronavirus 2
NNTKGSLPINVIVFDGKSKC

SEQ ID NO: 250 - protein, 18 aa, Severe acute respiratory syndrome coronavirus 2
CQPILLLDQALVSDVGDS

SEQ ID NO: 251 - protein, 17 aa, Severe acute respiratory syndrome coronavirus 2
VKMFDAYVNTFSSTFNV

SEQ ID NO: 252 - protein, 22 aa, Severe acute respiratory syndrome coronavirus 2
LDNVLSTFISAARQGFVDSDVE

SEQ ID NO: 253 - protein, 23 aa, Severe acute respiratory syndrome coronavirus 2
KDFMSLSEQLRKQIRSAAKKNNL

SEQ ID NO: 254 - protein, 24 aa, Severe acute respiratory syndrome coronavirus 2
FKLTCATTRQVVNVVTTKIALKGG

SEQ ID NO: 255 - protein, 17 aa, Severe acute respiratory syndrome coronavirus 2
VFLFVAAIFYLITPVHV

SEQ ID NO: 256 - protein, 29 aa, Severe acute respiratory syndrome coronavirus 2
YTNDKACPLIAAVITREVGFVVPGLPGTI

SEQ ID NO: 257 - protein, 28 aa, Severe acute respiratory syndrome coronavirus 2
TTNGDFLHFLPRVFSAVGNICYTPSKLI

SEQ ID NO: 258 - protein, 18 aa, Severe acute respiratory syndrome coronavirus 2
FATSACVLAAECTIFKDA

SEQ ID NO: 259 - protein, 21 aa, Severe acute respiratory syndrome coronavirus 2
GKPVPYCYDTNVLEGSVAYES

SEQ ID NO: 260 - protein, 23 aa, Severe acute respiratory syndrome coronavirus 2
PLIQPIGALDISASIVAGGIVAI

SEQ ID NO: 261 - protein, 19 aa, Severe acute respiratory syndrome coronavirus 2
AHIQWMVMFTPLVPFWITI

SEQ ID NO: 262 - protein, 41 aa, Severe acute respiratory syndrome coronavirus 2; identical to SEQ ID NO: 157

SEQ ID NO: 263 - protein, 15 aa, Severe acute respiratory syndrome coronavirus 2
NRYLALYNKYKYFSG

SEQ ID NO: 264 - protein, 23 aa, Severe acute respiratory syndrome coronavirus 2
GQTFSVLACYNGSPSGVYQCAMR

SEQ ID NO: 265 - protein, 21 aa, Severe acute respiratory syndrome coronavirus 2
IKGSFLNGSCGSVGFNIDYDC

SEQ ID NO: 266 - protein, 22 aa, Severe acute respiratory syndrome coronavirus 2
LAWLYAAVINGDRWFLNRFTTT

SEQ ID NO: 267 - protein, 18 aa, Severe acute respiratory syndrome coronavirus 2
KYNYEPLTQDHVDILGPL

SEQ ID NO: 268 - protein, 17 aa, Severe acute respiratory syndrome coronavirus 2
TSLLVLVQSTQWSLFFF

SEQ ID NO: 269 - protein, 16 aa, Severe acute respiratory syndrome coronavirus 2
ATVAYFNMVYMPASWV

SEQ ID NO: 270 - protein, 37 aa, Severe acute respiratory syndrome coronavirus 2; identical to SEQ ID NO: 165

SEQ ID NO: 271 - protein, 19 aa, Severe acute respiratory syndrome coronavirus 2
SEDKRAKVTSAMQTMLFTM

SEQ ID NO: 272 - protein, 19 aa, Severe acute respiratory syndrome coronavirus 2
WEIQQVVDADSKIVQLSEI

SEQ ID NO: 273 - protein, 25 aa, Severe acute respiratory syndrome coronavirus 2
MDNSPNLAWPLIVTALRANSAVKLQ

SEQ ID NO: 274 - protein, 18 aa, Severe acute respiratory syndrome coronavirus 2
QMSCAAGTTQTACTDDNA

SEQ ID NO: 275 - protein, 17 aa, Severe acute respiratory syndrome coronavirus 2
KVKYLYFIKGLNNLNRG

SEQ ID NO: 276 - protein, 45 aa, Severe acute respiratory syndrome coronavirus 2; identical to SEQ ID NO: 171

SEQ ID NO: 277 - protein, 22 aa, Severe acute respiratory syndrome coronavirus 2
CHIDHPNPKGFCDLKGKYVQIP

SEQ ID NO: 278 - protein, 23 aa, Severe acute respiratory syndrome coronavirus 2
PISFPLCANGQVFGLYKNTCVGS

SEQ ID NO: 279 - protein, 21 aa, Severe acute respiratory syndrome coronavirus 2
NRNYVFTGYRVTKNSKVQIGE

SEQ ID NO: 280 - protein, 19 aa, Severe acute respiratory syndrome coronavirus 2
LVPQEHYVRITGLYPTLNI

SEQ ID NO: 281 - protein, 15 aa, Severe acute respiratory syndrome coronavirus 2
FSSNVANYQKVGMQK

SEQ ID NO: 282 - protein, 34 aa, Severe acute respiratory syndrome coronavirus 2; identical to SEQ ID NO: 177

SEQ ID NO: 283 - protein, 22 aa, Severe acute respiratory syndrome coronavirus 2
PAPRTLLTKGTLEPEYFNSVCR

SEQ ID NO: 284 - protein, 22 aa, Severe acute respiratory syndrome coronavirus 2
HDVSSAINRPQIGVVREFLTRN

SEQ ID NO: 285 - protein, 18 aa, Severe acute respiratory syndrome coronavirus 2
DYVIFTQTTETAHSCNVN

SEQ ID NO: 286 - protein, 16 aa, Severe acute respiratory syndrome coronavirus 2
PPGDQFKHLIPLMYKG

SEQ ID NO: 287 - protein, 17 aa, Severe acute respiratory syndrome coronavirus 2
WAHGFELTSMKYFVKIG

SEQ ID NO: 288 - protein, 16 aa, Severe acute respiratory syndrome coronavirus 2
DYVYNPFMIDVQQWGF

SEQ ID NO: 289 - protein, 17 aa, Severe acute respiratory syndrome coronavirus 2
VLHDIGNPKAIKCVPQA

SEQ ID NO: 290 - protein, 15 aa, Severe acute respiratory syndrome coronavirus 2
ANSIVCRFDTRVLSN

SEQ ID NO: 291 - protein, 15 aa, Severe acute respiratory syndrome coronavirus 2
NKHAFHTPAFDKSAF

SEQ ID NO: 292 - protein, 25 aa, Severe acute respiratory syndrome coronavirus 2
GKQVVSDIDYVPLKSATCITRCNLG

SEQ ID NO: 293 - protein, 18 aa, Severe acute respiratory syndrome coronavirus 2
QQLPETYFTQSRNLQEFK

SEQ ID NO: 294 - protein, 23 aa, Severe acute respiratory syndrome coronavirus 2
EFIERYKLEGYAFEHIVYGDFSH

SEQ ID NO: 295 - protein, 20 aa, Severe acute respiratory syndrome coronavirus 2
PKGIMMNVAKYTQLCQYLNT

SEQ ID NO: 296 - protein, 19 aa, Severe acute respiratory syndrome coronavirus 2
GTLLVDSDLNDFVSDADST

SEQ ID NO: 297 - protein, 20 aa, Severe acute respiratory syndrome coronavirus 2
WWTAFVTNVNASSSEAFLIG

SEQ ID NO: 298 - protein, 17 aa, Severe acute respiratory syndrome coronavirus 2
FDMSKFPLKLRGTAVMS

SEQ ID NO: 299 - protein, 26 aa, Severe acute respiratory syndrome coronavirus 2
SKTQSLLIVNNATNVVIKVCEFQFCN

SEQ ID NO: 300 - protein, 22 aa, Severe acute respiratory syndrome coronavirus 2
REFVFKNIDGYFKIYSKHTPIN

SEQ ID NO: 301 - protein, 22 aa, Severe acute respiratory syndrome coronavirus 2
LEPLVDLPIGINITRFQTLLAL

SEQ ID NO: 302 - protein, 44 aa, Severe acute respiratory syndrome coronavirus 2; identical to SEQ ID NO: 197

SEQ ID NO: 303 - protein, 45 aa, Severe acute respiratory syndrome coronavirus 2; identical to SEQ ID NO: 198

SEQ ID NO: 304 - protein, 19 aa, Severe acute respiratory syndrome coronavirus 2
SPTKLNDLCFTNVYADSFV

SEQ ID NO: 305 - protein, 21 aa, Severe acute respiratory syndrome coronavirus 2
IADYNYKLPDDFTGCVIAWNS

SEQ ID NO: 306 - protein, 35 aa, Severe acute respiratory syndrome coronavirus 2; identical to SEQ ID NO: 201

SEQ ID NO: 307 - protein, 24 aa, Severe acute respiratory syndrome coronavirus 2
FLPFQQFGRDIADTTDAVRDPQTL

SEQ ID NO: 308 - protein, 29 aa, Severe acute respiratory syndrome coronavirus 2
ILDITPCSFGGVSVITPGTNTSNQVAVLY

SEQ ID NO: 309 - protein, 19 aa, Severe acute respiratory syndrome coronavirus 2
VNNSYECDIPIGAGICASY

SEQ ID NO: 310 - protein, 36 aa, Severe acute respiratory syndrome coronavirus 2; identical to SEQ ID NO: 205

SEQ ID NO: 311 - protein, 22 aa, Severe acute respiratory syndrome coronavirus 2
MQMAYRFNGIGVTQNVLYENQK

SEQ ID NO: 312 - protein, 58 aa, Severe acute respiratory syndrome coronavirus 2; identical to SEQ ID NO: 207

SEQ ID NO: 313 - protein, 22 aa, Severe acute respiratory syndrome coronavirus 2
QTYVTQQLIRAAEIRASANLAA

SEQ ID NO: 314 - protein, 19 aa, Severe acute respiratory syndrome coronavirus 2
KMSECVLGQSKRVDFCGKG

SEQ ID NO: 315 - protein, 20 aa, Severe acute respiratory syndrome coronavirus 2
GTLIVNSVLLFLAFVVFLLV

SEQ ID NO: 316 - protein, 27 aa, Severe acute respiratory syndrome coronavirus 2
ASFRLFARTRSMWSFNPETNILLNVPL

SEQ ID NO: 317 - protein, 17 aa, Severe acute respiratory syndrome coronavirus 2
LTENKYSQLDEEQPMEI

SEQ ID NO: 318 - protein, 15 aa, Severe acute respiratory syndrome coronavirus 2
LSPRWYFYYLGTGPE

SEQ ID NO: 319 - protein, 17 aa, Severe acute respiratory syndrome coronavirus 2
QAFGRRGPEQTQGNFGD

SEQ ID NO: 320 - protein, 16 aa, Severe acute respiratory syndrome coronavirus 2
PSASAFFGMSRIGMEV

SEQ ID NO: 321 - DNA, 9871 bp, Artificial sequence
  Synthesized
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgagtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaactttaatacgactcactataggggccgccaccaagcttggtaccatgtttgtgttcctggtgctgctgccactggtgtccagccagtgtgtgaacctgaccaccaggacccaacttcctcctgcctacaccaactccttcaccaggggagtctactaccctgacaaggtgttcaggtcctctgtgctgcacagcacccaggacctgttcctgccattcttcagcaatgtgacctggttccatgccatccatgtgtctggcaccaatggcaccaagaggtttgacaaccctgtgctgccattcaatgatggagtctactttgccagcacagagaagagcaacatcatcaggggctggatttttggcaccaccctggacagcaagacccagtccctgctgattgtgaacaatgccaccaatgtggtgattaaggtgtgtgagttccagttctgtaatgacccattcctgggagtctactaccacaagaacaacaagtcctggatggagtctgagttcagggtctactcctctgccaacaactgtacctttgaatatgtgagccaaccattcctgatggacttggagggcaagcagggcaacttcaagaacctgagggagtttgtgttcaagaacattgatggctacttcaagatttacagcaaacacacaccaatcaacctggtgagggacctgccacagggcttctctgccttggaaccactggtggacctgccaattggcatcaacatcaccaggttccagaccctgctggctctgcacaggtcctacctgacacctggagactcctcctctggctggacagcaggagcagcagcctactatgtgggctacctccaaccaaggaccttcctgctgaaatacaatgagaatggcaccatcacagatgctgtggactgtgccctggacccactgtctgagaccaagtgtaccctgaaatccttcacagtggagaagggcatctaccagaccagcaacttcagggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtggagggcttcaactgttactttccactccaatcctatggcttccaaccaaccaatggagtgggctaccaaccatacagggtggtggtgctgtcctttgaactgctccatgcccctgccacagtgtgtggaccaaagaagagcaccaacctggtgaagaacaagtgtgtgaacttcaacttcaatggactgacaggcacaggagtgctgacagagagcaacaagaagttcctgccattccaacagtttggcagggacattgctgacaccacagatgctgtgagggacccacagaccttggagattctggacatcacaccatgttcctttggaggagtgtctgtgattacacctggcaccaacaccagcaaccaggtggctgtgctctaccaggatgtgaactgtactgaggtgcctgtggctatccatgctgaccaacttacaccaacctggagggtctacagcacaggcagcaatgtgttccagaccagggctggctgtctgattggagcagagcatgtgaacaactcctatgagtgtgacatcccaattggagcaggcatctgtgcctcctaccagacccagaccaacagcccaaggagggcaaggtctgtggcaagccagagcatcattgcctacacaatgagtctgggagcagagaactctgtggcttacagcaacaacagcattgccatcccaaccaacttcaccatctctgtgaccacagagattctgcctgtgagtatgaccaagacctctgtggactgtacaatgtatatctgtggagacagcacagagtgtagcaacctgctgctccaatatggctccttctgtacccaacttaacagggctctgacaggcattgctgtggaacaggacaagaacacccaggaggtgtttgcccaggtgaagcagatttacaagacacctccaatcaaggactttggaggcttcaacttcagccagattctgcctgacccaagcaagccaagcaagaggtccttcattgaggacctgctgttcaacaaggtgaccctggctgatgctggcttcatcaagcaatatggagactgtctgggagacattgctgccagggacctgatttgtgcccagaagttcaatggactgacagtgctgcctccactgctgacagatgagatgattgcccaatacacctctgccctgctggctggcaccatcacctctggctggacctttggagcaggagcagccctccaaatcccatttgctatgcagatggcttacaggttcaatggcattggagtgacccagaatgtgctctatgagaaccagaaactgattgccaaccagttcaactctgccattggcaagattcaggactccctgtccagcacagcctctgccctgggcaaactccaagatgtggtgaaccagaatgcccaggctctgaacaccctggtgaagcaactttccagcaactttggagccatctcctctgtgctgaatgacatcctgagcagactggacaaggtggaggctgaggtccagattgacagactgattacaggcagactccaatccctccaaacctatgtgacccaacaacttatcagggctgctgagattagggcatctgccaacctggctgccaccaagatgagtgagtgtgtgctgggacaaagcaagagggtggacttctgtggcaagggctaccacctgatgagttttccacagtctgcccctcatggagtggtgttcctgcatgtgacctatgtgcctgcccaggagaagaacttcaccacagcccctgccatctgccatgatggcaaggctcactttccaagggagggagtgtttgtgagcaatggcacccactggtttgtgacccagaggaacttctatgaaccacagattatcaccacagacaacacctttgtgtctggcaactgtgatgtggtgattggcattgtgaacaacacagtctatgacccactccaacctgaactggactccttcaaggaggaactggacaaatacttcaagaaccacaccagccctgatgtggacctgggagacatctctggcatcaatgcctctgtggtgaacatccagaaggagattgacagactgaatgaggtggctaagaacctgaatgagtccctgattgacctccaagaactgggcaaatatgaacaatacatcaagtggccatggtacatctggctgggcttcattgctggactgattgccattgtgatggtgaccataatgctgtgttgtatgacctcctgttgttcctgtctgaaaggctgttgttcctgtggctcctgttgtaagtttgatgaggatgactctgaacctgtgctgaaaggagtgaaactgcactacacctgaatctagagcggccgccgaattcgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 322 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 323 - protein, 375 aa, Homo sapiens
MCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 324 - protein, 13 aa, Homo sapiens
CASSLGGNTEAFF

SEQ ID NO: 325 - protein, 13 aa, Homo sapiens
CAWSVGQSYEQYF

SEQ ID NO: 326 - protein, 14 aa, Homo sapiens
CASSGDRGNTEAFF

SEQ ID NO: 327 - protein, 12 aa, Homo sapiens
CASSPGNTEAFF

SEQ ID NO: 328 - protein, 13 aa, Homo sapiens
CASSLRQNTEAFF

SEQ ID NO: 329 - protein, 11 aa, Homo sapiens
CASSYYNEQFF

SEQ ID NO: 330 - protein, 12 aa, Homo sapiens
CASSVGGNTIYF

SEQ ID NO: 331 - protein, 13 aa, Homo sapiens
CASSLAWGNEQFF

SEQ ID NO: 332 - protein, 12 aa, Homo sapiens
CASSLMNTEAFF

SEQ ID NO: 333 - protein, 15 aa, Homo sapiens
CASSQARTNTGELFF

SEQ ID NO: 334 - protein, 15 aa, Homo sapiens
CATSRDPGSNQPQHF

SEQ ID NO: 335 - protein, 13 aa, Homo sapiens
CASSLAGGYEQYF

SEQ ID NO: 336 - protein, 12 aa, Homo sapiens
CASSLGGYGYTF

SEQ ID NO: 337 - protein, 12 aa, Homo sapiens
CSAREREYEQYF

SEQ ID NO: 338 - protein, 11 aa, Homo sapiens
CASSVSYEQYF

SEQ ID NO: 339 - protein, 14 aa, Homo sapiens
CASSLGQGAYEQYF

SEQ ID NO: 340 - protein, 15 aa, Homo sapiens
CASSPWSSGANVLTF

SEQ ID NO: 341 - protein, 13 aa, Homo sapiens
CSARGQNTGELFF

SEQ ID NO: 342 - protein, 13 aa, Homo sapiens
CASSPRGSTEAFF

SEQ ID NO: 343 - protein, 16 aa, Homo sapiens
CASSFRQGVNTGELFF

SEQ ID NO: 344 - protein, 13 aa, Homo sapiens
CASSWDTSYEQYF

SEQ ID NO: 345 - protein, 13 aa, Homo sapiens
CASSLAGNYEQYF

SEQ ID NO: 346 - protein, 13 aa, Homo sapiens
CASSYSGGTEAFF

SEQ ID NO: 347 - protein, 14 aa, Homo sapiens
CASSLGTGTDTQYF

SEQ ID NO: 348 - protein, 13 aa, Homo sapiens
CASSSRGYYEQYF

SEQ ID NO: 349 - protein, 13 aa, Homo sapiens
CSARDGNTGELFF

SEQ ID NO: 350 - protein, 14 aa, Homo sapiens
CSARDPGQAYEQYF

SEQ ID NO: 351 - protein, 14 aa, Homo sapiens
CASSLGGAPYEQYF

SEQ ID NO: 352 - protein, 14 aa, Homo sapiens
CASSLGGLNTEAFF

SEQ ID NO: 353 - protein, 14 aa, Homo sapiens
CASSLNPNTGELFF

SEQ ID NO: 354 - protein, 12 aa, Homo sapiens
CASSVGTGELFF

SEQ ID NO: 355 - protein, 13 aa, Homo sapiens
CASSLQNTGELFF

SEQ ID NO: 356 - protein, 13 aa, Homo sapiens
CASSLSGANVLTF

SEQ ID NO: 357 - protein, 13 aa, Homo sapiens
CATSDWTGNEQFF

SEQ ID NO: 358 - protein, 12 aa, Homo sapiens
CASSQETGELFF

SEQ ID NO: 359 - protein, 16 aa, Homo sapiens
CASSLGGEGFNQPQHF

SEQ ID NO: 360 - protein, 15 aa, Homo sapiens
CASSLGASSGNTIYF

SEQ ID NO: 361 - protein, 15 aa, Homo sapiens
CASSQERGGPDTQYF

SEQ ID NO: 362 - protein, 13 aa, Homo sapiens
CASSLGIGYEQYF

SEQ ID NO: 363 - protein, 13 aa, Homo sapiens
CASSVGGDQPQHF

SEQ ID NO: 364 - protein, 11 aa, Homo sapiens
CASSQGPEAFF

SEQ ID NO: 365 - protein, 14 aa, Homo sapiens
CASSQDSVQETQYF

SEQ ID NO: 366 - protein, 15 aa, Homo sapiens
CASSVGQGYNSPLHF

SEQ ID NO: 367 - protein, 14 aa, Homo sapiens
CSARLAGDTGELFF

SEQ ID NO: 368 - protein, 13 aa, Homo sapiens
CSVEGLRNTEAFF

SEQ ID NO: 369 - protein, 13 aa, Homo sapiens
CASSYRQNTEAFF

SEQ ID NO: 370 - protein, 13 aa, Homo sapiens
CASSFGGYQPQHF

SEQ ID NO: 371 - protein, 14 aa, Homo sapiens
CASSLERLQETQYF

SEQ ID NO: 372 - protein, 12 aa, Homo sapiens
CATSDFADTQYF

SEQ ID NO: 373 - protein, 15 aa, Homo sapiens
CASSQTGTNTGELFF

SEQ ID NO: 374 - protein, 12 aa, Homo sapiens
CASRTASYEQYF

SEQ ID NO: 375 - protein, 15 aa, Homo sapiens
CASSFRTVSSYEQYF

SEQ ID NO: 376 - protein, 10 aa, Homo sapiens
CASSFNEQFF

SEQ ID NO: 377 - protein, 11 aa, Homo sapiens
CASSLTETQYF

SEQ ID NO: 378 - protein, 12 aa, Homo sapiens
CASSLGGYGYTF

SEQ ID NO: 379 - protein, 13 aa, Homo sapiens
CASSLGENTEAFF

SEQ ID NO: 380 - protein, 12 aa, Homo sapiens
CASSLAGGEQYF

SEQ ID NO: 381 - protein, 12 aa, Homo sapiens
CASSHGGYEQYF

SEQ ID NO: 382 - protein, 13 aa, Homo sapiens
CASSFGVSYEQYF

SEQ ID NO: 383 - protein, 15 aa, Homo sapiens
CASSWPSGISYEQYF

SEQ ID NO: 384 - protein, 11 aa, Homo sapiens
CASSFTGGYTF

SEQ ID NO: 385 - protein, 11 aa, Homo sapiens
CASSLGDGYTF

SEQ ID NO: 386 - protein, 11 aa, Homo sapiens
CASSVTDTQYF

SEQ ID NO: 387 - protein, 13 aa, Homo sapiens
CASSSTGGYGYTF

SEQ ID NO: 388 - protein, 13 aa, Homo sapiens
CASSLGGYQPQHF

SEQ ID NO: 389 - protein, 13 aa, Homo sapiens
CSARVLAGNEQFF

SEQ ID NO: 390 - protein, 13 aa, Homo sapiens
CASSLSGNTEAFF

SEQ ID NO: 391 - protein, 14 aa, Homo sapiens
CASSSGQLQETQYF

SEQ ID NO: 392 - protein, 13 aa, Homo sapiens
CASSVGLNTEAFF

SEQ ID NO: 393 - protein, 16 aa, Homo sapiens
CASSPRGLAGYNEQFF

SEQ ID NO: 394 - protein, 11 aa, Homo sapiens
CASSFTGGYTF

SEQ ID NO: 395 - protein, 13 aa, Homo sapiens
CASSQAGGYEQYF

SEQ ID NO: 396 - protein, 14 aa, Homo sapiens
CASSPGGSGNTIYF

SEQ ID NO: 397 - protein, 14 aa, Homo sapiens
CASSYSGGNQPQHF

SEQ ID NO: 398 - protein, 13 aa, Homo sapiens
CASSLYSNQPQHF

SEQ ID NO: 399 - protein, 13 aa, Homo sapiens
CASSRVGNQPQHF

SEQ ID NO: 400 - protein, 14 aa, Homo sapiens
CASSLGVGLYEQYF

SEQ ID NO: 401 - protein, 13 aa, Homo sapiens
CASSLDSYNEQFF

SEQ ID NO: 402 - protein, 11 aa, Homo sapiens
CASRLNTEAFF

SEQ ID NO: 403 - protein, 13 aa, Homo sapiens
CASSLQGNTEAFF

SEQ ID NO: 404 - protein, 12 aa, Homo sapiens
CASSGNTGELFF

SEQ ID NO: 405 - protein, 13 aa, Homo sapiens
CASSSPDSYEQYF

SEQ ID NO: 406 - protein, 14 aa, Homo sapiens
CASSVTGGTDTQYF

SEQ ID NO: 407 - protein, 12 aa, Homo sapiens
CASSLAGGELFF

SEQ ID NO: 408 - protein, 12 aa, Homo sapiens
CASSLNSYEQYF

SEQ ID NO: 409 - protein, 13 aa, Homo sapiens
CSARDGVNTEAFF

SEQ ID NO: 410 - protein, 13 aa, Homo sapiens
CASSLGSTDTQYF

SEQ ID NO: 411 - protein, 13 aa, Homo sapiens
CASSLGTSNEQYF

SEQ ID NO: 412 - protein, 13 aa, Homo sapiens
CASSLSRNSPLHF

SEQ ID NO: 413 - protein, 11 aa, Homo sapiens
CASSQGYEQYF

SEQ ID NO: 414 - protein, 13 aa, Homo sapiens
CASSYSTDYGYTF

SEQ ID NO: 415 - protein, 13 aa, Homo sapiens
CASSPGGFYGYTF

SEQ ID NO: 416 - protein, 12 aa, Homo sapiens
CASSLVGYEQYF

SEQ ID NO: 417 - protein, 14 aa, Homo sapiens
CASSEGTVVYEQYF

SEQ ID NO: 418 - protein, 14 aa, Homo sapiens
CASSFVAGTDTQYF

SEQ ID NO: 419 - protein, 11 aa, Homo sapiens
CSAGASYEQYF

SEQ ID NO: 420 - protein, 15 aa, Homo sapiens
CASSLGTGGTDTQYF

SEQ ID NO: 421 - protein, 14 aa, Homo sapiens
CASSLTGVDYGYTF

SEQ ID NO: 422 - protein, 13 aa, Homo sapiens
CASSKDRYNEQFF

SEQ ID NO: 423 - protein, 14 aa, Homo sapiens
CASGTGDSNQPQHF

SEQ ID NO: 424 - protein, 12 aa, Homo sapiens
CASSFAGDTQYF

SEQ ID NO: 425 - protein, 16 aa, Homo sapiens
CASSQIQGAGNQPQHF

SEQ ID NO: 426 - protein, 12 aa, Homo sapiens
CASSIGVSEAFF

SEQ ID NO: 427 - protein, 12 aa, Homo sapiens
CATSDLTGELFF

SEQ ID NO: 428 - protein, 13 aa, Homo sapiens
CAISDDLNTEAFF

SEQ ID NO: 429 - protein, 11 aa, Homo sapiens
CASSSSFEAFF

SEQ ID NO: 430 - protein, 15 aa, Homo sapiens
CASSQARTNTGELFF

SEQ ID NO: 431 - protein, 14 aa, Homo sapiens
CASSGGYSNQPQHF

SEQ ID NO: 432 - protein, 12 aa, Homo sapiens
CASSVDVGELFF

SEQ ID NO: 433 - protein, 12 aa, Homo sapiens
CASSFGNTEAFF

SEQ ID NO: 434 - protein, 11 aa, Homo sapiens
CASSYQETQYF

SEQ ID NO: 435 - protein, 12 aa, Homo sapiens
CASSLGGNEQFF

SEQ ID NO: 436 - protein, 13 aa, Homo sapiens
CASSLVGSYEQYF

SEQ ID NO: 437 - protein, 13 aa, Homo sapiens
CASSPGSYNEQFF

SEQ ID NO: 438 - protein, 14 aa, Homo sapiens
CASSIQGSTDTQYF

SEQ ID NO: 439 - protein, 14 aa, Homo sapiens
CASRLAGFTGELFF

SEQ ID NO: 440 - protein, 12 aa, Homo sapiens
CAISESSYEQYF

SEQ ID NO: 441 - protein, 15 aa, Homo sapiens
CASSQVGQGTYEQYF

SEQ ID NO: 442 - protein, 13 aa, Homo sapiens
CASSLQGGQPQHF

SEQ ID NO: 443 - protein, 11 aa, Homo sapiens
CASSSSYEQYF

SEQ ID NO: 444 - protein, 11 aa, Homo sapiens
CASSVADTQYF

SEQ ID NO: 445 - protein, 15 aa, Homo sapiens
CASSEGLAGSYEQYF

SEQ ID NO: 446 - protein, 13 aa, Homo sapiens
CASSHSLGREQYF

SEQ ID NO: 447 - protein, 13 aa, Homo sapiens
CASSYGGNQPQHF

SEQ ID NO: 448 - protein, 13 aa, Homo sapiens
CASSSGGNQPQHF

SEQ ID NO: 449 - protein, 13 aa, Homo sapiens
CASSLGGGTEAFF

SEQ ID NO: 450 - protein, 14 aa, Homo sapiens
CASSLVQGSYEQYF

SEQ ID NO: 451 - protein, 11 aa, Homo sapiens
CASSLSGPQHF

SEQ ID NO: 452 - protein, 14 aa, Homo sapiens
CASSLDGGNTEAFF

SEQ ID NO: 453 - protein, 11 aa, Homo sapiens
CASSFTDTQYF

SEQ ID NO: 454 - protein, 11 aa, Homo sapiens
CASSYGYEQYF

SEQ ID NO: 455 - protein, 14 aa, Homo sapiens
CASSIVGSYNEQFF

SEQ ID NO: 456 - protein, 15 aa, Homo sapiens
CASSQGLAGGNEQFF

SEQ ID NO: 457 - protein, 13 aa, Homo sapiens
CASSTSSTGELFF

SEQ ID NO: 458 - protein, 14 aa, Homo sapiens
CASSRHSGANVLTF

SEQ ID NO: 459 - protein, 15 aa, Homo sapiens
CASSPGQGTNTEAFF

SEQ ID NO: 460 - protein, 12 aa, Homo sapiens
CASSPGQETQYF

SEQ ID NO: 461 - protein, 11 aa, Homo sapiens
CASSQAGELFF

SEQ ID NO: 462 - protein, 14 aa, Homo sapiens
CASSLAGGTDTQYF

SEQ ID NO: 463 - protein, 13 aa, Homo sapiens
CASSLGQGYEQYF

SEQ ID NO: 464 - protein, 11 aa, Homo sapiens
CASSLADEQFF

SEQ ID NO: 465 - protein, 12 aa, Homo sapiens
CASSQGGNEQFF

SEQ ID NO: 466 - protein, 14 aa, Homo sapiens
CASSFVAGTDTQYF

SEQ ID NO: 467 - protein, 12 aa, Homo sapiens
CASSPGGNEQFF

SEQ ID NO: 468 - protein, 12 aa, Homo sapiens
CASSGDSYEQYF

SEQ ID NO: 469 - protein, 14 aa, Homo sapiens
CASSGGSSTDTQYF

SEQ ID NO: 470 - protein, 14 aa, Homo sapiens
CASSSGQANEKLFF

SEQ ID NO: 471 - protein, 11 aa, Homo sapiens
CSASSYNEQFF

SEQ ID NO: 472 - protein, 13 aa, Homo sapiens
CASSVGGTGELFF

SEQ ID NO: 473 - protein, 13 aa, Homo sapiens
CASSVGDNSPLHF

SEQ ID NO: 474 - protein, 12 aa, Homo sapiens
CASSLGLYEQYF

SEQ ID NO: 475 - protein, 10 aa, Homo sapiens
CSARYTEAFF

SEQ ID NO: 476 - protein, 14 aa, Homo sapiens
CASSLGLAPYEQYF

SEQ ID NO: 477 - protein, 13 aa, Homo sapiens
CASSLGGGTEAFF

SEQ ID NO: 478 - protein, 14 aa, Homo sapiens
CASSASYSNQPQHF

SEQ ID NO: 479 - protein, 13 aa, Homo sapiens
CASSSGQGYEQYF

SEQ ID NO: 480 - protein, 11 aa, Homo sapiens
CASSFTGGYTF

SEQ ID NO: 481 - protein, 11 aa, Homo sapiens
CASSLVNEQFF

SEQ ID NO: 482 - protein, 13 aa, Homo sapiens
CASSPYSNQPQHF

SEQ ID NO: 483 - protein, 15 aa, Homo sapiens
CASSLELAGTGELFF

SEQ ID NO: 484 - protein, 13 aa, Homo sapiens
CASSSERYNEQFF

SEQ ID NO: 485 - protein, 12 aa, Homo sapiens
CASSLGGGELFF

SEQ ID NO: 486 - protein, 15 aa, Homo sapiens
CASSLAGGGGTEAFF

SEQ ID NO: 487 - protein, 13 aa, Homo sapiens
CASSLSGDYEQYF

SEQ ID NO: 488 - protein, 14 aa, Homo sapiens
CASSVGQGTYEQYF

SEQ ID NO: 489 - protein, 14 aa, Homo sapiens
CASSPTATNEKLFF

SEQ ID NO: 490 - protein, 12 aa, Homo sapiens
CASSLDRETQYF

SEQ ID NO: 491 - protein, 13 aa, Homo sapiens
CSARTGANYGYTF

SEQ ID NO: 492 - protein, 13 aa, Homo sapiens
CSARDSDSYEQYF

SEQ ID NO: 493 - protein, 15 aa, Homo sapiens
CASSLAGGGGTEAFF

SEQ ID NO: 494 - protein, 13 aa, Homo sapiens
CASSLAGEETQYF

SEQ ID NO: 495 - protein, 13 aa, Homo sapiens
CASSPRSTDTQYF

SEQ ID NO: 496 - protein, 13 aa, Homo sapiens
CSARTSGTYEQYF

SEQ ID NO: 497 - protein, 16 aa, Homo sapiens
CASRTSGSGYTGELFF

SEQ ID NO: 498 - protein, 11 aa, Homo sapiens
CSARGGYEQYF

SEQ ID NO: 499 - protein, 13 aa, Homo sapiens
CASSLAGTYEQYF

SEQ ID NO: 500 - protein, 13 aa, Homo sapiens
CASSYSGNTEAFF

SEQ ID NO: 501 - protein, 13 aa, Homo sapiens
CASSSGQGYEQYF

SEQ ID NO: 502 - protein, 11 aa, Homo sapiens
CASSFPDTQYF

SEQ ID NO: 503 - protein, 13 aa, Homo sapiens
CASSSGQGYEQYF

SEQ ID NO: 504 - protein, 11 aa, Homo sapiens
CASSEGYEQYF

SEQ ID NO: 505 - protein, 14 aa, Homo sapiens
CASSLLQGNQPQHF

SEQ ID NO: 506 - protein, 14 aa, Homo sapiens
CASSPTPLNTEAFF

SEQ ID NO: 507 - protein, 12 aa, Homo sapiens
CASRTASYEQYF

SEQ ID NO: 508 - protein, 12 aa, Homo sapiens
CASSLGYNEQFF

SEQ ID NO: 509 - protein, 13 aa, Homo sapiens
CASSELNTGELFF

SEQ ID NO: 510 - protein, 14 aa, Homo sapiens
CASSVGTGTYEQYF

SEQ ID NO: 511 - protein, 14 aa, Homo sapiens
CASSEDGMNTEAFF

SEQ ID NO: 512 - protein, 12 aa, Homo sapiens
CASSYGGNEQFF

SEQ ID NO: 513 - protein, 12 aa, Homo sapiens
CASSFGTDTQYF

SEQ ID NO: 514 - protein, 11 aa, Homo sapiens
CASSGSYEQYF

SEQ ID NO: 515 - protein, 15 aa, Homo sapiens
CASSVGGSSTDTQYF

SEQ ID NO: 516 - protein, 14 aa, Homo sapiens
CASSFQGSTDTQYF

SEQ ID NO: 517 - protein, 12 aa, Homo sapiens
CASSFGTDTQYF

SEQ ID NO: 518 - protein, 13 aa, Homo sapiens
CASSFGGGTEAFF

SEQ ID NO: 519 - protein, 13 aa, Homo sapiens
CASSYGQGYEQYF

SEQ ID NO: 520 - protein, 12 aa, Homo sapiens
CASSLGAYEQYF

SEQ ID NO: 521 - protein, 11 aa, Homo sapiens
CASSYSYEQYF

SEQ ID NO: 522 - protein, 13 aa, Homo sapiens
CASSQGMNTEAFF

SEQ ID NO: 523 - protein, 11 aa, Homo sapiens
CASSVGGELFF

SEQ ID NO: 524 - protein, 13 aa, Homo sapiens
CASSLTGSYEQYF

SEQ ID NO: 525 - protein, 12 aa, Homo sapiens
CASSYSGNEQFF

SEQ ID NO: 526 - protein, 12 aa, Homo sapiens
CASSIGSYGYTF

SEQ ID NO: 527 - protein, 11 aa, Homo sapiens
CASRDSYGYTF

SEQ ID NO: 528 - protein, 11 aa, Homo sapiens
CASSYYNEQFF

SEQ ID NO: 529 - protein, 12 aa, Homo sapiens
CASSLGGNEQFF

SEQ ID NO: 530 - protein, 15 aa, Homo sapiens
CSARPLAGLYNEQFF

SEQ ID NO: 531 - protein, 15 aa, Homo sapiens
CASSVGGQGAYEQYF

SEQ ID NO: 532 - protein, 15 aa, Homo sapiens
CASSLVGGSGNEQFF

SEQ ID NO: 533 - protein, 12 aa, Homo sapiens
CASSLLGDEQFF

SEQ ID NO: 534 - protein, 14 aa, Homo sapiens
CASSLAGVTDTQYF

SEQ ID NO: 535 - protein, 12 aa, Homo sapiens
CASSSTTDTQYF

SEQ ID NO: 536 - protein, 12 aa, Homo sapiens
CASSLSGYGYTF

SEQ ID NO: 537 - protein, 13 aa, Homo sapiens
CASSVDMNTEAFF

SEQ ID NO: 538 - protein, 11 aa, Homo sapiens
CASSFTDTQYF

SEQ ID NO: 539 - protein, 12 aa, Homo sapiens
CASSPLGDTQYF

SEQ ID NO: 540 - protein, 14 aa, Homo sapiens
CASSVGQGAYEQYF

SEQ ID NO: 541 - protein, 14 aa, Homo sapiens
CASSGDRGNTEAFF

SEQ ID NO: 542 - protein, 15 aa, Homo sapiens
CASSQARTNTGELFF

SEQ ID NO: 543 - protein, 12 aa, Homo sapiens
CASSLGSYEQYF

SEQ ID NO: 544 - protein, 17 aa, Homo sapiens
CASSQGTILLSGNTIYF

SEQ ID NO: 545 - protein, 14 aa, Homo sapiens
CASSLVQGSYEQYF

SEQ ID NO: 546 - protein, 16 aa, Homo sapiens
CASSQGGTSGTDTQYF

SEQ ID NO: 547 - protein, 11 aa, Homo sapiens
CASSESYEQYF

SEQ ID NO: 548 - protein, 14 aa, Homo sapiens
CASSAGTGSYEQYF

SEQ ID NO: 549 - protein, 13 aa, Homo sapiens
CASSQARNTEAFF

SEQ ID NO: 550 - protein, 14 aa, Homo sapiens
CSASSESSYNEQFF

SEQ ID NO: 551 - protein, 12 aa, Homo sapiens
CASSYRGTEAFF

SEQ ID NO: 552 - protein, 12 aa, Homo sapiens
CASSLRGVEQYF

SEQ ID NO: 553 - protein, 12 aa, Homo sapiens
CSAGDSSYEQYF

SEQ ID NO: 554 - protein, 15 aa, Homo sapiens
CASSVGQGASYEQYF

SEQ ID NO: 555 - protein, 14 aa, Homo sapiens
CASSLGGGNTEAFF

SEQ ID NO: 556 - protein, 14 aa, Homo sapiens
CASSSQGAGTEAFF

SEQ ID NO: 557 - protein, 11 aa, Homo sapiens
CASSESYEQYF

SEQ ID NO: 558 - protein, 16 aa, Homo sapiens
CASSLIGVSSYNEQFF

SEQ ID NO: 559 - protein, 14 aa, Homo sapiens
CASSPGGEIYEQYF

SEQ ID NO: 560 - protein, 13 aa, Homo sapiens
CSVGLADYNEQFF

SEQ ID NO: 561 - protein, 13 aa, Homo sapiens
CAWSVQQNYGYTF

SEQ ID NO: 562 - protein, 11 aa, Homo sapiens
CASGDSYEQYF

SEQ ID NO: 563 - protein, 13 aa, Homo sapiens
CASSFGVGYEQYF

SEQ ID NO: 564 - protein, 12 aa, Homo sapiens
CASSYSGSGYTF

SEQ ID NO: 565 - protein, 14 aa, Homo sapiens
CATSREGANTEAFF

SEQ ID NO: 566 - protein, 13 aa, Homo sapiens
CASSFGQIYGYTF

SEQ ID NO: 567 - protein, 16 aa, Homo sapiens
CASSFRQGANTGELFF

SEQ ID NO: 568 - protein, 14 aa, Homo sapiens
CASSLDGSSYEQYF

SEQ ID NO: 569 - protein, 15 aa, Homo sapiens
CASSTGLGMNTEAFF

SEQ ID NO: 570 - protein, 13 aa, Homo sapiens
CSARDAESYEQYF

SEQ ID NO: 571 - protein, 14 aa, Homo sapiens
CASSLLVSNQPQHF

SEQ ID NO: 572 - protein, 15 aa, Homo sapiens
CASSPGTSGAYEQYF

SEQ ID NO: 573 - protein, 14 aa, Homo sapiens
CASSPGGGSYEQYF

SEQ ID NO: 574 - protein, 14 aa, Homo sapiens
CASSTGYSNQPQHF

SEQ ID NO: 575 - protein, 13 aa, Homo sapiens
CSASRDSQETQYF

SEQ ID NO: 576 - protein, 12 aa, Homo sapiens
CAWSVTDNEQFF

SEQ ID NO: 577 - protein, 12 aa, Homo sapiens
CASRTGDTEAFF

SEQ ID NO: 578 - protein, 13 aa, Homo sapiens
CASSLGGDQPQHF

SEQ ID NO: 579 - protein, 13 aa, Homo sapiens
CASSLAGNTEAFF

SEQ ID NO: 580 - protein, 12 aa, Homo sapiens
CSARQGGAEAFF

SEQ ID NO: 581 - protein, 15 aa, Homo sapiens
CASSPQGVSGNTIYF

SEQ ID NO: 582 - protein, 12 aa, Homo sapiens
CASSTSSYEQYF

SEQ ID NO: 583 - protein, 15 aa, Homo sapiens
CASSQAGALNTEAFF

SEQ ID NO: 584 - protein, 13 aa, Homo sapiens
CASSIRSSYEQYF

SEQ ID NO: 585 - protein, 12 aa, Homo sapiens
CASSIGLYGYTF

SEQ ID NO: 586 - protein, 12 aa, Homo sapiens
CASRGVSYEQYF

SEQ ID NO: 587 - protein, 13 aa, Homo sapiens
CASSLGGRTEAFF

SEQ ID NO: 588 - protein, 11 aa, Homo sapiens
CASSLTDTQYF

SEQ ID NO: 589 - protein, 13 aa, Homo sapiens
CASSIRSSYEQYF

SEQ ID NO: 590 - protein, 12 aa, Homo sapiens
CASSAGGYEQYF

SEQ ID NO: 591 - protein, 15 aa, Homo sapiens
CASSQETGMNTEAFF

SEQ ID NO: 592 - protein, 11 aa, Homo sapiens
CASRRGTEAFF

SEQ ID NO: 593 - protein, 12 aa, Homo sapiens
CASSLVGETQYF

SEQ ID NO: 594 - protein, 14 aa, Homo sapiens
CASMGGSSYNEQFF

SEQ ID NO: 595 - protein, 13 aa, Homo sapiens
CASSFFLGEKLFF

SEQ ID NO: 596 - protein, 14 aa, Homo sapiens
CASSQDRGAYEQYF

SEQ ID NO: 597 - protein, 13 aa, Homo sapiens
CASSEAGNTEAFF

SEQ ID NO: 598 - protein, 14 aa, Homo sapiens
CASSLSSSQETQYF

SEQ ID NO: 599 - protein, 14 aa, Homo sapiens
CASSYSSRNTEAFF

SEQ ID NO: 600 - protein, 13 aa, Homo sapiens
CASSLGPNYEQYF

SEQ ID NO: 601 - protein, 11 aa, Homo sapiens
CASSSSYEQYF

SEQ ID NO: 602 - protein, 13 aa, Homo sapiens
CSARTGLAYEQYF

SEQ ID NO: 603 - protein, 14 aa, Homo sapiens
CASTLGGFGQPQHF

SEQ ID NO: 604 - protein, 12 aa, Homo sapiens
CASSLGVYEQYF

SEQ ID NO: 605 - protein, 15 aa, Homo sapiens
CASSEGWVTNTEAFF

SEQ ID NO: 606 - protein, 12 aa, Homo sapiens
CASSLGSYGYTF

SEQ ID NO: 607 - protein, 12 aa, Homo sapiens
CASSYPGQPQHF

SEQ ID NO: 608 - protein, 13 aa, Homo sapiens
CASSYSGGTEAFF

SEQ ID NO: 609 - protein, 12 aa, Homo sapiens
CSARRDSYEQYF

SEQ ID NO: 610 - protein, 10 aa, Homo sapiens
CAGGDYGYTF

SEQ ID NO: 611 - protein, 12 aa, Homo sapiens
CSARRDSYEQYF

SEQ ID NO: 612 - protein, 14 aa, Homo sapiens
CASSYGTGGTEAFF

SEQ ID NO: 613 - protein, 13 aa, Homo sapiens
CASSQDLGYGYTF

SEQ ID NO: 614 - protein, 13 aa, Homo sapiens
CASSLMGNQPQHF

SEQ ID NO: 615 - protein, 11 aa, Homo sapiens
CSARTGYEQYF

SEQ ID NO: 616 - protein, 13 aa, Homo sapiens
CASSYDSSYEQYF

SEQ ID NO: 617 - protein, 14 aa, Homo sapiens
CASSLDSMNTEAFF

SEQ ID NO: 618 - protein, 13 aa, Homo sapiens
CASSLGSSYEQYF

SEQ ID NO: 619 - protein, 14 aa, Homo sapiens
CASSYGTGGTEAFF

SEQ ID NO: 620 - protein, 15 aa, Homo sapiens
CASSLVGLAAYEQYF

SEQ ID NO: 621 - protein, 13 aa, Homo sapiens
CASSFQGFYEQYF

SEQ ID NO: 622 - protein, 14 aa, Homo sapiens
CASSLSGPNEKLFF

SEQ ID NO: 623 - protein, 14 aa, Homo sapiens
CASSLDPSSYEQYF

SEQ ID NO: 624 - protein, 12 aa, Homo sapiens
CASSVGAGELFF

SEQ ID NO: 625 - protein, 12 aa, Homo sapiens
CASSPGDYGYTF

SEQ ID NO: 626 - protein, 11 aa, Homo sapiens
CASSLGYEQYF

SEQ ID NO: 627 - protein, 13 aa, Homo sapiens
CASSLGVTGELFF

SEQ ID NO: 628 - protein, 15 aa, Homo sapiens
CASSRSGLAEETQYF

SEQ ID NO: 629 - protein, 11 aa, Homo sapiens
CASSLGSEQYF

SEQ ID NO: 630 - protein, 13 aa, Homo sapiens
CASSPGNSYEQYF

SEQ ID NO: 631 - protein, 14 aa, Homo sapiens
CASSVGQGTYEQYF

SEQ ID NO: 632 - protein, 15 aa, Homo sapiens
CASSLTGNSGNTIYF

SEQ ID NO: 633 - protein, 11 aa, Homo sapiens
CASSIGYEQYF

SEQ ID NO: 634 - protein, 12 aa, Homo sapiens
CSASGRQETQYF

SEQ ID NO: 635 - protein, 15 aa, Homo sapiens
CASSPGFSRHYEQYF

SEQ ID NO: 636 - protein, 14 aa, Homo sapiens
CSARDLGSSYEQYF

SEQ ID NO: 637 - protein, 14 aa, Homo sapiens
CASSFGQLNQPQHF

SEQ ID NO: 638 - protein, 13 aa, Homo sapiens
CASSLTSTDTQYF

SEQ ID NO: 639 - protein, 12 aa, Homo sapiens
CSAGQGRYEQYF

SEQ ID NO: 640 - protein, 15 aa, Homo sapiens
CASSELAGSYNEQFF

SEQ ID NO: 641 - protein, 14 aa, Homo sapiens
CASSYGTGNQPQHF

SEQ ID NO: 642 - protein, 14 aa, Homo sapiens
CASSFGQGDTEAFF

SEQ ID NO: 643 - protein, 12 aa, Homo sapiens
CASSSLGGELFF

SEQ ID NO: 644 - protein, 13 aa, Homo sapiens
CASSQGYNSPLHF

SEQ ID NO: 645 - protein, 13 aa, Homo sapiens
CASSVVGNTEAFF

SEQ ID NO: 646 - protein, 13 aa, Homo sapiens
CSAHGGSSYEQYF

SEQ ID NO: 647 - protein, 13 aa, Homo sapiens
CASSQGYNSPLHF

SEQ ID NO: 648 - protein, 13 aa, Homo sapiens
CASSLAGAYEQYF

SEQ ID NO: 649 - protein, 11 aa, Homo sapiens
CSVDRNTEAFF

SEQ ID NO: 650 - protein, 12 aa, Homo sapiens
CASSWGYNEQFF

SEQ ID NO: 651 - protein, 12 aa, Homo sapiens
CASSPSSYEQYF

SEQ ID NO: 652 - protein, 13 aa, Homo sapiens
CASSLGRTYEQYF

SEQ ID NO: 653 - protein, 15 aa, Homo sapiens
CSARDLAGGLNEQFF

SEQ ID NO: 654 - protein, 13 aa, Homo sapiens
CASSQGYNSPLHF

SEQ ID NO: 655 - protein, 13 aa, Homo sapiens
CSARLADYNEQFF

SEQ ID NO: 656 - protein, 12 aa, Homo sapiens
CASSYGGYGYTF

SEQ ID NO: 657 - protein, 14 aa, Homo sapiens
CASSLSGNHNEQFF

SEQ ID NO: 658 - protein, 12 aa, Homo sapiens
CASSFGGNEQFF

SEQ ID NO: 659 - protein, 12 aa, Homo sapiens
CASSVGGDTQYF

SEQ ID NO: 660 - protein, 11 aa, Homo sapiens
CASSFGYEQYF

SEQ ID NO: 661 - protein, 12 aa, Homo sapiens
CATSRGNQPQHF

SEQ ID NO: 662 - protein, 11 aa, Homo sapiens
CSVDRNTEAFF

SEQ ID NO: 663 - protein, 11 aa, Homo sapiens
CASTYYNEQFF

SEQ ID NO: 664 - protein, 14 aa, Homo sapiens
CASSLGQGDTEAFF

SEQ ID NO: 665 - protein, 13 aa, Homo sapiens
CATSRDRGTEAFF

SEQ ID NO: 666 - protein, 15 aa, Homo sapiens
CASSIAGTSYNEQFF

SEQ ID NO: 667 - protein, 13 aa, Homo sapiens
CASSLAGAQPQHF

SEQ ID NO: 668 - protein, 11 aa, Homo sapiens
CASSLSGTQYF

SEQ ID NO: 669 - protein, 16 aa, Homo sapiens
CASSLIGVSSYNEQFF

SEQ ID NO: 670 - protein, 14 aa, Homo sapiens
CASSPTGGTGELFF

SEQ ID NO: 671 - protein, 12 aa, Homo sapiens
CASSLEAYEQYF

SEQ ID NO: 672 - protein, 13 aa, Homo sapiens
CASSLGTGYGYTF

SEQ ID NO: 673 - protein, 13 aa, Homo sapiens
CSARGQGDYEQYF

SEQ ID NO: 674 - protein, 12 aa, Homo sapiens
CASSLWGYEQYF

SEQ ID NO: 675 - protein, 15 aa, Homo sapiens
CASSLAGANQETQYF

SEQ ID NO: 676 - protein, 13 aa, Homo sapiens
CATSDLASDTQYF

SEQ ID NO: 677 - protein, 12 aa, Homo sapiens
CASSEGGQPQHF

SEQ ID NO: 678 - protein, 11 aa, Homo sapiens
CASSLAYEQYF

SEQ ID NO: 679 - protein, 13 aa, Homo sapiens
CASSLGGNTEAFF

SEQ ID NO: 680 - protein, 12 aa, Homo sapiens
CASSLGNTEAFF

SEQ ID NO: 681 - protein, 11 aa, Homo sapiens
CASSSNSPLHF

SEQ ID NO: 682 - protein, 14 aa, Homo sapiens
CASSFVAGTDTQYF

SEQ ID NO: 683 - protein, 13 aa, Homo sapiens
CASSLTRNTEAFF

SEQ ID NO: 684 - protein, 14 aa, Homo sapiens
CASSLGQGANEQYF

SEQ ID NO: 685 - protein, 14 aa, Homo sapiens
CSAGSGSSYNEQFF

SEQ ID NO: 686 - protein, 12 aa, Homo sapiens
CASSLSVYEQYF

SEQ ID NO: 687 - protein, 14 aa, Homo sapiens
CASSLGQGGYEQYF

SEQ ID NO: 688 - protein, 13 aa, Homo sapiens
CASSIADYNEQFF

SEQ ID NO: 689 - protein, 13 aa, Homo sapiens
CSARVQLNTEAFF

SEQ ID NO: 690 - protein, 13 aa, Homo sapiens
CASSLGSNYEQYF

SEQ ID NO: 691 - protein, 12 aa, Homo sapiens
CASSLGLNEQFF

SEQ ID NO: 692 - protein, 13 aa, Homo sapiens
CASSLGQGYEQYF

SEQ ID NO: 693 - protein, 12 aa, Homo sapiens
CASSPTAYEQYF

SEQ ID NO: 694 - protein, 12 aa, Homo sapiens
CASSFGGAEAFF

SEQ ID NO: 695 - protein, 14 aa, Homo sapiens
CASSGTGLNTEAFF

SEQ ID NO: 696 - protein, 14 aa, Homo sapiens
CSARRDSRTDTQYF

SEQ ID NO: 697 - protein, 11 aa, Homo sapiens
CASSSTYEQYF

SEQ ID NO: 698 - protein, 14 aa, Homo sapiens
CSARRDSRTDTQYF

SEQ ID NO: 699 - protein, 14 aa, Homo sapiens
CSAKGQGSNQPQHF

SEQ ID NO: 700 - protein, 11 aa, Homo sapiens
CATSEGYEQYF

SEQ ID NO: 701 - protein, 14 aa, Homo sapiens
CSARDRANTGELFF

SEQ ID NO: 702 - protein, 13 aa, Homo sapiens
CASSPGSTYEQYF

SEQ ID NO: 703 - protein, 14 aa, Homo sapiens
CATSAGQGNTEAFF

SEQ ID NO: 704 - protein, 12 aa, Homo sapiens
CASSFGNTEAFF

SEQ ID NO: 705 - protein, 15 aa, Homo sapiens
CASSRAGGSTDTQYF

SEQ ID NO: 706 - protein, 15 aa, Homo sapiens
CASSPGLAGGYEQFF

SEQ ID NO: 707 - protein, 13 aa, Homo sapiens
CSATDRVGYGYTF

SEQ ID NO: 708 - protein, 13 aa, Homo sapiens
CASSLRGDYGYTF

SEQ ID NO: 709 - protein, 12 aa, Homo sapiens
CASSLVAEKLFF

SEQ ID NO: 710 - protein, 12 aa, Homo sapiens
CASSLVAEKLFF

SEQ ID NO: 711 - protein, 16 aa, Homo sapiens
CASSLIGVSSYNEQFF

SEQ ID NO: 712 - protein, 13 aa, Homo sapiens
CASSLGQQETQYF

SEQ ID NO: 713 - protein, 13 aa, Homo sapiens
CASSYSGNQPQHF

SEQ ID NO: 714 - protein, 16 aa, Homo sapiens
CASSFRQGANTGELFF

SEQ ID NO: 715 - protein, 13 aa, Homo sapiens
CASSLLGSYEQYF

SEQ ID NO: 716 - protein, 14 aa, Homo sapiens
CASSEGASNQPQHF

SEQ ID NO: 717 - protein, 15 aa, Homo sapiens
CASSQELAGTDTQYF

SEQ ID NO: 718 - protein, 13 aa, Homo sapiens
CASSYRGDYGYTF

SEQ ID NO: 719 - protein, 14 aa, Homo sapiens
CASSLAGVTDTQYF

SEQ ID NO: 720 - protein, 12 aa, Homo sapiens
CASSLVAEKLFF

SEQ ID NO: 721 - protein, 14 aa, Homo sapiens
CSARRDSRTDTQYF

SEQ ID NO: 722 - protein, 15 aa, Homo sapiens
CASSLSGLAGDEQYF

SEQ ID NO: 723 - protein, 9 aa, Homo sapiens
CSASSGYTF

SEQ ID NO: 724 - protein, 13 aa, Homo sapiens
CASSLTGNYGYTF

SEQ ID NO: 725 - protein, 12 aa, Homo sapiens
CAISESSYEQYF

SEQ ID NO: 726 - protein, 15 aa, Homo sapiens
CASSRAGGSTDTQYF

SEQ ID NO: 727 - protein, 16 aa, Homo sapiens
CATSDPRQGDNQPQHF

SEQ ID NO: 728 - protein, 11 aa, Homo sapiens
CASSQTYEQYF

SEQ ID NO: 729 - protein, 15 aa, Homo sapiens
CASSATGDSNQPQHF

SEQ ID NO: 730 - protein, 13 aa, Homo sapiens
CASSLRGAVGYTF

SEQ ID NO: 731 - protein, 14 aa, Homo sapiens
CASSGTGLNTEAFF

SEQ ID NO: 732 - protein, 14 aa, Homo sapiens
CASSLSSPTDTQYF

SEQ ID NO: 733 - protein, 14 aa, Homo sapiens
CASSEGPSSQPQHF

SEQ ID NO: 734 - protein, 14 aa, Homo sapiens
CASSSTGSGNTIYF

SEQ ID NO: 735 - protein, 13 aa, Homo sapiens
CSVGTGWPYEQYF

SEQ ID NO: 736 - protein, 13 aa, Homo sapiens
CASSLTGNYGYTF

SEQ ID NO: 737 - protein, 14 aa, Homo sapiens
CASSEGPSSQPQHF

SEQ ID NO: 738 - protein, 11 aa, Homo sapiens
CASSLTYEQYF

SEQ ID NO: 739 - protein, 13 aa, Homo sapiens
CASRRAGSYEQYF

SEQ ID NO: 740 - protein, 13 aa, Homo sapiens
CSARDGTTYEQYF

SEQ ID NO: 741 - protein, 13 aa, Homo sapiens
CASSPNSTYEQYF

SEQ ID NO: 742 - protein, 11 aa, Homo sapiens
CASSYTYEQYF

SEQ ID NO: 743 - protein, 14 aa, Homo sapiens
CASSLWKRNTEAFF

SEQ ID NO: 744 - protein, 15 aa, Homo sapiens
CASSVVASGQETQYF

SEQ ID NO: 745 - protein, 14 aa, Homo sapiens
CASSPTGSNQPQHF

SEQ ID NO: 746 - protein, 14 aa, Homo sapiens
CASSLSGVTDTQYF

SEQ ID NO: 747 - protein, 12 aa, Homo sapiens
CASSPDGYGYTF

SEQ ID NO: 748 - protein, 14 aa, Homo sapiens
CASSEGASNQPQHF

SEQ ID NO: 749 - protein, 13 aa, Homo sapiens
CAWSVQQNYGYTF

SEQ ID NO: 750 - protein, 12 aa, Homo sapiens
CASKGGGRGYTF

SEQ ID NO: 751 - protein, 16 aa, Homo sapiens
CASSLRDRANTGELFF

SEQ ID NO: 752 - protein, 13 aa, Homo sapiens
CASSLTGNYGYTF

SEQ ID NO: 753 - protein, 11 aa, Homo sapiens
CASSQGVGYTF

SEQ ID NO: 754 - protein, 15 aa, Homo sapiens
CASSRAGGSTDTQYF

SEQ ID NO: 755 - protein, 12 aa, Homo sapiens
CAWSVTDNEQFF

SEQ ID NO: 756 - protein, 11 aa, Homo sapiens
CASSSTYEQYF

SEQ ID NO: 757 - protein, 13 aa, Homo sapiens
CASSFGGNYGYTF

SEQ ID NO: 758 - protein, 14 aa, Homo sapiens
CASSEGFSNQPQHF

SEQ ID NO: 759 - protein, 14 aa, Homo sapiens
CASSLTATNEKLFF

SEQ ID NO: 760 - protein, 11 aa, Homo sapiens
CASSLTDTQYF

SEQ ID NO: 761 - protein, 11 aa, Homo sapiens
CASSPTYEQYF

SEQ ID NO: 762 - protein, 15 aa, Homo sapiens
CASSRSTGDQETQYF

SEQ ID NO: 763 - protein, 13 aa, Homo sapiens
CASSLTGNYGYTF

SEQ ID NO: 764 - protein, 14 aa, Homo sapiens
CASSLSGVTDTQYF

SEQ ID NO: 765 - protein, 15 aa, Homo sapiens
CASSRAGGSTDTQYF

SEQ ID NO: 766 - protein, 14 aa, Homo sapiens
CASSPGGGSYEQYF

SEQ ID NO: 767 - protein, 15 aa, Homo sapiens
CASSLSSGSYNEQFF

SEQ ID NO: 768 - protein, 14 aa, Homo sapiens
CSAKGQGGNQPQHF

SEQ ID NO: 769 - protein, 14 aa, Homo sapiens
CASSLAGGPDEQFF

SEQ ID NO: 770 - protein, 12 aa, Homo sapiens
CSARQGGAEAFF

SEQ ID NO: 771 - protein, 11 aa, Homo sapiens
CASSQTYEQYF

SEQ ID NO: 772 - protein, 15 aa, Homo sapiens
CASSLAGGSTDTQYF

SEQ ID NO: 773 - protein, 15 aa, Homo sapiens
CSARRDRGDNSPLHF

SEQ ID NO: 774 - protein, 15 aa, Homo sapiens
CASSRAGGSTDTQYF

SEQ ID NO: 775 - protein, 15 aa, Homo sapiens
CASSLGLAGGDTQYF

SEQ ID NO: 776 - protein, 13 aa, Homo sapiens
CSARGEGNYGYTF

SEQ ID NO: 777 - protein, 11 aa, Homo sapiens
CASSLNYEQYF

SEQ ID NO: 778 - protein, 11 aa, Homo sapiens
CASSPTYEQYF

SEQ ID NO: 779 - protein, 15 aa, Homo sapiens
CSATRDASSYNEQFF

SEQ ID NO: 780 - protein, 11 aa, Homo sapiens
CASSLNYEQYF

SEQ ID NO: 781 - protein, 14 aa, Homo sapiens
CASSEGASNQPQHF

SEQ ID NO: 782 - protein, 14 aa, Homo sapiens
CATSAGQGNTEAFF

SEQ ID NO: 783 - protein, 13 aa, Homo sapiens
CASRDSGANVLTF

SEQ ID NO: 784 - protein, 15 aa, Homo sapiens
CASSQSGAGTDTQYF

SEQ ID NO: 785 - protein, 13 aa, Homo sapiens
CASSYSGGQPQHF

SEQ ID NO: 786 - protein, 12 aa, Homo sapiens
CASKQGGRGYTF

SEQ ID NO: 787 - protein, 15 aa, Homo sapiens
CASSSGLAGVETQYF

SEQ ID NO: 788 - protein, 12 aa, Homo sapiens
CATKGGGRGYTF

SEQ ID NO: 789 - protein, 12 aa, Homo sapiens
CASSQVAGEQFF

SEQ ID NO: 790 - protein, 15 aa, Homo sapiens
CASSYEGGRNTEAFF

SEQ ID NO: 791 - protein, 15 aa, Homo sapiens
CASSEGGRGPDTQYF

SEQ ID NO: 792 - protein, 15 aa, Homo sapiens
CSATQGAATNEKLFF

SEQ ID NO: 793 - protein, 15 aa, Homo sapiens
CASSLGGVGNQPQHF

SEQ ID NO: 794 - protein, 11 aa, Homo sapiens
CSARRGTEAFF

SEQ ID NO: 795 - protein, 14 aa, Homo sapiens
CASSPTGSNQPQHF

SEQ ID NO: 796 - protein, 14 aa, Homo sapiens
CASSEGASNQPQHF

SEQ ID NO: 797 - protein, 15 aa, Homo sapiens
CASSLGGVGNQPQHF

SEQ ID NO: 798 - protein, 14 aa, Homo sapiens
CASSPGQGDTEAFF

SEQ ID NO: 799 - protein, 13 aa, Homo sapiens
CSARQGVDQPQHF

SEQ ID NO: 800 - protein, 14 aa, Homo sapiens
CASSEGASNQPQHF

SEQ ID NO: 801 - protein, 11 aa, Homo sapiens
CASSPTYEQYF

SEQ ID NO: 802 - protein, 14 aa, Homo sapiens
CSARRDSRTDTQYF

SEQ ID NO: 803 - protein, 14 aa, Homo sapiens
CSARRDSRTDTQYF

SEQ ID NO: 804 - protein, 14 aa, Homo sapiens
CSARRDSRTDTQYF

SEQ ID NO: 805 - protein, 14 aa, Homo sapiens
CSARRDSRTDTQYF

SEQ ID NO: 806 - protein, 15 aa, Homo sapiens
CASSEGGRGPDTQYF

SEQ ID NO: 807 - protein, 13 aa, Homo sapiens
CSATDRVGYGYTF

SEQ ID NO: 808 - protein, 11 aa, Homo sapiens
CSASRGNEQFF

SEQ ID NO: 809 - protein, 14 aa, Homo sapiens
CASSESAGQETQYF

SEQ ID NO: 810 - protein, 11 aa, Homo sapiens
CASSSTYEQYF

SEQ ID NO: 811 - protein, 12 aa, Homo sapiens
CASSQVAGEQYF

SEQ ID NO: 812 - protein, 14 aa, Homo sapiens
CSAKGQGSNQPQHF

SEQ ID NO: 813 - protein, 12 aa, Homo sapiens
CASSQVAGEQYF

SEQ ID NO: 814 - protein, 15 aa, Homo sapiens
CASSQDPGLNTEAFF

SEQ ID NO: 815 - protein, 14 aa, Homo sapiens
CASSEGSSNQPQHF

SEQ ID NO: 816 - protein, 15 aa, Homo sapiens
CASSRSGLAEETQYF

SEQ ID NO: 817 - protein, 12 aa, Homo sapiens
CASSLVAEKLFF

SEQ ID NO: 818 - protein, 12 aa, Homo sapiens
CASSLVSDTQYF

SEQ ID NO: 819 - protein, 14 aa, Homo sapiens
CASSGTGLNTEAFF

SEQ ID NO: 820 - protein, 14 aa, Homo sapiens
CSASRDASNQPQHF

SEQ ID NO: 821 - protein, 11 aa, Homo sapiens
CASSLRRGYTF

SEQ ID NO: 822 - protein, 16 aa, Homo sapiens
CATSDPRQGDNQPQHF

SEQ ID NO: 823 - protein, 14 aa, Homo sapiens
CASSEGASNQPQHF

SEQ ID NO: 824 - protein, 14 aa, Homo sapiens
CSARRDSRTDTQYF

SEQ ID NO: 825 - protein, 11 aa, Homo sapiens
CSASRGNEQFF

SEQ ID NO: 826 - protein, 11 aa, Homo sapiens
CASSQGVGYTF

SEQ ID NO: 827 - protein, 12 aa, Homo sapiens
CASSQVAGEQYF

SEQ ID NO: 828 - protein, 11 aa, Homo sapiens
CASSSTYEQYF

SEQ ID NO: 829 - protein, 12 aa, Homo sapiens
CASKQGGRGYTF

SEQ ID NO: 830 - protein, 11 aa, Homo sapiens
CASSPTYEQYF

SEQ ID NO: 831 - protein, 14 aa, Homo sapiens
CASSEGFSNQPQHF

SEQ ID NO: 832 - protein, 15 aa, Homo sapiens
CSATRGAGGQETQYF

SEQ ID NO: 833 - protein, 11 aa, Homo sapiens
CASSYTYEQYF

SEQ ID NO: 834 - protein, 11 aa, Homo sapiens
CASSQTYEQYF

SEQ ID NO: 835 - protein, 11 aa, Homo sapiens
CASSQGVGYTF

SEQ ID NO: 836 - protein, 15 aa, Homo sapiens
CASSRSGAGTDTQYF

SEQ ID NO: 837 - protein, 11 aa, Homo sapiens
CASSPTYEQYF

SEQ ID NO: 838 - protein, 12 aa, Homo sapiens
CASSQVAGEQYF

SEQ ID NO: 839 - protein, 15 aa, Homo sapiens
CSATRGAGSTDTQYF

SEQ ID NO: 840 - protein, 14 aa, Homo sapiens
CASSESAGQETQYF

SEQ ID NO: 841 - protein, 13 aa, Homo sapiens
CASSSLGTGELFF

SEQ ID NO: 842 - protein, 14 aa, Homo sapiens
CSARDRANTGELFF

SEQ ID NO: 843 - protein, 15 aa, Homo sapiens
CASSQSGAGTDTQYF

SEQ ID NO: 844 - protein, 15 aa, Homo sapiens
CASSLGGVGNQPQHF

SEQ ID NO: 845 - protein, 11 aa, Homo sapiens
CASSQGVGYTF

SEQ ID NO: 846 - protein, 14 aa, Homo sapiens
CASSEGASNQPQHF

SEQ ID NO: 847 - protein, 11 aa, Homo sapiens
CASSQTYEQYF

SEQ ID NO: 848 - protein, 11 aa, Homo sapiens
CSARRGTEAFF

SEQ ID NO: 849 - protein, 14 aa, Homo sapiens
CSARRDSRTDTQYF

SEQ ID NO: 850 - protein, 14 aa, Homo sapiens
CSARRDSRTDTQYF

SEQ ID NO: 851 - protein, 13 aa, Homo sapiens
CSASDRVGYGYTF

SEQ ID NO: 852 - protein, 14 aa, Homo sapiens
CASSEGASNQPQHF

SEQ ID NO: 853 - protein, 14 aa, Homo sapiens
CSAKGQGSNQPQHF

SEQ ID NO: 854 - protein, 14 aa, Homo sapiens
CASSGTGLNTEAFF

SEQ ID NO: 855 - protein, 14 aa, Homo sapiens
CSARDRANTGELFF

SEQ ID NO: 856 - protein, 11 aa, Homo sapiens
CAWSTYYGYTF

SEQ ID NO: 857 - protein, 15 aa, Homo sapiens
CSATRDRRTYNEQFF

SEQ ID NO: 858 - protein, 11 aa, Homo sapiens
CASSSRREQFF

SEQ ID NO: 859 - protein, 11 aa, Homo sapiens
CASSPTYEQYF

SEQ ID NO: 860 - protein, 11 aa, Homo sapiens
CASSLRRGYTF

SEQ ID NO: 861 - protein, 15 aa, Homo sapiens
CASSLGLAGGDTQYF

SEQ ID NO: 862 - protein, 13 aa, Homo sapiens
CASSLDLANEQFF

SEQ ID NO: 863 - protein, 14 aa, Homo sapiens
CSARRDSRTDTQYF

SEQ ID NO: 864 - protein, 15 aa, Homo sapiens
CASSRSGGSTDTQYF

SEQ ID NO: 865 - protein, 14 aa, Homo sapiens
CSARRDSRTDTQYF

SEQ ID NO: 866 - protein, 13 aa, Homo sapiens
CASSWTGGGGYTF

SEQ ID NO: 867 - protein, 16 aa, Homo sapiens
CASSPRLAGATGELFF

SEQ ID NO: 868 - protein, 13 aa, Homo sapiens
CASSQGYNSPLHF

SEQ ID NO: 869 - protein, 14 aa, Homo sapiens
CASSGTGLNTEAFF

SEQ ID NO: 870 - protein, 14 aa, Homo sapiens
CSARRDSRTDTQYF

SEQ ID NO: 871 - protein, 14 aa, Homo sapiens
CSARRDSRGDTQYF

SEQ ID NO: 872 - protein, 11 aa, Homo sapiens
CSARRGTEAFF

SEQ ID NO: 873 - protein, 12 aa, Homo sapiens
CASSWGYNEQFF

SEQ ID NO: 874 - protein, 15 aa, Homo sapiens
CSASRQASSTDTQYF

SEQ ID NO: 875 - protein, 14 aa, Homo sapiens
CSARRDSRTDTQYF

SEQ ID NO: 876 - protein, 11 aa, Homo sapiens
CSARRGTEAFF

SEQ ID NO: 877 - protein, 14 aa, Homo sapiens
CSARRDSRTDTQYF

SEQ ID NO: 878 - protein, 14 aa, Homo sapiens
CSARRDSRTDTQYF

SEQ ID NO: 879 - protein, 14 aa, Homo sapiens
CSARRDSRTDTQYF

SEQ ID NO: 880 - protein, 14 aa, Homo sapiens
CASSEGASNQPQHF

SEQ ID NO: 881 - protein, 15 aa, Homo sapiens
CASSRSGGSTDTQYF

SEQ ID NO: 882 - protein, 17 aa, Homo sapiens
CSVVGLAGGSSYNEQFF

SEQ ID NO: 883 - protein, 16 aa, Homo sapiens
CASSLSVPSSGNTIYF

SEQ ID NO: 884 - protein, 13 aa, Homo sapiens
CASSLSGGETQYF

SEQ ID NO: 885 - protein, 14 aa, Homo sapiens
CSARRDSRTDTQYF

SEQ ID NO: 886 - protein, 15 aa, Homo sapiens
CSATRGAGGQETQYF

SEQ ID NO: 887 - protein, 15 aa, Homo sapiens
CASSLAGGSTDTQYF

SEQ ID NO: 888 - protein, 13 aa, Homo sapiens
CASSVVGNTEAFF

SEQ ID NO: 889 - protein, 15 aa, Homo sapiens
CASSRSGGSTDTQYF

SEQ ID NO: 890 - protein, 15 aa, Homo sapiens
CASSLSGTNTGELFF

SEQ ID NO: 891 - protein, 15 aa, Homo sapiens
CASSLWTGVSYEQYF

SEQ ID NO: 892 - protein, 13 aa, Homo sapiens
CASSPGQGYEQYF

SEQ ID NO: 893 - protein, 15 aa, Homo sapiens
CASSLAGGSTDTQYF

SEQ ID NO: 894 - protein, 14 aa, Homo sapiens
CASSVATGGYEQYF

SEQ ID NO: 895 - protein, 16 aa, Homo sapiens
CASSPRLAGGTGELFF

SEQ ID NO: 896 - protein, 11 aa, Homo sapiens
CSARRGTEAFF

SEQ ID NO: 897 - protein, 14 aa, Homo sapiens
CSARRDSRTDTQYF

SEQ ID NO: 898 - protein, 14 aa, Homo sapiens
CSVVSRGRTDTQYF

SEQ ID NO: 899 - protein, 15 aa, Homo sapiens
CASSRAGGSTDTQYF

SEQ ID NO: 900 - protein, 12 aa, Homo sapiens
CASSLVAEKLFF

SEQ ID NO: 901 - protein, 14 aa, Homo sapiens
CASSEGASNQPQHF

SEQ ID NO: 902 - protein, 14 aa, Homo sapiens
CASSPRTGWTEAFF

SEQ ID NO: 903 - protein, 14 aa, Homo sapiens
CASSEGASNQPQHF

SEQ ID NO: 904 - protein, 15 aa, Homo sapiens
CASSRAGGSTDTQYF

SEQ ID NO: 905 - protein, 14 aa, Homo sapiens
CSARRDSRTDTQYF

SEQ ID NO: 906 - protein, 15 aa, Homo sapiens
CASSRAGGSTDTQYF

SEQ ID NO: 907 - protein, 14 aa, Homo sapiens
CASSLVTGNSPLHF

SEQ ID NO: 908 - protein, 14 aa, Homo sapiens
CASSEGASNQPQHF

SEQ ID NO: 909 - protein, 15 aa, Homo sapiens
CASSRAGGSTDTQYF

SEQ ID NO: 910 - protein, 13 aa, Homo sapiens
CASSSEPNTEAFF

SEQ ID NO: 911 - protein, 13 aa, Homo sapiens
CASSLGSSYEQYF

SEQ ID NO: 912 - protein, 15 aa, Homo sapiens
CASSSDRSSYNEQFF

SEQ ID NO: 913 - protein, 11 aa, Homo sapiens
CSARRGTEAFF

SEQ ID NO: 914 - protein, 12 aa, Homo sapiens
CASSVDSYGYTF

SEQ ID NO: 915 - protein, 14 aa, Homo sapiens
CSARRDSRTDTQYF

SEQ ID NO: 916 - protein, 11 aa, Homo sapiens
CASSSTYEQYF

SEQ ID NO: 917 - protein, 12 aa, Homo sapiens
CASSLVSDTQYF

SEQ ID NO: 918 - protein, 14 aa, Homo sapiens
CASSLGQGGYEQYF

SEQ ID NO: 919 - protein, 14 aa, Homo sapiens
CASSEGSSNQPQHF

SEQ ID NO: 920 - protein, 13 aa, Homo sapiens
CASSVGQGDGYTF

SEQ ID NO: 921 - protein, 13 aa, Homo sapiens
CASSLAGGYEQYF

SEQ ID NO: 922 - protein, 16 aa, Homo sapiens
CASSLIGVSSYNEQFF

SEQ ID NO: 923 - protein, 12 aa, Homo sapiens
CASSPGADTQYF

SEQ ID NO: 924 - protein, 14 aa, Homo sapiens
CASSLGQGGYEQYF

SEQ ID NO: 925 - protein, 13 aa, Homo sapiens
CASSLGTGYGYTF

SEQ ID NO: 926 - protein, 12 aa, Homo sapiens
CASSPTAYEQYF

SEQ ID NO: 927 - protein, 13 aa, Homo sapiens
CASSYSRTGELFF

SEQ ID NO: 928 - protein, 11 aa, Homo sapiens
CASSLSGELFF

SEQ ID NO: 929 - protein, 16 aa, Homo sapiens
CASSLGPPPGTDTQYF

SEQ ID NO: 930 - protein, 12 aa, Homo sapiens
CASSLGTTEAFF

SEQ ID NO: 931 - protein, 13 aa, Homo sapiens
CAISESIGTEAFF

SEQ ID NO: 932 - protein, 13 aa, Homo sapiens
CASSVVSGDEQFF

SEQ ID NO: 933 - protein, 16 aa, Homo sapiens
CASSIGLAGAYNEQFF

SEQ ID NO: 934 - protein, 13 aa, Homo sapiens
CASSLLGSYEQYF

SEQ ID NO: 935 - DNA, 87 bp, Homo sapiens
ttggagatccagcgcacagagcagggggactcggccatgtatctctgtgccagcagcttaggaaccactgaagctttctttggacaa

SEQ ID NO: 936 - DNA, 87 bp, Homo sapiens
ttggagatccagcgcacagagcagggggactcggccatgtatctctgtgccagcagcttagggacaactgaagctttctttggacaa

SEQ ID NO: 937 - DNA, 87 bp, Homo sapiens
actctggagtccgctaccagctcccagacatctgtgtacttctgtgccatcagtgagtcgatagggactgaagctttctttggacaa

SEQ ID NO: 938 - DNA, 87 bp, Homo sapiens
aatgtgagcaccttggagctgggggactcggccctttatctttgcgccagcagcttgctagggtcctacgagcagtacttcgggccg

SEQ ID NO: 939 - DNA, 87 bp, Homo sapiens
tcggcccaaaagaacccgacagctttctatctctgtgccagtagtatcggactagcgggtgcctacaatgagcagttcttcgggcca

SEQ ID NO: 940 - DNA, 87 bp, Homo sapiens
aacctgagctctctggagctgggggactcagctttgtatttctgtgccagcagcgtggtgagtggggatgagcagttcttcgggcca

SEQ ID NO: 941 - protein, 9 aa, Homo sapiens
RLDKVEAEV

SEQ ID NO: 942 - protein, 9 aa, Homo sapiens
RLFRKSNLK

SEQ ID NO: 943 - DNA, 78 bp, Severe acute respiratory syndrome coronavirus 2; identical to SEQ ID NO: 89

SEQ ID NO: 944 - DNA, 66 bp, Severe acute respiratory syndrome coronavirus 2; identical to SEQ ID NO: 90

SEQ ID NO: 945 - DNA, 66 bp, Severe acute respiratory syndrome coronavirus 2; identical to SEQ ID NO: 91

SEQ ID NO: 946 - DNA, 132 bp, Severe acute respiratory syndrome coronavirus 2; identical to SEQ ID NO: 92

SEQ ID NO: 947 - DNA, 135 bp, Severe acute respiratory syndrome coronavirus 2; identical to SEQ ID NO: 93

SEQ ID NO: 948 - DNA, 57 bp, Severe acute respiratory syndrome coronavirus 2; identical to SEQ ID NO: 94

SEQ ID NO: 949 - DNA, 63 bp, Severe acute respiratory syndrome coronavirus 2; identical to SEQ ID NO: 95

SEQ ID NO: 950 - DNA, 75 bp, Severe acute respiratory syndrome coronavirus 2
tgtatagattgtttaggaagtctaatctcaaaccttttgagagagatatttcaactgaaatctatcaggccggta

SEQ ID NO: 951 - DNA, 105 bp, Severe acute respiratory syndrome coronavirus 2; identical to SEQ ID NO: 96

SEQ ID NO: 952 - DNA, 72 bp, Severe acute respiratory syndrome coronavirus 2; identical to SEQ ID NO: 97

SEQ ID NO: 953 - DNA, 87 bp, Severe acute respiratory syndrome coronavirus 2; identical to SEQ ID NO: 98

SEQ ID NO: 954 - DNA, 57 bp, Severe acute respiratory syndrome coronavirus 2; identical to SEQ ID NO: 99

SEQ ID NO: 955 - DNA, 108 bp, Severe acute respiratory syndrome coronavirus 2; identical to SEQ ID NO: 100

SEQ ID NO: 956 - DNA, 66 bp, Severe acute respiratory syndrome coronavirus 2; identical to SEQ ID NO: 101

SEQ ID NO: 957 - DNA, 174 bp, Severe acute respiratory syndrome coronavirus 2; identical to SEQ ID NO: 102

SEQ ID NO: 958 - DNA, 66 bp, Severe acute respiratory syndrome coronavirus 2; identical to SEQ ID NO: 103

SEQ ID NO: 959 - DNA, 57 bp, Severe acute respiratory syndrome coronavirus 2; identical to SEQ ID NO: 104

SEQ ID NO: 960 - DNA, 54 bp, Severe acute respiratory syndrome coronavirus 2
actagtactgatgtcgtatacagggcttttgacatctacaatgataaagtagct

SEQ ID NO: 961 - DNA, 51 bp, Severe acute respiratory syndrome coronavirus 2
atggcagacctcgtctatgctttaaggcattttgatgaaggtaattgtgac

SEQ ID NO: 962 - DNA, 75 bp, Severe acute respiratory syndrome coronavirus 2
gtgagaaaaatatttgttgatggtgttccatttgtagtttcaactggataccacttcagagagctaggtgttgta

SEQ ID NO: 963 - DNA, 63 bp, Severe acute respiratory syndrome coronavirus 2
atcgtcaacaacctagacaaatcagctggttttccatttaataaatggggtaaggctagactt

SEQ ID NO: 964 - DNA, 159 bp, Severe acute respiratory syndrome coronavirus 2
gcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgcc

SEQ ID NO: 965 - DNA, 87 bp, Severe acute respiratory syndrome coronavirus 2
ggttcactatatgttaaaccaggtggaacctcatcaggagatgccacaactgcttatgctaatagtgtttttaacatttgtcaagct

SEQ ID NO: 966 - DNA, 51 bp, Severe acute respiratory syndrome coronavirus 2
gtcacggccaatgttaatgcacttttatctactgatggtaacaaaattgcc

SEQ ID NO: 967 - DNA, 84 bp, Severe acute respiratory syndrome coronavirus 2
gagttttacgcatatttgcgtaaacatttctcaatgatgatactctctgacgatgctgttgtgtgtttcaatagcacttatgca

SEQ ID NO: 968 - DNA, 51 bp, Severe acute respiratory syndrome coronavirus 2
actaaaggacctcatgaattttgctctcaacatacaatgctagttaaacag

SEQ ID NO: 969 - DNA, 57 bp, Severe acute respiratory syndrome coronavirus 2
ccttacccagatccatcaagaatcctaggggccggctgttttgtagatgatatcgta

SEQ ID NO: 970 - DNA, 69 bp, Severe acute respiratory syndrome coronavirus 2
gtaaaaacagatggtacacttatgattgaacggttcgtgtctttagctatagatgcttacccacttact

SEQ ID NO: 971 - protein, 26 aa, Severe acute respiratory syndrome coronavirus 2
SKTQSLLIVNNATNVVIKVCEFQFCN

SEQ ID NO: 972 - protein, 22 aa, Severe acute respiratory syndrome coronavirus 2
REFVFKNIDGYFKIYSKHTPIN

SEQ ID NO: 973 - protein, 22 aa, Severe acute respiratory syndrome coronavirus 2
LEPLVDLPIGINITRFQTLLAL

SEQ ID NO: 974 - protein, 44 aa, Severe acute respiratory syndrome coronavirus 2; identical to SEQ ID NO: 197

SEQ ID NO: 975 - protein, 45 aa, Severe acute respiratory syndrome coronavirus 2; identical to SEQ ID NO: 198

SEQ ID NO: 976 - protein, 19 aa, Severe acute respiratory syndrome coronavirus 2
SPTKLNDLCFTNVYADSFV

SEQ ID NO: 977 - protein, 21 aa, Severe acute respiratory syndrome coronavirus 2
IADYNYKLPDDFTGCVIAWNS

SEQ ID NO: 978 - protein, 24 aa, Severe acute respiratory syndrome coronavirus 2
YRLFRKSNLKPFERDISTEIYQAG

SEQ ID NO: 979 - protein, 35 aa, Severe acute respiratory syndrome coronavirus 2; identical to SEQ ID NO: 201

SEQ ID NO: 980 - protein, 24 aa, Severe acute respiratory syndrome coronavirus 2
FLPFQQFGRDIADTTDAVRDPQTL

SEQ ID NO: 981 - protein, 29 aa, Severe acute respiratory syndrome coronavirus 2
ILDITPCSFGGVSVITPGTNTSNQVAVLY

SEQ ID NO: 982 - protein, 19 aa, Severe acute respiratory syndrome coronavirus 2
VNNSYECDIPIGAGICASY

SEQ ID NO: 983 - protein, 36 aa, Severe acute respiratory syndrome coronavirus 2; identical to SEQ ID NO: 205

SEQ ID NO: 984 - protein, 22 aa, Severe acute respiratory syndrome coronavirus 2
MQMAYRFNGIGVTQNVLYENQK

SEQ ID NO: 985 - protein, 58 aa, Severe acute respiratory syndrome coronavirus 2; identical to SEQ ID NO: 207

SEQ ID NO: 986 - protein, 22 aa, Severe acute respiratory syndrome coronavirus 2
QTYVTQQLIRAAEIRASANLAA

SEQ ID NO: 987 - protein, 19 aa, Severe acute respiratory syndrome coronavirus 2
KMSECVLGQSKRVDFCGKG

SEQ ID NO: 988 - protein, 18 aa, Severe acute respiratory syndrome coronavirus 2
TSTDVVYRAFDIYNDKVA

SEQ ID NO: 989 - protein, 17 aa, Severe acute respiratory syndrome coronavirus 2
MADLVYALRHFDEGNCD

SEQ ID NO: 990 - protein, 25 aa, Severe acute respiratory syndrome coronavirus 2
VRKIFVDGVPFVVSTGYHFRELGVV

SEQ ID NO: 991 - protein, 21 aa, Severe acute respiratory syndrome coronavirus 2
IVNNLDKSAGFPFNKWGKARL

SEQ ID NO: 992 - protein, 53 aa, Severe acute respiratory syndrome coronavirus 2
AYTKRNVIPTITQMNLKYAISAKNRARTVAGVSICSTMTNRQFHQKLLKSIAA

SEQ ID NO: 993 - protein, 29 aa, Severe acute respiratory syndrome coronavirus 2
GSLYVKPGGTSSGDATTAYANSVFNICQA

SEQ ID NO: 994 - protein, 17 aa, Severe acute respiratory syndrome coronavirus 2
VTANVNALLSTDGNKIA

SEQ ID NO: 995 - protein, 28 aa, Severe acute respiratory syndrome coronavirus 2
EFYAYLRKHFSMMILSDDAVVCFNSTYA

SEQ ID NO: 996 - protein, 17 aa, Severe acute respiratory syndrome coronavirus 2
TKGPHEFCSQHTMLVKQ

SEQ ID NO: 997 - protein, 19 aa, Severe acute respiratory syndrome coronavirus 2
PYPDPSRILGAGCFVDDIV

SEQ ID NO: 998 - protein, 23 aa, Severe acute respiratory syndrome coronavirus 2
VKTDGTLMIERFVSLAIDAYPLT

SEQ ID NO: 999 - protein, 26 aa, Severe acute respiratory syndrome coronavirus 2
SKTQSLLIVNNATNVVIKVCEFQFCN

SEQ ID NO: 1000 - protein, 22 aa, Severe acute respiratory syndrome coronavirus 2
REFVFKNIDGYFKIYSKHTPIN

SEQ ID NO: 1001 - protein, 22 aa, Severe acute respiratory syndrome coronavirus 2
LEPLVDLPIGINITRFQTLLAL

SEQ ID NO: 1002 - protein, 44 aa, Severe acute respiratory syndrome coronavirus 2; identical to SEQ ID NO: 197

SEQ ID NO: 1003 - protein, 45 aa, Severe acute respiratory syndrome coronavirus 2; identical to SEQ ID NO: 198

SEQ ID NO: 1004 - protein, 19 aa, Severe acute respiratory syndrome coronavirus 2
SPTKLNDLCFTNVYADSFV

SEQ ID NO: 1005 - protein, 21 aa, Severe acute respiratory syndrome coronavirus 2
IADYNYKLPDDFTGCVIAWNS

SEQ ID NO: 1006 - protein, 24 aa, Severe acute respiratory syndrome coronavirus 2
YRLFRKSNLKPFERDISTEIYQAG

SEQ ID NO: 1007 - protein, 35 aa, Severe acute respiratory syndrome coronavirus 2; identical to SEQ ID NO: 201

SEQ ID NO: 1008 - protein, 24 aa, Severe acute respiratory syndrome coronavirus 2
FLPFQQFGRDIADTTDAVRDPQTL

SEQ ID NO: 1009 - protein, 29 aa, Severe acute respiratory syndrome coronavirus 2
ILDITPCSFGGVSVITPGTNTSNQVAVLY

SEQ ID NO: 1010 - protein, 19 aa, Severe acute respiratory syndrome coronavirus 2
VNNSYECDIPIGAGICASY

SEQ ID NO: 1011 - protein, 36 aa, Severe acute respiratory syndrome coronavirus 2; identical to SEQ ID NO: 205

SEQ ID NO: 1012 - protein, 22 aa, Severe acute respiratory syndrome coronavirus 2
MQMAYRFNGIGVTQNVLYENQK

SEQ ID NO: 1013 - protein, 58 aa, Severe acute respiratory syndrome coronavirus 2; identical to SEQ ID NO: 207

SEQ ID NO: 1014 - protein, 22 aa, Severe acute respiratory syndrome coronavirus 2
QTYVTQQLIRAAEIRASANLAA

SEQ ID NO: 1015 - protein, 19 aa, Severe acute respiratory syndrome coronavirus 2
KMSECVLGQSKRVDFCGKG

SEQ ID NO: 1016 - protein, 18 aa, Severe acute respiratory syndrome coronavirus 2
TSTDVVYRAFDIYNDKVA

SEQ ID NO: 1017 - protein, 17 aa, Severe acute respiratory syndrome coronavirus 2
MADLVYALRHFDEGNCD

SEQ ID NO: 1018 - protein, 25 aa, Severe acute respiratory syndrome coronavirus 2
VRKIFVDGVPFVVSTGYHFRELGVV

SEQ ID NO: 1019 - protein, 21 aa, Severe acute respiratory syndrome coronavirus 2
IVNNLDKSAGFPFNKWGKARL

SEQ ID NO: 1020 - protein, 53 aa, Severe acute respiratory syndrome coronavirus 2; identical to SEQ ID NO: 992

SEQ ID NO: 1021 - protein, 29 aa, Severe acute respiratory syndrome coronavirus 2
GSLYVKPGGTSSGDATTAYANSVFNICQA

SEQ ID NO: 1022 - protein, 17 aa, Severe acute respiratory syndrome coronavirus 2
VTANVNALLSTDGNKIA

SEQ ID NO: 1023 - protein, 28 aa, Severe acute respiratory syndrome coronavirus 2
EFYAYLRKHFSMMILSDDAVVCFNSTYA

SEQ ID NO: 1024 - protein, 17 aa, Severe acute respiratory syndrome coronavirus 2
TKGPHEFCSQHTMLVKQ

SEQ ID NO: 1025 - protein, 19 aa, Severe acute respiratory syndrome coronavirus 2
PYPDPSRILGAGCFVDDIV

SEQ ID NO: 1026 - protein, 23 aa, Severe acute respiratory syndrome coronavirus 2
VKTDGTLMIERFVSLAIDAYPLT

(19) United States
(12) Patent Application Publication
(10) Pub. No.: US 2024/0269262 A1
(43) Pub. Date: Aug. 15, 2024

(54) METHODS, KITS, AND APPROACHES FOR VIRAL VACCINES

(71) Applicant: University of Florida Research Foundation, Incorporated, Gainesville, FL (US)

(72) Inventors: Duane Mitchell, Gainesville, FL (US); Hector Mendez-Gomez, Gainesville, FL (US); Changlin Yang, Gainesville, FL (US); Elias Sayour, Gainesville, FL (US); Paul Antonio Castillo Caro, Gainesville, FL (US); Elizabeth Ogando-Rivas, Gainesville, FL (US); Fernanda Pohl-Guimaraes, Gainesville, FL (US); Vrunda S. Trivedi, 223 Grinter Hall, FL (US)

(21) Appl. No.: 18/286,025

(22) PCT Filed: Apr. 7, 2022
(86) PCT No.: PCT/US2022/023772

§ 371 Date: Oct. 6, 2023

Related U.S. Application Data
(60) Provisional application No. 63/173,286, filed on Apr. 9, 2021.

(51) Int. Cl. A61K 39/215; C07K 14/005; C12N 9/12

(52) U.S. Cl. CPC A61K 39/215; C07K 14/005; C12N 9/127; C12N 2770/20022; C12N 2770/20034

(57) ABSTRACT

The invention provides methods of making vaccines against viruses, including against SARS-Cov-2. Such methods entail identifying areas of a viral genome that are highly conserved and making vaccines that target the highly conserved areas. The invention provides a polypeptide vaccine comprising a SARS-Cov-2 polypeptide or an immunogenic fragment thereof and a pharmaceutically acceptable excipient. The invention provides a polynucleotide vaccine comprising a polynucleotide encoding a SARS-Cov-2 polypeptide or immunogenic fragment thereof linked to a heterologous promoter and a pharmaceutically acceptable excipient. The invention provides methods for effecting prophylaxis of or treating SARS-Cov-2 infection comprising a step of administering a polypeptide vaccine and/or a polynucleotide vaccine to a subject in need thereof.

### CROSS REFERENCE TO RELATED APPLICATIONS

This application claims the benefit of U.S. Provisional Application 63/173.286 filed Apr. 9, 2021, which is incorporated by reference in its entirety for all purposes.

### REFERENCE TO A SEQUENCE LISTING

This application includes an electronic sequence listing in a file named 576514SEQLST.TXT, created on Apr. 4, 2022 and containing 286,125 bytes, which is hereby incorporated by reference in its entirety for all purposes.

### BACKGROUND OF THE INVENTION

Vaccines targeting SARS-Cov-2, the virus that causes COVID-19, have been remarkably effective (>90% effective) in reducing symptomatic illness and almost 100% effective in preventing hospitalization and death due to infection. However, due to mutations in the protein targeted by vaccines, the spike protein, the virus has shown ability to evolve mutations that significantly evade vaccine immunity, reduce the binding of vaccine-induced antibodies by as much as 10- to 20-fold, and increase the infectious potential of the virus. There is a need to generate second-generation SARS-Cov-2 vaccines that can protect against current and future variants and to develop an approach that can tackle the difficult task of keeping the world's population immune to the emerging variants of SARS-Cov-2 and responding to future pandemic threats.

Neutralizing antibodies are produced against surface proteins such as SARS-Cov-2 spike protein in SARS-Cov-2 infected individuals or in vaccinated individuals. T cells also participate in the immune response against SARS-Cov-2, and may mediate long-term protection against the virus (Kared H. et al. bioRxiv. 2020 Oct. 8; 2020.10.08.330688 Preprint). T-cell mediated responses to SARS-Cov-2 infection target epitopes from internal and/or non-structural virus proteins, as well as from structural proteins (Kared H. et al; Tarke A., et al. bioRxiv 2021 Mar. 1; 2021.02.27.433180 Preprint).

### BRIEF SUMMARY OF THE CLAIMED INVENTION

In one aspect, the invention provides a method for making a vaccine against a virus, the method comprising: (a) aligning genome sequences of at least two isolates, optionally, 1000, 10,000 or 100,000 isolates, of the virus; (b) identifying genomic areas with no or very little viral sequence variation at a nucleotide level; (c) identifying amino acid sequence areas with little or no sequence variation at an amino acid sequence level and aligning the identified amino acid sequence areas with the identified genomic areas; and (d) making a vaccine against the identified amino acid sequence areas with little or no sequence variation.

In some methods, the identified genomic and amino acid areas each constitute less than 10%, 5%, or 1% of the total genomic and amino acid areas. In some methods, the identified genomic and amino acid areas each have less than 10%, 5% or 1% of the mean variation in genomic and amino acid areas of the same length throughout the genome. In some methods, the virus is SARS-Cov-2. In some methods, the identified amino acid sequence area is any one of SEQ ID NOs: 1, 4-5, 111-320, 322, 941-942, and 971-1026. In some methods, the identified amino acid sequence area is SARS-Cov-2 RNA-dependent RNA polymerase (SEQ ID NO:1). In some methods, the identified amino acid sequence area is SARS-Cov-2 spike glycoprotein (SEQ ID NO:322).

In another aspect the invention provides a polypeptide vaccine comprising a SARS-Cov-2 polypeptide of any one of SEQ ID NOs: 1, 4-5, 111-320, 322, 941-942, and 971-1026 or an immunogenic fragment thereof and a pharmaceutically acceptable excipient. In some polypeptide vaccines, the SARS-Cov-2 polypeptide is a SARS-Cov-2 RNA-dependent RNA polymerase (SEQ ID NO:1) or an immunogenic fragment thereof. Some polypeptide vaccines comprise a SARS-Cov-2 polypeptide of any one of SEQ ID NOs: 1, 988-998, and 1016-1026 or an immunogenic fragment thereof and a pharmaceutically acceptable excipient. In some polypeptide vaccines, the SARS-Cov-2 polypeptide is a SARS-Cov-2 spike glycoprotein (SEQ ID NO:322) or an immunogenic fragment thereof. Some polypeptide vaccines comprise a SARS-Cov-2 polypeptide of any one of SEQ ID NOs:322, 941-942, 971-987, and 999-1015 or an immunogenic fragment thereof and a pharmaceutically acceptable excipient.

In some polypeptide vaccines, the SARS-Cov-2 polypeptide or fragment is linked to a heterologous carrier to help elicit and immune response. In some polypeptide vaccines, the pharmaceutically acceptable excipient is an adjuvant that helps elicit and immune response. In some polypeptide vaccines, the fragment has up to 10, 25, 50 or 100 contiguous amino acids of any one of SEQ ID NOs: 1, 4-5, 111-320, 322, 941-942, and 971-1026. In some polypeptide vaccines, the fragment has up to 10, 25, 50 or 100 contiguous amino acids of SEQ ID NO:1. In some polypeptide vaccines, the fragment has up to 10, 25, 50 or 100 contiguous amino acids of SEQ ID NO:322.

In another aspect, the invention provides a polynucleotide vaccine comprising a polynucleotide encoding a SARS-Cov-2 polypeptide of any one of SEQ ID NOs:1, 4-5, 111-320, 322, 941-942, and 971-1026 or immunogenic fragment thereof linked to a heterologous promoter and a pharmaceutically acceptable excipient. In some polynucleotide vaccines, the polynucleotide encodes a SARS-Cov-2 RNA-dependent RNA polymerase (SEQ ID NO:1) or immunogenic fragment thereof. In some polynucleotide vaccines, the polynucleotide encodes a SARS-Cov-2 spike glycoprotein (SEQ ID NO:322) or immunogenic fragment thereof.

Some polynucleotide vaccines are a DNA vaccine. Some polynucleotide vaccines are an RNA vaccine. In some polynucleotide vaccines, the SARS-Cov-2 polypeptide or fragment is linked to a heterologous carrier to help elicit an immune response. In some polynucleotide vaccines, the pharmaceutically acceptable excipient is an adjuvant that helps elicit an immune response.

In some polynucleotide vaccines, the fragment has up to 10, 25, 50 or 100 contiguous amino acids of any one of SEQ ID NOs: 1, 4-5, 111-320, 322, 941-942, and 971-1026. In some polynucleotide vaccines, the fragment has up to 10, 25, 50 or 100 contiguous amino acids of SEQ ID NO:1. In some polynucleotide vaccines, the fragment has up to 10, 25, 50 or 100 contiguous amino acids of SEQ ID NO:322. In some polynucleotide vaccines, the polynucleotide and heterologous promoter are components of a viral vector. In some polynucleotide vaccines, the polynucleotide and heterologous promoter are components of a bacterial vector or genome.

In another aspect, the invention provides a method for effecting prophylaxis of or treating SARS-Cov-2 infection comprising a step of administering any of the polypeptide vaccines and/or polynucleotide vaccines described herein.

### BRIEF DESCRIPTION OF THE DRAWINGS

FIG. 1 depicts SARS-Cov-2 RNA genome sequence variation for all mutations.

FIG. 2 depicts SARS-Cov-2 RNA genome sequence variation for nonsynonymous mutations.

FIGS. 3A-G is a table of cold spot SARS-Cov-2 genomic regions (names derived from NCBI Virus), with CDS_NT polynucleotide sequences, amino acid sequences translated from CDR_NT, and amino acid sequences Extracted from protein.

FIGS. 4A-B is a table of additional cold spot SARS-Cov-2 genomic regions (names derived from NIH Virus and GISAID), with CDS_NT polynucleotide sequences, amino acid sequences translated from CDR_NT, and amino acid sequences Extracted_from_protein.

FIG. 5A is a Simpson clonality boxplot representation of TCR beta clonotype sequences of Healthy donor (HD) or covid-19 recovered patients (RP)-expanded T cells with full-length spike (S) mRNA electroporated PBMCs collected at day 16 (2nd) and day 23 (3rd) after mRNA stimulation, and of baseline (UT) HD or RP PBMCs. FIGS. 5B, and 5C depict INF-gamma secretion by expanded T cells with full-length spike (S) mRNA electroporated PBMCs collected at day 16 (2nd) and day 23 (3rd), and by baseline (UT) HD or RP PBMCs, from healthy donors (HD) (FIG. 5B) and COVID-19 recovered patients (RP) (FIG. 5C), after 36 hr peptide stimulation with peptides spike S1, peptides spike S2, or control Actin peptides. 2nd, day 16 of stimulation; 3rd, day 23 of stimulation; HD, healthy donor; RP & Pt COVID-19 recovered patient; UT, untreated.

FIGS. 6A, 6B, 6C, and 6D are word cloud illustrations of HCDR3 region sequences from Healthy donor (HD) or covid-19 recovered patients (RP)-expanded T cells with full-length spike protein (S) mRNA electroporated PBMCs collected at day 16 and 23 after RNA stimulation, or from baseline (UT) HD or RP PBMCs, shown for HD at baseline (FIG. 5A), HD after day 16 and 23 after mRNA stimulation (FIG. 5B), RP at baseline (FIG. 5C), and RP after day 16 and 23 after mRNA stimulation (FIG. 5D). The number of TCR restricted hypomutated epitopes identified, after the mapping of HCDR3 sequences with Adaptive Biotechnologies ImmuneCODE database, are annotated in balloons. Healthy donor (n=13) and recovered patients (n=5).

FIG. 7 is a schematic showing determination of SARS-CoV2 hypomutated epitope from TCR profiling output, ImmuneCODE database analysis, and SARS-CoV2 hypomutated regions from FIG. 4.

### BRIEF DESCRIPTION OF THE SEQUENCES

SEQ ID NO: 1 sets forth the amino acid sequence of SARS-Cov-2 RNA-dependent RNA polymerase (Non-structural protein 12, nsp12) (GenBank Acc. NC_045512.2).

SEQ ID NO:2 sets forth the nucleotide sequence encoding SARS-Cov-2 RNA-dependent RNA polymerase (Non-structural protein 12, nsp12) (GenBank Acc. NC_045512.2).

SEQ ID NO:3 sets forth the nucleotide sequence of cold spot RNA-dependent_RNA_polymerase_sp|P0DTD1|R1AB_SARS2:6-932:CDS_NT.

SEQ ID NO:4 sets forth the amino acid sequence of RNA-dependent_RNA_polymerase_sp|P0DTD1|R1AB_SARS2:6-932: Translated from_CDS_NT.

SEQ ID NO:5 sets forth the amino acid sequence of Cold_spot_name: RNA-dependent_RNA_polymerase_sp|P0DTD1|R1AB_SARS2:6-932: Extracted_from protein.

SEQ ID NOs:6-110 set forth nucleotide sequences of cold spot regions identified in SARS-Cov-2 genomic sequences.

SEQ ID NOs: 111-215 set forth amino acid sequences translated from cold spot regions identified in SARS-Cov-2 genomic sequences (Translated_from_CDS_NT).

SEQ ID NOs:216-320 set forth amino acid sequences from cold spot regions identified in SARS-Cov-2 genomic sequences (Extracted_from_protein).

SEQ ID NO:321 sets forth the nucleotide sequence of a SARS-COV-2 (2019-nCOV) Spike ORF mammalian expression plasmid.

SEQ ID NO:322 sets forth the amino acid sequence of Spike glycoprotein (Swiss-Prot ID: P0DTC2) of SARS-COV-2 (Severe Acute Respiratory Syndrome-related coronavirus 2).

SEQ ID NO:323 sets forth the amino acid sequence of Actin, alpha skeletal muscle (Swiss-Prot ID: P68133) of Homo sapiens (Human).

SEQ ID NOs:324-484 set forth Untreated Healthy Donors' HCDR3 amino acid sequences.

SEQ ID NOs:485-555 set forth Stimulated Healthy Donors' HCDR3 amino acid sequences.

SEQ ID NOs:556-694 set forth Untreated Recovered Patients' HCDR3 amino acid sequences.

SEQ ID NOs:695-929 set forth Stimulated Recovered Patients' HCDR3 amino acid sequences.

SEQ ID NOs:930-934 set forth amino acid sequences of HCDR3 for TCR rearrangement (Table 5).

SEQ ID NOs:935-940 set forth nucleotide sequences of TCR rearrangement (Table 5).

SEQ ID NOs:941-942 set forth amino acid sequences of hypomutated epitopes from spike protein. (Table 5).

SEQ ID NOs: 943-970 set forth nucleotide sequences of additional cold spot regions identified in SARS-Cov-2 genomic sequences.

SEQ ID NOs:971-998 set forth amino acid sequences translated from additional cold spot regions identified in SARS-Cov-2 genomic sequences (Translated_from_CDS_NT).

SEQ ID NOs: 999-1026 set forth amino acid sequences from additional cold spot regions identified in SARS-Cov-2 genomic sequences (Extracted_from_protein).

### DEFINITIONS

The term “polypeptide” refers to a polymer of amino acids of any length including natural full length proteins, fragments and synthetic peptides. The term also include a polymer that has been modified, such as a polypeptide having a modified peptide backbone.

The term “polynucleotide” refers to a polymer of nucleotides of any length, including ribonucleotides, deoxyribonucleotides, or analogs or modified versions thereof. A polynucleotide may be single-, double-, and multi-stranded DNA or RNA, genomic DNA, cDNA, DNA-RNA hybrids, and a pol comprising purine bases, pyrimidine bases, or other natural, chemically modified, biochemically modified, non-natural, or derivatized nucleotide bases.

The term “fragment” when referring to a polypeptide means a polypeptide that is shorter or has fewer amino acids than the full-length polypeptide. The term “fragment” when referring to a polynucleotide means a polynucleotide that is shorter or has fewer nucleotides than the full-length polynucleotide. A fragment can be, for example, an N-terminal fragment (i.e., removal of a portion of the C-terminal end of the protein), a C-terminal fragment (i.e., removal of a portion of the N-terminal end of the protein), or an internal fragment. A fragment can also be, for example, a functional fragment or an immunogenic fragment.

The term “vaccine” refers to a pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a polynucleotide encoding a disclosed antigen), a polypeptide (such as a disclosed antigen), a virus, a cell or one or more cellular constituents.

The term “antibody” includes intact antibodies and binding fragments thereof. Typically, fragments compete with the intact antibody from which they were derived for specific binding to the target including separate heavy chains, light chains Fab, Fab′, F(ab′)₂, F(ab)c, Dabs, nanobodies, and Fv. Fragments can be produced by recombinant DNA techniques, or by enzymatic or chemical separation of intact immunoglobulins.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. See generally, Fundamental Immunology, Paul, W., ed., 2nd

Specific binding of an antibody to its target antigen means an affinity and/or avidity of at least 10⁶, 10⁷, 10⁸, 10⁹, 10¹⁰, 10¹¹, or 10¹² M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody binds one and only one target.

An “antigen” is an entity to which an antibody specifically binds.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

The term “pharmaceutically acceptable” means that the carrier, diluent, excipient, or auxiliary is compatible with the other ingredients of the formulation and not substantially deleterious to the recipient thereof.

The term “patient” includes human and other mammalian subjects that receive either prophylactic or therapeutic treatment.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease as perceived by the subject. A “sign” refers to objective evidence of a disease as observed by a physician.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally Ausubel et al., supra). One example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (NCBI) website. Typically, default program parameters can be used to perform the sequence comparison, although customized parameters can also be used. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89, 10915 (1989)).

For purposes of classifying amino acids substitutions as conservative or non-conservative, amino acids are grouped as follows: Group I (hydrophobic sidechains): norleucine, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Therapeutic agents of the invention are typically substantially pure from undesired contaminant. This means that an agent is typically at least about 50% w/w (weight/weight) purity, as well as being substantially free from interfering proteins and contaminants. Sometimes the agents are at least about 80% w/w and, more preferably at least 90 or about 95% w/w purity. However, using conventional protein purification techniques, homogeneous polypeptides of at least 99% w/w can be obtained.

The term “immunological” or “immune” response is the development of a beneficial humoral (antibody mediated) and/or a cellular (mediated by antigen-specific T cells or their secretion products) response directed against a polypeptide in a recipient patient. Such a response can be an active response induced by administration of immunogen or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays (see Burke, supra; Tigges, supra). The relative contributions of humoral and cellular responses to the protective or therapeutic effect of an immunogen can be distinguished by separately isolating antibodies and T-cells from an immunized syngeneic animal and measuring protective or therapeutic effect in a second subject.

An “immunogenic agent” or “immunogen” is capable of inducing an immunological response against itself on administration to a mammal, optionally in conjunction with an adjuvant.

The term “all-D” refers to polypeptides having ≥75%, ≥80%, ≥85%, ≥90%, ≥95%, and 100% D-configuration amino acids.

The term “naked polynucleotide” refers to a polynucleotide not complexed with colloidal materials. Naked polynucleotides are sometimes cloned in a plasmid vector.

The term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

Compositions or methods “comprising” or “including” one or more recited elements may include other elements not specifically recited. For example, a composition that “comprises” or “includes” a polypeptide fragment may contain the polypeptide fragment alone or in combination with other ingredients. When the disclosure refers to a feature comprising specified elements, the disclosure should alternatively be understood as referring to the feature consisting essentially of or consisting of the specified elements.

Designation of a range of values includes all integers within or defining the range, and all subranges defined by integers within the range.

Unless otherwise apparent from the context, the term “about” encompasses insubstantial variations, such as values within a standard margin of error of measurement (e.g., SEM) of a stated value.

Statistical significance means p≤0.05.

The singular forms of the articles “a,” “an,” and “the” include plural references unless the context clearly dictates otherwise. For example, the term “a compound” or “at least one compound” can include a plurality of compounds, including mixtures thereof.

### DETAILED DESCRIPTION

### I. General

The invention provides a universal vaccine platform capable of inducing effective protective immunity against currently identified variants of SARS-Cov-2, as well as future variants of SARS-Cov-2. The invention also provides a platform technology approach capable of providing similar protection against other known and unknown viral threats to the human species.

Coronaviruses (and all viruses) have vulnerabilities, revealed by one or more spots in the viral genome where the virus cannot mutate and still maintain viability. Vaccines targeting these molecular vulnerabilities where the virus is ‘fixed’ in its genomic evolution are effective in eradicating the virus from the human population. SARS-Cov-2 possesses an immunologic vulnerability that can be effectively targeted for protective immunity against current and future viral mutants. Vaccines prepared against this spot or spots in the viral genome where the virus cannot mutate and still maintain viability are more effective than art vaccines which may become ineffective as viral variants in the targeted gene appear. Virus persists in the human population when vaccines are not effective towards emerging viral variants. Highly virulent strains of virus may emerge when virus persists in the human population.

### II. Identification of Vaccine Targets in Viral Genomes

Vaccine targets can be determined by analyzing sequences of viral genomes of a clinically significant virus, for example SARS-Cov-2. Exemplary viruses include Adenoviridae, Picornaviridae, Herpesviridae, Hepadnaviridae, Coronaviridae, Flaviviridae, Retroviridae, Orthomyxoviridae, Paramyxoviridae, Papovaviridae, Polyomavirus, Poxviridae, Rhabdoviridae, and Togaviridae. Genome sequences of multiple SARS-Cov-2 viral genomes are aligned. An exemplary SARS-Cov-2 viral genomic sequence is GenBank Acc. No. NC_045512.2. Other exemplary SARS-Cov-2 viral genomic sequences are provided in public databases such as GenBank. GenBank identifiers for exemplary SARS-Cov-2 viral genomic sequences are in Example 2. Genomic sequences of at least two viral isolates can be aligned, optionally 1000, 10,000 or 100,000 isolates. Areas that show no viral sequence variation or very little evidence of variation are identified as viral “cold spots” where viral survival is fixed at these regions. Amino acid sequences of multiple SARS-Cov-2 viral isolates are aligned, and areas where the protein sequence also does not change between the viral isolates are identified and aligned with genomic “cold spots.” The identified genomic and amino acid areas can each constitute, for example, less than 10%, 5%, or 1% of the total genomic and amino acid areas. The identified genomic and amino acid areas can each have, for example, less than 10%, 5% or 1% of the mean variation in genomic and amino acid areas of the same length throughout the genome.

Areas that cannot mutate or change are identified as vaccine targets useful against current and future viral strains as areas are predicted to be conserved in both current and future viral strains. Prior infectious disease vaccines, including the current SARS-Cov-2 vaccines rely on generation of neutralizing antibodies. The vaccines of the invention directed toward highly conserved regions of the virus generate immunity against fixed viral vulnerabilities regardless of whether immunity produces neutralizing antibodies or not.

After administering the vaccines of the invention to a human or non-human subject, a protective immune response is induced, which is broadly effective against multiple virus variants.

### III. Vaccines

A human or non-human animal (for example, mouse, guinea pig, primate, rabbit or rat) can be vaccinated by immunizing with a full length viral polypeptide identified as encoded by a “cold spot” region or fragment thereof. Such an immunogen can be obtained from a natural source, by peptide synthesis, or by recombinant expression. Optionally, the immunogen can be administered fused or otherwise complexed with a carrier protein. Optionally, the immunogen can be administered with an adjuvant. Polypeptides for use as immunogens are preferably attached to a heterologous conjugate molecule that helps elicit an antibody response to the polypeptide. Attachment can be direct or via a spacer peptide or amino acid. Cysteine is used as a spacer amino acid because its free SH group facilitates attachment of a carrier molecule. A polyglycine linker (e.g., 2-6 glycines), with or without a cysteine residue between the glycines and the polypeptide can also be used. The carrier molecule serves to provide a T-cell epitope that helps elicit an antibody response against the polypeptide. Several carriers are commonly used particularly keyhole limpet hemocyanin (KLH), ovalbumin and bovine serum albumin (BSA). Peptide spacers can be added to polypeptide immunogen as part of solid phase peptide synthesis. Carriers are typically added by chemical cross-linking. Some examples of chemical crosslinkers that can be used include cross-N-maleimido-6-aminocaproyl ester or m-maleimidobenzoyl-N-hydroxysuccinimide ester (MBS) (see for example, Harlow, E. et al., Antibodies: A Laboratory Manual, Cold Spring Harbor Laboratory Press, Cold Spring Harbor, N.Y. 1988; Sinigaglia et al., Nature, 336:778-780 (1988); Chicz et al., J. Exp. Med., 178:27-47 (1993); Hammer et al., Cell 74:197-203 (1993); Falk K. et al., Immunogenetics, 39:230-242 (1994); WO 98/23635; and, Southwood et al. J. Immunology, 160:3363-3373 (1998)). The carrier and spacer if present can be attached to either end of the immunogen. Several types of adjuvant can be used as described below. Complete Freund's adjuvant followed by incomplete adjuvant is preferred for immunization of laboratory animals. Antibodies are screened for specific binding to a full length viral polypeptide identified as encoded by a “cold spot” region or fragment thereof. Binding can be assessed, for example, by Western blot, FACS or ELISA.

The heterologous carrier and adjuvant may be selected for pharmaceutical suitability for use in humans. Suitable carriers include serum albumins, keyhole limpet hemocyanin, immunoglobulin molecules, thyroglobulin, ovalbumin, tetanus toxoid, or a toxoid from other pathogenic bacteria, such as diphtheria (e.g., CRM197), E. coli, cholera, or H. pylori, or an attenuated toxin derivative. T cell epitopes are also suitable carrier molecules. Some conjugates can be formed by linking agents of the invention to an immunostimulatory polymer molecule (e.g., tripalmitoyl-S-glycerine cysteine (Pam₃Cys), mannan (a mannose polymer), or glucan (a β 1→2 polymer)), cytokines (e.g., IL-1, IL-1 alpha and β peptides, IL-2, γ-INF, IL-10, GM-CSF), and chemokines (e.g., MIP1-α and β, and RANTES). Immunogens may be linked to the carriers with or without spacers amino acids (e.g., gly-gly). Additional carriers include virus-like particles. Virus-like particles (VLPs), also called pseudovirions or virus-derived particles, represent subunit structures composed of multiple copies of a viral capsid and/or envelope protein capable of self-assembly into VLPs of defined spherical symmetry in vivo. (Powilleit, et al., (2007) PLOS ONE 2(5):e415.) Alternatively, polypeptide immunogens can be linked to at least one artificial T-cell epitope capable of binding a large proportion of MHC Class II molecules., such as the pan DR epitope (“PADRE”). PADRE is described in US 5,736,142, WO 95/07707, and Alexander J et al, Immunity, 1:751-761 (1994). Active immunogens can be presented in multimeric form in which multiple copies of an immunogen and/or its carrier are presented as a single covalent molecule.

Fragments are often administered with pharmaceutically acceptable adjuvants. The adjuvant increases the titer of induced antibodies and/or the binding affinity of induced antibodies relative to the situation if the polypeptide were used alone. A variety of adjuvants can be used in combination with a viral polypeptide or fragment thereof identified as encoded by a “cold spot” region to elicit an immune response. Preferred adjuvants augment the intrinsic response to an immunogen without causing conformational changes in the immunogen that affect the qualitative form of the response. Preferred adjuvants include aluminum salts, such as aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA; now Antigenics, Inc., New York, NY). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Ribi adjuvants are oil-in-water emulsions. Ribi contains a metabolizable oil (squalene) emulsified with saline containing Tween 80. Ribi also contains refined mycobacterial products which act as immunostimulants and bacterial monophosphoryl lipid A. Another adjuvant is CpG (WO 98/40100). Adjuvants can be administered as a component of a therapeutic composition with an immunogenic polypeptide or can be administered separately, before, concurrently with, or after administration of the immunogenic polypeptide.

Analogs of natural fragments of a full length viral polypeptide identified as encoded by a “cold spot” region that induce antibodies against SARS-Cov-2 can also be used. For example, one or more or all L-amino acids can be substituted with D amino acids in such polypeptides. Also the order of amino acids can be reversed (retro peptide). Optionally a polypeptide includes all D-amino acids in reverse order (retro-inverso peptide). Polypeptides and other compounds that do not necessarily have a significant amino acid sequence similarity with viral polypeptides or peptides but nevertheless serve as mimetics of viral polypeptides and induce a similar immune response.

Polypeptides (and optionally a carrier fused to the polypeptide) can also be administered in the form of a polynucleotide encoding the polypeptide and expressed in situ in a patient. Such polynucleotides can be DNA or RNA. A polynucleotide segment encoding an immunogen is typically linked to regulatory elements, such as a promoter and enhancer that allow expression of the DNA segment in the intended target cells of a patient. For expression in blood cells, as is desirable for induction of an immune response, promoter and enhancer elements from light or heavy chain immunoglobulin genes or the CMV major intermediate early promoter and enhancer are suitable to direct expression. The linked regulatory elements and coding sequences are often cloned into a vector.

The polynucleotide can be delivered in naked form (i.e., without colloidal or encapsulating materials). Alternatively, a number of viral vector systems can be used including retroviral systems (see, e.g., Lawrie and Tumin, Cur. Opin. Genet. Develop. 3, 102-109 (1993)); adenoviral vectors {see, e.g., Bett et al, J. Virol. 67, 591 1 (1993)) including retrovirus derived vectors such MMLV, HIV-1, and ALV; adeno-associated virus vectors {see, e.g., Zhou et al., J. Exp. Med. 179, 1867 (1994)), lentiviral vectors such as those based on HIV or FIV gag sequences, viral vectors from the pox family including vaccinia virus and the avian pox viruses, viral vectors from the alpha virus genus such as those derived from Sindbis and Semliki Forest Viruses (see, e.g., Dubensky et al., J. Virol. 70, 508-519 (1996)), Venezuelan equine encephalitis virus (see U.S. Pat. No. 5,643,576) and rhabdoviruses, such as vesicular stomatitis virus (see WO 96/34625) and papillomaviruses (Ohe et al., Human Gene Therapy 6, 325-333 (1995); Woo et al, WO 94/12629 and Xiao & Brandsma, Nucleic Acids. Res. 24, 2630-2622 (1996)).

In a further variation, an immunogenic polypeptide, such as a fragment of a viral polypeptide identified as encoded by a “cold spot” region, can be presented by bacteria as part of an immunogenic composition. A polynucleotide encoding the immunogenic polypeptide is incorporated into a genome or episome of the bacteria. Optionally, the polynucleotide is incorporated in such a manner that the immunogenic polypeptide is expressed as a secreted protein or as a fusion protein with a transmembrane protein of bacteria so that the polypeptide is displayed. Bacteria used in such methods should be nonpathogenic or attenuated. Suitable bacteria include Salmonella and Shigella.

A polynucleotide encoding an immunogen, or a vector containing the same, can be packaged into liposomes. Suitable lipids and related analogs are described by U.S. Pat. Nos. 5,208,036, 5,264,618, 5,279,833, and 5,283,185. Vectors, DNA, or RNA encoding an immunogen, can also be adsorbed to or associated with particulate carriers, examples of which include polymethyl methacrylate polymers and polylactides and poly(lactide-co-glycolides), (see, e.g., McGee et al., J. Micro Encap. 1996).

### IV. Pharmaceutical Compositions and Methods of Use

Patients amenable to treatment include individuals at risk of a virus infection, but not showing symptoms, as well as patients presently showing symptoms. Such virus infections include infection with SARS-Cov-2, Adenoviridae, Picornaviridae, Herpesviridae, Hepadnaviridae, Coronaviridae, Flaviviridae, Retroviridae, Orthomyxoviridae, Paramyxoviridae, Papovaviridae, Polyomavirus, Poxviridae, Rhabdoviridae, and Togaviridae. Optionally, presence or absence of symptoms, signs or risk factors of a disease is determined before beginning treatment.

In prophylactic applications, an agent for inducing an antibody or a pharmaceutical composition the same is administered to a patient susceptible to, or otherwise at risk of a disease (e.g., COVID-19) in regime (dose, frequency and route of administration) effective to reduce the risk, lessen the severity, or delay the onset of at least one sign or symptom of the disease. In therapeutic applications, an antibody or agent to induce an antibody is administered to a patient suspected of, or already suffering from a disease (e.g., COVID-19) in a regime (dose, frequency and route of administration) effective to ameliorate or at least inhibit further deterioration of at least one sign or symptom of the disease.

A regime is considered therapeutically or prophylactically effective if an individual treated patient achieves an outcome more favorable than the mean outcome in a control population of comparable patients not treated by methods of the invention, or if a more favorable outcome is demonstrated in treated patients versus control patients in a controlled clinical trial (e.g., a phase II, phase II/III or phase III trial) at the p<0.05 or 0.01 or even 0.001 level.

Effective doses of vary depending on many different factors, such as means of administration, target site, physiological state of the patient, whether the patient is human or an animal, other medications administered, and whether treatment is prophylactic or therapeutic.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen.

Effective amount of a vaccine or other agent that is sufficient to generate a desired response, such as reduce or eliminate a sign or symptom of a condition or disease. For instance, this can be the amount necessary to inhibit viral replication or to measurably alter outward symptoms of the viral infection. In general, this amount will be sufficient to measurably inhibit virus (for example, SARS-COV-2) replication or infectivity. When administered to a subject, a dosage will generally be used that will achieve target tissue concentrations that has been shown to achieve in vitro inhibition of viral replication. In some embodiments, an “effective amount” is one that treats (including prophylaxis) one or more symptoms and/or underlying causes of any of a disorder or disease, for example to treat a coronavirus infection. In some embodiments, an effective amount is a therapeutically effective amount. In some embodiments, an effective amount is an amount that prevents one or more signs or symptoms of a particular disease or condition from developing, such as one or more signs or symptoms associated with coronaviral infections.

The pharmaceutical compositions of the invention can be readily employed in a variety of therapeutic or prophylactic applications, e.g., for treating SARS-Cov-2 infection or eliciting an immune response to SARS-Cov-2 in a subject. In various embodiments, the vaccine compositions can be used for treating or preventing infections caused by a pathogen from which the displayed immunogenic polypeptide is derived. Thus, the vaccine compositions of the invention can be used in diverse clinical settings for treating or preventing infections caused by various viruses. As exemplification, a SARS-Cov-2 vaccine composition can be administered to a subject to induce an immune response to SARS-COV-2, e.g., to induce production of antibodies to the virus. For subjects at risk of developing an SARS-COV-2 infection, a vaccine composition of the invention can be administered to provide prophylactic protection against viral infection. Depending on the specific subject and conditions, pharmaceutical compositions of the invention can be administered to subjects by a variety of administration modes known to the person of ordinary skill in the art, for example, topical, intravenous, oral, subcutaneous, intraarterial, intra-articular, intracranial, intrathecal, intraperitoneal, intranasal, intraocular, parenteral, or intramuscular routes. A subcutaneous or intramuscular injection is most typically performed in the arm or leg muscles.

For prophylactic applications, the immunogenic composition is provided in advance of any symptom, for example in advance of infection. The prophylactic administration of the immunogenic compositions serves to prevent or ameliorate any subsequent infection. Thus, in some embodiments, a subject to be treated is one who has, or is at risk for developing, an infection (e.g., SARS-Cov-2 infection), for example because of exposure or the possibility of exposure to the virus (e.g., SARS-Cov-2). Following administration of a therapeutically effective amount of the disclosed therapeutic compositions, the subject can be monitored for an infection (e.g., SARS-Cov-2 infection), symptoms associated with an infection (e.g., SARS-Cov-2 infection), or both.

For therapeutic applications, the immunogenic composition is provided at or after the onset of a symptom of disease or infection, for example after development of a symptom of infection (e.g., SARS-Cov-2 infection), or after diagnosis of the infection. The immunogenic composition can thus be provided prior to the anticipated exposure to the virus so as to attenuate the anticipated severity, duration or extent of an infection and/or associated disease symptoms, after exposure or suspected exposure to the virus, or after the actual initiation of an infection. The pharmaceutical composition of the invention can be combined with other agents known in the art for treating or preventing infections by a relevant pathogen (e.g., SARS-Cov-2 infection).

### V. Kits

The invention further provides kits (e.g., containers) comprising polypeptide vaccines, polynucleotide vaccines disclosed herein and related materials, such as instructions for use (e.g., package insert). The instructions for use may contain, for example, instructions for administration of the vaccine and optionally one or more additional agents. The containers of antibody may be unit doses, bulk packages (e.g., multi-dose packages), or sub-unit doses.

Package insert refers to instructions customarily included in commercial packages of therapeutic products that contain information about the indications, usage, dosage, administration, contraindications and/or warnings concerning the use of such therapeutic products

Kits can also include a second container comprising a pharmaceutically-acceptable buffer, such as bacteriostatic water for injection (BWFI), phosphate-buffered saline, Ringer's solution and dextrose solution. It can also include other materials desirable from a commercial and user standpoint, including other buffers, diluents, filters, needles, and syringes.

### EXAMPLES

### Example 1: Identification of Cold Spot Regions in SARS-Cov-2 Genomic Sequences

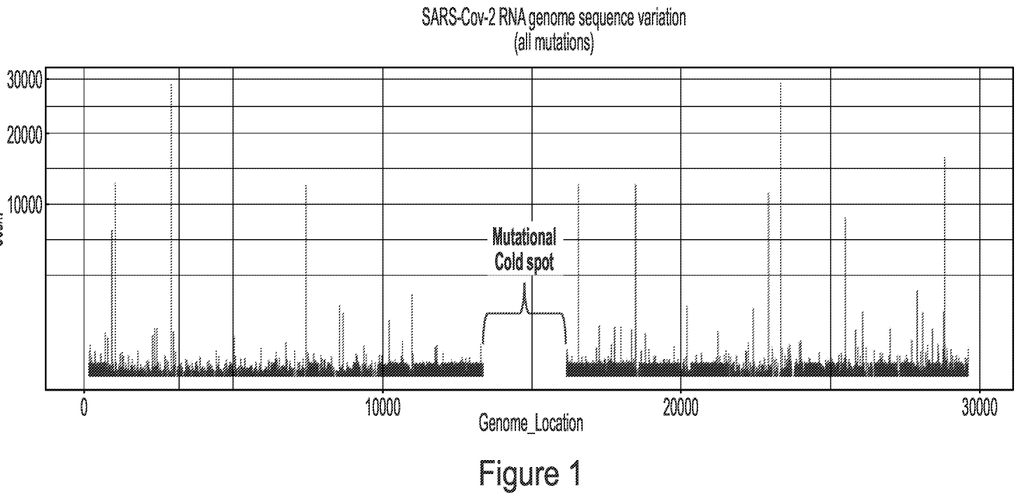

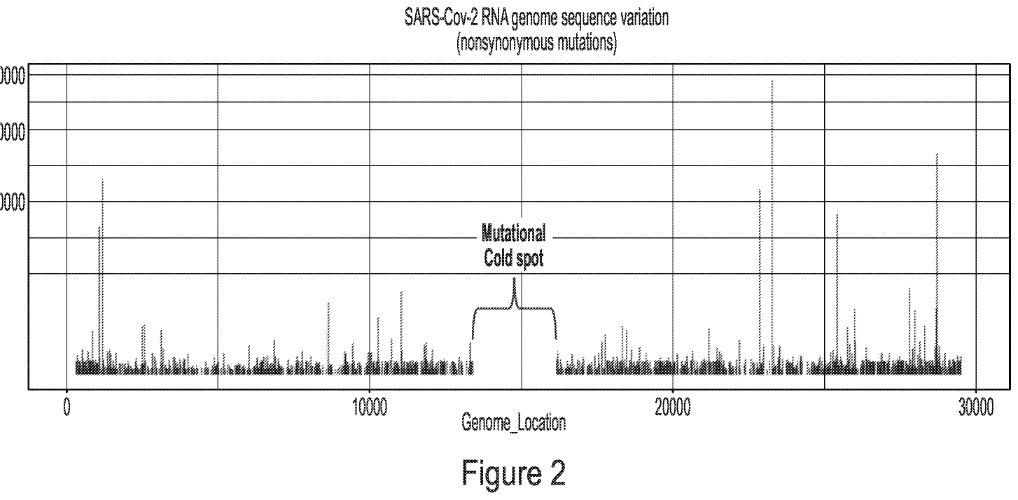

Sequence alignment was performed for 254,328 viral isolates of SARS-Cov-2 [WorldWideWeb ncbi.nlm.nih.gov/genbank/sars-cov-2-seqs/ (254,328 SRA runs); WorldWideWeb ncbi.nlm.nih.gov/sra/?term=txid2697049[Organism:noexp] NOT 0[Mbases]]. Nucleotide variation at each position of the viral genome was examined and plotted across all isolates. Analysis of synonymous mutations (does not change amino acid sequence) (FIG. 1) and nonsynonymous mutations (results in a change in amino acid sequence) (FIG. 2) were analyzed separately. WorldWideWeb_ncbi.nlm.nih.gov/labs/virus/vssi/#/scov2_snp was used for mutation call.

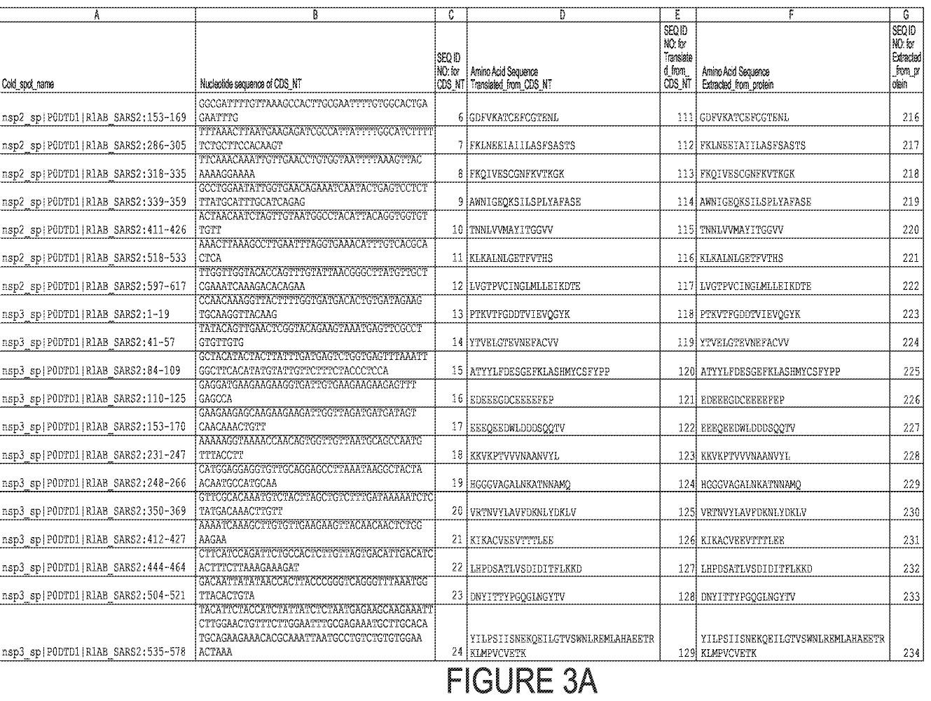

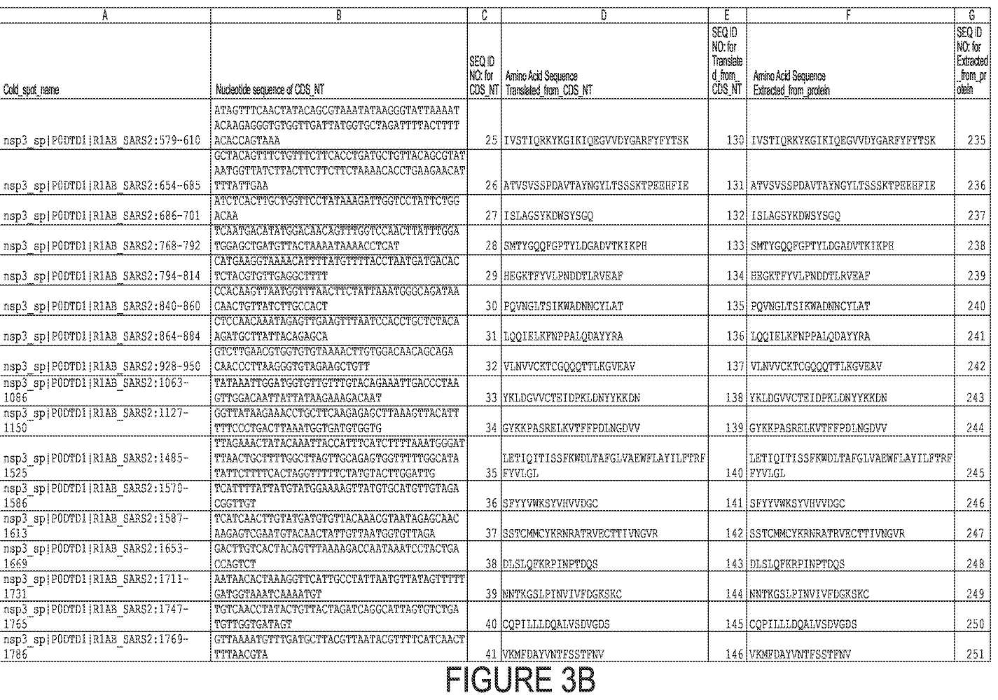

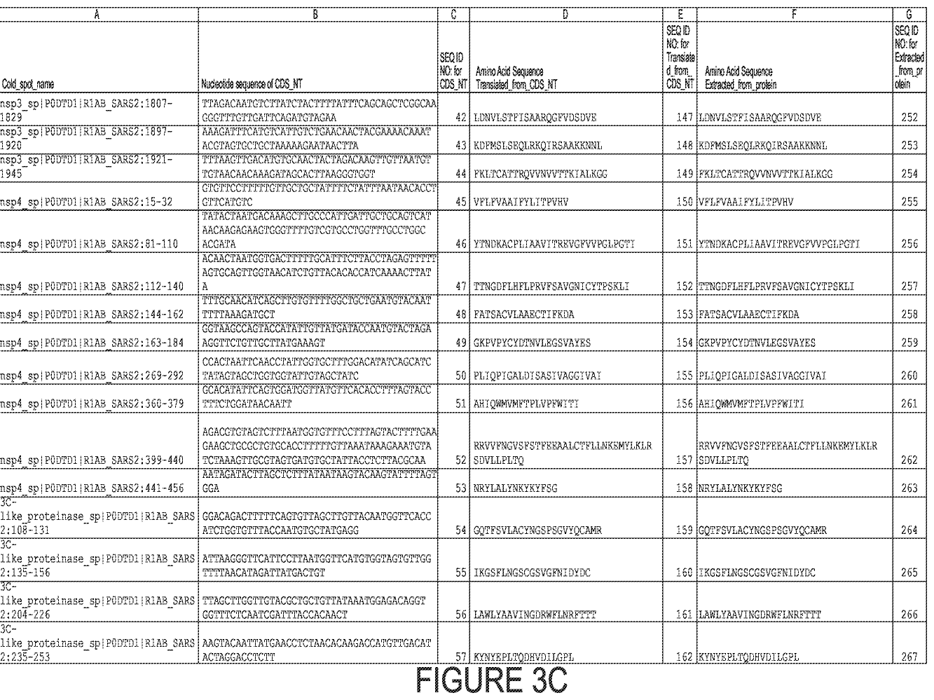

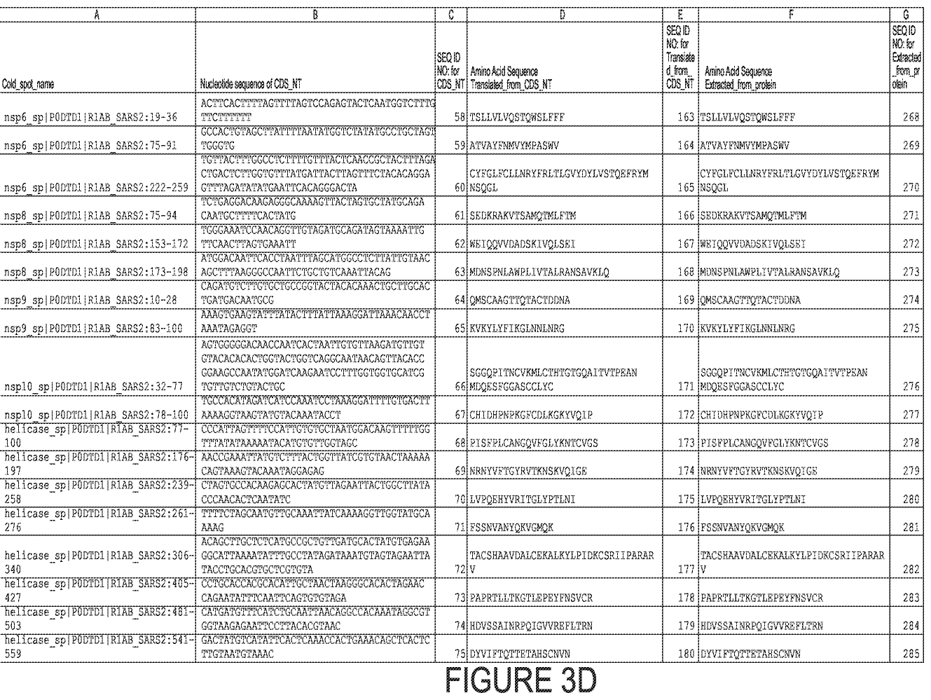

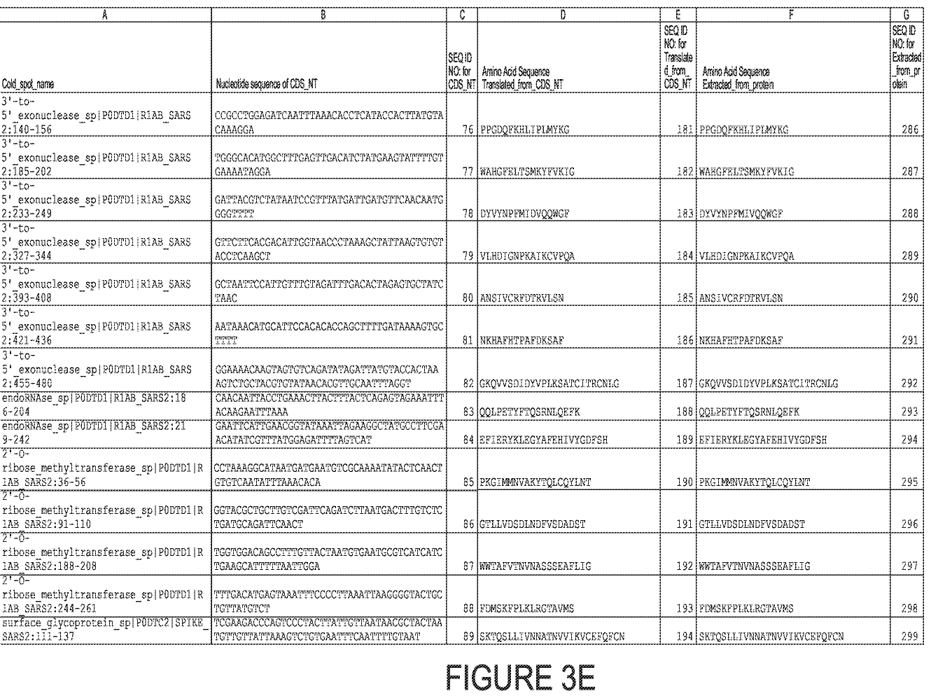

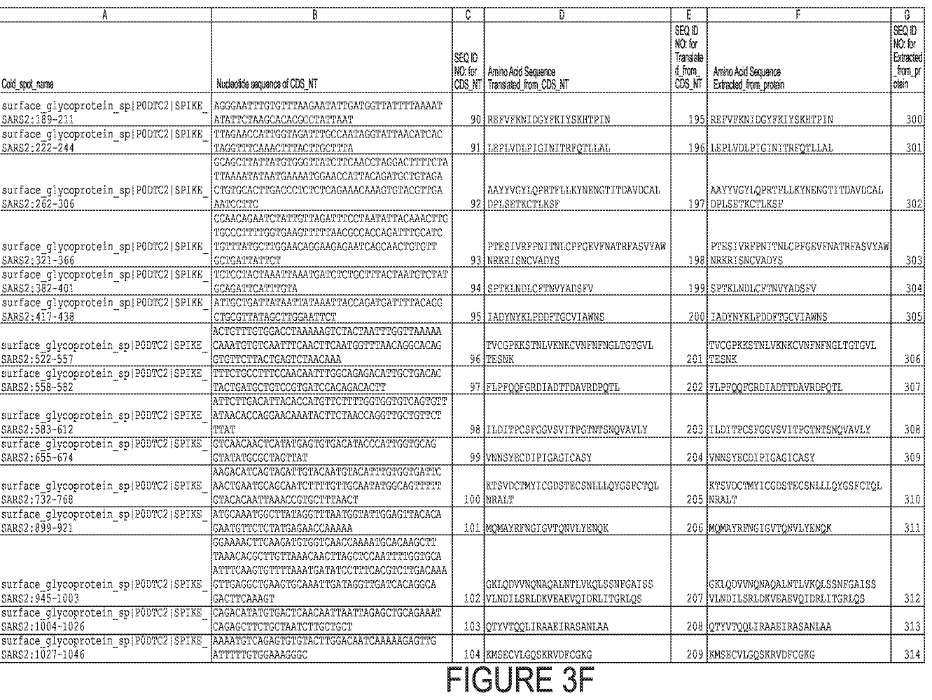

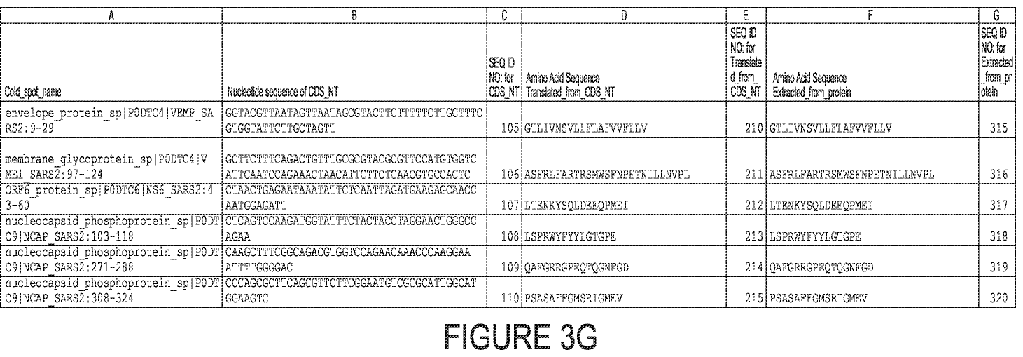

High intolerance of changes was found within 106 regions (cold spots) from NCBI Virus data. Name, polynucleotide sequence CDS_NT, amino acid sequence (Translated_from_CDS_NT), and amino acid sequence (Extracted_from_protein) for each of the 106 cold spot regions are depicted in FIGS. 3A-G and in SEQ ID NOs:3-5 (Cold_spot_name: RNA-dependent_RNA_polymerase_sp|P0DTD1|R1AB_SARS2:6-932). For example, in FIG. 3A, nsp2_sp|P0DTD1|R1AB_SARS2:153-169, regions are 153-169 AA from the ORF1ab/R1AB protein.

Graphical display of genomic and amino acid variability across the SARS-Cov-2 viral genome revealed high intolerance of changes within the region of nucleotides 13,442 to 16,236 which encodes the viral RNA-dependent RNA polymerase (RDRP) or Non-structural protein 12 (nsp12). The amino acid sequence of nsp12 is SEQ ID NO: 1 (GenBank Acc. No. NC_045512.). A nucleotide sequence encoding nsp12 is SEQ ID NO:2 (GenBank Acc. No. NC_045512.). Genomic and immunologic analysis of RDRP compared to all proteins of the human genome and peptidome reveals no overlapping sequences, homologies, or cross-reactive peptides with the human species.

### Example 2: Identification of Additional Cold Spot Regions in SARS-Cov-2 Genomic Sequences

SARS-CoV2 protein sequences of COVID-19 patients were downloaded from the NIH Virus and GISAID (Global Initiative on Sharing Avian Influenza Data) websites on Jul. 12, 2021. The mutations were identified by a customized script, which compared COVID-19 patients protein sequence with SARS_CoV2 isolated Wuhan-Hu-1. The frequency of the point mutations was calculated. The hypomutated regions were defined between two hypermutations (mutations frequency equal to or over than 100). The hypomutated regions are defined as the regions between two hypermutations regions (considering 100 or more patients harbored certain mutations). For example, if mutations were found in a Spike protein between the positions 1 and 16, and they are detected equally or more than in 100 patients, then the region between the Spike protein positions 2 to 15 is defined as a hypomutated region.

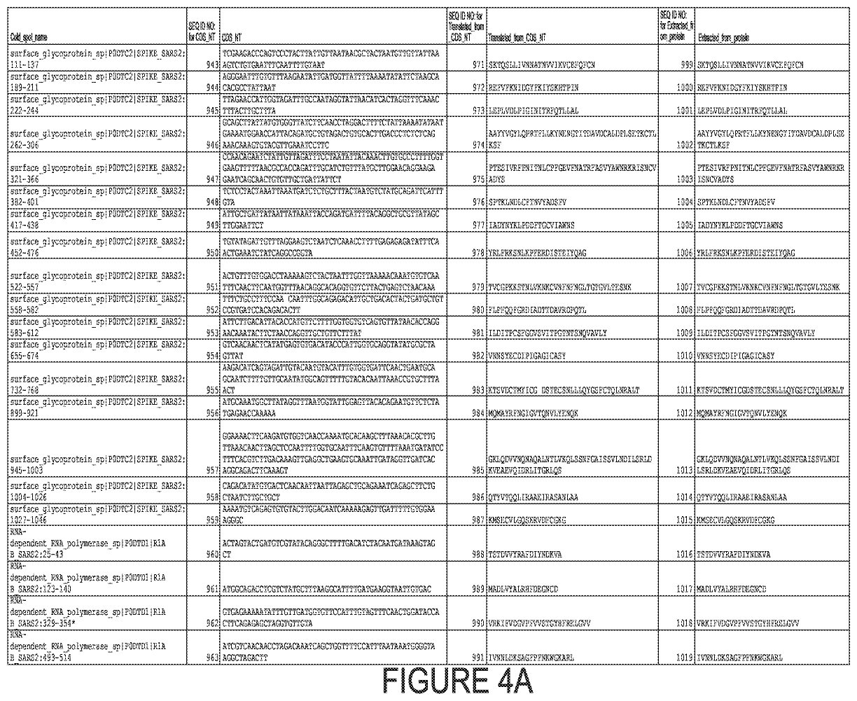

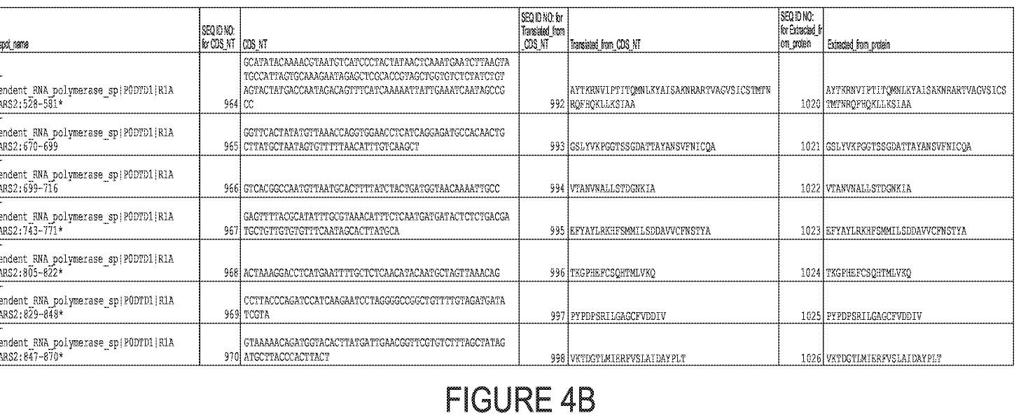

High intolerance of changes was found within 28 regions (cold spots) from NIH Virus and GISAID data. Name, polynucleotide sequence CDS_NT, amino acid sequence (Translated_from_CDS_NT), and amino acid sequence (Extracted_from_protein) for each of the 28 cold spot regions are depicted in FIGS. 4A-B. For example, in FIG. 4A, surface_glycoprotein_sp|P0DTC2|SPIKE_SARS2: 111-137, regions are 111-137 AA from the Spike glycoprotein.

### Example 3: Exemplary SARS-Cov-2 Viral Genomic Sequences

GenBank identifiers for exemplary SARS-Cov-2 viral genomic sequences are:

MT081067, MT614350, MT415833, MT598149, MT232872, MT729388,
MT503040, MW512913, MT324683, MT843231, MT415370, MT911468,
MW039602, MT798848, MT937009, MT598150, MT598152, MT598153,
MT263148, MT843238, MT936991, MT936992, MT936994, MT936999,
MT937001, MT937006, MT937008, MW281571, MT843232, MT936989,
MT936995, MT936998, 7ACT_B, MW441315, MT843235, MT843239,
MT937015, MW040511, MW040515, 7AAP_P, MW040520, MW040524,
MW040527, MW040525, MT163737, MT503009, MT502986, MT502991,
MT502995, MT502996, MT502999, MT503047, MT503057, MT502954,
MT503046, MT936990, MT503087, MT502945, MT503083, MT502947,
MT111896, MT503002, MT503003, MT502963, MT502988, MW040501,
MT503012, MT860463, MT502944, MT502949, MT502958, MT324682,
MT502913, MT081065, MT324681, MT502932, MW040523, MW040502,
MT875583, MT704954, MW039533, MT502990, MT186682, MT450426,
MT729366, MT042777, MT875590, MT503014, MT186677, MT676411,
MT457389, MW512911, MW512915, MT937020, MW055436, MT186676,
MT263143, MT263144, MT502925, 7B3B_P, 7B3C_P, MT503019,
MT729380, MT598148, MT937025, MT937010, MT937018, MT937021,
MT937011, MT937014, MW471658, MW368453, MW368452, MW368451,
MT232870, MT860462, MW040510, MW040512, MW040521, MT111895,
MW368460, MW368454, MW368456, MW368457, MT232871, MT127115,
6XRZ_A, MT359231, MW040514, MW113232, MT911470, MT729386,
MT614352, MW450960, 6X2G_F, MT502931, MT434818, MT503086,
MT503099, MT502998, MT415374, MT415375, MT503050, MW440437,
MT415376, MW440431, MT050415, MT503066, MT503068, MT502943,
MT502953, MW440434, MW440435, MW440440, MW440441, MT502983,
MT503016, MT344135, MT503010, MT503021, MT503025, MT503032,
MT503092, MT503094, MT503090, MT502957, MT502960, MT502970,
MT502985, MT503006, MT502946, MT502951, MT502971, MT502976,
MT502982, MT502993, MT502994, MT503013, MT503067, MT502972,
MT729372, MW292557, MW292549, MT903461, MW039449, MW455118,
MT502965, MT415373, MT724344, MT724343, MT729382, MT729362,
MT729379, MT415840, MT502935, MT856443, MT373161, MT875582,
MT614354, MW397519, MW397512, MT186679, MW512914, MT956635,
MT956636, MT903460, 7JYY_F, MT598137, MT598143, MT598147,
MT598155, MT937012, MT708233, MT050414, MT503060, MT186681,
MW110903, MW362749, MW362759, MW362755, MW362748, MT081063,
MT911469, MT373157, MW090853, MW090850, MW090904, MT415839,
MW015951, MT072668, MW560269, MW114446, MT127114, MT415834,
MT450427, MW362246, MT415836, MT415845, MT447189, MT415841,
MW113237, MT081068, MT503051, MW111539, MT042773, MW397520,
MT415843, MT415371, LC571018, MT796265, MT729378, MT729385,
MT081064, MW303957, 6XEZ_P, MT503035, MT614351, MW440432,
MT880778, MW521144, MW052550, MW165494, MT937019, MT937024,
LC523807, MT503024, 6XQB_E, MT373160, MW132940, MT503059,
MW548637, MW045453, MW045454, MW045459, MW291652, MW307301,
MW548607, MW548612, MT729361, MW292556, MW291946, MT724347,
MT502933, MT502934, MW548631, MW321848, MW321488, MW321591,
MT502923, MW320670, MW320680, MW320668, MT724345, MW320672,
MW320673, MT860465, MW321481, MW405788, MT598151, MW440442,
MW440439, MW440436, MT415367, MT415368, MT415369, MT415377,
MT415372, MW093140, MW397514, MW397517, MW397518, MW362757,
MW405787, MT502962, MT614353, MT729381, MT415844, MT903462,
MW093135, MW397513, MW362751, MW362754, MW362761, MW362758,
MT502910, MT450431, MW362750, MW362756, MW362760, MW362762,
MW362763, MW362752, MT911471, MT742647, MW362746, MW362747,
MW291651, MT903411, MW320721, 7B3D_T, MW362753, MW362764,
MT937003, MW063476, 7B3B_T, MT008023, MT503028, MT503034,
MT503061, MT503064, MT503071, MT503063, MT503097, MT503096,
MT503081, MW321435, MT503026, MT503039, MT503005, MT503055,
MT503095, MW015950, MT860726, MW321439, MT502942, MT503084,
MT503085, MT503093, MT503077, MT503079, MT503089, MT503073,
MT503074, MT503075, MT503076, MT373159, MT503058, MT503049,
MT503053, MT503022, MT503038, MT503023, MT503031, MT502975,
MT503027, MT503045, MT502989, MT502992, MT503018, MT503043,
MT503008, MT503044, MT373158, MT503001, MT503004, MT503011,
MT502940, MT502941, MT502948, MT502952, MT502956, MT502964,
MT502967, MT502968, MT502977, MT502980, MT502981, MT503033,
MT503037, MT503041, MW320685, MT872198, 7B3C_T, MW362225,
MW165492, MW165493, MW322566, MT544306, LC571026, MT127113,
MW548608, MT502984, 7JYY_E, MT856692, MT598135, MT598138,
MW405786, MW397516, MT308701, MT373163, MW111529, MW093139,
MT502961, MT503000, MT903463, MT503088, MT502900, MT273658,
MW321480, MT956634, MW320686, MT872190, MW320682, MT872199,
MW321595, MW321436, MT860464, MW320666, MT544614, MW114806,
MW113670, MW113679, MW362226, MW114306, MW362224, MT502927,
MW362227, MT880777, MT503036, MT502901, MW165496, MT066159,
MT503080, MW548638, MW548629, MW397515, LC571033, MW368449,
MT502907, MT042778, MT911467, MW368459, MW368455, MW512912,
MT544307, MT502922, MW368461, MW040516, MW368450, MW368458,
MT875587, MW040500, MT415846, MT066157, MW291693, 7B3D_P,
MW426075, MT502928, MT614355, MT192758, 7ACS_B, 7ACS_C,
MT008022, MT081059, MT503017, MW368765, MT450430, MT503082,
MT503091, MT502966, MT502973, MT502950, MT503015, MT503020,
MT503065, MT503070, MT502974, MT503078, MT503030, MW440438,
MT742648, MW015954, LC570964, MT163738, MT598142, MW281573,
MT860469, MT502906, MT066158, MT502936, MT729375, MW116727,
MT503048, MT263141, MW039576, MT502969, MT502955, MT502978,
MT503056, MW015949, MW517286, MW192920, MT081060, MT324679,
MT544615, MT161607, MT598134, MT502919, MT502924, MT502905,
MT187977, MT502987, MT163712, MT127116, MW114305, MW114664,
MW114660, MT856477, MT729383, MT425184, MW192921, MW136445,
MW127516, MW291959, MW045445, MW045461, MW045462, MW045468,
MW045470, MW045474, MT729368, MT903416, MT903455, MT425183,
MT502926, MW132908, LC523809, MT903406, MT875584, MW063479,
MT072667, MW045464, MW090866, MW090920, MW548639, MW548589,
MW548606, MW548611, MW548632, MW548636, MW548595, MW548609,
MW548633, MT415366, MT159778, MN938387, MN938389, MN975263,
MT539157, MW040519, MT598157, MT729377, MW032265, MT380727,
MT152900, MT712208, MT415835, MT042774, MT729370, MT502979,
MT729371, MT503098, MT350234, MT081066, MT729376, MT450429,
MT502930, MT502929, MN970004, MT293547, MT875588, MT081062,
MT502920, MT429168, MT263146, MT263147, MT263145, MT502939,
MW055437, MT450436, MT502915, MT598145, MT163714, MT598139,
MT450434, MT502916, MT875589, MT598133, MT450438, MT502911,
MT798849, MT875586, MT415838, MN975264, MN975265, MN975266,
MN938385, MN938386, MT050416, MT050417, MN938390, MN975267,
MN938388, MT875585, MT415842, MT703956, MT956633, MT502912,
MT598140, MT796264, MT502938, MW040503, MT324680, MT729364,
MT502902, MT380726, MT263142, MT042776, MT724348, MT872189,
MT872188, MW015952, MT503052, MT872187, MW090867, MW092768,
MW192919, MW090851, MW090852, MW090862, MW090871, LC571001,
MT729369, MW045452, MT502909, MT450428, MT502959, MT503069,
MW114523, MT415837, MT186678, MW136260, MW136261, MW136267,
MW136350, MW136351, MT186680, MT450435, MT450433, MW045463,
MW045471, MT502903, MW055258, MT843229, MT937007, MT937000,
MT502914, MT729373, MT072695, MT729363, MT729374, MW132931,
MW136352, MT843233, MT843236, MT843237, MT843240, MT936993,
MT936996, MT937004, MT937005, MT937016, MT843234, MT937022,
MT936997, MW281570, MW281572, 7JZ0_E, MT843230, MT598136,
MT598146, MT598156, MT598141, MT598144, MT937002, MT936988,
MT937013, MT937017, MT937023, MW320727, MW015953, MW320683,
MW320724, MT502904, MW320675, MW320671, MW320676, MT521348,
LC570966, MW320665, MT503042, MT373162, MT502997, MT373156,
MT503007, MW015947, MW321483, MW321496, MW322030, MW320677,
MW321438, MW321593, MT860461, MT503062, MT503072, MT724341,
MT724346, MT502908, MT502937, MT724342, MW015948, MW362247,
MT729367, MT232869, MT729365, MW015946, MT450432, MW362249,
MW362248, MN970003, 6XQB_F, 6XEZ_T, MW309428, 7JZ0_F, 7AAP_T,
MW301357, MW127502, MW127506, MW136446, MT729387, MT502918,
MW136262, MW301356, MT502917, MT450437, MT450425, MT544616,
MT163715, MT503029, MT729384, MW648379, MW648380, MW650650,
MW648381, MW646466, MW644687, MW642504, MW644688, MW644689,
MW642506, MW644690, MW672599, MW672618, MW672619, MW673635,
MW673636, MW673641, MW673642, MW673643, MW673644, MW673645,
MW673646, MW673648, MW673649, MW673650, MW673657, MW673637,
MW673640, MW673651, MW673652, MW673654, MW673655, MW672596,
LC571016, MW135333, LC571021, MW165491, MW165495, MW165497,
MW132896, MW626149, MW626150, MW626151, MW626152, MW626153,
MW626154, MW626155, MW626156, MW626157, MT502921, MW063481,
MW599251, MW599237, MW599954, MW599860, MW269404, MW269555,
MW617306, MW617312, MW617321, MW617324, MW617325, MW623400,
MW623409, MW623410, MW623418, MW623422, MW623424, MW623428,
MW623386, MW623387, MW623388, MW623389, MW623390, MW623395,
MW623396, MW623398, MW623402, MW623403, MW623405, MW623407,
MW623412, MW623414, MW623416, MW623421, MW624437, MW623397,
MW623399, MW623404, MW623406, MW623408, MW623413, MW623415,
MW623417, MW624436, MW623425, MW623426, MW623427, MW617298,
MW623369, MW623371, MW623372, MW623419, MW623420, MW623423,
MW623376, MW623377, MW623380, MW623381, MW623382, MW623383,
MW623384, MW623391, MW623393, MW623401, MW623411, MW623385,
MW623392, MW623394, MW624438, MW624439, MW617293, MW623370,
MW623373, MW623374, MW623375, MW623378, MW623379, LC571027,
MW132946, MW132928, MW084948, MW090854, MW090856, MW090859,
MW090900, MW055425, MW055435, MW090872, MW090902, MW090921,
MW055257, MW055259, MW045467, MW192918, MW090849, MW090857,
MW090863, MW090879, MW045477, MW090858, MW090877, MW063482,
MW161041, MW173090, MW173089, LC571015, MW063474, MW055255,
LC523808, LC522350, MW127501, MW192917, MW055367, MW055256,
MW127528, MW127548, MW126756, MW673638, MW673639, MW673647,
MW673653, MW672572, MW672602, MW672604, MW672605, MT447188,
LC571029, 6X2G_E, MN975268, MT042775, MT081061, MT503054,
MT544308, MT598154, MT708232, MW687138, MW687140, MW687139,
MW672525, MW709429, MW709393, LR991262, LR991263, LR991527,
LR991545, LR991546, LR991558, LR991175, LR991501, LR991502,
LR991503, LR991505, LR991509, LR991510, LR991511, LR991617,
LR991618, LR991619, LR991531, LR991535, LR991555, LR991194,
LR991195, LR991204, LR991205, LR991206, LR991524, LR991203,
LR991176, LR991202, LR991261, LR991526, LR991585, LR991569,
LR991570, LR991578, LR991579, LR991113, LR991148, LR991152,
LR991153, LR991155, LR991159, LR991160, LR991161, LR991164,
LR991165, LR991169, LR991171, LR991172, LR991199, LR991126,
LR991132, LR991137, LR991210, LR991150, LR991212, LR991216,
LR991158, LR991162, LR991226, LR991166, LR991229, LR991207,
LR991240, LR991209, LR991250, LR991251, LR991217, LR991542,
LR991220, LR991222, LR991223, LR991580, LR991227, LR991596,
LR991592, LR991231, LR991232, LR991238, LR991239, LR991536,
LR991537, LR991538, LR991539, LR991540, LR991541, LR991547,
LR991548, LR991549, LR991550, LR991551, LR991552, LR991559,
LR991560, LR991561, LR991562, LR991563, LR991568, LR991144,
LR991146, LR991211, LR991151, LR991213, LR991157, LR991219,
LR991225, LR991163, LR991228, LR991167, LR991233, LR991208,
LR991241, LR991214, LR991215, LR991256, LR991218, LR991553,
LR991221, LR991564, LR991566, LR991224, LR991230, LR991588,
LR991598, LR991605, LR991168, LR991170, LR991173, LR991174,
LR991200, LR991201, LR991593, LR991594, LR991595, LR991599,
LR991600, LR991601, LR991602, LR991603, LR991604, LR991397,
LR991406, LR991411, LR991416, LR991418, LR991419, LR991467,
LR991468, LR991472, LR991491, LR991493, LR991496, LR991470,
LR991485, LR991339, LR991488, LR991355, LR991370, LR991371,
LR991388, LR991389, LR991390, LR991399, LR991400, LR991407,
LR991412, LR991413, LR991414, LR991415, LR991417, LR991479,
LR991492, LR991474, LR991315, LR991490, LR991494, LR991495,
LR991497, LR991476, LR991316, LR991486, LR991489, LR991314,
LR991318, LR991319, LR991323, LR991336, LR991341, LR991342,
LR991343, LR991345, LR991351, LR991356, LR991357, LR991366,
LR991196, LR991197, LR991198, LR991571, LR991572, LR991573,
LR991575, LR991576, LR991317, LR991320, LR991322, LR991337,
LR991340, LR991346, LR991352, LR991354, LR991358, LR991359,
LR991361, LR991365, LR991498, LR963157, LR991177, LR991178,
LR991184, LR991280, LR991287, LR991186, LR991289, LR991290,
LR963149, LR963164, LR991525, LR963150, LR963152, LR963146,
LR963154, LR963158, LR963166, LR963167, LR963168, LR991244,
LR991254, LR991255, LR991190, LR991185, LR991288, LR991187,
LR991188, LR991265, LR991269, LR991272, LR991281, LR963318,
LR963337, LR991303, LR991307, LR963346, LR991278, LR991283,
LR991284, LR991286, LR991291, LR991293, LR991294, LR991179,
LR991180, LR991181, LR991182, LR991191, LR991277, LR991282,
LR991285, LR991292, LR963165, LR991296, LR991297, LR991312,
LR963360, LR963365, LR963366, LR991295, LR991299, LR991248,
LR991301, LR991304, LR991305, LR991306, LR991308, LR991309,
LR991298, LR991237, LR991300, LR991192, LR991193, LR991330,
LR991331, LR991334, LR991335, LR991321, LR991338, LR991353,
LR991582, LR991584, LR991506, LR991507, LR991508, LR991513,
LR991612, LR991613, LR991614, LR991615, LR991616, LR991620,
LR991621, LR991622, LR991623, LR991326, LR991327, LR991313,
LR991606, LR991607, LR991608, LR991609, LR991610, LR991611,
LR963142, LR963143, LR963144, LR963145, LR963160, LR963161,
LR963162, LR963163, LR963356, LR963362, LR963364, LR991243,
LR991252, LR991234, LR963355, LR963363, LR991235, LR991236,
LR991246, LR991247, LR991253, LR991259, LR991260, LR991543,
LR991554, LR991257, LR991258, LR991310, LR991311, MW699627,
MW699628, MW699629, MW699630, LR991328, LR878228, LR991372,
LR991374, LR991376, LR991378, LR991387, LR991391, LR991392,
LR991393, LR991395, LR991396, LR991408, LR991409, LR991410,
7BV2_P, 7BV2_T, 7CXM_I, 7CXM_J, 7CXM_L, 7CXN_J, 7CTT_Q, 7CTT_T
7CXN_L, 7CYQ_J, 7CYQ_I, 7CXN_I, MW732675, MW730759, MW730760,
MW730761, MW730765, MW730767, MW732667, MW732668, MW732669,
MW732670, MW732671, MW732672, MW732678, MW732679, MW732680,
MW732681, MW732676, MW730757, MW730758, MW730762, MW730763,
MW730764, MW730766, MW730768, LR963156, LR991333, LR991398,
LR899055, LR991332, LR991529, LR991944, LR991946, LR991947,
LR991951, LR991952, LR899040, LR899086, LR899099, LR899104,
LR899106, LR899044, LR899057, LR899068, LR899063, LR899105,
MW732673, MW732674, MW732677, LR991329, LR899064, LR899066,
LR899021, LR899039, LR899101, LR899080, LR877184, LR992037,
LR992033, LR899069, LR991500, LR991557, LR991577, LR991583,
LR991597, LR991945, LR991377, LR963147, LR963358, LR963359,
LR991183, LR991189, LR991302, LR991394, LR991473, LR991499,
LR991469, LR991556, LR991967, HG994305, HG994325, MW730769,
HG994186, HG994344, HG994263, HG994271, HG994335, MW672600,
MP929159, MW767175, MW237664, MW237663, MW693606, MW693918,
MW693927, MW693950, MW693951, MW693959, MW693964, MW693966,
MW693968, MW693976, MW693982, MW693986, MW693987, MW696571,
MW696573, MW696589, MW696590, MW696592, MW696597, MW696598,
MW696599, MW696600, MW696602, MW696616, MW696619, MW696626,
MW696628, MW696630, MW696638, MW696644, MW696654, MW696656,
MW693576, MW693550, MW693560, MW693605, MW693907, MW693909,
MW693571, MW693599, MW693602, MW693913, MW693931, MW693932,
MW693941, MW693945, MW693947, MW693952, MW693953, MW694001,
MW694011, MW693596, MW693908, MW693917, MW693926, MW693955,
MW693961, MW693967, MW693969, MW693975, MW693977, MW693983,
MW693984, MW693985, MW693988, MW693989, MW693990, MW693995,
MW694014, MW694015, MW694021, MW693996, MW694002, MW694003,
MW694004, MW694005, MW694006, MW694012, MW694013, MW693940,
MW693942, MW693946, MW693948, MW693949, MW693957, MW693958,
MW693978, MW693960, MW693970, MW693979, MW693980, MW694000,
MW646957, MW689161, MW689171, MW689185, MW689186, MW689187,
MW689688, MW689689, MW689694, MW689695, MW689696, MW689697,
MW689698, MW689702, MW689704, MW689705, MW689707, MW689711,
MW689713, MW689721, MW689723, MW689724, MW689731, MW689732,
MW689734, MW689736, MW689742, MW689744, MW689806, MW687317,
MW687336, MW687343, MW689179, MW689180, MW689181, MW689687,
MW689701, MW689703, MW689706, MW689708, MW689712, MW689714,
MW689715, MW689722, MW689725, MW689733, MW689735, MW689741,
MW689743, MW689805, MW421995, MW421996, MW422003, MW687485,
MW687493, MW687475, MW422014, MW687492, MW687504, MW422030,
MW681053, MW681054, MW681055, MW687245, MW687253, MW687254,
MW665756, MW665757, MW672754, MW672755, MW672756, MW672757,
MW672758, MW672759, MW672764, MW672765, MW672766, MW672767,
MW672774, MW672775, MW672776, MW672981, MW673025, MW673078,
MW673086, MW673087, MW673088, MW673093, MW673094, MW673095,
MW673096, MW673097, MW673102, MW673103, MW673104, MW673105,
MW673106, MW673113, MW673114, MW673115, MW673116, MW673123,
MW673124, MW673125, MW673126, MW673130, MW673131, MW673132,
MW673133, MW673134, MW673679, MW673705, MW674249, MW672982,
MW674258, MW672991, MW672997, MW672998, MW672999, MW673000,
MW673001, MW673008, MW673009, MW673010, MW673015, MW673019,
MW673021, MW673026, MW673027, MW673029, MW673030, MW673031,
MW673034, MW673038, MW673039, MW673040, MW673044, MW673046,
MW673047, MW673055, MW673056, MW665758, MW672353, MW672354,
MW672355, MW672356, MW672362, MW672363, MW672372, MW672373,
MW672374, MW672382, MW672383, MW673022, MW673032, MW673041,
MW673042, MW673048, MW673049, MW673050, MW673058, MW673059,
MW673065, MW673066, MW673067, MW673142, MW673143, MW673144,
MW673145, MW673152, MW673153, MW673154, MW673161, MW673162,
MW673163, MW673171, MW665735, MW665736, MW665737, MW665738,
MW673689, MW665744, MW673697, MW673698, MW673699, MW665748,
MW673707, MW665750, MW665751, MW665752, MW665753, MW665754,
MW674261, MW665760, MW673626, MW673627, MW673670, MW673677,
MW673683, MW673685, MW673686, MW673691, MW673701, MW673703,
MW674246, MW674247, MW674248, MW673576, MW673594, MW673595,
MW673596, MW673601, MW673603, MW673611, MW673617, MW673622,
MW673625, MW673673, MW673681, MW664351, MW664352, MW664359,
MW664360, MW664361, MW664362, MW664370, MW664371, MW664373,
MW673240, MW673249, MW673250, MW673256, MW673257, MW673261,
MW673262, MW673263, MW673264, MW673265, MW673269, MW673270,
MW673271, MW673272, MW673273, MW673274, MW673275, MW673279,
MW673280, MW673281, MW673282, MW673283, MW673284, MW673288,
MW673289, MW673290, MW673291, MW673292, MW673298, MW673299,
MW673300, MW673301, MW673308, MW673309, MW673310, MW673318,
MW673320, MW673328, MW673266, MW673267, MW673276, MW673277,
MW673285, MW673286, MW673287, MW673294, MW673295, MW673296,
MW673297, MW673302, MW673303, MW673304, MW673305, MW673306,
MW673307, MW673311, MW673312, MW673313, MW673314, MW673315,
MW673316, MW673321, MW673322, MW673323, MW673324, MW673325,
MW673326, MW673329, MW673330, MW673331, MW673332, MW673338,
MW673339, MW673340, MW673341, MW673348, MW673349, MW673523,
MW673524, MW673566, MW673575, MW673583, MW673592, MW673600,
MW673610, MW673618, MW673619, MW673629, MW665734, MW673172,
MW673181, MW673680, MW665743, MW673690, MW665745, MW665746,
MW665747, MW673706, MW665749, MW674251, MW674252, MW674259,
MW674260, MW665755, MW665761, MW665762, MW673675, MW673676,
MW673678, MW673682, MW673684, MW673687, MW673688, MW673692,
MW673693, MW673694, MW673695, MW673696, MW673702, MW673704,
MW674256, MW674257, MW665733, MW665742, MW673567, MW673585,
MW673586, MW673587, MW673602, MW673607, MW673614, MW673615,
MW673616, MW673621, MW673630, MW673633, MW673634, MW673669,
MW673674, MW672972, MW674250, MW672990, MW674266, MW672992,
MW673002, MW673011, MW673012, MW673016, MW673017, MW673018,
MW673020, MW673028, MW673035, MW673036, MW673037, MW673043,
MW673045, MW673053, MW673054, MW673057, MW673063, MW673064,
MW673141, MW673151, MW672813, MW672833, MW672842, MW672843,
MW672855, MW672856, MW672857, MW672863, MW672864, MW672865,
MW672866, MW672867, MW672870, MW672871, MW672873, MW672874,
MW672878, MW672881, MW672883, MW672885, MW672889, MW672890,
MW672900, MW672902, MW672903, MW672908, MW672911, MW672918,
MW672920, MW672929, MW672938, MW672789, MW672793, MW672801,
MW672802, MW672807, MW672808, MW672819, MW672821, MW672822,
MW672829, MW672839, MW672840, MW672853, MW672854, MW672872,
MW672875, MW672876, MW672879, MW672880, MW672882, MW672884,
MW672888, MW672891, MW672892, MW672893, MW672898, MW672899,
MW672901, MW672909, MW672910, MW672912, MW672919, MW672928,
MW672788, MW672790, MW672791, MW672792, MW672800, MW672803,
MW672806, MW672809, MW672810, MW672811, MW672820, MW672830,
MW672831, MW672832, MW672837, MW672838, MW672845, MW672847,
MW672858, MW672860, MW672862, MW672869, MW681251, MW681259,
MW681260, MW681261, MW681267, MW681268, MW681277, MW681278,
MW681279, MW681287, MW684568, MW684576, MW684577, MW684578,
MW684584, MW684585, MW684594, MW684595, MW684601, MW684602,
MW689813, MW689814, MW689820, MW689821, MW689822, MW689823,
MW689824, MW689830, MW689831, MW689832, MW689833, MW689840,
MW689841, MW689842, MW689848, MW689849, MW689850, MW689851,
MW689857, MW689858, MW689859, MW689860, MW689866, MW689867,
MW689868, MW689869, MW689876, MW689877, MW689878, MW689885,
MW680950, MW681252, MW681262, MW681269, MW681280, MW681288,
MW681290, MW681297, MW681364, MW684597, MW689834, MW689852,
MW689853, MW689861, MW689862, MW689863, MW689864, MW689870,
MW689872, MW689873, MW689880, MW689881, MW689887, MW689889,
MW689895, MW689896, MW689905, MW689907, MW689908, MW689915,
MW689919, MW681312, MW681313, MW681315, MW681322, MW681325,
MW681330, MW681331, MW681332, MW681333, MW681334, MW681343,
MW681382, MW681391, MW681416, MW681417, MW681426, MW681011,
MW681014, MW681015, MW681017, MW681021, MW681022, MW681023,
MW681024, MW681028, MW681029, MW681030, MW681031, MW681036,
MW681038, MW681039, MW681040, MW681041, MW681042, MW681047,
MW681048, MW681049, MW681050, MW681056, MW681057, MW681058,
MW681059, MW681067, MW681068, MW681069, MW681076, MW681078,
MW681086, MW681087, MW681351, MW681352, MW681353, MW681357,
MW681358, MW681359, MW681362, MW681363, MW689904, MW689912,
MW689914, MW692116, MW681007, MW681008, MW681012, MW681016,
MW681019, MW681020, MW681026, MW681027, MW681349, MW681350,
MW681354, MW689874, MW689890, MW689892, MW689901, MW689903,
MW689909, MW689910, MW689913, MW689917, MW689920, MW692113,
MW692115, MW692119, MW687483, MW687484, MW681393, MW681403,
MW681409, MW681410, MW681411, MW681412, MW681413, MW681419,
MW681420, MW681421, MW681422, MW681423, MW681427, MW681428,
MW681429, MW681430, MW681431, MW681432, MW681433, MW681437,
MW681438, MW681439, MW681440, MW681441, MW681442, MW681446,
MW681447, MW681448, MW681449, MW681450, MW681451, MW681454,
MW681455, MW681456, MW681457, MW681458, MW681459, MW681464,
MW681465, MW681466, MW681467, MW681468, MW681469, MW681474,
MW681475, MW681476, MW681477, MW681478, MW685641, MW685651,
MW681452, MW681460, MW681463, MW681472, MW681473, MW681479,
MW681488, MW681489, MW681490, MW681491, MW681508, MW681510,
MW681523, MW681533, MW681543, MW685644, MW685648, MW685652,
MW685653, MW685654, MW685656, MW681182, MW681183, MW681192,
MW681193, MW681201, MW681202, MW681203, MW681210, MW681211,
MW681212, MW681213, MW681218, MW681219, MW681220, MW681221,
MW681222, MW681223, MW681229, MW681230, MW681231, MW681232,
MW681233, MW681236, MW681237, MW681238, MW681239, MW681240,
MW681241, MW681244, MW681245, MW681246, MW681247, MW681248,
MW681249, MW681250, MW684208, MW684209, MW684210, MW684211,
MW684212, MW684218, MW684219, MW684220, MW684221, MW684228,
MW684229, MW684230, MW684231, MW684238, MW684239, MW681243,
MW681525, MW681528, MW681535, MW681536, MW681537, MW681544,
MW681546, MW684205, MW684213, MW684222, MW684232, MW684233,
MW684241, MW684249, MW684528, MW685650, MW681181, MW681217,
MW681228, MW684216, MW684217, MW684225, MW684226, MW684227,
MW684235, MW684236, MW684237, MW684243, MW684244, MW684245,
MW684246, MW684247, MW684250, MW684254, MW684255, MW684256,
MW684257, MW684509, MW684510, MW684512, MW684513, MW684514,
MW684515, MW684516, MW684517, MW684519, MW684520, MW684521,
MW684522, MW684523, MW684524, MW684529, MW684530, MW684531,
MW684532, MW684533, MW684539, MW684540, MW684541, MW684542,
MW684543, MW684549, MW684550, MW684551, MW684559, MW684560,
MW681257, MW681258, MW684511, MW684518, MW684526, MW684535,
MW684536, MW684552, MW684556, MW684558, MW684561, MW684562,
MW684564, MW684565, MW684566, MW684567, MW684572, MW684573,
MW684574, MW684575, MW684579, MW684580, MW684582, MW684589,
MW684591, MW684592, MW684593, MW681254, MW681255, MW681256,
MW681263, MW681264, MW681265, MW681266, MW681272, MW681273,
MW681274, MW681275, MW681276, MW681282, MW681283, MW681284,
MW681285, MW681286, MW681291, MW681292, MW681293, MW681294,
MW681295, MW681296, MW681299, MW681300, MW681301, MW681302,
MW681303, MW681304, MW681305, MW681307, MW681308, MW681309,
MW681310, MW681311, MW681317, MW681318, MW681319, MW681320,
MW681321, MW681327, MW681328, MW681329, MW681337, MW681338,
MW681347, MW681365, MW684588, MW684596, MW684598, MW681133,
MW681142, MW681143, MW681152, MW681153, MW681160, MW681161,
MW681162, MW681163, MW681167, MW681168, MW681169, MW681170,
MW681171, MW681172, MW681173, MW681175, MW681176, MW681177,
MW681178, MW681179, MW681180, MW681185, MW681186, MW681187,
MW681188, MW681189, MW681190, MW681191, MW681195, MW681196,
MW681197, MW681198, MW681199, MW681200, MW681204, MW681205,
MW681206, MW681207, MW681208, MW681209, MW681214, MW681215,
MW681216, MW681224, MW681225, MW681226, MW681227, MW681234,
MW681235, MW681063, MW681064, MW681065, MW681072, MW681073,
MW681074, MW681075, MW681082, MW681083, MW681090, MW681091,
MW681092, MW681093, MW681132, MW681135, MW681136, MW681137,
MW681138, MW681139, MW681140, MW681141, MW681144, MW681145,
MW681146, MW681147, MW681148, MW681149, MW681150, MW681151,
MW681154, MW681155, MW681156, MW681157, MW681158, MW681159,
MW681164, MW681165, MW681166, MW681174, MW681184, MW681194,
MW687585, MW689114, MW689124, MW689131, MW689690, MW689699,
MW689700, MW689709, MW689710, MW689716, MW689717, MW689718,
MW689719, MW689720, MW689726, MW689727, MW689728, MW689729,
MW689730, MW689737, MW689738, MW689739, MW689740, MW689800,
MW689801, MW689802, MW689803, MW689804, MW689807, MW689808,
MW689809, MW689810, MW689811, MW689812, MW689815, MW689816,
MW689817, MW689818, MW689819, MW689825, MW689826, MW689827,
MW689828, MW689829, MW689835, MW689836, MW689837, MW689838,
MW689845, MW689846, MW689855, MW689856, MW689865, MW681253,
MW681270, MW681271, MW681281, MW681289, MW681298, MW684586,
MW684587, MW689843, MW689844, MW689854, MW689871, MW689879,
MW689882, MW689886, MW689888, MW689897, MW689898, MW689906,
MW681306, MW681324, MW681339, MW681341, MW681366, MW681367,
MW681368, MW681369, MW681374, MW681375, MW681376, MW681383,
MW681384, MW681408, MW681424, MW681435, MW681445, MW672139,
MW672140, MW672141, MW672142, MW672143, MW672144, MW672145,
MW672150, MW672151, MW672152, MW672153, MW672154, MW672160,
MW672161, MW672162, MW672170, MW672171, MW672621, MW672631,
MW672632, MW685700, MW685708, MW685709, MW685717, MW685718,
MW685726, MW685727, MW685733, MW685734, MW685735, MW685741,
MW685742, MW685743, MW685744, MW685750, MW685751, MW685753,
MW685754, MW685760, MW685761, MW685762, MW685763, MW685764,
MW685769, MW685770, MW685771, MW686383, MW686384, MW686385,
MW686386, MT991088, MT991089, MW580244, MW672149, MW672156,
MW672158, MW672159, MW672163, MW672164, MW672166, MW672167,
MW672168, MW672169, MW672172, MW672174, MW672175, MW672176,
MW672617, MW672625, MW672627, MW674794, MW674798, MW674804,
MW674806, MW674807, MW674808, MW674814, MW674833, MW674853,
MW674820, MW674821, MW674822, MW674828, MW674829, MW674830,
MW674831, MW674832, MW674840, MW674841, MW674842, MW674847,
MW674848, MW674849, MW674850, MW674851, MW677609, MW677610,
MW677611, MW677612, MW677617, MW677618, MW677619, MW677620,
MW677621, MW677622, MW677626, MW677964, MW677965, MW677966,
MW677967, MW677968, MW677973, MW677974, MW677975, MW677976,
MW677977, MW680485, MW680486, MW680495, MW680496, MW684659,
MW684668, MW684677, MW684685, MW684705, MW685447, MW685448,
MW677613, MW677623, MW677969, MW677978, MW677979, MW680477,
MW680478, MW680479, MW680480, MW680482, MW680483, MW680487,
MW680489, MW680491, MW680492, MW680497, MW680500, MW680501,
MW680502, MW680506, MW680507, MW680509, MW680511, MW680515,
MW680516, MW680519, MW680526, MW680529, MW680530, MW680535,
MW680538, MW556283, MW672622, MW672623, MW672629, MW672639,
MW672640, MW672641, MW672650, MW672651, MW672660, MW672661,
MW672668, MW672669, MW672670, MW672675, MW672676, MW672677,
MW672678, MW672683, MW672684, MW672685, MW672686, MW672692,
MW672722, MW685701, MW685710, MW685711, MW685720, MW685721,
MW685728, MW685729, MW685730, MW672138, MW685737, MW672679,
MW685745, MW685746, MW672687, MW672688, MW685756, MW672690,
MW685758, MW672693, MW685766, MW672695, MW672696, MW672697,
MW672707, MW672712, MW672713, MW672714, MW672716, MW672717,
MW672723, MW672724, MW672725, MW672726, MW672727, MW672734,
MW672735, MW672742, MW672743, MW673070, MW685740, MW685749,
MW681497, MW681507, MW681521, MW681522, MW681529, MW681530,
MW681531, MW681532, MW681539, MW681540, MW681541, MW681542,
MW681547, MW681548, MW681549, MW681550, MW681551, MW681554,
MW681555, MW681556, MW681557, MW683336, MW684178, MW684179,
MW684182, MW684183, MW684184, MW684185, MW684186, MW684187,
MW684192, MW684193, MW684194, MW684195, MW684196, MW684197,
MW684202, MW684203, MW684204, MW684603, MW684604, MW684605,
MW684610, MW684611, MW684612, MW684613, MW684620, MW684621,
MW684622, MW684630, MW674862, MW674863, MW681552, MW684189,
MW684199, MW684607, MW684616, MW684623, MW684631, MW684633,
MW684640, MW684669, MW684688, MW684689, MW684696, MW684697,
MW684706, MW685444, MW685449, MW685451, MW685464, MW685466,
MW685604, MW685612, MW685614, MW685629, MW685630, MW685631,
MW685632, MW685633, MW685634, MW685639, MW685640, MW685657,
MW685658, MW685659, MW685676, MW685677, MW685678, MW685679,
MW685680, MW685681, MW685687, MW685688, MW685689, MW685690,
MW685691, MW685693, MW685694, MW685695, MW685696, MW685697,
MW685698, MW685699, MW685702, MW685703, MW685704, MW685705,
MW685706, MW685707, MW685712, MW685713, MW685714, MW685715,
MW685716, MW685722, MW685723, MW685724, MW685725, MW685732,
MW685752, MW556267, MW556268, MW556269, MW556274, MW556275,
MW556276, MW556284, MW556285, MW672624, MW672642, MW680484,
MW680493, MW680512, MW680520, MW680521, MW680539, MW680559,
MW680560, MW680567, MW680568, MW680569, MW680570, MW680577,
MW680578, MW680579, MW680580, MW680587, MW680588, MW680589,
MW680590, MW680595, MW680596, MW680597, MW680598, MW680599,
MW680604, MW680605, MW680606, MW680607, MW680608, MW680609,
MW680614, MW680615, MW680616, MW680617, MW680618, MW680619,
MW680621, MW680622, MW680623, MW556270, MW556271, MW556272,
MW556273, MW556277, MW556278, MW556279, MW556280, MW556281,
MW556282, MW556286, MW556287, MW556288, MW672565, MW672566,
MW672567, MW672633, MW672634, MW672635, MW672636, MW672637,
MW672638, MW672643, MW672644, MW672645, MW672646, MW672647,
MW672648, MW672649, MW672652, MW672653, MW672654, MW672655,
MW672656, MW672657, MW672658, MW672659, MW672662, MW672663,
MW672664, MW672665, MW672666, MW672667, MW672672, MW672673,
MW672674, MW672682, MW680600, MW680610, MW680620, MW672620,
MW674801, MW674802, MW674812, MW674815, MW674816, MW674817,
MW674818, MW674819, MW674824, MW674825, MW674826, MW674827,
MW674834, MW674835, MW674836, MW674837, MW674838, MW674839,
MW684680, MW674844, MW684682, MW674846, MW684684, MW684690,
MW684691, MW684692, MW684693, MW677616, MW677624, MW677625,
MW684700, MW677972, MW684702, MW684703, MW684704, MW685446,
MW684609, MW684617, MW684626, MW684628, MW684634, MW684635,
MW684642, MW684651, MW684652, MW685736, MW672671, MW685738,
MW672680, MW672681, MW685747, MW685748, MW685755, MW672689,
MW685757, MW672691, MW685765, MW672694, MW685767, MW685772,
MW685773, MW672702, MW672703, MW672704, MW672705, MW672715,
MW672732, MW672733, MW672736, MW673069, MW673071, MW673072,
MW685731, MW685759, MW685768, MW686007, MW684665, MW684666,
MW684667, MW684671, MW684672, MW684673, MW684674, MW684675,
MW684676, MW674843, MW684681, MW674845, MW684683, MW677559,
MW677560, MW677607, MW677608, MW677614, MW677615, MW684694,
MW684695, MW677971, MW684701, MW684190, MW684191, MW684201,
MW684608, MW684618, MW684627, MW664348, MW664349, MW664353,
MW664354, MW664355, MW664356, MW664357, MW664363, MW664364,
MW664365, MW673180, MW673190, MW673199, MW673200, MW673206,
MW673207, MW673208, MW673209, MW673210, MW673215, MW673216,
MW673217, MW673218, MW673219, MW673220, MW673224, MW673225,
MW673226, MW673227, MW673228, MW673229, MW673230, MW673233,
MW673234, MW673235, MW673236, MW673237, MW673238, MW673241,
MW673242, MW673243, MW673244, MW673245, MW673251, MW673252,
MW673253, MW673254, MW673255, MW673258, MW672752, MW672753,
MW672763, MW672769, MW672770, MW672772, MW672773, MW672779,
MW672780, MW672781, MW672784, MW672785, MW672794, MW672795,
MW672796, MW672804, MW672814, MW672817, MW672824, MW672825,
MW672826, MW672835, MW673098, MW673107, MW673117, MW673127,
MW673135, MW673247, MW672977, MW672978, MW672979, MW672980,
MW672986, MW672987, MW672988, MW672989, MW672993, MW672994,
MW672995, MW672996, MW673004, MW673005, MW673006, MW673007,
MW673013, MW673014, MW673023, MW673024, MW673033, MW673051,
MW673060, MW673061, MW673068, MW673137, MW673146, MW673147,
MW673155, MW673156, MW673164, MW673165, MW673173, MW673174,
MW673182, MW673183, MW673478, MW673487, MW673488, MW673497,
MW673498, MW673505, MW673506, MW673507, MW673508, MW673513,
MW673514, MW673515, MW673516, MW673517, MW673062, MW673149,
MW673150, MW673159, MW673160, MW673167, MW673175, MW673176,
MW673178, MW673179, MW673184, MW673186, MW673188, MW673192,
MW673193, MW673194, MW673195, MW673196, MW673197, MW673198,
MW673453, MW673454, MW673459, MW673460, MW680494, MW680503,
MW680504, MW680513, MW680514, MW680522, MW680523, MW680524,
MW680531, MW680532, MW680533, MW680540, MW672967, MW673357,
MW680546, MW673366, MW680548, MW673368, MW680550, MW673377,
MW680571, MW673382, MW673383, MW673385, MW673388, MW673389,
MW673390, MW673396, MW673397, MW673402, MW673403, MW673410,
MW673412, MW673419, MW673423, MW673429, MW673433, MW673450,
MW673418, MW673446, MW673461, MW673469, MW673479, MW680534,
MW680552, MW680555, MW680558, MW680563, MW680565, MW680566,
MW680574, MW680583, MW680584, MW680586, MW680593, MW680601,
MW680602, MW680603, MW672737, MW672738, MW672739, MW672741,
MW672744, MW672745, MW672746, MW672747, MW672748, MW672750,
MW672751, MW672936, MW672946, MW672953, MW672961, MW672969,
MW673073, MW673074, MW673075, MW673077, MW673079, MW673080,
MW673081, MW673082, MW673083, MW673085, MW673089, MW673090,
MW673091, MW673099, MW673109, MW673111, MW673119, MW673369,
MW672749, MW672937, MW672947, MW672956, MW672962, MW672964,
MW672970, MW673101, MW673110, MW673121, MW673129, MW673354,
MW673355, MW673361, MW673362, MW673365, MW673370, MW673371,
MW673372, MW673373, MW673379, MW673381, MW680541, MW680542,
MW673358, MW680547, MW673367, MW680549, MW673376, MW680561,
MW673378, MW680572, MW680581, MW673384, MW673386, MW673387,
MW673391, MW673392, MW673394, MW673395, MW673398, MW673404,
MW673405, MW673409, MW673411, MW673413, MW673414, MW673420,
MW673421, MW673422, MW673430, MW673431, MW673432, MW673439,
MW673440, MW673441, MW673449, MW673427, MW673445, MW673455,
MW673456, MW673462, MW673470, MW673480, MW680544, MW680556,
MW680562, MW680564, MW680573, MW680575, MW680576, MW680582,
MW680592, MW680612, MW673202, MW673203, MW673204, MW673205,
MW673211, MW673213, MW673214, MW673221, MW673222, MW673223,
MW673231, MW673232, MW673496, MW673499, MW673500, MW673501,
MW673502, MW673503, MW673504, MW673509, MW673510, MW673511,
MW673512, MW672849, MW672850, MW673401, MW673406, MW673407,
MW673415, MW673416, MW673426, MW673434, MW674869, MW677552,
MW677553, MW677554, MW681316, MW681326, MW681344, MW681371,
MW681387, MW681388, MW681404, MW685453, MW685484, MW685485,
MW685494, MW685498, MW685573, MW685575, MW685579, MW685580,
MW685581, MW685589, MW685590, MW685598, MW685605, MW685606,
MW685615, MW685635, MW674866, MW674874, MW677556, MW677557,
MW685462, MW685469, MW685472, MW685475, MW685478, MW685480,
MW685482, MW672983, MW672984, MW673447, MW673448, MW673458,
MW673464, MW673465, MW673466, MW673467, MW673472, MW673473,
MW673475, MW673483, MW673485, MW673489, MW673490, MW673492,
MW673493, MW673494, MW673495, MW694204, MW694214, MW694224,
MW694234, MW694239, MW694240, MW694241, MW694242, MW694243,
MW694245, MW694246, MW694247, MW694248, MW694249, MW694254,
MW694255, MW694256, MW694257, MW694258, MW694262, MW694263,
MW694264, MW694265, MW694266, MW694267, MW694268, MW694273,
MW694275, MW694276, MW694277, MW694354, MW694357, MW694358,
MW694361, MW694362, MW694363, MW694366, MW694367, MW694371,
MW694372, MW694373, MW694377, MW694378, MW694381, MW694382,
MW694451, MW694457, MW694458, MW696554, MW696555, MW693514,
MW693515, MW693517, MW693521, MW693530, MW693532, MW693548,
MW693549, MW693558, MW694145, MW694155, MW694163, MW694164,
MW694172, MW694174, MW694175, MW694180, MW694182, MW694184,
MW694189, MW694190, MW694192, MW694193, MW696612, MW696572,
MW696591, MW696593, MW696596, MW696608, MW696611, MW696618,
MW696620, MW696621, MW696624, MW696634, MW696635, MW696636,
MW696645, MW696655, MW693516, MW696370, MW696378, MW696379,
MW696380, MW696385, MW696386, MW696387, MW696388, MW696389,
MW696390, MW696395, MW696396, MW696397, MW696398, MW696399,
MW696400, MW696404, MW696405, MW696406, MW696407, MW696408,
MW696409, MW696410, MW696415, MW696416, MW696417, MW696418,
MW696419, MW696420, MW696424, MW696425, MW696426, MW696427,
MW696428, MW696429, MW696431, MW696433, MW696434, MW696435,
MW696436, MW696441, MW696442, MW696443, MW696444, MW696445,
MW696446, MW696451, MW696452, MW697782, MW696437, MW696439,
MW696440, MW696447, MW696449, MW696453, MW696454, MW696461,
MW696463, MW696469, MW697784, MW697785, MW697789, MW697790,
MW697791, MW697792, MW697796, MW697800, MW697806, MW697810,
MW697812, MW697813, MW697815, MW697820, MW697821, MW697822,
MW697823, MW695390, MW695391, MW695392, MW696205, MW696206,
MW696207, MW696208, MW696212, MW696213, MW696214, MW696215,
MW696216, MW696217, MW696218, MW696219, MW696220, MW696221,
MW696222, MW696223, MW696224, MW696230, MW696231, MW696232,
MW696233, MW696234, MW696235, MW696239, MW696240, MW696241,
MW696242, MW696243, MW696244, MW696249, MW696250, MW696251,
MW696334, MW696335, MW696336, MW696341, MW696342, MW696343,
MW696344, MW696345, MW696346, MW696351, MW696352, MW696353,
MW696354, MW696361, MW696362, MW693556, MW693581, MW693582,
MW693589, MW693591, MW693608, MW693609, MW693912, MW694251,
MW694252, MW694253, MW694259, MW694261, MW694269, MW694270,
MW694271, MW694356, MW694467, MW694365, MW694375, MW694471,
MW693565, MW693572, MW694465, MW694474, MW695375, MW694462,
MW694463, MW696769, MW696815, MW695364, MW696817, MW695368,
MW695369, MW696826, MW695373, MW696878, MW697842, MW697851,
MW697852, MW697858, MW697859, MW697860, MW697861, MW697862,
MW697866, MW697867, MW697868, MW697869, MW697870, MW697871,
MW697872, MW697873, MW697874, MW697875, MW697876, MW697877,
MW697878, MW697879, MW697883, MW697884, MW697885, MW697886,
MW697887, MW697888, MW697889, MW697893, MW697894, MW697895,
MW697896, MW697897, MW697898, MW697910, MW697911, MW697912,
MW697913, MW697919, MW697920, MW697921, MW697929, MW697930,
MW697931, MW697939, MW696528, MW696547, MW696887, MW696895,
MW697004, MW697006, MW697007, MW697013, MW697015, MW697016,
MW697021, MW697035, MW697042, MW697060, MW697061, MW697062,
MW697063, MW698085, MW698094, MW698095, MW698100, MW698103,
MW698109, MW697009, MW697017, MW697018, MW697019, MW697027,
MW697028, MW697045, MW697048, MW697054, MW697064, MW697066,
MW697068, MW697074, MW697756, MW697762, MW697763, MW697771,
MW697824, MW697843, MW696359, MW696368, MW696376, MW696382,
MW696392, MW696412, MW696421, MW697069, MW697757, MW697758,
MW697759, MW697760, MW697765, MW697766, MW697767, MW697768,
MW697769, MW697770, MW697775, MW697776, MW697777, MW697778,
MW697779, MW697826, MW697827, MW697828, MW697829, MW697830,
MW697831, MW697835, MW697836, MW697837, MW697838, MW697839,
MW697844, MW697845, MW697846, MW697847, MW697848, MW697849,
MW697850, MW697853, MW697854, MW697855, MW697856, MW697857,
MW697863, MW697864, MW697865, MW697780, MW697832, MW697841,
MW696456, MW696457, MW696458, MW696464, MW696465, MW696466,
MW696467, MW696468, MW696470, MW696471, MW696472, MW696473,
MW696474, MW696475, MW696479, MW696480, MW696481, MW696482,
MW696483, MW696490, MW696491, MW696492, MW696493, MW696494,
MW696499, MW696500, MW696501, MW696502, MW696509, MW696510,
MW696511, MW696519, MW696520, MW696529, MW696539, MW696540,
MW696722, MW696772, MW696820, MW696831, MW697797, MW697798,
MW697799, MW697807, MW697808, MW697809, MW697816, MW697817,
MW697818, MW697819, MW696476, MW696477, MW696478, MW696484,
MW696485, MW696486, MW696487, MW696488, MW696495, MW696496,
MW696497, MW696498, MW696503, MW696504, MW696505, MW696506,
MW696507, MW696512, MW696513, MW696514, MW696515, MW696516,
MW696517, MW696518, MW696521, MW696522, MW696523, MW696524,
MW696525, MW696526, MW696527, MW696530, MW696531, MW696532,
MW696533, MW696534, MW696535, MW696536, MW696537, MW696541,
MW696542, MW696543, MW696544, MW696545, MW696546, MW696549,
MW696885, MW696886, MW696894, MW696904, MW693991, MW693992,
MW693993, MW693994, MW693997, MW693998, MW693999, MW694007,
MW694008, MW694009, MW694010, MW694016, MW696508, MW696905,
MW697005, MW697012, MW697014, MW697020, MW697022, MW697023,
MW697024, MW697025, MW697030, MW697031, MW697032, MW697033,
MW697034, MW697040, MW697041, MW697043, MW697050, MW697051,
MW697052, MW697053, MW698099, MW698101, MW698102, MW698104,
MW698105, MW697008, MW697029, MW697037, MW697044, MW697056,
MW697057, MW697058, MW697059, MW697070, MW697073, MW697764,
MW697772, MW697773, MW697774, MW697781, MW697825, MW697834,
MW694080, MW694081, MW694082, MW694088, MW694089, MW694090,
MW694091, MW694092, MW694098, MW694099, MW694100, MW694101,
MW694102, MW694108, MW694109, MW694110, MW694111, MW694112,
MW694116, MW694117, MW694118, MW694119, MW694202, MW694203,
MW694208, MW694209, MW694210, MW694211, MW694212, MW694213,
MW694217, MW694218, MW694219, MW694220, MW694221, MW694222,
MW694223, MW694225, MW694226, MW694227, MW694228, MW694229,
MW694230, MW694231, MW694232, MW694233, MW694235, MW694236,
MW694237, MW694238, MW696257, MW696258, MW696259, MW696265,
MW696266, MW696267, MW696268, MW696274, MW696275, MW696276,
MW696277, MW696285, MW696286, MW696287, MW696293, MW696294,
MW696295, MW696296, MW696303, MW696304, MW696305, MW696311,
MW696312, MW696313, MW696314, MW696322, MW696323, MW696324,
MW696603, MW696622, MW696623, MW696631, MW696632, MW696640,
MW696641, MW696646, MW696647, MW696648, MW696649, MW696657,
MW696658, MW696659, MW696664, MW696665, MW696666, MW696674,
MW696675, MW696684, MW696694, MW696642, MW696643, MW696650,
MW696651, MW696652, MW696653, MW696663, MW696668, MW696669,
MW696677, MW696680, MW696681, MW696682, MW696685, MW696686,
MW694466, MW696673, MW696683, MW696692, MW696693, MW696700,
MW696701, MW696702, MW696703, MW696710, MW696712, MW696713,
MW696718, MW696719, MW696720, MW696721, MW696723, MW696727,
MW696728, MW696729, MW696730, MW696731, MW696732, MW696733,
MW696735, MW696736, MW696737, MW696738, MW696739, MW696766,
MW696767, MW696770, MW696771, MW696773, MW696774, MW696814,
MW696819, MW696821, MW696822, MW696823, MW696824, MW696829,
MW696830, MW696832, MW696833, MW696834, MW696839, MW696849,
MW696850, MW694274, MW694359, MW694360, MW694368, MW694369,
MW694370, MW694379, MW694380, MW694452, MW694453, MW694454,
MW694455, MW694459, MW694460, MW694461, MW696550, MW696551,
MW696552, MW696553, MW696556, MW696557, MW696558, MW696559,
MW696560, MW696561, MW696562, MW696563, MW696564, MW696565,
MW696566, MW696567, MW696568, MW696569, MW696570, MW696574,
MW696575, MW696576, MW696577, MW696578, MW696579, MW696584,
MW696585, MW696586, MW696587, MW696594, MW696595, MW696604,
MW696605, MW696606, MW696614, MW694017, MW694018, MW694019,
MW694020, MW694022, MW696252, MW696253, MW696254, MW696255,
MW696256, MW696260, MW696261, MW696262, MW696263, MW696264,
MW696270, MW696271, MW696272, MW696273, MW696280, MW696281,
MW696282, MW696283, MW696284, MW696290, MW696291, MW696292,
MW696300, MW696301, MW696302, MW696310, MW696320, MW696321,
MW696836, MW696837, MW696838, MW696840, MW696841, MW696842,
MW696843, MW696844, MW696846, MW696847, MW695380, MW695383,
MW696853, MW695386, MW695387, MW696863, MW695394, MW695395,
MW696845, MW696858, MW696864, MW696865, MW696875, MW696876,
MW696228, MW696237, MW696238, MW696337, MW696339, MW696347,
MW696348, MW696365, MW696371, MW696381, MW696422, MW696687,
MW696688, MW696689, MW696690, MW696691, MW696695, MW696696,
MW696697, MW696698, MW696699, MW696704, MW696705, MW696706,
MW696707, MW696708, MW696709, MW696714, MW696715, MW696716,
MW696717, MW696724, MW696725, MW696726, MW696734, MW693513,
MW693518, MW693519, MW693520, MW693522, MW693527, MW693528,
MW693529, MW693531, MW693537, MW693538, MW693539, MW693540,
MW693541, MW693547, MW693557, MW694162, MW694165, MW694173,
MW694181, MW694183, MW694185, MW694191, MW694194, MW694200,
MW694201, MW696580, MW696581, MW696582, MW696583, MW696588,
MW696601, MW696607, MW696609, MW696610, MW696615, MW696625,
MW696627, MW696629, MW696637, MW696639, MW696835, MW695379,
MW696848, MW696851, MW696852, MW695384, MW695385, MW696856,
MW696869, MW696202, MW696880, MW696881, MW696857, MW696862,
MW696870, MW696873, MW696877, MW696225, MW696226, MW696236,
MW696246, MW696247, MW696340, MW696350, MW696355, MW696356,
MW696369, MW696374, MW696401, MW696402, MW696403, MW696423,
MW696854, MW696855, MW696859, MW696860, MW695388, MW695393,
MW696871, MW696872, MW696879, MW696209, MW696882, MW696884,
MW696861, MW696866, MW696867, MW696868, MW696874, MW696883,
MW696338, MW696357, MW696358, MW696366, MW696393, MW687165,
MW687172, MW687173, MW687174, MW687175, MW687180, MW687181,
MW687182, MW687183, MW687184, MW687185, MW687190, MW687191,
MW687192, MW687193, MW687194, MW687195, MW687199, MW687200,
MW687201, MW687202, MW687203, MW687204, MW687207, MW687208,
MW687209, MW687210, MW687211, MW687212, MW687213, MW687216,
MW687217, MW687218, MW687219, MW687220, MW687221, MW687222,
MW687226, MW687227, MW687228, MW687229, MW687230, MW687231,
MW687232, MW687236, MW687237, MW687238, MW687239, MW687246,
MW687247, MW687214, MW687223, MW687233, MW687235, MW687240,
MW687241, MW687242, MW687243, MW687250, MW687252, MW687256,
MW687267, MW687268, MW687269, MW687270, MW687277, MW687286,
MW687287, MW687296, MW697918, MW697926, MW697936, MW697945,
MW697946, MW699189, MW699197, MW687144, MW687145, MW687152,
MW687153, MW687154, MW687155, MW687162, MW687168, MW687169,
MW687171, MW687178, MW687186, MW696980, MW696981, MW696982,
MW696983, MW696990, MW696991, MW696992, MW696993, MW696998,
MW696999, MW697000, MW697001, MW697002, MW697003, MW698047,
MW698048, MW698049, MW698050, MW698051, MW698052, MW698053,
MW698057, MW698058, MW698059, MW698060, MW698061, MW698062,
MW698063, MW698064, MW698067, MW698068, MW698069, MW698070,
MW698071, MW698072, MW698073, MW698077, MW698078, MW698079,
MW698080, MW698081, MW698082, MW698086, MW698087, MW698088,
MW698089, MW698090, MW698091, MW698096, MW698106, MW697995,
MW698003, MW698004, MW698010, MW698012, MW698019, MW698020,
MW698024, MW698037, MW698038, MW698039, MW698040, MW698083,
MW698097, MW698098, MW698111, MW698113, MW698123, MW687272,
MW687273, MW687274, MW687275, MW687280, MW687281, MW687282,
MW687283, MW687284, MW687285, MW687288, MW687289, MW687290,
MW687291, MW687292, MW687293, MW687297, MW687298, MW687299,
MW687300, MW698785, MW698786, MW698791, MW698792, MW698793,
MW698794, MW698795, MW698796, MW698797, MW698800, MW698801,
MW698802, MW698803, MW698804, MW698810, MW698811, MW698969,
MW698970, MW698971, MW698979, MW698980, MW696903, MW696913,
MW696921, MW696922, MW696923, MW696930, MW696931, MW696932,
MW696943, MW696944, MW696945, MW696946, MW696949, MW696950,
MW696951, MW696952, MW696953, MW696959, MW696960, MW696961,
MW696962, MW696963, MW696966, MW696967, MW696968, MW696969,
MW696970, MW696971, MW696972, MW696973, MW696974, MW696975,
MW696976, MW696977, MW696978, MW696979, MW696984, MW696985,
MW696986, MW696987, MW696988, MW696989, MW696994, MW696995,
MW696996, MW696997, MW698046, MW698056, MW698066, MW698076,
MW698005, MW698022, MW698023, MW698025, MW698032, MW698033,
MW698034, MW698035, MW698041, MW698042, MW698043, MW698044,
MW698045, MW698114, MW698115, MW698116, MW698117, MW698118,
MW698119, MW698124, MW698125, MW698126, MW698127, MW698128,
MW698130, MW698131, MW698132, MW698133, MW698134, MW698135,
MW698136, MW698140, MW698141, MW698142, MW698143, MW698144,
MW698145, MW698150, MW698151, MW698152, MW698153, MW698154,
MW698155, MW698160, MW698161, MW698162, MW698163, MW698170,
MW698171, MW698180, MW696548, MW696889, MW696890, MW696891,
MW696892, MW696893, MW696897, MW696898, MW696899, MW696900,
MW696901, MW696902, MW696907, MW696908, MW696909, MW696910,
MW696911, MW696912, MW696914, MW696915, MW696916, MW696917,
MW696918, MW696919, MW696920, MW696924, MW696925, MW696926,
MW696927, MW696928, MW696929, MW696933, MW696934, MW696935,
MW696936, MW696937, MW696938, MW696939, MW696940, MW696941,
MW696942, MW696947, MW696948, MW696954, MW696955, MW696956,
MW696957, MW696958, MW696964, MW696965, MW687205, MW687215,
MW687224, MW687225, MW687234, MW687244, MW687248, MW687249,
MW687251, MW687257, MW687258, MW687259, MW687260, MW687266,
MW687271, MW687276, MW687278, MW687279, MW697908, MW697927,
MW697935, MW698790, MW699190, MW699206, MW687150, MW687161,
MW687164, MW687170, MW687176, MW687187, MW687196, MW687197,
MW687206, MW698137, MW698146, MW698164, MW698165, MW698181,
MW698750, MW698759, MW698783, MW698129, MW698138, MW698147,
MW698156, MW698157, MW698172, MW698173, MW698182, MW698775,
MW698784, MW687294, MW687295, MW687308, MW687318, MW687325,
MW687319, MW687344, MW687327, MW687360, MW687329, MW698787,
MW687339, MW698789, MW687346, MW687347, MW687348, MW698807,
MW687354, MW698809, MW687356, MW698962, MW687358, MW687361,
MW698966, MW687363, MW698973, MW687365, MW698975, MW687367,
MW698981, MW687371, MW698983, MW687373, MW698985, MW687375,
MW698990, MW687380, MW698992, MW687382, MW687384, MW699000,
MW687391, MW699002, MW687400, MW699010, MW687402, MW699173,
MW687410, MW687411, MW646960, MW646961, MW687419, MW687426,
MW687436, MW689753, MW689771, MW689779, MW689787, MW689796,
MW689797, MW698139, MW698148, MW698149, MW698158, MW698159,
MW698166, MW698167, MW698168, MW698169, MW698174, MW698175,
MW698176, MW698177, MW698178, MW698179, MW698183, MW698184,
MW698185, MW698186, MW698187, MW698746, MW698747, MW698748,
MW698749, MW698751, MW698752, MW698753, MW698754, MW698755,
MW698760, MW698761, MW698766, MW698767, MW698768, MW698769,
MW698770, MW698776, MW698777, MW698778, MW646965, MW687147,
MW687148, MW687157, MW687159, MW687160, MW687420, MW687438,
MW689755, MW689773, MW689780, MW689781, MW689782, MW689788,
MW689798, MW698745, MW698756, MW698758, MW698762, MW698764,
MW698771, MW698773, MW687309, MW687337, MW687326, MW687353,
MW687328, MW687370, MW687338, MW698788, MW687345, MW698798,
MW698799, MW698805, MW698806, MW687349, MW698808, MW687355,
MW698961, MW687357, MW698963, MW687359, MW698965, MW687362,
MW698972, MW687364, MW698974, MW687366, MW698976, MW687368,
MW698982, MW687372, MW698984, MW687374, MW698989, MW687376,
MW698991, MW687381, MW698993, MW687383, MW698999, MW687390,
MW699001, MW687392, MW699009, MW687401, MW699172, MW687403,
MW687412, MW689756, MW687444, MW687445, MW687453, MW687454,
MW687455, MW687463, MW687464, MW687465, MW687471, MW687472,
MW687473, MW687479, MW687480, MW687481, MW687487, MW687488,
MW687489, MW687490, MW687496, MW687498, MW687499, MW687500,
MW687506, MW687507, MW687508, MW687509, MW687516, MW687530,
MW687539, MW687568, MW687577, MW687578, MW687587, MW687594,
MW689096, MW689097, MW689106, MW689107, MW689108, MW689109,
MW689115, MW689116, MW689125, MW689126, MW689127, MW689132,
MW689133, MW689134, MW687421, MW687422, MW687423, MW687424,
MW687425, MW687429, MW687430, MW687431, MW687432, MW687433,
MW687434, MW687435, MW687439, MW687440, MW687442, MW687443,
MW687446, MW687447, MW687448, MW687449, MW687450, MW687451,
MW687452, MW687456, MW687457, MW687458, MW687459, MW687460,
MW687461, MW687462, MW687466, MW687467, MW687468, MW687469,
MW687470, MW687476, MW687477, MW687478, MW687486, MW689774,
MW689775, MW689783, MW689784, MW689785, MW689791, MW689792,
MW689793, MW689794, MW689795, MW687378, MW687379, MW687385,
MW687386, MW687387, MW687388, MW687389, MW687393, MW687394,
MW687395, MW687396, MW687397, MW687398, MW687399, MW687404,
MW687405, MW687407, MW687408, MW687409, MW687413, MW687414,
MW687415, MW687416, MW687417, MW687418, MW689747, MW689748,
MW689749, MW689750, MW689751, MW689752, MW689757, MW689758,
MW689759, MW689760, MW689761, MW689762, MW689763, MW689766,
MW689767, MW689768, MW689769, MW689770, MW689776, MW689777,
MW689778, MW689786, MW646962, MW646964, MW687146, MW687149,
MW687156, MW687158, MW687166, MW687427, MW687428, MW687437,
MW689754, MW689764, MW689765, MW689772, MW689789, MW689790,
MW689799, MW698744, MW698757, MW698763, MW698765, MW698772,
MW698774, MW698779, MW698780, MW698781, MW698782, MW687255,
MW687261, MW687262, MW687263, MW687264, MW687265, MW646959,
MW687142, MW687143, MW687151, MW687163, MW687167, MW687177,
MW687179, MW687188, MW687189, MW687198, MW681123, MW689150,
MW689151, MW689159, MW689160, MW689170, MW689178, MW689183,
MW689184, MW689691, MW689692, MW689693, MW698967, MW698968,
MW698977, MW698978, MW698986, MW698987, MW698988, MW698995,
MW698996, MW698997, MW698998, MW699003, MW699004, MW699005,
MW699006, MW699011, MW699012, MW699013, MW699167, MW699168,
MW699169, MW699174, MW699175, MW699176, MW699177, MW699182,
MW699183, MW699249, MW699250, MW699251, MW699252, MW699257,
MW699258, MW699259, MW699267, MW699268, MW699277, MW699278,
MW681102, MW681110, MW681119, MW689148, MW689149, MW689157,
MW689158, MW689166, MW689168, MW689172, MW689174, MW689175,
MW699170, MW699178, MW699254, MW699260, MW699261, MW422028,
MW422036, MW422037, MW422038, MW422039, MW422054, MW422046,
MW422048, MW422064, MW422051, MW422052, MW422053, MW422058,
MW422059, MW422060, MW422061, MW422062, MW422063, MW422085,
MW422069, MW422090, MW422071, MW422092, MW422073, MW422094,
MW422100, MW422080, MW422087, MW422089, MW422108, MW422099,
MW687517, MW422112, MW422113, MW422114, MW422117, MW687521,
MW687523, MW687527, MW422129, MW687529, MW646948, MW687310,
MW687525, MW422096, MW422122, MW422131, MW646945, MW646952,
MW687303, MW687304, MW687311, MW687314, MW687316, MW687324,
MW687331, MW687333, MW687340, MW687352, MW422045, MW422047,
MW422056, MW422049, MW422065, MW422066, MW422074, MW422075,
MW422076, MW422082, MW422083, MW422084, MW422068, MW422086,
MW422070, MW422091, MW422072, MW422093, MW422077, MW422095,
MW422079, MW422101, MW422081, MW422103, MW422088, MW422105,
MW422097, MW422098, MW422109, MW422111, MW687518, MW687519,
MW687520, MW422118, MW687522, MW422120, MW422121, MW422127,
MW422128, MW687528, MW422130, MW646949, MW646950, MW646958,
MW687301, MW687524, MW422116, MW422124, MW646944, MW646947,
MW646951, MW646953, MW646955, MW687302, MW687312, MW687313,
MW687315, MW687320, MW687321, MW687322, MW687332, MW687334,
MW687342, MW681094, MW681095, MW681096, MW681098, MW681099,
MW681104, MW681105, MW681106, MW681107, MW681108, MW681114,
MW681115, MW681116, MW681117, MW681124, MW681125, MW681126,
MW681127, MW687588, MW689098, MW689099, MW689110, MW689111,
MW689112, MW689117, MW689118, MW689119, MW689128, MW689129,
MW689135, MW689136, MW689137, MW689138, MW689139, MW689140,
MW689141, MW689142, MW689143, MW689144, MW689145, MW689146,
MW689152, MW689153, MW689154, MW689155, MW689163, MW689164,
MW421986, MW421987, MW421988, MW421989, MW421997, MW421998,
MW421999, MW422005, MW422006, MW422007, MW422008, MW422009,
MW422015, MW422016, MW422018, MW422019, MW422023, MW422031,
MW422032, MW422033, MW422034, MW422035, MW422040, MW422041,
MW422043, MW422044, MW422050, MW422057, MW687526, MW699171,
MW699180, MW699181, MW699255, MW699262, MW699263, MW699272,
MW699273, MW699281, MW699282, MW699283, MW699287, MW699290,
MW699291, MW699297, MW699298, MW681100, MW681101, MW681103,
MW681109, MW681111, MW681113, MW681118, MW681120, MW681121,
MW681122, MW681128, MW681129, MW681130, MW681131, MW689147,
MW689156, MW689167, MW689169, MW689173, MW689176, MW689177,
MW689182, MW699007, MW699008, MW699179, MW699253, MW699269,
MW699270, MW699271, MW699279, MW699280, MW699289, MW422106,
MW422115, MW422123, MW422125, MW646943, MW646946, MW646954,
MW646956, MW687305, MW687306, MW687307, MW687323, MW687330,
MW687335, MW687341, MW687350, MW687351, MW687369, MW687377,
MW687482, MW687491, MW421983, MW687495, MW421990, MW687505,
MW687511, MW422002, MW422010, MW687532, MW687536, MW687537,
MW681025, MW687541, MW687546, MW681044, MW687549, MW681051,
MW687555, MW687557, MW681071, MW687564, MW681080, MW687567,
MW681089, MW699265, MW687576, MW699274, MW687584, MW699295,
MW699292, MW689101, MW689102, MW689104, MW689120, MW687494,
MW421984, MW687501, MW421993, MW687514, MW422001, MW422020,
MW681033, MW681043, MW687554, MW681061, MW687556, MW687558,
MW687560, MW687561, MW687562, MW687566, MW681088, MW687573,
MW687579, MW687580, MW699275, MW687583, MW699284, MW699286,
MW699288, MW699293, MW699294, MW689103, MW687534, MW422012,
MW422013, MW687540, MW687544, MW681034, MW681035, MW687543,
MW681045, MW687550, MW687551, MW681062, MW681070, MW687559,
MW687572, MW687571, MW699256, MW699264, MW687582, MW699266,
MW689100, MW689122, MW699285, MW687591, MW687592, MW687593,
MW689113, MW689121, MW689123, MW689130, MW421991, MW421992,
MW687510, MW422000, MW687515, MW687531, MW422011, MW687535,
MW687538, MW687545, MW687542, MW687547, MW687548, MW681052,
MW687552, MW687553, MW681079, MW687565, MW687563, MW687569,
MW687570, MW681134, MW687574, MW687575, MW687589, MW687581,
MW699276, MW687590, MW699296, MW689105, MW681009, MW681010,
MW681018, MW681037, MW681348, MW681355, MW681356, MW681360,
MW681361, MW689839, MW689847, MW689883, MW689884, MW689891,
MW689893, MW689894, MW689899, MW689900, MW689902, MW689911,
MW689916, MW689918, MW692114, MW692117, MW692118, MW422004,
MW422022, MW687502, MW687503, MW687512, MW687513, MW687474,
MW687533, MW681314, MW681323, MW681340, MW681342, MW681370,
MW681377, MW681385, MW681386, MW681392, MW681394, MW681395,
MW681401, MW681402, MW681418, MW681425, MW681436, MW681444,
MW666453, MW666456, MW666466, MW666943, MW666944, MW666945,
MW666953, MW666954, MW666955, MW666961, MW666962, MW666963,
MW666964, MW666965, MW666970, MW666971, MW666972, MW666973,
MW666974, MW666975, MW666978, MW666979, MW666980, MW666981,
MW666982, MW666983, MW666984, MW666988, MW666989, MW666990,
MW666991, MW666992, MW666993, MW666996, MW666997, MW666998,
MW666999, MW667000, MW667006, MW667007, MW667008, MW667016,
MW667017, MW667018, MW667019, MW667026, MW667027, MW667028,
MW667036, MW666416, MW666426, MW666434, MW666436, MW666444,
MW666445, MW666446, MW666449, MW666451, MW666454, MW666455,
MW666465, MW666469, MW666471, MW666941, MW666946, MW666947,
MW666956, MW666957, MW666958, MW666959, MW666960, MW666967,
MW666968, MW666969, MW666976, MW666396, MW666406, MW666413,
MW667001, MW667002, MW667009, MW667012, MW667015, MW666458,
MW667022, MW667479, MW669055, MW667030, MW667031, MW667037,
MW669091, MW666362, MW666364, MW666366, MW666372, MW666373,
MW666374, MW666376, MW666380, MW666383, MW666388, MW666390,
MW666394, MW666395, MW666402, MW666403, MW666407, MW666411,
MW666419, MW666420, MW666430, MW666438, MW666757, MW666776,
MW666785, MW666794, MW666795, MW666803, MW666805, MW666813,
MW666815, MW666822, MW666823, MW666824, MW666831, MW666832,
MW666834, MW666836, MW666837, MW666838, MW666839, MW666840,
MW666850, MW667327, MW667328, MW667336, MW666250, MW667347,
MW667356, MW666261, MW666270, MW666291, MW666294, MW666295,
MW666296, MW666355, MW666363, MW666382, MW666397, MW666825,
MW666833, MW666835, MW666841, MW666852, MW666853, MW666870,
MW667329, MW667330, MW667339, MW667340, MW667358, MW667359,
MW666472, MW666473, MW666474, MW666475, MW666476, MW666477,
MW666481, MW666482, MW666483, MW666484, MW666485, MW666486,
MW666487, MW666490, MW666491, MW666492, MW666493, MW666494,
MW666495, MW666496, MW666500, MW666501, MW666502, MW666503,
MW666504, MW666510, MW666511, MW666512, MW666513, MW666520,
MW667065, MW667074, MW666211, MW666218, MW667080, MW666220,
MW667082, MW667083, MW666226, MW667085, MW666228, MW667092,
MW666230, MW667094, MW666235, MW667100, MW666237, MW666238,
MW666244, MW666245, MW666246, MW666252, MW666256, MW666257,
MW666258, MW666262, MW666263, MW666265, MW666274, MW666282,
MW666480, MW666714, MW666716, MW667055, MW667064, MW667072,
MW667089, MW667099, MW666488, MW666497, MW666498, MW666517,
MW666519, MW666522, MW666523, MW666524, MW666526, MW666532,
MW666533, MW666543, MW666544, MW666560, MW666562, MW666728,
MW668562, MW668563, MW668571, MW668572, MW668573, MW668578,
MW668579, MW668580, MW668581, MW668582, MW668583, MW668587,
MW668588, MW668589, MW668590, MW668591, MW668592, MW668597,
MW668598, MW668599, MW668600, MW668601, MW668602, MW668604,
MW668606, MW668607, MW668608, MW668609, MW668614, MW668615,
MW668616, MW668617, MW668618, MW668619, MW668620, MW668624,
MW668625, MW668626, MW668812, MW668813, MW668814, MW668815,
MW668819, MW668820, MW668821, MW668829, MW668830, MW668831,
MW668832, MW668986, MW668995, MW668996, MW669005, MW669006,
MW669007, MW669014, MW669015, MW669016, MW669022, MW669023,
MW669024, MW669025, MW669026, MW669033, MW669034, MW669035,
MW669036, MW669041, MW669042, MW669043, MW669044, MW669045,
MW669046, MW669050, MW669051, MW669052, MW669053, MW669054,
MW669057, MW669058, MW669059, MW669060, MW669061, MW669062,
MW669067, MW669068, MW669069, MW669070, MW669071, MW669077,
MW669078, MW669079, MW669087, MW669088, MW669089, MW669097,
MW669098, MW669107, MW669108, MW666754, MW666756, MW666765,
MW666774, MW666775, MW666791, MW666792, MW666800, MW666802,
MW666812, MW666819, MW666820, MW666829, MW668966, MW668975,
MW668976, MW668982, MW668983, MW668984, MW668985, MW668991,
MW668992, MW668993, MW668994, MW668998, MW669000, MW669002,
MW669003, MW669004, MW669009, MW669010, MW669011, MW669012,
MW669013, MW669018, MW669019, MW669020, MW669021, MW669028,
MW669029, MW669030, MW669031, MW669032, MW669037, MW669038,
MW669039, MW669040, MW669047, MW669048, MW669049, MW667507,
MW667508, MW667516, MW667517, MW667518, MW667525, MW667526,
MW667528, MW667535, MW667536, MW667538, MW667543, MW667544,
MW667546, MW667547, MW667548, MW667553, MW667554, MW667556,
MW667557, MW667558, MW668479, MW668482, MW668483, MW668484,
MW668485, MW668486, MW668487, MW668488, MW668489, MW668490,
MW668561, MW668564, MW668566, MW668567, MW668568, MW668569,
MW668570, MW668574, MW668575, MW668576, MW668577, MW668584,
MW668585, MW668586, MW668594, MW668595, MW668596, MW667486,
MW667487, MW667488, MW667492, MW667493, MW667494, MW667495,
MW667496, MW667497, MW667498, MW667501, MW667502, MW667503,
MW667504, MW667505, MW667506, MW667509, MW667510, MW667511,
MW667512, MW667513, MW667514, MW667515, MW667519, MW667520,
MW667521, MW667522, MW667523, MW667524, MW667529, MW667530,
MW667531, MW667532, MW667533, MW667534, MW667539, MW667540,
MW667541, MW667542, MW667549, MW667550, MW667551, MW667552,
MW667559, MW667560, MW667561, MW668478, MW669116, MW669125,
MW669126, MW666727, MW666735, MW666736, MW666737, MW666744,
MW666745, MW666746, MW666747, MW666748, MW666749, MW666750,
MW666751, MW666752, MW666753, MW666755, MW666760, MW666761,
MW666762, MW666763, MW666764, MW666768, MW666769, MW666770,
MW666771, MW666772, MW666773, MW666786, MW666787, MW666788,
MW666789, MW666790, MW666793, MW666796, MW666797, MW666798,
MW666799, MW666801, MW666808, MW666809, MW666810, MW666811,
MW666816, MW666817, MW666818, MW666826, MW666827, MW666828,
MW666830, MW668999, MW669008, MW666271, MW666279, MW666280,
MW666281, MW666288, MW666289, MW666290, MW666297, MW666298,
MW666299, MW666300, MW666306, MW666307, MW666353, MW666354,
MW666356, MW666358, MW666359, MW666360, MW666361, MW666365,
MW666368, MW666369, MW666370, MW666371, MW666375, MW666378,
MW666379, MW666384, MW666385, MW666387, MW666389, MW666392,
MW666393, MW666398, MW666399, MW666400, MW666401, MW666408,
MW666409, MW666410, MW666417, MW666418, MW666428, MW666429,
MW666437, MW666767, MW666804, MW666814, MW666821, MW645538,
MW645539, MW647945, MW647946, MW647957, MW647958, MW647959,
MW647966, MW647967, MW647968, MW647988, MW647976, MW647977,
MW647978, MW647979, MW647981, MW648007, MW647983, MW648009,
MW647985, MW648015, MW648016, MW647991, MW648018, MW647993,
MW648023, MW647995, MW648025, MW648026, MW648001, MW648028,
MW648003, MW648035, MW648036, MW648006, MW648038, MW648011,
MW648043, MW648044, MW648129, MW648020, MW648131, MW648022,
MW648030, MW648031, MW648139, MW648033, MW648144, MW648041,
MW648146, MW648135, MW648156, MW648159, MW648166, MW648167,
MW648169, MW642642, MW642651, MW642652, MW642660, MW642668,
MW642669, MW642671, MW642674, MW642675, MW642685, MW642691,
MW642692, MW642701, MW642702, MW642711, MW642712, MW642720,
MW643171, MW643196, MW643207, MW643231, MW643241, MW643242,
MW643248, MW643249, MW643250, MW643257, MW643259, MW643599,
MW643600, MW643601, MW643602, MW643609, MW643610, MW643611,
MW643615, MW643617, MW643618, MW643619, MW643628, MW643629,
MW646615, MW646625, MW646626, MW646644, MW646652, MW642637,
MW642638, MW642644, MW642646, MW642647, MW642649, MW642655,
MW642656, MW642657, MW642664, MW642666, MW642667, MW642670,
MW642681, MW642684, MW642690, MW642693, MW642721, MW646636,
MW646637, MW646645, MW646646, MW646647, MW643181, MW643189,
MW643190, MW643197, MW643200, MW643202, MW643209, MW643210,
MW643211, MW643219, MW643221, MW643229, MW643235, MW642689,
MW642698, MW642699, MW642707, MW642708, MW642709, MW642716,
MW642717, MW642718, MW642719, MW642729, MW642737, MW642727,
MW642747, MW642748, MW642749, MW642736, MW642744, MW642758,
MW643081, MW643082, MW642750, MW642752, MW643086, MW643088,
MW643089, MW643092, MW643093, MW646013, MW643095, MW646424,
MW646425, MW646426, MW646427, MW646599, MW646600, MW646610,
MW646611, MW643108, MW646613, MW643110, MW646622, MW643112,
MW643118, MW646631, MW643120, MW646640, MW646642, MW643129,
MW646649, MW643137, MW643138, MW643140, MW642559, MW642605,
MW642616, MW642624, MW642625, MW642626, MW642633, MW645667,
MW645675, MW645677, MW645683, MW645688, MW645714, MW645715,
MW642469, MW642484, MW642485, MW642553, MW642562, MW642598,
MW642759, MW642760, MW642767, MW642777, MW642788, MW643105,
MW643124, MW643132, MW647213, MW642606, MW642623, MW642773,
MW642797, MW642807, MW645668, MW645678, MW645685, MW645686,
MW645687, MW645694, MW645695, MW645697, MW645698, MW645703,
MW645704, MW645706, MW645707, MW645708, MW645713, MW645716,
MW645717, MW645718, MW645721, MW645722, MW645725, MW645726,
MW645730, MW645731, MW645732, MW645734, MW645735, MW645736,
MW645740, MW645741, MW645743, MW645744, MW645745, MW645746,
MW645751, MW645753, MW645754, MW645755, MW645756, MW645759,
MW645760, MW645761, MW645869, MW642756, MW642766, MW642775,
MW642776, MW642784, MW642786, MW642795, MW642798, MW642800,
MW642802, MW642803, MW642811, MW642817, MW642819, MW642820,
MW642821, MW642822, MW642828, MW642837, MW642838, MW642839,
MW642872, MW643263, MW643265, MW643266, MW643271, MW643272,
MW643273, MW643274, MW643275, MW643279, MW643280, MW643281,
MW643282, MW643283, MW643284, MW643288, MW643289, MW643290,
MW643291, MW643292, MW643293, MW643294, MW643297, MW643298,
MW643299, MW643300, MW643301, MW643306, MW643308, MW643309,
MW643310, MW643314, MW643318, MW643350, MW643389, MW643646,
MW643655, MW643656, MW643663, MW643664, MW643665, MW643666,
MW644077, MW644078, MW644080, MW644081, MW644082, MW644087,
MW642805, MW642806, MW642814, MW642815, MW642823, MW642824,
MW642825, MW642831, MW642832, MW642833, MW642834, MW642835,
MW642836, MW642842, MW642843, MW642844, MW642845, MW642850,
MW642865, MW642867, MW642868, MW642873, MW642874, MW642875,
MW642876, MW642877, MW642881, MW642882, MW642883, MW642884,
MW642885, MW642886, MW642891, MW642892, MW642893, MW642894,
MW642901, MW642902, MW642903, MW642911, MW642912, MW643180,
MW643195, MW643223, MW643267, MW643277, MW643285, MW643286,
MW643287, MW643295, MW643296, MW643302, MW643303, MW643304,
MW643307, MW643311, MW643312, MW643313, MW643316, MW643317,
MW643319, MW643320, MW643321, MW643322, MW643325, MW643326,
MW643327, MW643328, MW643329, MW643330, MW643331, MW643334,
MW643335, MW643338, MW643339, MW643343, MW643344, MW643351,
MW643352, MW643353, MW643354, MW643358, MW643381, MW643382,
MW643383, MW643384, MW643390, MW643391, MW643392, MW643399,
MW643400, MW643409, MW642851, MW642852, MW642861, MW642862,
MW642863, MW645559, MW645569, MW645578, MW645579, MW645586,
MW645587, MW645588, MW645593, MW645594, MW645595, MW645602,
MW645603, MW645604, MW645613, MW645614, MW645615, MW645620,
MW645621, MW645622, MW645630, MW645640, MW645641, MW645642,
MW645650, MW645651, MW645652, MW645946, MW645947, MW645948,
MW645956, MW645957, MW645958, MW645965, MW645966, MW645967,
MW645973, MW645974, MW645975, MW645982, MW645988, MW645989,
MW645990, MW645996, MW645997, MW645998, MW642853, MW642854,
MW642855, MW642856, MW642864, MW645968, MW645977, MW645978,
MW645983, MW645984, MW645985, MW645991, MW645992, MW645993,
MW645994, MW645999, MW646000, MW646001, MW646002, MW646003,
MW646004, MW646005, MW646006, MW646007, MW646008, MW646009,
MW646010, MW646011, MW646375, MW646377, MW646422, MW646423,
MW646428, MW646429, MW646430, MW646431, MW646597, MW646603,
MW646609, MW646618, MW646619, MW646620, MW646628, MW646629,
MW646638, MW646639, MW642728, MW642725, MW642726, MW642738,
MW642739, MW642734, MW642735, MW642753, MW642757, MW642745,
MW642746, MW643083, MW643085, MW643087, MW643090, MW646012,
MW643094, MW646014, MW643096, MW643097, MW643098, MW643099,
MW643100, MW643101, MW643102, MW643103, MW643107, MW646612,
MW646621, MW643111, MW646623, MW643117, MW646630, MW646632,
MW643121, MW646641, MW643128, MW646648, MW643130, MW646650,
MW642596, MW642615, MW642622, MW642632, MW645676, MW645680,
MW645681, MW645684, MW645689, MW645692, MW645693, MW645696,
MW645700, MW645702, MW645719, MW645739, MW642763, MW642764,
MW642768, MW642771, MW642772, MW642779, MW642781, MW643104,
MW643135, MW647222, MW642919, MW642920, MW642929, MW642930,
MW642937, MW642938, MW642945, MW642947, MW642956, MW642957,
MW642964, MW642965, MW642971, MW642972, MW642981, MW643305,
MW643323, MW643324, MW643340, MW643341, MW643349, MW643359,
MW643708, MW643719, MW643729, MW643738, MW643739, MW643748,
MW643749, MW643756, MW643757, MW643758, MW643765, MW643767,
MW643768, MW643774, MW643775, MW643776, MW643782, MW643783,
MW643799, MW644407, MW644408, MW643333, MW643709, MW643715,
MW643716, MW643723, MW643726, MW643727, MW643728, MW643734,
MW643736, MW643743, MW643745, MW643746, MW643747, MW643752,
MW643753, MW643755, MW643759, MW643763, MW643769, MW643780,
MW643784, MW643789, MW643801, MW643500, MW643501, MW643510,
MW643528, MW643529, MW644061, MW644062, MW644063, MW644068,
MW644069, MW644070, MW644090, MW644093, MW644099, MW644107,
MW644109, MW644115, MW644116, MW644118, MW644125, MW644127,
MW644128, MW644134, MW644135, MW644136, MW644144, MW644145,
MW644146, MW644151, MW644153, MW644155, MW644156, MW644157,
MW644159, MW644160, MW644167, MW644168, MW644169, MW644170,
MW644179, MW644180, MW644187, MW644188, MW643942, MW643976,
MW643993, MW644003, MW644010, MW644030, MW644047, MW644049,
MW644058, MW644100, MW644114, MW644119, MW644120, MW644124,
MW644133, MW644138, MW644139, MW644221, MW644256, MW644262,
MW643518, MW644022, MW644041, MW644059, MW644065, MW644066,
MW644088, MW644094, MW644098, MW644111, MW644112, MW644142,
MW643778, MW643779, MW643786, MW643787, MW643791, MW643792,
MW643793, MW643798, MW643802, MW643803, MW643804, MW643808,
MW643809, MW643810, MW643811, MW643812, MW643816, MW643817,
MW643818, MW643819, MW643820, MW643821, MW643826, MW643827,
MW643828, MW643834, MW643835, MW643836, MW643842, MW643843,
MW643844, MW643850, MW643851, MW643852, MW643859, MW643860,
MW643861, MW643869, MW643870, MW643879, MW644405, MW644411,
MW644412, MW644414, MW644421, MW644422, MW644423, MW643336,
MW643345, MW642960, MW642966, MW642968, MW643356, MW642970,
MW643385, MW642976, MW642977, MW642978, MW642979, MW642982,
MW642985, MW643396, MW643397, MW643398, MW642992, MW643402,
MW642994, MW643404, MW642996, MW643406, MW643407, MW643002,
MW643411, MW643412, MW643008, MW643414, MW643020, MW643360,
MW643361, MW643363, MW643364, MW643366, MW643369, MW643370,
MW643429, MW643373, MW643431, MW643376, MW643377, MW643378,
MW643440, MW643450, MW643459, MW643458, MW643468, MW643477,
MW643478, MW643486, MW643488, MW643495, MW643496, MW643497,
MW643498, MW643889, MW643892, MW643893, MW643896, MW643897,
MW643899, MW643900, MW643901, MW643902, MW643904, MW643905,
MW643907, MW643908, MW643909, MW643910, MW643915, MW643916,
MW643917, MW643918, MW643919, MW643924, MW643926, MW643927,
MW643928, MW643935, MW643936, MW643937, MW643944, MW643954,
MW643956, MW643957, MW642948, MW642950, MW643346, MW643347,
MW642969, MW643357, MW642975, MW643386, MW643387, MW643393,
MW643394, MW642986, MW642987, MW642991, MW643401, MW642993,
MW643403, MW642995, MW643405, MW643000, MW643001, MW643410,
MW643003, MW643006, MW643007, MW643413, MW643009, MW643415,
MW643021, MW643417, MW643419, MW643362, MW643421, MW643422,
MW643423, MW643424, MW643425, MW643371, MW643430, MW643432,
MW643433, MW643439, MW643379, MW643449, MW643837, MW643838,
MW643845, MW643846, MW643847, MW643848, MW643853, MW643854,
MW643855, MW643856, MW643857, MW643858, MW643862, MW643863,
MW643864, MW643865, MW643866, MW643867, MW643872, MW643873,
MW643874, MW643875, MW643876, MW643880, MW643881, MW643882,
MW643883, MW643884, MW644189, MW644193, MW644194, MW644196,
MW644198, MW644199, MW644204, MW644205, MW644206, MW644207,
MW644208, MW644209, MW644214, MW644215, MW644216, MW644223,
MW644224, MW643315, MW644417, MW644418, MW644419, MW643717,
MW643718, MW643724, MW643735, MW643737, MW643742, MW643744,
MW643751, MW643754, MW643761, MW643762, MW643764, MW643770,
MW643771, MW643772, MW643773, MW643785, MW643790, MW643800,
MW644409, MW644420, MW643941, MW643949, MW643950, MW643977,
MW644001, MW644013, MW644039, MW644040, MW644064, MW644073,
MW644091, MW644092, MW644096, MW644101, MW644106, MW644108,
MW644110, MW644121, MW644129, MW644137, MW644149, MW644247,
MW644273, MW643508, MW644023, MW644031, MW644051, MW644053,
MW644071, MW644089, MW644113, MW644122, MW644123, MW644130,
MW644132, MW644140, MW644143, MW644161, MW644178, MW642869,
MW642904, MW643553, MW643561, MW643565, MW643570, MW643574,
MW643578, MW643585, MW643593, MW643594, MW643595, MW643673,
MW643675, MW643677, MW643681, MW643682, MW643683, MW643700,
MW643702, MW643704, MW643760, MW643547, MW643571, MW643577,
MW643590, MW643669, MW643678, MW643684, MW643687, MW643696,
MW642870, MW642880, MW642887, MW642896, MW643720, MW642915,
MW642928, MW642936, MW642941, MW642944, MW642952, MW642955,
MW642961, MW648029, MW648039, MW648051, MW648052, MW648053,
MW648054, MW648060, MW648061, MW648062, MW648063, MW648064,
MW648070, MW648071, MW648072, MW648073, MW648074, MW648080,
MW648081, MW648082, MW648083, MW648089, MW648090, MW648091,
MW648092, MW648098, MW648099, MW648100, MW648101, MW648102,
MW648107, MW648108, MW648109, MW648110, MW648115, MW648116,
MW648117, MW648118, MW648119, MW648125, MW648133, MW648141,
MW648142, MW648148, MW648160, MW648170, MW648171, MW648176,
MW648177, MW648186, MW648196, MW648152, MW648154, MW648155,
MW648164, MW648172, MW648174, MW648175, MW648178, MW648179,
MW648182, MW648184, MW648188, MW648189, MW648190, MW648191,
MW648193, MW648199, MW648203, MW648209, MW648210, MW648212,
MW648249, MW648255, MW643507, MW643516, MW643517, MW643526,
MW643536, MW643537, MW643542, MW643543, MW643544, MW643550,
MW643551, MW643552, MW643558, MW643559, MW643560, MW643568,
MW643569, MW643588, MW643680, MW643903, MW643911, MW643912,
MW643913, MW643920, MW643921, MW643922, MW643923, MW643930,
MW643931, MW643938, MW643939, MW643940, MW643945, MW643946,
MW643947, MW643948, MW643955, MW643958, MW643959, MW643960,
MW643966, MW643968, MW643974, MW643975, MW643984, MW642878,
MW642913, MW642921, MW643545, MW643557, MW643564, MW643566,
MW643567, MW643572, MW643573, MW643580, MW643582, MW643583,
MW643584, MW643589, MW643591, MW643672, MW643676, MW643685,
MW643686, MW643688, MW643692, MW643693, MW643694, MW643701,
MW643554, MW643555, MW643563, MW643579, MW643586, MW643587,
MW643596, MW643671, MW643679, MW643689, MW642846, MW643707,
MW642895, MW643712, MW643713, MW643714, MW642905, MW642909,
MW643732, MW642914, MW642916, MW642922, MW642924, MW642925,
MW642926, MW642933, MW642951, MW633895, MW633905, MW633915,
MW633916, MW643788, MW643794, MW643795, MW643796, MW643797,
MW643806, MW643807, MW643813, MW643814, MW643815, MW643830,
MW643831, MW643832, MW643833, MW643839, MW643840, MW643849,
MW643992, MW643999, MW644000, MW644008, MW644009, MW644026,
MW644035, MW644036, MW644045, MW644046, MW644054, MW644313,
MW644314, MW644323, MW644346, MW644347, MW644415, MW644425,
MW633913, MW643943, MW643953, MW643962, MW643965, MW643971,
MW643978, MW643988, MW643989, MW643996, MW643998, MW644006,
MW644007, MW644016, MW644034, MW644044, MW644331, MW644324,
MW644342, MW644355, MW644356, MW644357, MW644358, MW644365,
MW644368, MW644378, MW631845, MW631846, MW631855, MW631856,
MW643506, MW643525, MW643541, MW644176, MW644186, MW644232,
MW644240, MW644241, MW644242, MW644251, MW644252, MW644258,
MW644259, MW644266, MW644267, MW644268, MW644269, MW644270,
MW644271, MW644272, MW644276, MW644277, MW644278, MW644279,
MW644280, MW644281, MW644287, MW644288, MW644289, MW644290,
MW644291, MW644292, MW644294, MW644295, MW644296, MW644297,
MW644298, MW644299, MW644300, MW644303, MW644305, MW644306,
MW631843, MW631844, MW631847, MW631848, MW631853, MW631865,
MW631875, MW631876, MW631877, MW631878, MW631879, MW631884,
MW631886, MW631892, MW631907, MW631911, MW631912, MW631919,
MW631928, MW644302, MW644307, MW631893, MW631894, MW631903,
MW631904, MW631913, MW631914, MW631920, MW631921, MW631922,
MW631923, MW631924, MW631929, MW632192, MW632193, MW632194,
MW632195, MW632196, MW632200, MW632201, MW632202, MW632203,
MW632204, MW632205, MW632206, MW632207, MW632208, MW632209,
MW632210, MW632211, MW632212, MW632213, MW632214, MW632215,
MW632217, MW632218, MW632219, MW632220, MW632221, MW632222,
MW632227, MW632228, MW632229, MW632230, MW633280, MW633322,
MW633323, MW633917, MW635615, MW644315, MW644316, MW632216,
MW632223, MW632224, MW632225, MW632226, MW632231, MW632232,
MW632233, MW632234, MW632235, MW632236, MW633324, MW633517,
MW633891, MW633892, MW633893, MW633896, MW633897, MW633899,
MW633900, MW633902, MW633903, MW633906, MW633907, MW633908,
MW633909, MW633911, MW633912, MW635616, MW644308, MW644309,
MW644310, MW644311, MW644312, MW644317, MW644318, MW644319,
MW644320, MW644321, MW644326, MW644327, MW644328, MW644329,
MW644330, MW644335, MW644336, MW644337, MW644363, MW644364,
MW644372, MW644373, MW644374, MW644381, MW644382, MW644383,
MW644384, MW644391, MW644392, MW644393, MW644394, MW644400,
MW644401, MW644403, MW644431, MW644432, MW644433, MW644434,
MW644435, MW644436, MW644437, MW644440, MW644441, MW644442,
MW644445, MW644446, MW644447, MW644448, MW644451, MW644452,
MW644456, MW644459, MW644460, MW644468, MW644469, MW644476,
MW644480, MW644488, MW644496, MW644498, MW644507, MW633894,
MW633904, MW633914, MW643933, MW643951, MW643952, MW643963,
MW643964, MW643969, MW643970, MW643973, MW643979, MW643980,
MW643981, MW643983, MW643987, MW643990, MW643994, MW643995,
MW643997, MW644004, MW644005, MW644015, MW644024, MW644025,
MW644338, MW644345, MW644334, MW644339, MW644340, MW644349,
MW644350, MW644353, MW644362, MW644366, MW644367, MW644225,
MW644227, MW644228, MW644229, MW644235, MW644236, MW644237,
MW644243, MW644244, MW644254, MW631778, MW631788, MW631798,
MW631805, MW631807, MW631814, MW631815, MW631824, MW631839,
MW633918, MW633919, MW633927, MW633947, MW634287, MW634293,
MW634294, MW634295, MW634296, MW634303, MW634304, MW634305,
MW634306, MW634310, MW634311, MW634312, MW634313, MW634314,
MW634315, MW634316, MW634727, MW634728, MW634729, MW634730,
MW634736, MW634737, MW634738, MW634739, MW634745, MW634746,
MW634747, MW634748, MW634749, MW634754, MW634755, MW634756,
MW634068, MW634764, MW634075, MW634766, MW634077, MW634084,
MW634085, MW634092, MW634095, MW634096, MW634102, MW634103,
MW634537, MW634540, MW634541, MW634548, MW634551, MW634557,
MW634558, MW634559, MW634567, MW634568, MW644449, MW644478,
MW644486, MW644506, MW631797, MW631806, MW631808, MW631816,
MW631817, MW631818, MW631826, MW631827, MW631828, MW631832,
MW631833, MW631834, MW631836, MW631837, MW631838, MW633920,
MW633921, MW633922, MW633923, MW633924, MW633925, MW633928,
MW633929, MW633930, MW633931, MW633932, MW633933, MW633934,
MW633937, MW633938, MW633939, MW633940, MW633941, MW633942,
MW633943, MW633948, MW633949, MW633950, MW633951, MW633952,
MW634030, MW634031, MW634032, MW634040, MW634041, MW634042,
MW634043, MW634050, MW634297, MW633936, MW633946, MW634027,
MW634028, MW634029, MW634033, MW634034, MW634036, MW634044,
MW634045, MW634047, MW634051, MW634052, MW634053, MW634054,
MW634055, MW634057, MW634060, MW634062, MW634065, MW634070,
MW634072, MW634073, MW634082, MW634091, MW634100, MW631854,
MW631864, MW631870, MW631871, MW631872, MW631873, MW631880,
MW631881, MW631882, MW631883, MW631887, MW631888, MW631889,
MW631890, MW631898, MW631899, MW631900, MW631901, MW631908,
MW631909, MW631910, MW631915, MW631916, MW631917, MW631918,
MW631925, MW631926, MW631927, MW632197, MW632198, MW632199,
MW634207, MW634215, MW634216, MW634225, MW634226, MW634233,
MW634238, MW634239, MW634242, MW634243, MW634252, MW634253,
MW634261, MW634262, MW634263, MW634268, MW634269, MW634278,
MW634217, MW634227, MW634240, MW634241, MW634246, MW634247,
MW634248, MW634249, MW634254, MW634255, MW634256, MW634257,
MW634264, MW634265, MW634266, MW634267, MW634270, MW634271,
MW634272, MW634273, MW634274, MW634275, MW634276, MW634277,
MW634279, MW634280, MW634281, MW634282, MW634283, MW634284,
MW634285, MW634286, MW634288, MW634289, MW634290, MW634291,
MW634292, MW634298, MW634299, MW634300, MW634301, MW634302,
MW634308, MW634309, MW634318, MW634319, MW634320, MW634734,
MW634735, MW634744, MW631734, MW631735, MW631736, MW631737,
MW642248, MW642255, MW642256, MW642257, MW644453, MW644454,
MW644462, MW644463, MW644464, MW644465, MW644470, MW644471,
MW644472, MW644473, MW644474, MW644475, MW644481, MW644482,
MW644483, MW644484, MW644489, MW644490, MW644491, MW644492,
MW644493, MW644494, MW644499, MW644500, MW644501, MW644502,
MW644503, MW644504, MW644508, MW644509, MW644510, MW644511,
MW644512, MW644513, MW644514, MW644516, MW644517, MW644518,
MW644519, MW644520, MW631730, MW631740, MW631741, MW634208,
MW634209, MW634210, MW634211, MW634213, MW634214, MW634218,
MW634220, MW634224, MW634229, MW634230, MW634232, MW634234,
MW634237, MW634259, MW634260, MW642250, MW642253, MW642254,
MW644485, MW644495, MW644505, MW644515, MW634049, MW634059,
MW634757, MW634069, MW634765, MW634076, MW634086, MW634087,
MW634093, MW634094, MW634101, MW634104, MW634538, MW634539,
MW634542, MW634547, MW634549, MW634550, MW644444, MW644450,
MW644457, MW644458, MW644467, MW644479, MW644487, MW644497,
MW631729, MW631731, MW631732, MW631733, MW631739, MW631742,
MW634212, MW634219, MW634221, MW634222, MW634223, MW634228,
MW634231, MW634235, MW634236, MW634244, MW634245, MW634250,
MW634251, MW634258, MW642249, MW642258, MW642259, MW633926,
MW633935, MW633944, MW633945, MW633953, MW634035, MW634037,
MW634038, MW634039, MW634046, MW634048, MW634056, MW634058,
MW634061, MW634063, MW634064, MW634066, MW634067, MW634071,
MW634074, MW634080, MW634081, MW634083, MW634090, MW644222,
MW644245, MW644253, MW644263, MW644284, MW644285, MW644388,
MW644396, MW644397, MW644398, MW644426, MW634332, MW634333,
MW634340, MW634341, MW634342, MW634343, MW634350, MW634351,
MW634352, MW634353, MW634359, MW634360, MW634361, MW634362,
MW634363, MW634369, MW634370, MW634371, MW634372, MW634373,
MW634380, MW634381, MW634382, MW634383, MW634389, MW634390,
MW634391, MW634392, MW634393, MW634394, MW634395, MW634396,
MW634397, MW634398, MW634399, MW634404, MW634405, MW634406,
MW634407, MW634408, MW634414, MW634424, MW634425, MW634433,
MW634434, MW634435, MW634997, MW634998, MW635007, MW635008,
MW634400, MW634401, MW634169, MW634179, MW634189, MW634198,
MW634199, MW634205, MW634206, MW634321, MW634322, MW634323,
MW634325, MW634326, MW634327, MW634328, MW634329, MW634330,
MW634331, MW634334, MW634335, MW634336, MW634337, MW634338,
MW634339, MW634344, MW634345, MW634346, MW634347, MW634348,
MW634349, MW634354, MW634355, MW634356, MW634357, MW634358,
MW634364, MW634365, MW634366, MW634367, MW634368, MW634374,
MW634375, MW634376, MW634377, MW634378, MW634379, MW634384,
MW634385, MW634386, MW634387, MW634388, MW634129, MW634136,
MW634137, MW634138, MW634139, MW634144, MW634145, MW634146,
MW634147, MW634148, MW634149, MW634153, MW634154, MW634155,
MW634156, MW634157, MW634158, MW634159, MW634163, MW634164,
MW634165, MW634166, MW634167, MW634168, MW634172, MW634173,
MW634174, MW634175, MW634176, MW634177, MW634178, MW634182,
MW634183, MW634184, MW634185, MW634186, MW634187, MW634188,
MW634192, MW634193, MW634194, MW634195, MW634196, MW634197,
MW634200, MW634201, MW634202, MW634203, MW634204, MW634324,
MW634499, MW634515, MW634519, MW634529, MW634779, MW634786,
MW634787, MW634788, MW634789, MW634795, MW634796, MW634797,
MW634798, MW634799, MW634800, MW634801, MW634802, MW634803,
MW634804, MW634805, MW634806, MW634810, MW634811, MW634812,
MW634813, MW634814, MW634820, MW634821, MW634822, MW634823,
MW634824, MW634825, MW634830, MW634831, MW634832, MW634833,
MW634834, MW634835, MW634840, MW634841, MW634842, MW634843,
MW634844, MW634850, MW634851, MW634852, MW634853, MW634860,
MW634861, MW634870, MW634402, MW634403, MW634409, MW634410,
MW634415, MW634416, MW634417, MW634418, MW634419, MW634420,
MW634426, MW634427, MW634428, MW634429, MW634430, MW634436,
MW634437, MW634438, MW634439, MW634488, MW634489, MW634498,
MW634513, MW634514, MW634517, MW634518, MW634528, MW634777,
MW634778, MW634783, MW634785, MW634792, MW634793, MW634794,
MW634999, MW635000, MW635001, MW635002, MW635003, MW635009,
MW635010, MW635011, MW635012, MW635017, MW635018, MW635019,
MW634807, MW634808, MW635273, MW634815, MW634816, MW635275,
MW635276, MW634826, MW634827, MW634828, MW635282, MW634837,
MW635284, MW634845, MW635286, MW634847, MW635291, MW634854,
MW635293, MW634856, MW635300, MW634859, MW634865, MW634869,
MW634873, MW634878, MW634880, MW634884, MW634892, MW634893,
MW635241, MW635250, MW635251, MW635249, MW635279, MW635295,
MW635297, MW635303, MW635304, MW635325, MW635333, MW635334,
MW635340, MW635344, MW635360, MW635361, MW635370, MW635371,
MW634118, MW634122, MW634125, MW634141, MW634151, MW634190,
MW634676, MW634686, MW634693, MW634694, MW634703, MW634705,
MW634706, MW634715, MW634724, MW634726, MW635277, MW635287,
MW635288, MW635296, MW635298, MW635306, MW635307, MW635310,
MW635313, MW635320, MW635322, MW635342, MW635343, MW635351,
MW635352, MW634114, MW634115, MW634117, MW634121, MW634126,
MW634128, MW634160, MW634695, MW634696, MW634714, MW634716,
MW634723, MW634124, MW634127, MW634130, MW634131, MW634132,
MW634134, MW634150, MW634161, MW634170, MW634685, MW634704,
MW634713, MW644341, MW644343, MW644344, MW644352, MW644361,
MW644369, MW644370, MW644375, MW644376, MW644377, MW642556,
MW642557, MW642558, MW642563, MW642564, MW642565, MW642566,
MW642594, MW642595, MW642599, MW642600, MW642601, MW642602,
MW642603, MW642604, MW642607, MW642608, MW642609, MW642610,
MW642611, MW642612, MW642613, MW642617, MW642618, MW642619,
MW642620, MW642621, MW642627, MW642629, MW642630, MW642631,
MW645669, MW645670, MW645671, MW645672, MW645674, MW645679,
MW643435, MW643436, MW643441, MW643442, MW643443, MW643444,
MW643445, MW643451, MW643452, MW643453, MW643454, MW643455,
MW643460, MW643461, MW643462, MW643463, MW643464, MW643469,
MW643470, MW643472, MW643473, MW643479, MW643480, MW643489,
MW643490, MW643491, MW643492, MW643499, MW643029, MW643037,
MW643038, MW643048, MW643049, MW643055, MW643056, MW643057,
MW643063, MW643064, MW643065, MW643067, MW643068, MW643144,
MW643145, MW643146, MW643154, MW643163, MW643173, MW643174,
MW643175, MW643184, MW643151, MW643169, MW643186, MW643205,
MW643214, MW643215, MW643447, MW643457, MW643465, MW643026,
MW643027, MW643030, MW643031, MW643032, MW643033, MW643034,
MW643035, MW643040, MW643041, MW643042, MW643043, MW643044,
MW643050, MW643051, MW643052, MW643053, MW643054, MW643060,
MW643061, MW643062, MW643070, MW643071, MW643072, MW643080,
MW643380, MW642973, MW642974, MW642983, MW642990, MW643367,
MW643372, MW643426, MW643427, MW643428, MW643434, MW643028,
MW643885, MW643036, MW643046, MW643047, MW643066, MW643069,
MW643073, MW643075, MW643076, MW643077, MW643078, MW643147,
MW643149, MW643150, MW643153, MW643155, MW643156, MW643157,
MW643158, MW643165, MW643183, MW643191, MW643152, MW643159,
MW643160, MW643162, MW643170, MW643176, MW643185, MW643188,
MW643193, MW643194, MW643203, MW643204, MW643213, MW642464,
MW642465, MW642467, MW642468, MW642482, MW642490, MW642492,
MW642552, MW642597, MW645728, MW645737, MW645871, MW645899,
MW646755, MW647192, MW647193, MW647200, MW647208, MW647210,
MW647211, MW647216, MW647217, MW647220, MW642635, MW642639,
MW642643, MW642648, MW642654, MW642658, MW642659, MW642663,
MW642672, MW642673, MW642676, MW642680, MW642682, MW642683,
MW642686, MW642700, MW642703, MW642710, MW642713, MW642740,
MW646617, MW646627, MW646635, MW643218, MW643220, MW643222,
MW643225, MW643228, MW643233, MW643234, MW643236, MW642640,
MW642641, MW642650, MW642662, MW643252, MW643261, MW643262,
MW643603, MW643604, MW643606, MW643613, MW643616, MW643620,
MW643623, MW643625, MW643631, MW643632, MW643633, MW643639,
MW643640, MW643641, MW643647, MW643648, MW643657, MW643658,
MW646601, MW646616, MW646633, MW646651, MW642679, MW642755,
MW642782, MW642783, MW642785, MW642790, MW642791, MW642792,
MW642793, MW642794, MW642796, MW642804, MW642808, MW642809,
MW642812, MW642813, MW642818, MW642829, MW642840, MW642847,
MW642848, MW642871, MW642645, MW642653, MW643251, MW643260,
MW643612, MW643614, MW643621, MW643622, MW643624, MW643630,
MW643634, MW643638, MW643650, MW646602, MW646614, MW646624,
MW646634, MW646643, MW642678, MW642694, MW642705, MW634586,
MW634595, MW634596, MW634604, MW634605, MW634606, MW634607,
MW634612, MW634613, MW634614, MW634615, MW634616, MW634617,
MW634618, MW634622, MW634623, MW634624, MW634625, MW634626,
MW634631, MW634632, MW634633, MW634634, MW634635, MW634636,
MW634641, MW634642, MW634643, MW634644, MW634645, MW634646,
MW634648, MW634649, MW634650, MW634651, MW634652, MW634653,
MW634654, MW634655, MW634657, MW634658, MW634659, MW634660,
MW634661, MW634667, MW634668, MW634677, MW634678, MW634687,
MW634717, MW634120, MW634143, MW634181, MW634662, MW634663,
MW634666, MW634669, MW634675, MW634682, MW634683, MW634689,
MW634692, MW634697, MW634698, MW634699, MW634701, MW634702,
MW634707, MW634708, MW634710, MW634719, MW634722, MW634459,
MW634464, MW634465, MW634466, MW634474, MW634476, MW634479,
MW634480, MW634482, MW634483, MW634485, MW634486, MW634487,
MW634491, MW634494, MW634495, MW634496, MW634497, MW634500,
MW634501, MW634505, MW634506, MW634507, MW634508, MW634510,
MW634511, MW634512, MW634516, MW634520, MW634522, MW634523,
MW634526, MW634527, MW634531, MW634535, MW634536, MW634585,
MW634594, MW634603, MW634611, MW634621, MW634629, MW634630,
MW634640, MW634647, MW634780, MW634781, MW634782, MW634790,
MW634791, MW634451, MW634452, MW634453, MW634454, MW634455,
MW634456, MW634457, MW634458, MW634460, MW634461, MW634462,
MW634463, MW634467, MW634468, MW634469, MW634471, MW634472,
MW634473, MW634475, MW634477, MW634478, MW634481, MW634484,
MW634490, MW634492, MW634493, MW634502, MW634503, MW634504,
MW634509, MW634521, MW634524, MW634525, MW634530, MW634532,
MW634533, MW634534, MW635663, MW635664, MW635665, MW635674,
MW635675, MW635683, MW635684, MW635685, MW635692, MW635693,
MW635694, MW635695, MW631796, MW635517, MW635518, MW635519,
MW635520, MW635521, MW635522, MW635526, MW635527, MW635528,
MW635529, MW635530, MW635531, MW635532, MW635535, MW635536,
MW635537, MW635539, MW635544, MW635545, MW635546, MW635547,
MW635548, MW635553, MW635554, MW635555, MW635556, MW635557,
MW635558, MW635564, MW635565, MW635566, MW635567, MW635569,
MW635571, MW635575, MW635576, MW635584, MW635585, MW635587,
MW635638, MW631767, MW631775, MW631785, MW631786, MW631795,
MW631804, MW631810, MW631829, MW635538, MW635551, MW635552,
MW635562, MW635582, MW635588, MW635633, MW635636, MW635637,
MW635639, MW635647, MW634440, MW634441, MW634442, MW634443,
MW634444, MW634445, MW634446, MW634447, MW634448, MW634449,
MW634450, MW634470, MW635635, MW635640, MW635641, MW635643,
MW635644, MW635645, MW635651, MW635652, MW635655, MW635656,
MW635657, MW635658, MW635659, MW635660, MW635661, MW635662,
MW635666, MW635667, MW635670, MW635671, MW635672, MW635676,
MW635677, MW635678, MW635679, MW635680, MW635681, MW635682,
MW635688, MW635689, MW635690, MW635691, MW631766, MW631768,
MW631776, MW631777, MW631787, MW631793, MW631794, MW631802,
MW631803, MW631811, MW631812, MW631813, MW631822, MW631823,
MW631830, MW631831, MW635540, MW635541, MW635550, MW635560,
MW635561, MW635570, MW635572, MW635580, MW635581, MW635646,
MW635649, MW634110, MW634111, MW634112, MW634152, MW634162,
MW634171, MW634191, MW634656, MW634664, MW634665, MW634670,
MW634671, MW634672, MW634673, MW634674, MW634679, MW634680,
MW634681, MW634688, MW634690, MW634691, MW634700, MW634709,
MW634711, MW634712, MW634718, MW634720, MW634721, MW634317,
MW634740, MW634741, MW634750, MW634758, MW634768, MW634775,
MW635451, MW635480, MW635491, MW635492, MW635511, MW635512,
MW635515, MW635523, MW635524, MW635563, MW635574, MW634078,
MW634079, MW634088, MW634089, MW634097, MW634098, MW634099,
MW634105, MW634106, MW634107, MW634108, MW634109, MW634543,
MW634544, MW634545, MW634546, MW634552, MW634553, MW634554,
MW634555, MW634556, MW634560, MW634561, MW634562, MW634563,
MW634564, MW634565, MW634566, MW634569, MW634570, MW634571,
MW634572, MW634573, MW634574, MW634575, MW634577, MW634578,
MW634579, MW634580, MW634581, MW634587, MW634588, MW634589,
MW634590, MW634591, MW634592, MW634597, MW634598, MW634600,
MW634637, MW634411, MW634412, MW634413, MW634421, MW634422,
MW634423, MW634431, MW634432, MW634994, MW634995, MW634996,
MW635004, MW635005, MW635014, MW635015, MW635016, MW635427,
MW635429, MW635431, MW635435, MW635436, MW635437, MW635438,
MW635439, MW635440, MW635443, MW635444, MW635445, MW635446,
MW635447, MW635453, MW635455, MW635456, MW635463, MW635464,
MW635466, MW635472, MW635473, MW635483, MW635484, MW635485,
MW635493, MW635494, MW635503, MW635504, MW635505, MW634576,
MW634584, MW634599, MW634601, MW634608, MW634609, MW634610,
MW634619, MW634620, MW634627, MW634638, MW634639, MW634993,
MW635006, MW635428, MW635430, MW635433, MW635441, MW635457,
MW635458, MW635469, MW635476, MW635477, MW635479, MW635487,
MW635495, MW635497, MW635506, MW631745, MW631746, MW631747,
MW631748, MW631752, MW631753, MW631754, MW631755, MW631756,
MW631757, MW631758, MW631760, MW631761, MW631762, MW631763,
MW631764, MW631765, MW631770, MW631771, MW631772, MW631773,
MW631774, MW631779, MW631780, MW631781, MW631782, MW631783,
MW631784, MW631789, MW631790, MW631791, MW631792, MW631799,
MW631800, MW631801, MW631809, MW631819, MW631820, MW631821,
MW635595, MW635604, MW635605, MW635613, MW635614, MW635617,
MW635624, MW635625, MW635626, MW635627, MW631743, MW631744,
MW631749, MW631750, MW631751, MW631759, MW631769, MW634743,
MW634751, MW634752, MW634753, MW634760, MW634761, MW634762,
MW634763, MW634769, MW634770, MW634771, MW634772, MW634773,
MW634776, MW635590, MW635592, MW635593, MW635594, MW635597,
MW635598, MW635599, MW635600, MW635601, MW635602, MW635606,
MW635607, MW635608, MW635609, MW635610, MW635618, MW635620,
MW635621, MW635622, MW635623, MW635629, MW635630, MW635631,
MW634307, MW634731, MW634732, MW634733, MW634742, MW634759,
MW634767, MW634774, MW635452, MW635461, MW635462, MW635470,
MW635471, MW635475, MW635481, MW635482, MW635489, MW635490,
MW635499, MW635500, MW635502, MW635509, MW635510, MW635514,
MW635516, MW635525, MW635543, MW635573, MW635596, MW634582,
MW634583, MW634593, MW634602, MW634628, MW635013, MW635434,
MW635442, MW635448, MW635449, MW635450, MW635468, MW635488,
MW635496, MW635498, MW635507, MW635508, MW635513, MW644379,
MW644380, MW644387, MW644389, MW644399, MW644427, MW644428,
MW672146, MW672147, MW672148, MW672155, MW672157, MW672165,
MW672173, MW672616, MW672626, MW672628, MW674795, MW674797,
MW674799, MW674800, MW674803, MW674805, MW674809, MW674810,
MW674813, MW681336, MW681345, MW681346, MW681372, MW685486,
MW685487, MW681373, MW685491, MW681390, MW685499, MW681400,
MW681405, MW685576, MW681407, MW681414, MW681434, MW685586,
MW685591, MW685588, MW685617, MW685636, MW685682, MW685685,
MW685692, MW685488, MW685489, MW685490, MW681380, MW685492,
MW685497, MW681399, MW685500, MW685577, MW685578, MW685585,
MW685583, MW685587, MW685592, MW685593, MW685594, MW685600,
MW685603, MW685607, MW685609, MW685610, MW685627, MW685637,
MW685660, MW685661, MW685674, MW685675, MW685684, MW681379,
MW681381, MW681389, MW681398, MW681406, MW681415, MW685584,
MW685595, MW685596, MW685601, MW685602, MW685608, MW685611,
MW685628, MW685638, MW685683, MW685686, MW681242, MW681485,
MW681486, MW681487, MW681494, MW681495, MW681496, MW681504,
MW681505, MW681506, MW681512, MW681513, MW681514, MW681515,
MW681526, MW681527, MW681538, MW681545, MW681553, MW684206,
MW684207, MW684214, MW684215, MW684223, MW684224, MW684234,
MW684240, MW684242, MW684248, MW684251, MW684252, MW684253,
MW685649, MW684629, MW684637, MW684639, MW684644, MW684646,
MW684649, MW684654, MW684655, MW684657, MW684661, MW684662,
MW684663, MW684664, MW684619, MW684636, MW684638, MW684643,
MW684645, MW684647, MW684648, MW684653, MW684656, MW684658,
MW684660, MW664392, MW664402, MW664409, MW664410, MW664411,
MW664412, MW664421, MW664422, MW664423, MW665565, MW665573,
MW665574, MW665575, MW665576, MW665577, MW665578, MW665580,
MW665581, MW665582, MW665583, MW665584, MW665585, MW665587,
MW665588, MW665589, MW665590, MW665591, MW665592, MW665593,
MW665597, MW665598, MW665599, MW665600, MW665601, MW665602,
MW665606, MW665607, MW665608, MW665609, MW664390, MW665611,
MW665616, MW664400, MW665618, MW664407, MW664408, MW664420,
MW666308, MW666309, MW666318, MW665579, MW665596, MW666597,
MW666607, MW666626, MW666627, MW666634, MW666636, MW666646,
MW666650, MW666653, MW666659, MW666660, MW666662, MW666669,
MW666670, MW666678, MW666679, MW666689, MW665729, MW665730,
MW665769, MW665770, MW665777, MW665778, MW665779, MW665780,
MW665785, MW665786, MW665787, MW665788, MW665789, MW665790,
MW665796, MW665797, MW665798, MW665800, MW665804, MW665805,
MW665806, MW665807, MW665808, MW665809, MW665815, MW665816,
MW665817, MW665818, MW665820, MW665821, MW665822, MW665823,
MW665824, MW665827, MW665831, MW665832, MW665833, MW665834,
MW665835, MW665837, MW665841, MW665842, MW665844, MW665845,
MW665851, MW665853, MW666184, MW666185, MW666192, MW666201,
MW673334, MW673335, MW673337, MW673345, MW673346, MW673347,
MW673518, MW673519, MW673520, MW673522, MW673526, MW673527,
MW673537, MW673539, MW673541, MW673544, MW673545, MW673552,
MW673553, MW673560, MW673561, MW673568, MW673569, MW673571,
MW673572, MW673579, MW665799, MW665810, MW665819, MW665825,
MW665826, MW665828, MW665829, MW665830, MW665836, MW665839,
MW665840, MW665843, MW665846, MW665848, MW665849, MW665850,
MW665852, MW666183, MW666186, MW666188, MW666189, MW666190,
MW666191, MW666193, MW666194, MW666195, MW666196, MW666197,
MW666198, MW666200, MW666202, MW666204, MW666205, MW666206,
MW666207, MW666210, MW666570, MW666571, MW666572, MW666573,
MW666579, MW666580, MW666581, MW666582, MW666588, MW666589,
MW666590, MW666598, MW666599, MW666608, MW666574, MW666575,
MW666576, MW666577, MW666583, MW666585, MW666592, MW666594,
MW666596, MW666600, MW666604, MW666605, MW666610, MW666611,
MW666614, MW666615, MW666619, MW666620, MW666621, MW666622,
MW666623, MW666630, MW666631, MW666632, MW666638, MW666640,
MW666641, MW665667, MW665668, MW665669, MW665670, MW665676

### Example 4: Methods

### PBMC Isolation and RNA-Stimulation

1. Healthy donor (HD) or recovered COVID-19 patients (RP)-PMBCs were isolated by density gradient using Ficoll from buffy coats provided by LifeSouth, Gainesville, Florida. A portion of the PMBCs from each HD and RP did not undergo any manipulation other than isolation and freezing, and were designated as baseline (untreated).

2. A portion of the isolated PBMCs were plated (24 well plate at a concentration of 1×10⁶ cells/ml of AIM-V) and cultured for 24 hours at 37° C. incubator supplied with 5% CO₂. Non-adherent and adherent fractions were collected, spun down, and adjusted to a concentration of 5 million cells/200 μl in OPTI-MEM. Cell electroporation (EP) was done with 20 μg of full-length spike (S) protein mRNA.

3. Electroporated cells were immediately plated into a 24 well plate at a concentration of 1×10⁶ cells/ml of AIM-V+5% human serum and 50 U/ml of IL2 for expansion. At 5th day, 50 U/ml of IL2 was added to each well for expansion (expanded T cells).

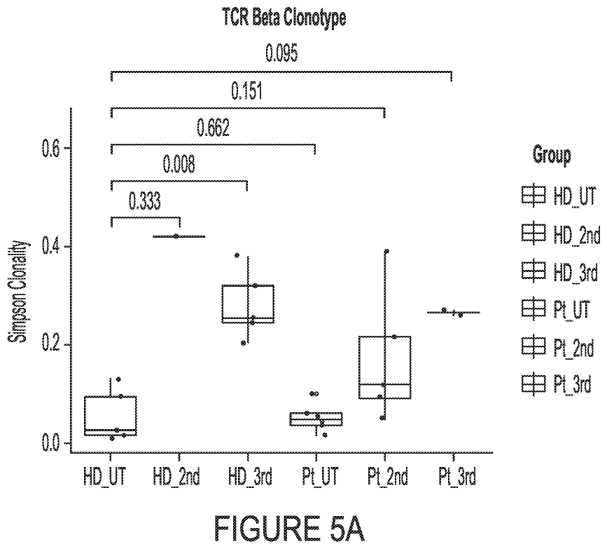

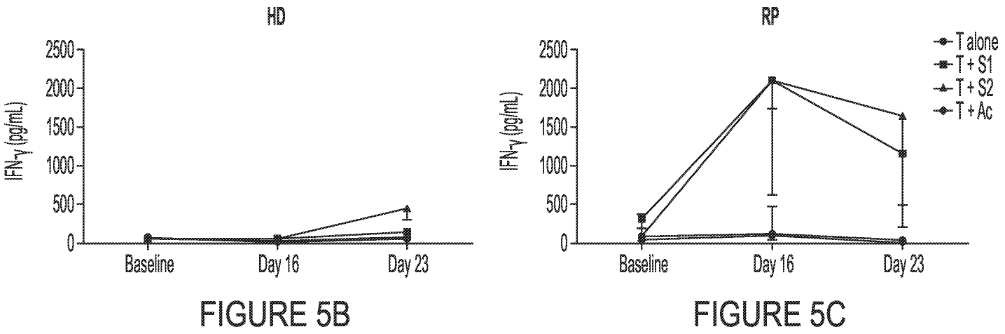

4. At day 9, PBMCs from each HD and RP were thawed, and step 2 was repeated for the second stimulation. At 24 hours after the second electroporation, cells were irradiated with 40 Gy. At day 10, irradiated S mRNA EP PBMCs were co-cultured with day 10 expanded T cells (step 3 above, prepared from same HD or RP as used for the PBMCs) at a ratio of 1:1 (1x106 cells each) in AIM-V+5% human serum and 50 U/ml of IL2. Five days after the second stimulation, IL2 was added at 50 U/ml. Cells were split if there was more than 90% of confluence. The cells produced in step 4 were designated “HD_2nd” and “Pt_2nd” in FIG. 5A and designated “Day 16” in FIGS. 5B-C.

5. A third stimulation of PBMCs with full-length spike (S) protein mRNA was performed at day 15 following similar steps as described in step 4 for the second stimulation and expansion lasted until day 23. Expanded T cells were as in step 3 above. The cells produced in step 5 were designated “HD_3rd” and “Pt_3rd” in FIG. 5A and designated “Day 23” in FIGS. 5B-C.

Preparation of Spike mRNA

Spike DNA sequence was extracted from SARS-COV-2 (2019-nCOV) Spike ORF mammalian expression plasmid (Codon Optimized); Cat: VG40589-UT; Sino Biological US Inc., Wayne, PA) using HindIII and BamHI restriction enzymes. The insert was subsequently cloned into a pGEM4z plasmid backbone, which has the required components for mRNA production. pGEM4z-Spike plasmid was grown in bacteria and purified using Zymo ZymoPure II Plasmid Maxiprep Kit (catalog #D4203, Zymo Research). Plasmid was opened using Spe I restriction enzyme and purified by ethanol precipitation method. Open pGEM4z-Spike plasmid was used as template to produce Spike mRNA using Invitrogen mMESSAGE mMACHINE T7Transcription Kit (catalog #AM1344, Invitrogen). Final Spike mRNA product was purified using QIAGEN RNeasy Maxi kit (catalog #75162, QIAGEN).

### INF-Gamma Secretion Protocol

Healthy donor (HD) or covid-19 recovered patients (RP)-expanded T cells with full-length S mRNA EP PBMCs, or baseline HD or RP PBMCs prepared as above, were co-cultured with spike protein peptides S1 (S1), spike protein peptides S2 (S2) or actin peptides (Ac) in AIM-V+5% human serum. Supernatant was collected at 36 hours post peptide stimulation. INF-gamma release was detected by ELISA.

### Peptides Used in INF-Gamma Secretion Experiments

Spike protein peptides S1 were a pool of 158 peptides derived from a peptide scan (15mers with 11 aa overlap) through residues 1-685 of Spike glycoprotein and spike protein peptides S2 were a pool of 157 peptides derived from a peptide scan (15mers with 11 aa overlap) through residues 686-1273 of Spike glycoprotein (Swiss-Prot ID: P0DTC2; SEQ ID NO:322) of SARS-COV-2 (Severe Acute Respiratory Syndrome-related coronavirus 2) (PepMix™ SARS-CoV-2 (Spike Glycoprotein); PM-WCPV-S-1; JPT Peptide Technologies GmbH, Berlin, Germany).

Actin peptides were a pool of 92 peptides derived from a peptide scan (15mers with 11 aa overlap) through Actin, alpha skeletal muscle (Swiss-Prot ID: P68133; SEQ ID NO:323) of Homo sapiens (Human) (PepMix™ Human (Actin), PM-ACTS; JPT Peptide Technologies GmbH, Berlin, Germany).

### Example 5: Results of INF-Gamma Secretion Experiments

Healthy donor (HD) or covid-19 recovered patients (RP)-expanded T cells with full-length S mRNA EP PBMCs collected at day 16 and 23 after mRNA stimulation, and baseline HD or RP PBMCs prepared as in Example 4, were stimulated with peptides spike S1 and S2. INF-gamma secretion was detected in the supernatant by ELISA at 36 hours post stimulation with peptide. FIGS. 5B and 5C show INF-gamma secretion by from baseline PMBCs and by expanded T cells from day 16 and 23 of HD (FIG. 4B) and COVID-19 RP (FIG. 4C) patients. (S1, spike protein S1; S2, spike protein S2; Ac, actin peptide; 2nd, day 16 of stimulation; 3rd, day 23 of stimulation; HD, healthy donor; RP & Pt, COVID-19 recovered patient; UT, untreated.)

The interferon gamma release in healthy donors increased after the third stimulation with full-length S mRNA (FIG. 5B). In recovered COVID-19 patients (FIG. 5C) the interferon gamma released was increased at the second stimulation with full-length S mRNA (Day 16) using Spike glycoprotein mRNA transfected PBMCs and decreased in the third stimulation with full-length S mRNA (Day 23).

### Example 6: TCR Beta Clonotypes of the Expanded T Cells and Identification of Hypomutated Epitopes of SARS-CoV2 Spike

Methods: Healthy donor (HD) or covid-19 recovered patients (RP)-expanded T cells with full-length S mRNA EP PBMCs were collected at day 16 and 23 after mRNA stimulation, and baseline HD or RP PBMCs prepared as in Example 4, and submitted for TCR profiling (Adaptive Biotechnologies). The heavy chain complementary-determining region 3 (HCDR3) sequences were mapped to SARS-CoV2 spike epitopes from Adaptive Biotechnologies ImmuneCODE database. SARS-CoV2 spike epitopes were compared to cold spot “hypomutated regions” presented in FIG. 4. For example SARS-CoV2 spike hypomutated region SEQ ID NO:1013 comprises hypomutated epitope SEQ ID NO:941, and SARS-CoV2 spike hypomutated region SEQ ID NO: 1006 comprises hypomutated epitope SEQ ID NO:942.

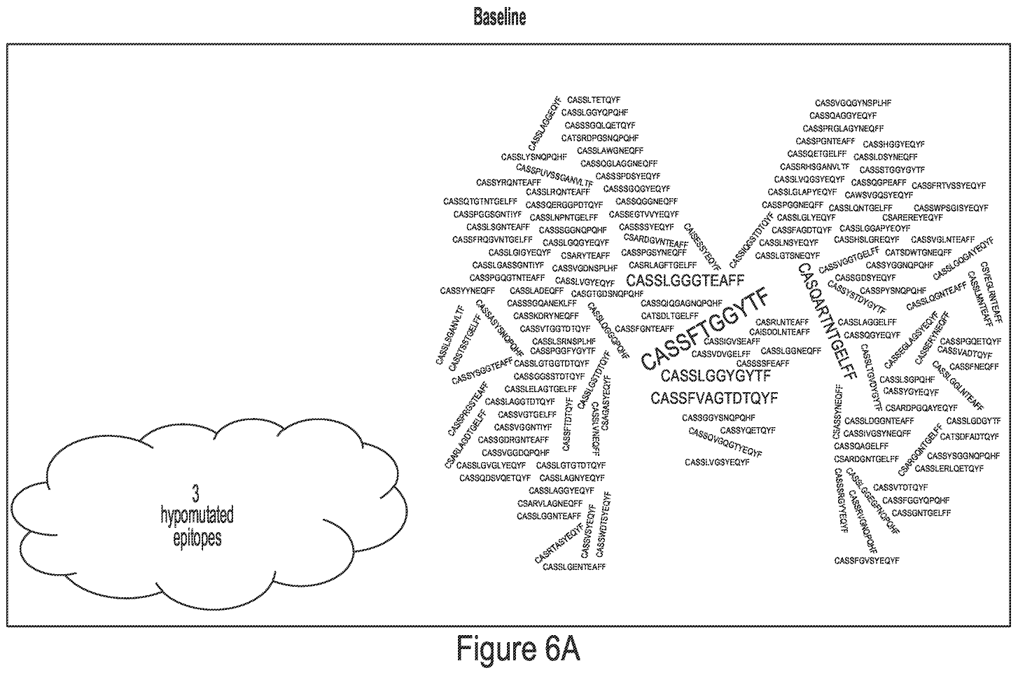

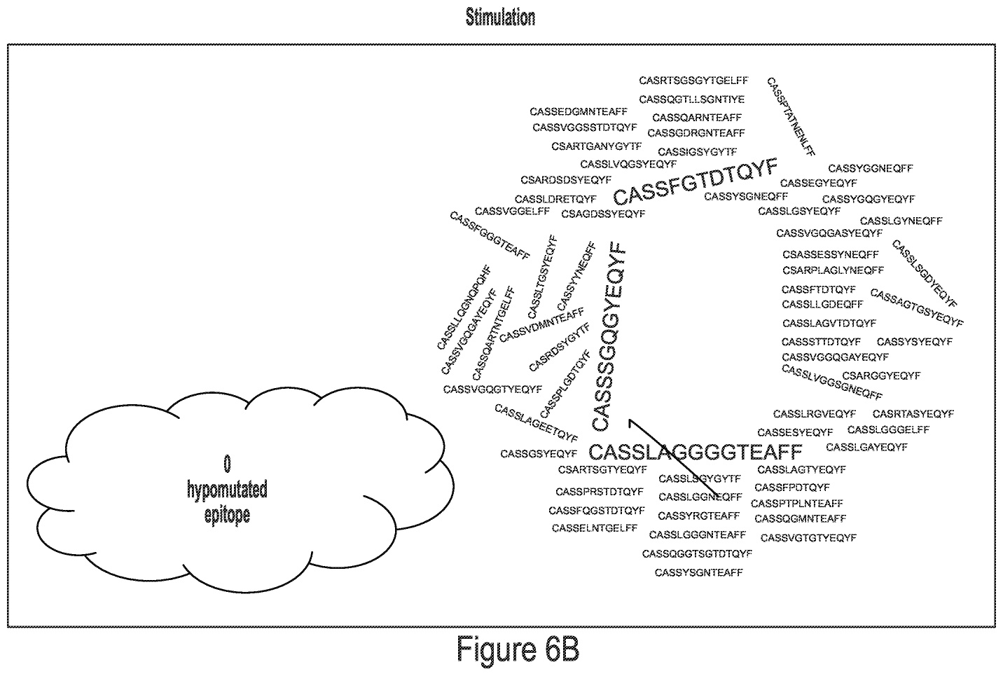

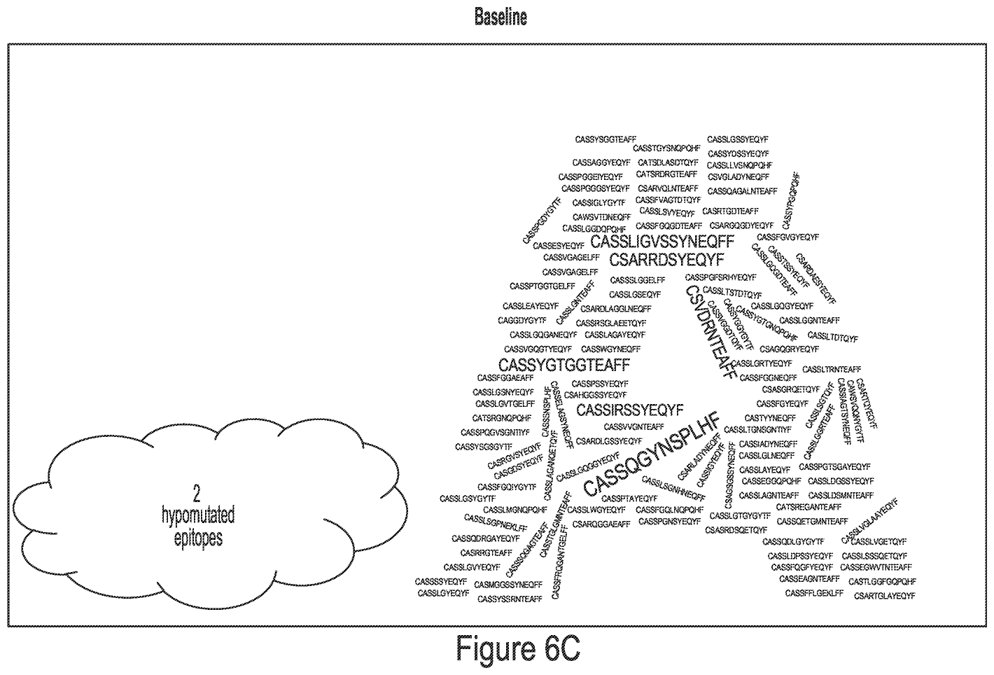

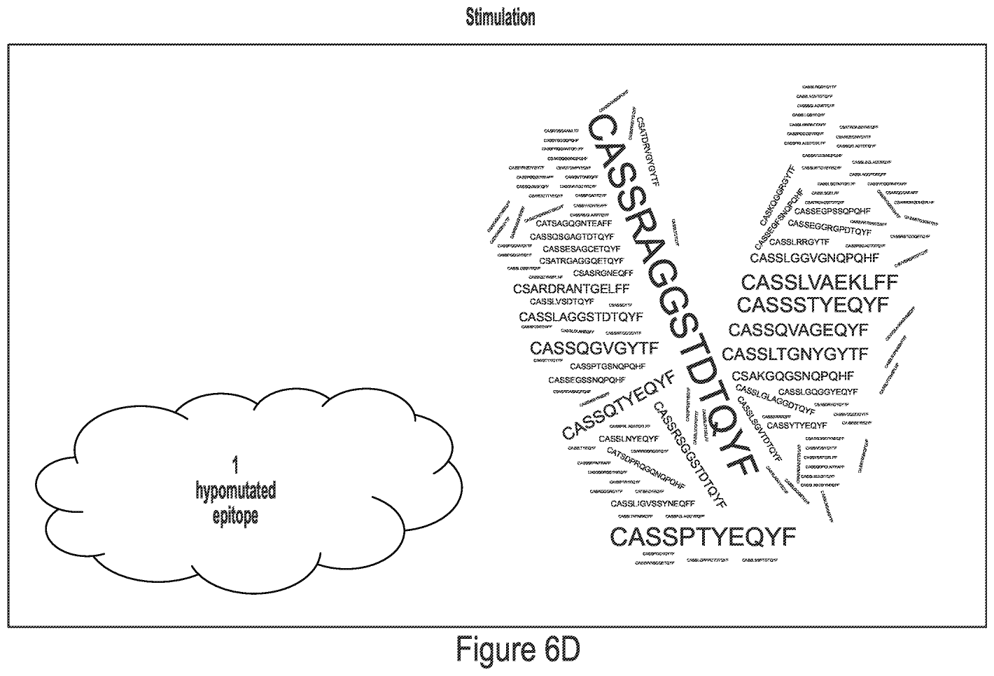

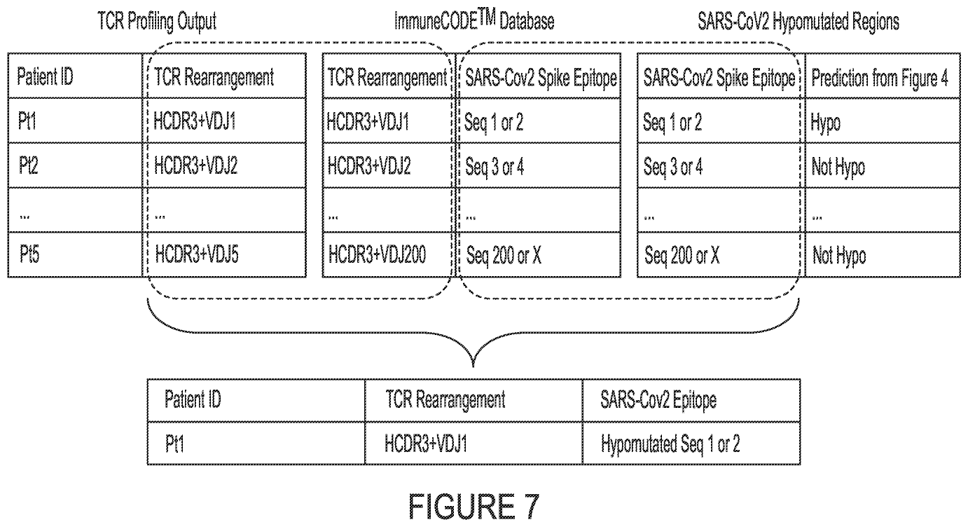

FIG. 7 is a schematic showing determination of SARS-CoV2 hypomutated epitope from TCR profiling output, ImmuneCODE database analysis, and SARS-CoV2 hypomutated regions from FIG. 4. Word cloud illustration (FIG. 6A-D) represents the predominant HCDR3/TCR clones, allowing the identification of selected hypomutated epitopes from patient samples (Table 5). The HCDR3/TCR clones and frequencies in FIGS. 6A-D are also presented in tabular form in Tables 1-4.

Results: Healthy donor (HD) or COVID-19 recovered patients (RP)-expanded T cells with full-length S mRNA EP PBMCs were collected at day 16 and 23 after mRNA stimulation, and baseline HD or RP PBMCs prepared as in Example 4. TCR beta clonotypes of the expanded T cells were analyzed by TCR profiling. FIG. 5A is a Simpson clonality boxplot representation of TCR beta clonotype sequences of HD and RP. (2nd, day 16 of stimulation; 3rd, day 23 of stimulation; HD, healthy donor; RP & Pt, COVID-19 recovered patient; UT, untreated.) Low frequency spike T cell beta clonotypes were expanded from healthy donors and from recovered COVID-19 patients.

Healthy donor (HD) or COVID-19 recovered patients (RP)-expanded T cells with full-length S mRNA EP PBMCs were collected at day 16 and 23 after mRNA stimulation, and baseline HD or RP PBMCs were prepared as in Example 4, and their TCR beta clonotypes were analyzed by TCR profiling (FIG. 5A). Word cloud illustration of HCDR3 region sequences are shown in (FIG. 6A) HD and (FIG. 6C) RP at baseline or (FIG. 6B) HD and RP (FIG. 6D) after mRNA stimulation with full-length S mRNA. The number of TCR restricted hypomutated epitopes identified, after the mapping of HCDR3 sequences with Adaptive Biotechnologies ImmuneCODE database, are annotated in balloons. Healthy donor (n=13) and recovered patients (n=5). The TCR rearrangement and corresponded Hypomutated epitopes determined are presented in Table 5.

TABLE 1
Untreated Healthy Donor HCDR3
Frequency and Sequences

| Frequency | HCDR3 | SEQ ID NO: |
|---|---|---|
| 524 | CASSLGGNTEAFF | 324 |
| 260 | CAWSVGQSYEQYF | 325 |
| 162 | CASSGDRGNTEAFF | 326 |
| 66 | CASSPGNTEAFF | 327 |
| 55 | CASSLRQNTEAFF | 328 |
| 51 | CASSYYNEQFF | 329 |
| 43 | CASSVGGNTIYF | 330 |
| 42 | CASSLAWGNEQFF | 331 |
| 40 | CASSLMNTEAFF | 332 |
| 35 | CASSQARTNTGELFF | 333 |
| 35 | CATSRDPGSNQPQHF | 334 |
| 34 | CASSLAGGYEQYF | 335 |
| 33 | CASSLGGYGYTF | 336 |
| 33 | CSAREREYEQYF | 337 |
| 29 | CASSVSYEQYF | 338 |
| 29 | CASSLGQGAYEQYF | 339 |
| 29 | CASSPWSSGANVLTF | 340 |
| 26 | CSARGQNTGELFF | 341 |
| 25 | CASSPRGSTEAFF | 342 |
| 24 | CASSFRQGVNTGELFF | 343 |
| 24 | CASSWDTSYEQYF | 344 |
| 24 | CASSLAGNYEQYF | 345 |
| 23 | CASSYSGGTEAFF | 346 |
| 23 | CASSLGTGTDTQYF | 347 |
| 22 | CASSSRGYYEQYF | 348 |
| 21 | CSARDGNTGELFF | 349 |
| 20 | CSARDPGQAYEQYF | 350 |
| 20 | CASSLGGAPYEQYF | 351 |
| 19 | CASSLGGLNTEAFF | 352 |
| 17 | CASSLNPNTGELFF | 353 |
| 17 | CASSVGTGELFF | 354 |
| 16 | CASSLONTGELFF | 355 |
| 16 | CASSLSGANVLTF | 356 |
| 16 | CATSDWTGNEQFF | 357 |
| 15 | CASSQETGELFF | 358 |
| 15 | CASSLGGEGFNQPQHF | 359 |
| 14 | CASSLGASSGNTIYF | 360 |
| 13 | CASSQERGGPDTQYF | 361 |
| 13 | CASSLGIGYEQYF | 362 |
| 13 | CASSVGGDQPQHF | 363 |
| 13 | CASSQGPEAFF | 364 |
| 13 | CASSQDSVQETQYF | 365 |
| 12 | CASSVGQGYNSPLHF | 366 |
| 12 | CSARLAGDTGELFF | 367 |
| 12 | CSVEGLRNTEAFF | 368 |
| 11 | CASSYRQNTEAFF | 369 |
| 11 | CASSFGGYQPQHF | 370 |
| 11 | CASSLERLQETQYF | 371 |
| 11 | CATSDFADTQYF | 372 |
| 11 | CASSQTGTNTGELFF | 373 |
| 11 | CASRTASYEQYF | 374 |
| 10 | CASSFRTVSSYEQYF | 375 |
| 10 | CASSFNEQFF | 376 |
| 10 | CASSLTETQYF | 377 |
| 10 | CASSLGGYGYTF | 378 |
| 10 | CASSLGENTEAFF | 379 |
| 10 | CASSLAGGEQYF | 380 |
| 10 | CASSHGGYEQYF | 381 |
| 10 | CASSFGVSYEQYF | 382 |
| 10 | CASSWPSGISYEQYF | 383 |
| 10 | CASSFTGGYTF | 384 |
| 9 | CASSLGDGYTF | 385 |
| 9 | CASSVTDTQYF | 386 |
| 9 | CASSSTGGYGYTF | 387 |
| 9 | CASSLGGYQPQHF | 388 |
| 9 | CSARVLAGNEQFF | 389 |
| 9 | CASSLSGNTEAFF | 390 |
| 9 | CASSSGQLQETQYF | 391 |
| 9 | CASSVGLNTEAFF | 392 |
| 9 | CASSPRGLAGYNEQFF | 393 |
| 9 | CASSFTGGYTF | 394 |
| 9 | CASSQAGGYEQYF | 395 |
| 9 | CASSPGGSGNTIYF | 396 |
| 8 | CASSYSGGNQPQHF | 397 |
| 8 | CASSLYSNQPQHF | 398 |
| 8 | CASSRVGNQPQHF | 399 |
| 8 | CASSLGVGLYEQYF | 400 |
| 8 | CASSLDSYNEQFF | 401 |
| 8 | CASRLNTEAFF | 402 |
| 8 | CASSLOGNTEAFF | 403 |
| 8 | CASSGNTGELFF | 404 |
| 8 | CASSSPDSYEQYF | 405 |
| 8 | CASSVTGGTDTQYF | 406 |
| 8 | CASSLAGGELFF | 407 |
| 8 | CASSLNSYEQYF | 408 |
| 7 | CSARDGVNTEAFF | 409 |
| 7 | CASSLGSTDTQYF | 410 |
| 7 | CASSLGTSNEQYF | 411 |
| 7 | CASSLSRNSPLHF | 412 |
| 7 | CASSQGYEQYF | 413 |
| 7 | CASSYSTDYGYTF | 414 |
| 7 | CASSPGGFYGYTF | 415 |
| 7 | CASSLVGYEQYF | 416 |
| 7 | CASSEGTVVYEQYF | 417 |
| 7 | CASSFVAGTDTQYF | 418 |
| 7 | CSAGASYEQYF | 419 |
| 7 | CASSLGTGGTDTQYF | 420 |
| 7 | CASSLTGVDYGYTF | 421 |
| 7 | CASSKDRYNEQFF | 422 |
| 7 | CASGTGDSNQPQHF | 423 |
| 7 | CASSFAGDTQYF | 424 |
| 7 | CASSQIQGAGNQPQHF | 425 |
| 6 | CASSIGVSEAFF | 426 |
| 6 | CATSDLTGELFF | 427 |
| 6 | CAISDDLNTEAFF | 428 |
| 6 | CASSSSFEAFF | 429 |
| 6 | CASSQARTNTGELFF | 430 |
| 6 | CASSGGYSNQPQHF | 431 |
| 6 | CASSVDVGELFF | 432 |
| 6 | CASSFGNTEAFF | 433 |
| 6 | CASSYQETQYF | 434 |
| 6 | CASSLGGNEQFF | 435 |
| 6 | CASSLVGSYEQYF | 436 |
| 6 | CASSPGSYNEQFF | 437 |
| 6 | CASSIQGSTDTQYF | 438 |
| 6 | CASRLAGFTGELFF | 439 |
| 6 | CAISESSYEQYF | 440 |
| 6 | CASSQVGQGTYEQYF | 441 |
| 6 | CASSLOGGQPQHF | 442 |
| 6 | CASSSSYEQYF | 443 |
| 6 | CASSVADTQYF | 444 |
| 6 | CASSEGLAGSYEQYF | 445 |
| 6 | CASSHSLGREQYF | 446 |
| 6 | CASSYGGNQPQHF | 447 |
| 6 | CASSSGGNQPQHF | 448 |
| 5 | CASSLGGGTEAFF | 449 |
| 5 | CASSLVQGSYEQYF | 450 |
| 5 | CASSLSGPQHF | 451 |
| 5 | CASSLDGGNTEAFF | 452 |
| 5 | CASSFTDTQYF | 453 |
| 5 | CASSYGYEQYF | 454 |
| 5 | CASSIVGSYNEQFF | 455 |
| 5 | CASSQGLAGGNEQFF | 456 |
| 5 | CASSTSSTGELFF | 457 |
| 5 | CASSRHSGANVLTF | 458 |
| 5 | CASSPGQGTNTEAFF | 459 |
| 5 | CASSPGQETQYF | 460 |
| 5 | CASSQAGELFF | 461 |
| 5 | CASSLAGGTDTQYF | 462 |
| 5 | CASSLGQGYEQYF | 463 |
| 5 | CASSLADEQFF | 464 |
| 5 | CASSQGGNEQFF | 465 |
| 5 | CASSFVAGTDTQYF | 466 |
| 5 | CASSPGGNEQFF | 467 |
| 5 | CASSGDSYEQYF | 468 |
| 5 | CASSGGSSTDTQYF | 469 |
| 5 | CASSSGQANEKLFF | 470 |
| 5 | CSASSYNEQFF | 471 |
| 5 | CASSVGGTGELFF | 472 |
| 5 | CASSVGDNSPLHF | 473 |
| 5 | CASSLGLYEQYF | 474 |
| 5 | CSARYTEAFF | 475 |
| 5 | CASSLGLAPYEQYF | 476 |
| 5 | CASSLGGGTEAFF | 477 |
| 5 | CASSASYSNQPQHF | 478 |
| 5 | CASSSGQGYEQYF | 479 |
| 5 | CASSFTGGYTF | 480 |
| 5 | CASSLVNEQFF | 481 |
| 5 | CASSPYSNQPQHF | 482 |
| 5 | CASSLELAGTGELFF | 483 |
| 5 | CASSSERYNEQFF | 484 |

TABLE 2
Stimulated Healthy Donors'
HCDR3 Frequency and Sequences

| Frequency | HCDR3 | SEQ ID NO: |
|---|---|---|
| 465 | CASSLGGGELFF | 485 |
| 436 | CASSLAGGGGTEAFF | 486 |
| 17 | CASSLSGDYEQYF | 487 |
| 6 | CASSVGQGTYEQYF | 488 |
| 487 | CASSPTATNEKLFF | 489 |
| 5 | CASSLDRETQYF | 490 |
| 6 | CSARTGANYGYTF | 491 |
| 130 | CSARDSDSYEQYF | 492 |
| 215 | CASSLAGGGGTEAFF | 493 |
| 6 | CASSLAGEETQYF | 494 |
| 5 | CASSPRSTDTQYF | 495 |
| 6 | CSARTSGTYEQYF | 496 |
| 35 | CASRTSGSGYTGELFF | 497 |
| 14 | CSARGGYEQYF | 498 |
| 294 | CASSLAGTYEQYF | 499 |
| 9 | CASSYSGNTEAFF | 500 |
| 5 | CASSSGQGYEQYF | 501 |
| 19 | CASSFPDTQYF | 502 |
| 17 | CASSSGQGYEQYF | 503 |
| 30 | CASSEGYEQYF | 504 |
| 141 | CASSLLQGNQPQHF | 505 |
| 46 | CASSPTPLNTEAFF | 506 |
| 21 | CASRTASYEQYF | 507 |
| 43 | CASSLGYNEQFF | 508 |
| 27 | CASSELNTGELFF | 509 |
| 1169 | CASSVGTGTYEQYF | 510 |
| 7 | CASSEDGMNTEAFF | 511 |
| 6 | CASSYGGNEQFF | 512 |
| 54 | CASSFGTDTQYF | 513 |
| 12 | CASSGSYEQYF | 514 |
| 6 | CASSVGGSSTDTQYF | 515 |
| 7 | CASSFQGSTDTQYF | 516 |
| 11 | CASSFGTDTQYF | 517 |
| 7 | CASSFGGGTEAFF | 518 |
| 10 | CASSYGQGYEQYF | 519 |
| 22 | CASSLGAYEQYF | 520 |
| 8 | CASSYSYEQYF | 521 |
| 5 | CASSQGMNTEAFF | 522 |
| 5 | CASSVGGELFF | 523 |
| 11 | CASSLTGSYEQYF | 524 |
| 5 | CASSYSGNEQFF | 525 |
| 6 | CASSIGSYGYTF | 526 |
| 86 | CASRDSYGYTF | 527 |
| 949 | CASSYYNEQFF | 528 |
| 19 | CASSLGGNEQFF | 529 |
| 6 | CSARPLAGLYNEQFF | 530 |
| 25 | CASSVGGQGAYEQYF | 531 |
| 17 | CASSLVGGSGNEQFF | 532 |
| 24 | CASSLLGDEQFF | 533 |
| 6 | CASSLAGVTDTQYF | 534 |
| 593 | CASSSTTDTQYF | 535 |
| 8 | CASSLSGYGYTF | 536 |
| 27 | CASSVDMNTEAFF | 537 |
| 11 | CASSFTDTQYF | 538 |
| 1501 | CASSPLGDTQYF | 539 |
| 56 | CASSVGQGAYEQYF | 540 |
| 35 | CASSGDRGNTEAFF | 541 |
| 13 | CASSQARTNTGELFF | 542 |
| 267 | CASSLGSYEQYF | 543 |
| 347 | CASSQGTILLSGNTIYF | 544 |
| 5 | CASSLVQGSYEQYF | 545 |
| 3807 | CASSQGGTSGTDTQYF | 546 |
| 44 | CASSESYEQYF | 547 |
| 54 | CASSAGTGSYEQYF | 548 |
| 131 | CASSQARNTEAFF | 549 |
| 31 | CSASSESSYNEQFF | 550 |
| 8 | CASSYRGTEAFF | 551 |
| 56 | CASSLRGVEQYF | 552 |
| 59 | CSAGDSSYEQYF | 553 |
| 142 | CASSVGQGASYEQYF | 554 |
| 42 | CASSLGGGNTEAFF | 555 |

TABLE 3
Untreated Recovered Patients'
HCDR3 Frequency and Sequences

| Frequency | HCDR3 | SEQ ID NO: |
|---|---|---|
| 24 | CASSSQGAGTEAFF | 556 |
| 5 | CASSESYEQYF | 557 |
| 615 | CASSLIGVSSYNEQFF | 558 |
| 5 | CASSPGGEIYEQYF | 559 |
| 6 | CSVGLADYNEQFF | 560 |
| 40 | CAWSVQQNYGYTF | 561 |
| 9 | CASGDSYEQYF | 562 |
| 7 | CASSFGVGYEQYF | 563 |
| 9 | CASSYSGSGYTF | 564 |
| 5 | CATSREGANTEAFF | 565 |
| 7 | CASSFGQIYGYTF | 566 |
| 44 | CASSFRQGANTGELFF | 567 |
| 12 | CASSLDGSSYEQYF | 568 |
| 5 | CASSTGLGMNTEAFF | 569 |
| 5 | CSARDAESYEQYF | 570 |
| 16 | CASSLLVSNQPQHF | 571 |
| 7 | CASSPGTSGAYEQYF | 572 |
| 9 | CASSPGGGSYEQYF | 573 |
| 5 | CASSTGYSNQPQHF | 574 |
| 8 | CSASRDSQETQYF | 575 |
| 7 | CAWSVTDNEQFF | 576 |
| 6 | CASRTGDTEAFF | 577 |
| 5 | CASSLGGDQPQHF | 578 |
| 20 | CASSLAGNTEAFF | 579 |
| 9 | CSARQGGAEAFF | 580 |
| 6 | CASSPQGVSGNTIYF | 581 |
| 56 | CASSTSSYEQYF | 582 |
| 6 | CASSQAGALNTEAFF | 583 |
| 5 | CASSIRSSYEQYF | 584 |
| 8 | CASSIGLYGYTF | 585 |
| 20 | CASRGVSYEQYF | 586 |
| 11 | CASSLGGRTEAFF | 587 |
| 13 | CASSLTDTQYF | 588 |
| 5 | CASSIRSSYEQYF | 589 |
| 222 | CASSAGGYEQYF | 590 |
| 35 | CASSQETGMNTEAFF | 591 |
| 5 | CASRRGTEAFF | 592 |
| 7 | CASSLVGETQYF | 593 |
| 5 | CASMGGSSYNEQFF | 594 |
| 5 | CASSFFLGEKLFF | 595 |
| 5 | CASSQDRGAYEQYF | 596 |
| 16 | CASSEAGNTEAFF | 597 |
| 101 | CASSLSSSQETQYF | 598 |
| 8 | CASSYSSRNTEAFF | 599 |
| 33 | CASSLGPNYEQYF | 600 |
| 8 | CASSSSYEQYF | 601 |
| 8 | CSARTGLAYEQYF | 602 |
| 5 | CASTLGGFGQPQHF | 603 |
| 6 | CASSLGVYEQYF | 604 |
| 97 | CASSEGWVTNTEAFF | 605 |
| 15 | CASSLGSYGYTF | 606 |
| 20 | CASSYPGQPQHF | 607 |
| 15 | CASSYSGGTEAFF | 608 |
| 5 | CSARRDSYEQYF | 609 |
| 611 | CAGGDYGYTF | 610 |
| 5 | CSARRDSYEQYF | 611 |
| 30 | CASSYGTGGTEAFF | 612 |
| 23 | CASSQDLGYGYTF | 613 |
| 7 | CASSLMGNQPQHF | 614 |
| 6 | CSARTGYEQYF | 615 |
| 10 | CASSYDSSYEQYF | 616 |
| 7 | CASSLDSMNTEAFF | 617 |
| 12 | CASSLGSSYEQYF | 618 |
| 6 | CASSYGTGGTEAFF | 619 |
| 17 | CASSLVGLAAYEQYR | 620 |
| 7 | CASSFQGFYEQYF | 621 |
| 223 | CASSLSGPNEKLFF | 622 |
| 9 | CASSLDPSSYEQYF | 623 |
| 9 | CASSVGAGELFF | 624 |
| 7 | CASSPGDYGYTF | 625 |
| 5 | CASSLGYEQYF | 626 |
| 9 | CASSLGVTGELFF | 627 |
| 5 | CASSRSGLAEETQYF | 628 |
| 5 | CASSLGSEQYF | 629 |
| 12 | CASSPGNSYEQYF | 630 |
| 7 | CASSVGQGTYEQYF | 631 |
| 6 | CASSLTGNSGNTIYF | 632 |
| 5 | CASSIGYEQYF | 633 |
| 5 | CSASGRQETQYF | 634 |
| 5 | CASSPGFSRHYEQYF | 635 |
| 5 | CSARDLGSSYEQYF | 636 |
| 6 | CASSFGQLNQPQHF | 637 |
| 5 | CASSLTSTDTQYF | 638 |
| 6 | CSAGQGRYEQYF | 639 |
| 41 | CASSELAGSYNEQFF | 640 |
| 8 | CASSYGTGNQPQHF | 641 |
| 12 | CASSFGQGDTEAFF | 642 |
| 11 | CASSSLGGELFF | 643 |
| 8 | CASSQGYNSPLHF | 644 |
| 7 | CASSVVGNTEAFF | 645 |
| 5 | CSAHGGSSYEQYF | 646 |
| 45 | CASSQGYNSPLHF | 647 |
| 6 | CASSLAGAYEQYF | 648 |
| 7 | CSVDRNTEAFF | 649 |
| 5 | CASSWGYNEQFF | 650 |
| 6 | CASSPSSYEQYF | 651 |
| 5 | CASSLGRTYEQYF | 652 |
| 16 | CSARDLAGGLNEQFF | 653 |
| 10 | CASSQGYNSPLHF | 654 |
| 5 | CSARLADYNEQFF | 655 |
| 6 | CASSYGGYGYTF | 656 |
| 6 | CASSLSGNHNEQFF | 657 |
| 8 | CASSFGGNEQFF | 658 |
| 13 | CASSVGGDTQYF | 659 |
| 6 | CASSFGYEQYF | 660 |
| 6 | CATSRGNQPQHF | 661 |
| 8 | CSVDRNTEAFF | 662 |
| 7 | CASTYYNEQFF | 663 |
| 9 | CASSLGQGDTEAFF | 664 |
| 7 | CATSRDRGTEAFF | 665 |
| 9 | CASSIAGTSYNEQFF | 666 |
| 5 | CASSLAGAQPQHF | 667 |
| 5 | CASSLSGTQYF | 668 |
| 407 | CASSLIGVSSYNEQFF | 669 |
| 18 | CASSPTGGTGELFF | 670 |
| 7 | CASSLEAYEQYF | 671 |
| 16 | CASSLGTGYGYTF | 672 |
| 7 | CSARGQGDYEQYF | 673 |
| 7 | CASSLWGYEQYF | 674 |
| 6 | CASSLAGANQETQYF | 675 |
| 9 | CATSDLASDTQYF | 676 |
| 5 | CASSEGGQPQHF | 677 |
| 5 | CASSLAYEQYF | 678 |
| 8 | CASSLGGNTEAFF | 679 |
| 6 | CASSLGNTEAFF | 680 |
| 5 | CASSSNSPLHF | 681 |
| 11 | CASSFVAGTDTQYF | 682 |
| 7 | CASSLTRNTEAFF | 683 |
| 7 | CASSLGQGANEQYF | 684 |
| 7 | CSAGSGSSYNEQFF | 685 |
| 8 | CASSLSVYEQYF | 686 |
| 18 | CASSLGQGGYEQYF | 687 |
| 5 | CASSIADYNEQFF | 688 |
| 6 | CSARVQLNTEAFF | 689 |
| 5 | CASSLGSNYEQYF | 690 |
| 8 | CASSLGLNEQFF | 691 |
| 6 | CASSLGQGYEQYF | 692 |
| 20 | CASSPTAYEQYF | 693 |
| 7 | CASSFGGAEAFF | 694 |

TABLE 4
Stimulated Recovered Patients'
HCDR3 Frequency and Sequences

| Frequency | HCDR3 | SEQ ID NO: |
|---|---|---|
| 31 | CASSGTGLNTEAFF | 695 |
| 8 | CSARRDSRTDTQYF | 696 |
| 90 | CASSSTYEQYF | 697 |
| 113 | CSARRDSRTDTQYF | 698 |
| 62 | CSAKGQGSNQPQHF | 699 |
| 43 | CATSEGYEQYF | 700 |
| 28 | CSARDRANTGELFF | 701 |
| 7 | CASSPGSTYEQYF | 702 |
| 16 | CATSAGOGNTEAFF | 703 |
| 11 | CASSFGNTEAFF | 704 |
| 22 | CASSRAGGSTDTQYF | 705 |
| 33 | CASSPGLAGGYEQFF | 706 |
| 5 | CSATDRVGYGYTF | 707 |
| 5 | CASSLRGDYGYTF | 708 |
| 52 | CASSLVAEKLFF | 709 |
| 44 | CASSLVAEKLFF | 710 |
| 176 | CASSLIGVSSYNEQFF | 711 |
| 37 | CASSLGQQETQYF | 712 |
| 29 | CASSYSGNQPQHF | 713 |
| 16 | CASSFRQGANTGELFF | 714 |
| 6 | CASSLLGSYEQYF | 715 |
| 978 | CASSEGASNQPQHF | 716 |
| 426 | CASSQELAGTDTQYF | 717 |
| 279 | CASSYRGDYGYTF | 718 |
| 37 | CASSLAGVTDTQYF | 719 |
| 31 | CASSLVAEKLFF | 720 |
| 16 | CSARRDSRTDTQYF | 721 |
| 6 | CASSLSGLAGDEQYF | 722 |
| 11 | CSASSGYTF | 723 |
| 46 | CASSLTGNYGYTF | 724 |
| 35 | CAISESSYEQYF | 725 |
| 38 | CASSRAGGSTDTQYF | 726 |
| 7 | CATSDPRQGDNQPQHF | 727 |
| 569 | CASSQTYEQYF | 728 |
| 141 | CASSATGDSNQPQHF | 729 |
| 56 | CASSLRGAVGYTF | 730 |
| 50 | CASSGTGLNTEAFF | 731 |
| 17 | CASSLSSPTDTQYF | 732 |
| 40 | CASSEGPSSQPQHF | 733 |
| 7 | CASSSTGSGNTIYF | 734 |
| 5 | CSVGTGWPYEQYF | 735 |
| 287 | CASSLTGNYGYTF | 736 |
| 126 | CASSEGPSSQPQHF | 737 |
| 91 | CASSLTYEQYF | 738 |
| 7 | CASRRAGSYEQYF | 739 |
| 6 | CSARDGTTYEQYF | 740 |
| 171 | CASSPNSTYEQYF | 741 |
| 47 | CASSYTYEQYF | 742 |
| 34 | CASSLWKRNTEAFF | 743 |
| 41 | CASSVVASGQETQYF | 744 |
| 27 | CASSPTGSNQPQHF | 745 |
| 15 | CASSLSGVTDTQYF | 746 |
| 9 | CASSPDGYGYTF | 747 |
| 76 | CASSEGASNQPQHF | 748 |
| 6299 | CAWSVQQNYGYTF | 749 |
| 119 | CASKGGGRGYTF | 750 |
| 50 | CASSLRDRANTGELFF | 751 |
| 36 | CASSLTGNYGYTF | 752 |
| 7 | CASSQGVGYTF | 753 |
| 13 | CASSRAGGSTDTQYF | 754 |
| 1338 | CAWSVTDNEQFF | 755 |
| 408 | CASSSTYEQYF | 756 |
| 186 | CASSFGGNYGYTF | 757 |
| 26 | CASSEGFSNQPQHF | 758 |
| 6 | CASSLTATNEKLFF | 759 |
| 6 | CASSLTDTQYF | 760 |
| 291 | CASSPTYEQYF | 761 |
| 230 | CASSRSTGDQETQYF | 762 |
| 37 | CASSLTGNYGYTF | 763 |
| 33 | CASSLSGVTDTQYF | 764 |
| 14 | CASSRAGGSTDTQYF | 765 |
| 13 | CASSPGGGSYEQYF | 766 |
| 6 | CASSLSSGSYNEQFF | 767 |
| 64 | CSAKGQGGNQPQHF | 768 |
| 14 | CASSLAGGPDEQFF | 769 |
| 463 | CSARQGGAEAFF | 770 |
| 125 | CASSQTYEQYF | 771 |
| 40 | CASSLAGGSTDTQYF | 772 |
| 45 | CSARRDRGDNSPLHF | 773 |
| 7 | CASSRAGGSTDTQYF | 774 |
| 6 | CASSLGLAGGDTQYF | 775 |
| 94 | CSARGEGNYGYTF | 776 |
| 1902 | CASSLNYEQYF | 777 |
| 592 | CASSPTYEQYF | 778 |
| 135 | CSATRDASSYNEQFF | 779 |
| 84 | CASSLNYEQYF | 780 |
| 42 | CASSEGASNQPQHF | 781 |
| 25 | CATSAGQGNTEAFF | 782 |
| 13 | CASRDSGANVLTF | 783 |
| 6 | CASSQSGAGTDTQYF | 784 |
| 5 | CASSYSGGQPQHF | 785 |
| 275 | CASKQGGRGYTF | 786 |
| 250 | CASSSGLAGVETQYF | 787 |
| 209 | CATKGGGRGYTF | 788 |
| 124 | CASSQVAGEQFF | 789 |
| 177 | CASSYEGGRNTEAFF | 790 |
| 60 | CASSEGGRGPDTQYF | 791 |
| 17 | CSATQGAATNEKLFF | 792 |
| 28 | CASSLGGVGNQPQHF | 793 |
| 1480 | CSARRGTEAFF | 794 |
| 350 | CASSPTGSNQPQHF | 795 |
| 107 | CASSEGASNQPQHF | 796 |
| 23 | CASSLGGVGNQPQHF | 797 |
| 11 | CASSPGQGDTEAFF | 798 |
| 249 | CSARQGVDQPQHF | 799 |
| 274 | CASSEGASNQPQHF | 800 |
| 74 | CASSPTYEQYF | 801 |
| 66 | CSARRDSRTDTQYF | 802 |
| 27 | CSARRDSRTDTQYF | 803 |
| 15 | CSARRDSRTDTQYF | 804 |
| 5 | CSARRDSRTDTQYF | 805 |
| 407 | CASSEGGRGPDTQYF | 806 |
| 28 | CSATDRVGYGYTF | 807 |
| 28 | CSASRGNEQFF | 808 |
| 12 | CASSESAGQETQYF | 809 |
| 1625 | CASSSTYEQYF | 810 |
| 690 | CASSQVAGEQYF | 811 |
| 758 | CSAKGQGSNQPQHF | 812 |
| 28 | CASSQVAGEQYF | 813 |
| 43 | CASSQDPGLNTEAFF | 814 |
| 6 | CASSEGSSNQPQHF | 815 |
| 3568 | CASSRSGLAEETQYF | 816 |
| 49 | CASSLVAEKLFF | 817 |
| 82 | CASSLVSDTQYF | 818 |
| 40 | CASSGTGLNTEAFF | 819 |
| 31 | CSASRDASNQPQHF | 820 |
| 7 | CASSLRRGYTF | 821 |
| 741 | CATSDPRQGDNQPQHF | 822 |
| 623 | CASSEGASNQPQHF | 823 |
| 34 | CSARRDSRTDTQYF | 824 |
| 13 | CSASRGNEQFF | 825 |
| 874 | CASSQGVGYTF | 826 |
| 714 | CASSQVAGEQYF | 827 |
| 570 | CASSSTYEQYF | 828 |
| 57 | CASKQGGRGYTF | 829 |
| 15 | CASSPTYEQYF | 830 |
| 14 | CASSEGFSNQPQHF | 831 |
| 1509 | CSATRGAGGQETQYF | 832 |
| 194 | CASSYTYEQYF | 833 |
| 89 | CASSQTYEQYF | 834 |
| 77 | CASSQGVGYTF | 835 |
| 44 | CASSRSGAGTDTQYF | 836 |
| 19 | CASSPTYEQYF | 837 |
| 514 | CASSQVAGEQYF | 838 |
| 390 | CSATRGAGSTDTQYF | 839 |
| 420 | CASSESAGQETQYF | 840 |
| 219 | CASSSLGTGELFF | 841 |
| 39 | CSARDRANTGELFF | 842 |
| 25 | CASSQSGAGTDTQYF | 843 |
| 9 | CASSLGGVGNQPQHF | 844 |
| 481 | CASSQGVGYTF | 845 |
| 301 | CASSEGASNQPQHF | 846 |
| 67 | CASSQTYEQYF | 847 |
| 47 | CSARRGTEAFF | 848 |
| 36 | CSARRDSRTDTQYF | 849 |
| 5 | CSARRDSRTDTQYF | 850 |
| 6 | CSASDRVGYGYTF | 851 |
| 7 | CASSEGASNQPQHF | 852 |
| 326 | CSAKGQGSNQPQHF | 853 |
| 256 | CASSGTGLNTEAFF | 854 |
| 195 | CSARDRANTGELFF | 855 |
| 16 | CAWSTYYGYTF | 856 |
| 36 | CSATRDRRTYNEQFF | 857 |
| 354 | CASSSRREQFF | 858 |
| 92 | CASSPTYEQYF | 859 |
| 25 | CASSLRRGYTF | 860 |
| 9 | CASSLGLAGGDTQYF | 861 |
| 13 | CASSLDLANEQFF | 862 |
| 711 | CSARRDSRTDTQYF | 863 |
| 232 | CASSRSGGSTDTQYF | 864 |
| 275 | CSARRDSRTDTQYF | 865 |
| 17 | CASSWTGGGGYTF | 866 |
| 46 | CASSPRLAGATGELFF | 867 |
| 8 | CASSQGYNSPLHF | 868 |
| 27 | CASSGTGLNTEAFF | 869 |
| 898 | CSARRDSRTDTQYF | 870 |
| 332 | CSARRDSRGDTQYF | 871 |
| 390 | CSARRGTEAFF | 872 |
| 163 | CASSWGYNEQFF | 873 |
| 13 | CSASRQASSTDTQYF | 874 |
| 6 | CSARRDSRTDTQYF | 875 |
| 60 | CSARRGTEAFF | 876 |
| 17 | CSARRDSRTDTQYF | 877 |
| 3139 | CSARRDSRTDTQYF | 878 |
| 661 | CSARRDSRTDTQYF | 879 |
| 438 | CASSEGASNQPQHF | 880 |
| 10 | CASSRSGGSTDTQYF | 881 |
| 7 | CSVVGLAGGSSYNEQFF | 882 |
| 6 | CASSLSVPSSGNTIYF | 883 |
| 7 | CASSLSGGETQYF | 884 |
| 2145 | CSARRDSRTDTQYF | 885 |
| 288 | CSATRGAGGQETQYF | 886 |
| 83 | CASSLAGGSTDTQYF | 887 |
| 10 | CASSVVGNTEAFF | 888 |
| 16 | CASSRSGGSTDTQYF | 889 |
| 6 | CASSLSGTNTGELFF | 890 |
| 159 | CASSLWTGVSYEQYF | 891 |
| 9 | CASSPGQGYEQYF | 892 |
| 5 | CASSLAGGSTDTQYF | 893 |
| 6 | CASSVATGGYEQYF | 894 |
| 14 | CASSPRLAGGTGELFF | 895 |
| 561 | CSARRGTEAFF | 896 |
| 489 | CSARRDSRTDTQYF | 897 |
| 163 | CSVVSRGRTDTQYF | 898 |
| 114 | CASSRAGGSTDTQYF | 899 |
| 46 | CASSLVAEKLFF | 900 |
| 16 | CASSEGASNQPQHF | 901 |
| 5 | CASSPRTGWTEAFF | 902 |
| 462 | CASSEGASNQPQHF | 903 |
| 50 | CASSRAGGSTDTQYF | 904 |
| 3706 | CSARRDSRTDTQYF | 905 |
| 23 | CASSRAGGSTDTQYF | 906 |
| 5 | CASSLVTGNSPLHF | 907 |
| 323 | CASSEGASNQPQHF | 908 |
| 151 | CASSRAGGSTDTQYF | 909 |
| 34 | CASSSEPNTEAFF | 910 |
| 7 | CASSLGSSYEQYF | 911 |
| 45 | CASSSDRSSYNEQFF | 912 |
| 24 | CSARRGTEAFF | 913 |
| 9 | CASSVDSYGYTF | 914 |
| 2781 | CSARRDSRTDTQYF | 915 |
| 19 | CASSSTYEQYF | 916 |
| 27 | CASSLVSDTQYF | 917 |
| 9 | CASSLGQGGYEQYF | 918 |
| 110 | CASSEGSSNQPQHF | 919 |
| 7 | CASSVGQGDGYTF | 920 |
| 123 | CASSLAGGYEQYF | 921 |
| 5 | CASSLIGVSSYNEQFF | 922 |
| 184 | CASSPGADTQYF | 923 |
| 10 | CASSLGQGGYEQYF | 924 |
| 6 | CASSLGTGYGYTF | 925 |
| 38 | CASSPTAYEQYF | 926 |
| 384 | CASSYSRTGELFF | 927 |
| 83 | CASSLSGELFF | 928 |
| 55 | CASSLGPPPGTDTQYF | 929 |

TABLE 5
TCR rearrangement and corresponded Hypomutated epitopes

| | | | | Hypo- |
|---|---|---|---|---|
| | | | | mutated |
| Group | TCR | Rearrangement | reference_orfs | Epitopes |
| HD_UT | CASSLGTTEAFF+TCRB | TTGGAGATCCAGCGCACAGAGCAG | surface_glyco- | RLDKVEAEV |
| | V07-09+TCRBJ01-01 | GGGGACTCGGCCATGTATCTCTGT | protein | (SEQ ID |
| | (SEQ ID NO: 930) | GCCAGCAGCTTAGGAACCACTGAA | | NO: 941) |
| | | GCTTTCTTTGGACAA (SEQ ID | | |
| | | NO: 935) | | |
| HD_UT | CASSLGTTEAFF+TCRB | TTGGAGATCCAGCGCACAGAGCAG | surface_glyco- | RLDKVEAEV |
| | V07-09+TCRBJ01-01 | GGGGACTCGGCCATGTATCTCTGT | protein | (SEQ ID |
| | (SEQ ID NO: 930) | GCCAGCAGCTTAGGGACAACTGAA | | NO: 941) |
| | | GCTTTCTTTGGACAA (SEQ ID | | |
| | | NO: 936) | | |
| HD_UT | CAISESIGTEAFF+TCR | ACTCTGGAGTCCGCTACCAGCTCC | surface_glyco- | RLFRKSNLK |
| | BV10-03+TCRBJ01- | CAGACATCTGTGTACTTCTGTGCC | protein | (SEQ ID |
| | 01 | TACAGTGAGTCGATAGGGACTGAA | | NO: 942) |
| | (SEQ ID NO: 931) | GCTTTCTTTGGACAA (SEQ ID | | |
| | | NO: 937) | | |
| PT_UT | CASSVVSGDEQFF+TCR | AATGTGAGCACCTTGGAGCTGGGG | surface_glyco- | RLDKVEAEV |
| | BV09-01+TCRBJ02- | GACTCGGCCCTTTATCTTTGCGCC | protein | (SEQ ID |
| | 01 | AGCAGCTTGCTAGGGTCCTACGAG | | NO: 941) |
| | (SEQ ID NO: 932) | CAGTACTTCGGGCCG (SEQ ID | | |
| | | NO: 938) | | |
| PT_UT | CASSIGLAGAYNEQFF+ | TCGGCCCAAAAGAACCCGACAGCT | surface_glyco- | RLFRKSNLK |
| | TCRBV19-01+-TCRBJ | TTCTATCTCTGTGCCAGTAGTATC | protein | (SEQ ID |
| | 0201 | GGACTAGCGGGTGCCTACAATGAG | | NO: 942) |
| | (SEQ ID NO: 933) | CAGTTCTTCGGGCCA (SEQ ID | | |
| | | NO: 939) | | |
| PT_Stim | CASSLLGSYEQYF+TCR | AACCTGAGCTCTCTGGAGCTGGGG | surface_glyco- | RLDKVEAEV |
| | BV05-01+TCRBJ02- | GACTCAGCTTTGTATTTCTGTGCC | protein | (SEQ ID |
| | 07 | AGCAGCGTGGTGAGTGGGGATGAG | | NO: 941) |
| | (SEQ ID NO: 934) | CAGTTCTTCGGGCCA (SEQ ID | | |
| | | NO: 940) | | |

### Listing of Sequences

SEQ ID NO:1: amino acid sequence of SARS-Cov-2 RNA-dependent RNA polymerase (Non-structural protein 12, nsp12) (GenBank Acc. #YP_009725307.1)

>YP_009725307.1 RNA-dependent RNA
polymerase [Severe acute respiratory
syndrome coronavirus 2]
SADAQSFLNRVCGVSAARLTPCGTGTSTDVVYRAFDIYNDKVAGFAKFLK
TNCCRFQEKDEDDNLIDSYFVVKRHTFSNYQHEETIYNLLKDCPAVAKHD
FFKFRIDGDMVPHISRORLTKYTMADLVYALRHFDEGNCDTLKEILVTYN
CCDDDYFNKKDWYDFVENPDILRVYANLGERVRQALLKTVQFCDAMRNAG
IVGVLTLDNQDLNGNWYDFGDFIQTTPGSGVPVVDSYYSLLMPILTLTRA
LTAESHVDTDLTKPYIKWDLLKYDFTEERLKLFDRYFKYWDQTYHPNCVN
CLDDRCILHCANFNVLFSTVFPPTSFGPLVRKIFVDGVPFVVSTGYHFRE
LGVVHNQDVNLHSSRLSFKELLVYAADPAMHAASGNLLLDKRTTCFSVAA
LTNNVAFQTVKPGNFNKDFYDFAVSKGFFKEGSSVELKHFFFAQDGNAAI
SDYDYYRYNLPTMCDIRQLLFVVEVVDKYFDCYDGGCINANQVIVNNLDK
SAGFPFNKWGKARLYYDSMSYEDQDALFAYTKRNVIPTITQMNLKYAISA
KNRARTVAGVSICSTMTNRQFHQKLLKSIAATRGATVVIGTSKFYGGWHN
MLKTVYSDVENPHLMGWDYPKCDRAMPNMLRIMASLVLARKHTTCCSLSH
RFYRLANECAQVLSEMVMCGGSLYVKPGGTSSGDATTAYANSVFNICQAV
TANVNALLSTDGNKIADKYVRNLQHRLYECLYRNRDVDTDFVNEFYAYLR
KHFSMMILSDDAVVCFNSTYASQGLVASIKNFKSVLYYQNNVFMSEAKCW
TETDLTKGPHEFCSQHTMLVKQGDDYVYLPYPDPSRILGAGCFVDDIVKT
DGTLMIERFVSLAIDAYPLTKHPNQEYADVFHLYLQYIRKLHDELTGHML
DMYSVMLTNDNTSRYWEPEFYEAMYTPHTVLQ

SEQ ID NO:2: nucleotide sequence encoding SARS-Cov-2 RNA-dependent RNA polymerase (Non-structural protein 12, nsp12) (GenBank Acc. #NC_045512.2:13441-16236)

>NC_045512.2:13441-16236
TCAGCTGATGCACAATCGTTTTTAAACGGGTTTGCGGTGTAAGTGCAGCC
CGTCTTACACCGTGCGGCACAGGCACTAGTACTGATGTCGTATACAGGGC
TTTTGACATCTACAATGATAAAGTAGCTGGTTTTGCTAAATTCCTAAAAA
CTAATTGTTGTCGCTTCCAAGAAAAGGACGAAGATGACAATTTAATTGAT
TCTTACTTTGTAGTTAAGAGACACACTTTCTCTAACTACCAACATGAAGA
AACAATTTATAATTTACTTAAGGATTGTCCAGCTGTTGCTAAACATGACT
TCTTTAAGTTTAGAATAGACGGTGACATGGTACCACATATATCACGTCAA
CGTCTTACTAAATACACAATGGCAGACCTCGTCTATGCTTTAAGGCATTT
TGATGAAGGTAATTGTGACACATTAAAAGAAATACTTGTCACATACAATT
GTTGTGATGATGATTATTTCAATAAAAAGGACTGGTATGATTTTGTAGAA
AACCCAGATATATTACGCGTATACGCCAACTTAGGTGAACGTGTACGCCA
AGCTTTGTTAAAAACAGTACAATTCTGTGATGCCATGCGAAATGCTGGTA
TTGTTGGTGTACTGACATTAGATAATCAAGATCTCAATGGTAACTGGTAT
GATTTCGGTGATTTCATACAAACCACGCCAGGTAGTGGAGTTCCTGTTGT
AGATTCTTATTATTCATTGTTAATGCCTATATTAACCTTGACCAGGGCTT
TAACTGCAGAGTCACATGTTGACACTGACTTAACAAAGCCTTACATTAAG
TGGGATTTGTTAAAATATGACTTCACGGAAGAGAGGTTAAAACTCTTTGA
CCGTTATTTTAAATATTGGGATCAGACATACCACCCAAATTGTGTTAACT
GTTTGGATGACAGATGCATTCTGCATTGTGCAAACTTTAATGTTTTATTC
TCTACAGTGTTCCCACCTACAAGTTTTGGACCACTAGTGAGAAAAATATT
TGTTGATGGTGTTCCATTTGTAGTTTCAACTGGATACCACTTCAGAGAGC
TAGGTGTTGTACATAATCAGGATGTAAACTTACATAGCTCTAGACTTAGT
TTTAAGGAATTACTTGTGTATGCTGCTGACCCTGCTATGCACGCTGCTTC
TGGTAATCTATTACTAGATAAACGCACTACGTGCTTTTCAGTAGCTGCAC
TTACTAACAATGTTGCTTTTCAAACTGTCAAACCCGGTAATTTTAACAAA
GACTTCTATGACTTTGCTGTGTCTAAGGGTTTCTTTAAGGAAGGAAGTTC
TGTTGAATTAAAACACTTCTTCTTTGCTCAGGATGGTAATGCTGCTATCA
GCGATTATGACTACTATCGTTATAATCTACCAACAATGTGTGATATCAGA
CAACTACTATTTGTAGTTGAAGTTGTTGATAAGTACTTTGATTGTTACGA
TGGTGGCTGTATTAATGCTAACCAAGTCATCGTCAACAACCTAGACAAAT
CAGCTGGTTTTCCATTTAATAAATGGGGTAAGGCTAGACTTTATTATGAT
TCAATGAGTTATGAGGATCAAGATGCACTTTTCGCATATACAAAACGTAA
TGTCATCCCTACTATAACTCAAATGAATCTTAAGTATGCCATTAGTGCAA
AGAATAGAGCTCGCACCGTAGCTGGTGTCTCTATCTGTAGTACTATGACC
AATAGACAGTTTCATCAAAAATTATTGAAATCAATAGCCGCCACTAGAGG
AGCTACTGTAGTAATTGGAACAAGCAAATTCTATGGTGGTTGGCACAACA
TGTTAAAAACTGTTTATAGTGATGTAGAAAACCCTCACCTTATGGGTTGG
GATTATCCTAAATGTGATAGAGCCATGCCTAACATGCTTAGAATTATGGC
CTCACTTGTTCTTGCTCGCAAACATACAACGTGTTGTAGCTTGTCACACC
GTTTCTATAGATTAGCTAATGAGTGTGCTCAAGTATTGAGTGAAATGGTC
ATGTGTGGCGGTTCACTATATGTTAAACCAGGTGGAACCTCATCAGGAGA
TGCCACAACTGCTTATGCTAATAGTGTTTTTAACATTTGTCAAGCTGTCA
CGGCCAATGTTAATGCACTTTTATCTACTGATGGTAACAAAATTGCCGAT
AAGTATGTCCGCAATTTACAACACAGACTTTATGAGTGTCTCTATAGAAA
TAGAGATGTTGACACAGACTTTGTGAATGAGTTTTACGCATATTTGCGTA
AACATTTCTCAATGATGATACTCTCTGACGATGCTGTTGTGTGTTTCAAT
AGCACTTATGCATCTCAAGGTCTAGTGGCTAGCATAAAGAACTTTAAGTC
AGTTCTTTATTATCAAAACAATGTTTTTATGTCTGAAGCAAAATGTTGGA
CTGAGACTGACCTTACTAAAGGACCTCATGAATTTTGCTCTCAACATACA
ATGCTAGTTAAACAGGGTGATGATTATGTGTACCTTCCTTACCCAGATCC
ATCAAGAATCCTAGGGGCCGGCTGTTTTGTAGATGATATCGTAAAAACAG
ATGGTACACTTATGATTGAACGGTTCGTGTCTTTAGCTATAGATGCTTAC
CCACTTACTAAACATCCTAATCAGGAGTATGCTGATGTCTTTCATTTGTA
CTTACAATACATAAGAAAGCTACATGATGAGTTAACAGGACACATGTTAG
ACATGTATTCTGTTATGCTTACTAATGATAACACTTCAAGGTATTGGGAA
CCTGAGTTTTATGAGGCTATGTACACACCGCATACAGTCTTACAG

SEQ ID NO:3: nucleotide sequence of Cold_spot_name: RNA-dependent_RNA_polymerase_sp|P0DTD1|R1AB_SARS2:6-932:CDS_NT

TTTTTAAACGGGTTTGCGGTGTAAGTGCAGCCCGTCTTACACCGTGCGGC
ACAGGCACTAGTACTGATGTCGTATACAGGGCTTTTGACATCTACAATGA
TAAAGTAGCTGGTTTTGCTAAATTCCTAAAAACTAATTGTTGTCGCTTCC
AAGAAAAGGACGAAGATGACAATTTAATTGATTCTTACTTTGTAGTTAAG
AGACACACTTTCTCTAACTACCAACATGAAGAAACAATTTATAATTTACT
TAAGGATTGTCCAGCTGTTGCTAAACATGACTTCTTTAAGTTTAGAATAG
ACGGTGACATGGTACCACATATATCACGTCAACGTCTTACTAAATACACA
ATGGCAGACCTCGTCTATGCTTTAAGGCATTTTGATGAAGGTAATTGTGA
CACATTAAAAGAAATACTTGTCACATACAATTGTTGTGATGATGATTATT
TCAATAAAAAGGACTGGTATGATTTTGTAGAAAACCCAGATATATTACGC
GTATACGCCAACTTAGGTGAACGTGTACGCCAAGCTTTGTTAAAAACAGT
ACAATTCTGTGATGCCATGCGAAATGCTGGTATTGTTGGTGTACTGACAT
TAGATAATCAAGATCTCAATGGTAACTGGTATGATTTCGGTGATTTCATA
CAAACCACGCCAGGTAGTGGAGTTCCTGTTGTAGATTCTTATTATTCATT
GTTAATGCCTATATTAACCTTGACCAGGGCTTTAACTGCAGAGTCACATG
TTGACACTGACTTAACAAAGCCTTACATTAAGTGGGATTTGTTAAAATAT
GACTTCACGGAAGAGAGGTTAAAACTCTTTGACCGTTATTTTAAATATTG
GGATCAGACATACCACCCAAATTGTGTTAACTGTTTGGATGACAGATGCA
TTCTGCATTGTGCAAACTTTAATGTTTTATTCTCTACAGTGTTCCCACCT
ACAAGTTTTGGACCACTAGTGAGAAAAATATTTGTTGATGGTGTTCCATT
TGTAGTTTCAACTGGATACCACTTCAGAGAGCTAGGTGTTGTACATAATC
AGGATGTAAACTTACATAGCTCTAGACTTAGTTTTAAGGAATTACTTGTG
TATGCTGCTGACCCTGCTATGCACGCTGCTTCTGGTAATCTATTACTAGA
TAAACGCACTACGTGCTTTTCAGTAGCTGCACTTACTAACAATGTTGCTT
TTCAAACTGTCAAACCCGGTAATTTTAACAAAGACTTCTATGACTTTGCT
GTGTCTAAGGGTTTCTTTAAGGAAGGAAGTTCTGTTGAATTAAAACACTT
CTTCTTTGCTCAGGATGGTAATGCTGCTATCAGCGATTATGACTACTATC
GTTATAATCTACCAACAATGTGTGATATCAGACAACTACTATTTGTAGTT
GAAGTTGTTGATAAGTACTTTGATTGTTACGATGGTGGCTGTATTAATGC
TAACCAAGTCATCGTCAACAACCTAGACAAATCAGCTGGTTTTCCATTTA
ATAAATGGGGTAAGGCTAGACTTTATTATGATTCAATGAGTTATGAGGAT
CAAGATGCACTTTTCGCATATACAAAACGTAATGTCATCCCTACTATAAC
TCAAATGAATCTTAAGTATGCCATTAGTGCAAAGAATAGAGCTCGCACCG
TAGCTGGTGTCTCTATCTGTAGTACTATGACCAATAGACAGTTTCATCAA
AAATTATTGAAATCAATAGCCGCCACTAGAGGAGCTACTGTAGTAATTGG
AACAAGCAAATTCTATGGTGGTTGGCACAACATGTTAAAAACTGTTTATA
GTGATGTAGAAAACCCTCACCTTATGGGTTGGGATTATCCTAAATGTGAT
AGAGCCATGCCTAACATGCTTAGAATTATGGCCTCACTTGTTCTTGCTCG
CAAACATACAACGTGTTGTAGCTTGTCACACCGTTTCTATAGATTAGCTA
ATGAGTGTGCTCAAGTATTGAGTGAAATGGTCATGTGTGGCGGTTCACTA
TATGTTAAACCAGGTGGAACCTCATCAGGAGATGCCACAACTGCTTATGC
TAATAGTGTTTTTAACATTTGTCAAGCTGTCACGGCCAATGTTAATGCAC
TTTTATCTACTGATGGTAACAAAATTGCCGATAAGTATGTCCGCAATTTA
CAACACAGACTTTATGAGTGTCTCTATAGAAATAGAGATGTTGACACAGA
CTTTGTGAATGAGTTTTACGCATATTTGCGTAAACATTTCTCAATGATGA
TACTCTCTGACGATGCTGTTGTGTGTTTCAATAGCACTTATGCATCTCAA
GGTCTAGTGGCTAGCATAAAGAACTTTAAGTCAGTTCTTTATTATCAAAA
CAATGTTTTTATGTCTGAAGCAAAATGTTGGACTGAGACTGACCTTACTA
AAGGACCTCATGAATTTTGCTCTCAACATACAATGCTAGTTAAACAGGGT
GATGATTATGTGTACCTTCCTTACCCAGATCCATCAAGAATCCTAGGGGC
CGGCTGTTTTGTAGATGATATCGTAAAAACAGATGGTACACTTATGATTG
AACGGTTCGTGTCTTTAGCTATAGATGCTTACCCACTTACTAAACATCCT
AATCAGGAGTATGCTGATGTCTTTCATTTGTACTTACAATACATAAGAAA
GCTACATGATGAGTTAACAGGACACATGTTAGACATGTATTCTGTTATGC
TTACTAATGATAACACTTCAAGGTATTGGGAACCTGAGTTTTATGAGGCT
ATGTACACACCGCATACAGTCTTACAG

SEQ ID NO:4: amino acid sequence of Cold_spot_name: RNA-dependent_RNA_polymerase_sp|P0DTD1|R1AB_SARS2:6-932: Translated_from_CDS_NT

FLNGFAV_VQPVLHRAAQALVLMSYTGLLTSTMIK_LVLLNS_KLIVVAS
KKRTKMTI_LILTL_LRDTLSLTTNMKKQFIIYLRIVQLLLNMTSLSLE_
TVTWYHIYHVNVLLNTQWQTSSML_GILMKVIVTH_KKYLSHTIVVMMII
SIKRTGMIL_KTQIYYAYTPT_VNVYAKLC_KQYNSVMPCEMLVLLVY_H
_IIKISMVTGMISVISYKPRQVVEFLL_ILIIHC_CLY_P_PGL_LQSHM
LTLT_QSLTLSGIC_NMTSRKRG_NSLTVILNIGIRHTTQIVLTVWMTDA
FCIVQTLMFYSLQCSHLQVLDH_EKYLLMVFHL_FQLDTTSES_VLYIIR
M_TYIALDLVLRNYLCMLLTLLCTLLLVIYY_INALRAFQ_LHLLTMLLF
KLSNPVILTKTSMTLLCLRVSLRKEVLLNNTSSLLRMVMLLSAIMTTIVI
IYQQCVISDNYYL_LKLLISTLIVTMVAVLMLTKSSSTT_TNQLVFHLIN
GVRLDFIMIQ_VMRIKMHFSHIQNVMSSLL_LK_ILSMPLVQRIELAP_L
VSLSVVL_PIDSFIKNY_NQ_PPLEELL_LEQANSMVVGTTC_KLFIVM_
KTLTLWVGIILNVIEPCLTCLELWPHLFLLANIQRVVACHTVSID_LMSV
LKY_VKWSCVAVHYMLNQVEPHQEMPQLLMLIVFLTFVKLSRPMLMHFYL
LMVTKLPISMSAIYNTDFMSVSIEIEMLTQTL_MSFTHICVNISQ_YSLT
MLLCVSIALMHLKV_WLA_RTLSQFFIIKTMFLCLKQNVGLRLTLLKDLM
NFALNIQC_LNRVMIMCTFLTQIHQES_GPAVL_MIS_KQMVHL_LNGSC
L_L_MLTHLLNILIRSMLMSFICTYNT_ESYMMS_QDTC_TCILLCLLMI
TLQGIGNLSFMRLCTHRIQSY

SEQ ID NO:5: amino acid sequence of Cold_spot_name: RNA-dependent_RNA_polymerase_sp|P0DTD1|R1AB_SARS2:6-932: Extracted_from_protein

FLNRVCGVSAARLTPCGTGTSTDVVYRAFDIYNDKVAGFAKFLKTNCCRF
QEKDEDDNLIDSYFVVKRHTFSNYQHEETIYNLLKDCPAVAKHDFFKFRI
DGDMVPHISRQRLTKYTMADLVYALRHFDEGNCDTLKEILVTYNCCDDDY
FNKKDWYDFVENPDILRVYANLGERVRQALLKTVQFCDAMRNAGIVGVLT
LDNQDLNGNWYDFGDFIQTTPGSGVPVVDSYYSLLMPILTLTRALTAESH
VDTDLTKPYIKWDLLKYDFTEERLKLFDRYFKYWDQTYHPNCVNCLDDRC
ILHCANFNVLFSTVFPPTSFGPLVRKIFVDGVPFVVSTGYHFRELGVVHN
QDVNLHSSRLSFKELLVYAADPAMHAASGNLLLDKRTTCFSVAALTNNVA
FQTVKPGNFNKDFYDFAVSKGFFKEGSSVELKHFFFAQDGNAAISDYDYY
RYNLPTMCDIRQLLFVVEVVDKYFDCYDGGCINANQVIVNNLDKSAGFPF
NKWGKARLYYDSMSYEDQDALFAYTKRNVIPTITQMNLKYAISAKNRART
VAGVSICSTMTNRQFHQKLLKSIAATRGATVVIGTSKFYGGWHNMLKTVY
SDVENPHLMGWDYPKCDRAMPNMLRIMASLVLARKHTTCCSLSHRFYRLA
NECAQVLSEMVMCGGSLYVKPGGTSSGDATTAYANSVFNICQAVTANVNA
LLSTDGNKIADKYVRNLQHRLYECLYRNRDVDTDFVNEFYAYLRKHFSMM
ILSDDAVVCFNSTYASQGLVASIKNFKSVLYYQNNVFMSEAKCWTETDLT
KGPHEFCSQHTMLVKQGDDYVYLPYPDPSRILGAGCFVDDIVKTDGTLMI
ERFVSLAIDAYPLTKHPNQEYADVFHLYLQYIRKLHDELTGHMLDMYSVM
LTNDNTSRYWEPEFYEAMYTPHTVLQ

SEQ ID NOs:6-110 nucleotide sequences of cold spot regions identified in SARS-Cov-2 genomic sequences. (FIGS. 3A-G)

SEQ ID NOs:111-215 amino acid sequences translated from cold spot regions identified in SARS-Cov-2 genomic sequences (Translated_from_CDS_NT). (FIGS. 3A-G)

SEQ ID NOs:216-320 amino acid sequences from cold spot regions identified in SARS-Cov-2 genomic sequences (Extracted_from_protein). (FIGS. 3A-G)

SEQ ID NO:321 nucleotide sequence of a SARS-COV-2 (2019-nCOV) Spike ORF mammalian expression plasmid

Italics Restriction Enzyme Sites
Bold: Target Spike Gene
GACGGATCGGGAGATCTCCCGATCCCCTATGGTGCACTCTCAGTACAATCTGCTCTGATGCCGC
ATAGTTAAGCCAGTATCTGCTCCCTGCTTGTGTGTTGGAGGTCGCTGAGTAGTGCGCGAGCAAA
ATTTAAGCTACAACAAGGCAAGGCTTGACCGACAATTGCATGAAGAATCTGCTTAGGGTTAGGC
GTTTTGCGCTGCTTCGCGAGTACATTTATATTGGCTCATGTCCAATATGACCGCCATGTTGACA
TTGATTATTGACTAGTTATTAATAGTAATCAATTACGGGGTCATTAGTTCATAGCCCATATATG
GAGTTCCGCGTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCATTGACGTCA
ATGGGTGGAGTATTTACGGTAAACTGCCCACTTGGCAGTACATCAAGTGTATCATATGCCAAGT
CCGCCCCCTATTGACGTCAATGACGGTAAATGGCCCGCCTGGCATTATGCCCAGTACATGACCT
TACGGGACTTTCCTACTTGGCAGTACATCTACGTATTAGTCATCGCTATTACCATGGTGATGCG
GTTTTGGCAGTACACCAATGGGCGTGGATAGCGGTTTGACTCACGGGGATTTCCAAGTCTCCAC
CCCATTGACGTCAATGGGAGTTTGTTTTGGCACCAAAATCAACGGGACTTTCCAAAATGTCGTA
ATAACCCCGCCCCGTTGACGCAAATGGGCGGTAGGCGTGTACGGTGGGAGGTCTATATAAGCAG
AGCTCGTTTAGTGAACCGTCAGATCCTCACTCTCTTCCGCATCGCTGTCTGCGAGGGCCAGCTG
TTGGGCTCGCGGTTGAGGACAAACTCTTCGCGGTCTTTCCAGTACTCTTGGATCGGAAACCCGT
CGGCCTCCGAACGGTACTCCGCCACCGAGGGACCTGAGCGAGTCCGCATCGACCGGATCGGAAA
ACCTCTCGAGAAAGGCGTCTAACCAGTCACAGTCGCAAGGTAGGCTGAGCACCGTGGCGGGCGG
CAGCGGGTGGCGGTCGGGGTTGTTTCTGGCGGAGGTGCTGCTGATGATGTAATTAAAGTAGGCG
GTCTTGAGACGGCGGATGGTCGAGGTGAGGTGTGGCAGGCTTGAGATCCAGCTGTTGGGGTGAG
TACTCCCTCTCAAAAGCGGGCATTACTTCTGCGCTAAGATTGTCAGTTTCCAAAAACGAGGAGG
ATTTGATATTCACCTGGCCCGATCTGGCCATACACTTGAGTGACAATGACATCCACTTTGCCTT
TCTCTCCACAGGTGTCCACTCCCAGGTCCAAGTTTAAACTTTAATACGACTCACTATAGGGGCC
GCCACCAAGCTTGGTACCATGTTTGTGTTCCTGGTGCTGCTGCCACTGGTGTCCAGCCAGTGTG
TGAACCTGACCACCAGGACCCAACTTCCTCCTGCCTACACCAACTCCTTCACCAGGGGAGTCTA
CTACCCTGACAAGGTGTTCAGGTCCTCTGTGCTGCACAGCACCCAGGACCTGTTCCTGCCATTC
TTCAGCAATGTGACCTGGTTCCATGCCATCCATGTGTCTGGCACCAATGGCACCAAGAGGTTTG
ACAACCCTGTGCTGCCATTCAATGATGGAGTCTACTTTGCCAGCACAGAGAAGAGCAACATCAT
CAGGGGCTGGATTTTTGGCACCACCCTGGACAGCAAGACCCAGTCCCTGCTGATTGTGAACAAT
GCCACCAATGTGGTGATTAAGGTGTGTGAGTTCCAGTTCTGTAATGACCCATTCCTGGGAGTCT
ACTACCACAAGAACAACAAGTCCTGGATGGAGTCTGAGTTCAGGGTCTACTCCTCTGCCAACAA
CTGTACCTTTGAATATGTGAGCCAACCATTCCTGATGGACTTGGAGGGCAAGCAGGGCAACTTC
AAGAACCTGAGGGAGTTTGTGTTCAAGAACATTGATGGCTACTTCAAGATTTACAGCAAACACA
CACCAATCAACCTGGTGAGGGACCTGCCACAGGGCTTCTCTGCCTTGGAACCACTGGTGGACCT
GCCAATTGGCATCAACATCACCAGGTTCCAGACCCTGCTGGCTCTGCACAGGTCCTACCTGACA
CCTGGAGACTCCTCCTCTGGCTGGACAGCAGGAGCAGCAGCCTACTATGTGGGCTACCTCCAAC
CAAGGACCTTCCTGCTGAAATACAATGAGAATGGCACCATCACAGATGCTGTGGACTGTGCCCT
GGACCCACTGTCTGAGACCAAGTGTACCCTGAAATCCTTCACAGTGGAGAAGGGCATCTACCAG
ACCAGCAACTTCAGGGTCCAACCAACAGAGAGCATTGTGAGGTTTCCAAACATCACCAACCTGT
GTCCATTTGGAGAGGTGTTCAATGCCACCAGGTTTGCCTCTGTCTATGCCTGGAACAGGAAGAG
GATTAGCAACTGTGTGGCTGACTACTCTGTGCTCTACAACTCTGCCTCCTTCAGCACCTTCAAG
TGTTATGGAGTGAGCCCAACCAAACTGAATGACCTGTGTTTCACCAATGTCTATGCTGACTCCT
TTGTGATTAGGGGAGATGAGGTGAGACAGATTGCCCCTGGACAAACAGGCAAGATTGCTGACTA
CAACTACAAACTGCCTGATGACTTCACAGGCTGTGTGATTGCCTGGAACAGCAACAACCTGGAC
AGCAAGGTGGGAGGCAACTACAACTACCTCTACAGACTGTTCAGGAAGAGCAACCTGAAACCAT
TTGAGAGGGACATCAGCACAGAGATTTACCAGGCTGGCAGCACACCATGTAATGGAGTGGAGGG
CTTCAACTGTTACTTTCCACTCCAATCCTATGGCTTCCAACCAACCAATGGAGTGGGCTACCAA
CCATACAGGGTGGTGGTGCTGTCCTTTGAACTGCTCCATGCCCCTGCCACAGTGTGTGGACCAA
AGAAGAGCACCAACCTGGTGAAGAACAAGTGTGTGAACTTCAACTTCAATGGACTGACAGGCAC
AGGAGTGCTGACAGAGAGCAACAAGAAGTTCCTGCCATTCCAACAGTTTGGCAGGGACATTGCT
GACACCACAGATGCTGTGAGGGACCCACAGACCTTGGAGATTCTGGACATCACACCATGTTCCT
TTGGAGGAGTGTCTGTGATTACACCTGGCACCAACACCAGCAACCAGGTGGCTGTGCTCTACCA
GGATGTGAACTGTACTGAGGTGCCTGTGGCTATCCATGCTGACCAACTTACACCAACCTGGAGG
GTCTACAGCACAGGCAGCAATGTGTTCCAGACCAGGGCTGGCTGTCTGATTGGAGCAGAGCATG
TGAACAACTCCTATGAGTGTGACATCCCAATTGGAGCAGGCATCTGTGCCTCCTACCAGACCCA
GACCAACAGCCCAAGGAGGGCAAGGTCTGTGGCAAGCCAGAGCATCATTGCCTACACAATGAGT
CTGGGAGCAGAGAACTCTGTGGCTTACAGCAACAACAGCATTGCCATCCCAACCAACTTCACCA
TCTCTGTGACCACAGAGATTCTGCCTGTGAGTATGACCAAGACCTCTGTGGACTGTACAATGTA
TATCTGTGGAGACAGCACAGAGTGTAGCAACCTGCTGCTCCAATATGGCTCCTTCTGTACCCAA
CTTAACAGGGCTCTGACAGGCATTGCTGTGGAACAGGACAAGAACACCCAGGAGGTGTTTGCCC
AGGTGAAGCAGATTTACAAGACACCTCCAATCAAGGACTTTGGAGGCTTCAACTTCAGCCAGAT
TCTGCCTGACCCAAGCAAGCCAAGCAAGAGGTCCTTCATTGAGGACCTGCTGTTCAACAAGGTG
ACCCTGGCTGATGCTGGCTTCATCAAGCAATATGGAGACTGTCTGGGAGACATTGCTGCCAGGG
ACCTGATTTGTGCCCAGAAGTTCAATGGACTGACAGTGCTGCCTCCACTGCTGACAGATGAGAT
GATTGCCCAATACACCTCTGCCCTGCTGGCTGGCACCATCACCTCTGGCTGGACCTTTGGAGCA
GGAGCAGCCCTCCAAATCCCATTTGCTATGCAGATGGCTTACAGGTTCAATGGCATTGGAGTGA
CCCAGAATGTGCTCTATGAGAACCAGAAACTGATTGCCAACCAGTTCAACTCTGCCATTGGCAA
GATTCAGGACTCCCTGTCCAGCACAGCCTCTGCCCTGGGCAAACTCCAAGATGTGGTGAACCAG
AATGCCCAGGCTCTGAACACCCTGGTGAAGCAACTTTCCAGCAACTTTGGAGCCATCTCCTCTG
TGCTGAATGACATCCTGAGCAGACTGGACAAGGTGGAGGCTGAGGTCCAGATTGACAGACTGAT
TACAGGCAGACTCCAATCCCTCCAAACCTATGTGACCCAACAACTTATCAGGGCTGCTGAGATT
AGGGCATCTGCCAACCTGGCTGCCACCAAGATGAGTGAGTGTGTGCTGGGACAAAGCAAGAGGG
TGGACTTCTGTGGCAAGGGCTACCACCTGATGAGTTTTCCACAGTCTGCCCCTCATGGAGTGGT
GTTCCTGCATGTGACCTATGTGCCTGCCCAGGAGAAGAACTTCACCACAGCCCCTGCCATCTGC
CATGATGGCAAGGCTCACTTTCCAAGGGAGGGAGTGTTTGTGAGCAATGGCACCCACTGGTTTG
TGACCCAGAGGAACTTCTATGAACCACAGATTATCACCACAGACAACACCTTTGTGTCTGGCAA
CTGTGATGTGGTGATTGGCATTGTGAACAACACAGTCTATGACCCACTCCAACCTGAACTGGAC
TCCTTCAAGGAGGAACTGGACAAATACTTCAAGAACCACACCAGCCCTGATGTGGACCTGGGAG
ACATCTCTGGCATCAATGCCTCTGTGGTGAACATCCAGAAGGAGATTGACAGACTGAATGAGGT
GGCTAAGAACCTGAATGAGTCCCTGATTGACCTCCAAGAACTGGGCAAATATGAACAATACATC
AAGTGGCCATGGTACATCTGGCTGGGCTTCATTGCTGGACTGATTGCCATTGTGATGGTGACCA
TAATGCTGTGTTGTATGACCTCCTGTTGTTCCTGTCTGAAAGGCTGTTGTTCCTGTGGCTCCTG
TTGTAAGTTTGATGAGGATGACTCTGAACCTGTGCTGAAAGGAGTGAAACTGCACTACACCTGA
ATCTAGAGCGGCCGCCGAATTCGGGCCCGTTTAAACCCGCTGATCAGCCTCGACTGTGCCTTCT
AGTTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAATAGCAGGCATGCT
GGGGATGCGGTGGGCTCTATGGCTTCTGAGGCGGAAAGAACCAGCTGGGGCTCTAGGGGGTATC
CCCACGCGCCCTGTAGCGGCGCATTAAGCGCGGCGGGTGTGGTGGTTACGCGCAGCGTGACCGC
TACACTTGCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGGCCATCGCCCTGATA
GACGGTTTTTCGCCCTTTGACGTTGGAGTCCACGTTCTTTAATAGTGGACTCTTGTTCCAAACT
GGAACAACACTCAACCCTATCTCGGTCTATTCTTTTGATTTATAAGGGATTTTGCCGATTTCGG
CCTATTGGTTAAAAAATGAGCTGATTTAACAAAAATTTAACGCGAATTAATTCTGTGGAATGTG
TGTCAGTTAGGGTGTGGAAAGTCCCCAGGCTCCCCAGCAGGCAGAAGTATGCAAAGCATGCATC
TCAATTAGTCAGCAACCAGGTGTGGAAAGTCCCCAGGCTCCCCAGCAGGCAGAAGTATGCAAAG
CATGCATCTCAATTAGTCAGCAACCATAGTCCCGCCCCTAACTCCGCCCATCCCGCCCCTAACT
CCGCCCAGTTCCGCCCATTCTCCGCCCCATGGCTGACTAATTTTTTTTATTTATGCAGAGGCCG
AGGCCGCCTCTGCCTCTGAGCTATTCCAGAAGTAGTGAGGAGGCTTTTTTGGAGGCCTAGGCTT
TTGCAAAAAGCTCCCGGGAGCTTGTATATCCATTTTCGGATCTGATCAGCACGTGATGAAAAAG
CCTGAACTCACCGCGACGTCTGTCGAGAAGTTTCTGATCGAAAAGTTCGACAGCGTCTCCGACC
TGATGCAGCTCTCGGAGGGCGAAGAATCTCGTGCTTTCAGCTTCGATGTAGGAGGGCGTGGATA
TGTCCTGCGGGTAAATAGCTGCGCCGATGGTTTCTACAAAGATCGTTATGTTTATCGGCACTTT
GCATCGGCCGCGCTCCCGATTCCGGAAGTGCTTGACATTGGGGAATTCAGCGAGAGCCTGACCT
ATTGCATCTCCCGCCGTGCACAGGGTGTCACGTTGCAAGACCTGCCTGAAACCGAACTGCCCGC
TGTTCTGCAGCCGGTCGCGGAGGCCATGGATGCGATCGCTGCGGCCGATCTTAGCCAGACGAGC
GGGTTCGGCCCATTCGGACCGCAAGGAATCGGTCAATACACTACATGGCGTGATTTCATATGCG
CGATTGCTGATCCCCATGTGTATCACTGGCAAACTGTGATGGACGACACCGICAGTGCGTCCGT
CGCGCAGGCTCTCGATGAGCTGATGCTTTGGGCCGAGGACTGCCCCGAAGTCCGGCACCTCGTG
CACGCGGATTTCGGCTCCAACAATGTCCTGACGGACAATGGCCGCATAACAGCGGTCATTGACT
GGAGCGAGGCGATGTTCGGGGATTCCCAATACGAGGTCGCCAACATCTTCTTCTGGAGGCCGTG
GTTGGCTTGTATGGAGCAGCAGACGCGCTACTTCGAGCGGAGGCATCCGGAGCTTGCAGGATCG
CCGCGGCTCCGGGCGTATATGCTCCGCATTGGTCTTGACCAACTCTATCAGAGCTTGGTTGACG
GCAATTTCGATGATGCAGCTTGGGCGCAGGGTCGATGCGACGCAATCGTCCGATCCGGAGCCGG
GACTGTCGGGCGTACACAAATCGCCCGCAGAAGCGCGGCCGTCTGGACCGATGGCTGTGTAGAA
GTACTCGCCGATAGTGGAAACCGACGCCCCAGCACTCGTCCGAGGGCAAAGGAATAGCACGTGC
TACGAGATTTCGATTCCACCGCCGCCTTCTATGAAAGGTTGGGCTTCGGAATCGTTTTCCGGGA
CGCCGGCTGGATGATCCTCCAGCGCGGGGATCTCATGCTGGAGTTCTTCGCCCACCCCAACTTG
TTTATTGCAGCTTATAATGGTTACAAATAAAGCAATAGCATCACAAATTTCACAAATAAAGCAT
TTTTTTCACTGCATTCTAGTTGTGGTTTGTCCAAACTCATCAATGTATCTTATCATGTCTGTAT
ACCGTCGACCTCTAGCTAGAGCTTGGCGTAATCATGGTCATAGCTGTTTCCTGTGTGAAATTGT
TATCCGCTCACAATTCCACACAACATACGAGCCGGAAGCATAAAGTGTAAAGCCTGGGGTGCCT
AATGAGTGAGCTAACTCACATTAATTGCGTTGCGCTCACTGCCCGCTTTCCAGTCGGGAAACCT
GTCGTGCCAGCTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAGGAAAGAACATGTG
AGCAAAAGGCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAACCCGACAG
GACTATAAAGATACCAGGCGTTTCCCCCTGGAAGCTCCCTCGTGCGCTCTCCTGTTCCGACCCT
GCCGCTTACCGGATACCTGTCCGCCTTTCTCCCTTCGGGAAGCGTGGCGCTTTCTCATAGCTCA
CGCTGTAGGTATCTCAGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGTCCAACCCGGTAAGACA
CGACTTATCGCCACTGGCAGCAGCCACTGGTAACAGGATTAGCAGAGCGAGGTATGTAGGCGGT
GCTACAGAGTTCTTGAAGTGGTGGCCTAACTACGGCTACACTAGAAGAACAGTATTTGGTATCT
GCGCTCTGCTGAAGCCAGTTACCTTCGGAAAAAGAGTTGGTAGCTCTTGATCCGGCAAACAAAC
CACCGCTGGTAGCGGTTTTTTTGTTTGCAAGCAGCAGATTACGCGCAGAAAAAAAGGATCTCAA
GAAGATCCTTTGATCTTTTCTACGGGGTCTGACGCTCAGTGGAACGAAAACTCACGTTAAGGGA
TTTTGGTCATGAGATTATCAAAAAGGATCTTCACCTAGATCCTTTTAAATTAAAAATGAAGTTT
TAAATCAATCTAAAGTATATATGAGTAAACTTGGTCTGACAGTTACCAATGCTTAATCAGTGAG
GCACCTATCTCAGCGATCTGTCTATTTCGTTCATCCATAGTTGCCTGACTCCCCGTCGTGTAGA
TAACTACGATACGGGAGGGCTTACCATCTGGCCCCAGTGCTGCAATGATACCGCGAGACCCACG
CTCACCGGCTCCAGATTTATCAGCAATAAACCAGCCAGCCGGAAGGGCCGAGCGCAGAAGTGGT
CCTGCAACTTTATCCGCCTCCATCCAGTCTATTAATTGTTGCCGGGAAGCTAGAGTAAGTAGTT
CGCCAGTTAATAGTTTGCGCAACGTTGTTGCCATTGCTACAGGCATCGTGGTGTCACGCTCGTC
GTTTGGTATGGCTTCATTCAGCTCCGGTTCCCAACGATCAAGGCGAGTTACATGATCCCCCATG
TTGTGCAAAAAAGCGGTTAGCTCCTTCGGTCCTCCGATCGTTGTCAGAAGTAAGTTGGCCGCAG
TGTTATCACTCATGGTTATGGCAGCACTGCATAATTCTCTTACTGTCATGCCATCCGTAAGATG
CTTTTCTGTGACTGGTGAGTACTCAACCAAGTCATTCTGAGAATAGTGTATGCGGCGACCGAGT
TGCTCTTGCCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGATCCAGTTC
GATGTAACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGAAATGTTGAA
TACTCATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCCCGAAAA
GTGCCACCTGACGTC

SEQ ID NO:322 amino acid sequence of Spike glycoprotein (Swiss-Prot ID: P0DTC2) of SARS-COV-2 (Severe Acute Respiratory Syndrome-related coronavirus 2).

S1: Represented in regular font
S2: Represented in bold font
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNERVQPTESIVRFPNITNLCPFGEVENATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGENCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLENKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

SEQ ID NO:323 amino acid sequence of Actin, alpha skeletal muscle (Swiss-Prot ID: P68133) of Homo sapiens (Human)

MCDEDETTALVCDNGSGLVKAGFAGDDAPRAVFPSIVGRPRHQGVMVGMG
QKDSYVGDEAQSKRGILTLKYPIEHGIITNWDDMEKIWHHTFYNELRVAP
EEHPTLLTEAPLNPKANREKMTQIMFETFNVPAMYVAIQAVLSLYASGRT
TGIVLDSGDGVTHNVPIYEGYALPHAIMRLDLAGRDLTDYLMKILTERGY
SFVTTAEREIVRDIKEKLCYVALDFENEMATAASSSSLEKSYELPDGQVI
TIGNERFRCPETLFQPSFIGMESAGIHETTYNSIMKCDIRKDLYANNVMS
GGTTMYPGIADRMQKEITALAPSTMKIKIIAPPERKYSVWIGGSILASLS
TFQQMWITKQEYDEAGPSIVHRKCF

SEQ ID NOs:324-484 Untreated Healthy Donors' HCDR3 amino acid sequences (Table 1)

SEQ ID NOs:485-555 Stimulated Healthy Donors' HCDR3 amino acid sequences (Table 2)

SEQ ID NOs:556-694 Untreated Recovered Patients' HCDR3 amino acid sequences (Table 3)

SEQ ID NOs:695-929 Stimulated Recovered Patients' HCDR3 amino acid sequences (Table 4)

SEQ ID NOs:930-934 amino acid sequences of HCDR3 for TCR rearrangement (Table 5)

SEQ ID NOs:935-940 nucleotide sequences of TCR rearrangement (Table 5)

SEQ ID NOs:941-942 amino acid sequences of hypomutated epitopes from spike protein. (Table 5)

SEQ ID NOs:943-970 nucleotide sequences of additional cold spot regions identified in SARS-Cov-2 genomic sequences. (FIGS. 4A-B)

SEQ ID NOs:971-998 amino acid sequences translated from additional cold spot regions identified in SARS-Cov-2 genomic sequences (Translated_from_CDS_NT). (FIGS. 4A-B)

SEQ ID NOs:999-1026 amino acid sequences from additional cold spot regions identified in SARS-Cov-2 genomic sequences (Extracted_from_protein). (FIGS. 4A-B)

What is claimed is:

1. A method for making a vaccine against a virus, the method comprising:
(a) aligning genome sequences of at least two isolates, optionally, 1000, 10,000 or 100,000 isolates, of the virus;
(b) identifying genomic areas with no or very little viral sequence variation at a nucleotide level;
(c) identifying amino acid sequence areas with little or no sequence variation at an amino acid sequence level and aligning the identified amino acid sequence areas with the identified genomic areas;
(d) making a vaccine against the identified amino acid sequence areas with little or no sequence variation.

2. The method of claim 1, wherein the identified genomic and amino acid areas each constitute less than 10%, 5%, or 1% of the total genomic and amino acid areas.

3. The method of claim 2, wherein the identified genomic and amino acid areas each have less than 10%, 5% or 1% of the mean variation in genomic and amino acid areas of the same length throughout the genome.

4. The method of claim 1, wherein the virus is SARS-Cov-2.

5. The method of claim 4, wherein the identified amino acid sequence area is any one of SEQ ID NOs: 1, 4-5, 111-320, 322, 941-942, and 971-1026.

6. The method of claim 5, wherein the identified amino acid sequence area is SARS-Cov-2 RNA-dependent RNA polymerase (SEQ ID NO:1).

7. The method of claim 5, wherein the identified amino acid sequence area is SARS-Cov-2 spike glycoprotein (SEQ ID NO:322).

8. A polypeptide vaccine comprising a SARS-Cov-2 polypeptide of any one of SEQ ID NOs: 1, 4-5, 111-320, 322, 941-942, and 971-1026 or an immunogenic fragment thereof and a pharmaceutically acceptable excipient.

9. The polypeptide vaccine of claim 8, wherein the SARS-Cov-2 polypeptide is a SARS-Cov-2 RNA-dependent RNA polymerase (SEQ ID NO:1) or an immunogenic fragment thereof.

10. The polypeptide vaccine of claim 8, comprising a SARS-Cov-2 polypeptide of any one of SEQ ID NOs: 1, 988-998, and 1016-1026 or an immunogenic fragment thereof and a pharmaceutically acceptable excipient.

11. The polypeptide vaccine of claim 8, wherein the SARS-Cov-2 polypeptide is a SARS-Cov-2 spike glycoprotein (SEQ ID NO:322) or an immunogenic fragment thereof.

12. The polypeptide vaccine of claim 8, comprising a SARS-Cov-2 polypeptide of any one of SEQ ID NOs:322, 941-942, 971-987, and 999-1015 or an immunogenic fragment thereof and a pharmaceutically acceptable excipient.

13. The polypeptide vaccine of any one of claims 8-12, wherein the SARS-Cov-2 polypeptide or fragment is linked to a heterologous carrier to help elicit and immune response.

14. The polypeptide vaccine of any one of claims 8-13, wherein the pharmaceutically acceptable excipient is an adjuvant that helps elicit and immune response.

15. The polypeptide vaccine of any one of claims 8-14, wherein the fragment has up to 10, 25, 50 or 100 contiguous amino acids of any one of SEQ ID NOs:1, 4-5, 111-320, 322, 941-942, and 971-1026.

16. The polypeptide vaccine of claim 15, wherein the fragment has up to 10, 25, 50 or 100 contiguous amino acids of SEQ ID NO:1.

17. The polypeptide vaccine of claim 15, wherein the fragment has up to 10, 25, 50 or 100 contiguous amino acids of SEQ ID NO:322.

18. A polynucleotide vaccine comprising a polynucleotide encoding a SARS-Cov-2 polypeptide of any one of SEQ ID NOs: 1, 4-5, 111-320, 322, 941-942, and 971-1026 or immunogenic fragment thereof linked to a heterologous promoter and a pharmaceutically acceptable excipient.

19. The polynucleotide vaccine of claim 18, wherein the polynucleotide encodes a SARS-Cov-2 RNA-dependent RNA polymerase (SEQ ID NO:1) or immunogenic fragment thereof.

20. The polynucleotide vaccine of claim 18, wherein the polynucleotide encodes a SARS-Cov-2 spike glycoprotein (SEQ ID NO:322) or immunogenic fragment thereof.

21. The polynucleotide vaccine of any one of claims 18-20, which is a DNA vaccine.

22. The polynucleotide vaccine of any one of claims 18-20, which is an RNA vaccine.

23. The polynucleotide vaccine of claim 18, wherein the SARS-Cov-2 polypeptide or fragment is linked to a heterologous carrier to help elicit an immune response.

24. The polynucleotide vaccine of claim 18, wherein the pharmaceutically acceptable excipient is an adjuvant that helps elicit an immune response.

25. The polynucleotide vaccine of claim 18, wherein the fragment has up to 10, 25, 50 or 100 contiguous amino acids of any one of SEQ ID NOs: 1, 4-5, 111-320, 322, 941-942, and 971-1026.

26. The polynucleotide vaccine of claim 25, wherein the fragment has up to 10, 25, 50 or 100 contiguous amino acids of SEQ ID NO:1.

27. The polynucleotide vaccine of claim 25, wherein the fragment has up to 10, 25, 50 or 100 contiguous amino acids of SEQ ID NO:322.

28. The polynucleotide vaccine of claim 18, wherein the polynucleotide and heterologous promoter are components of a viral vector.

29. The polynucleotide vaccine of claim 18, wherein the polynucleotide and heterologous promoter are components of a bacterial vector or genome.

30. A method for effecting prophylaxis of or treating SARS-Cov-2 infection comprising a step of administering the polypeptide vaccine of any one of claims 8-17 and/or the polynucleotide vaccine of any one of claims 18-29 to a subject in need thereof.
